SEQUENCE LISTING (84 sequences)

SEQ ID NO: 1 - DNA, 495 bp, Homo sapiens
  Human IL-22
atgggatggtcatgtatcatcctttttctagtagcaactgcaactggagtacattcagcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatt

SEQ ID NO: 2 - protein, 165 aa, Homo sapiens
  Human IL-22
MGWSCIILFLVATATGVHSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 3 - DNA, 438 bp, Homo sapiens
  IL-22 DNA (mature)
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatt

SEQ ID NO: 4 - protein, 146 aa, Homo sapiens
  IL-22 (mature)
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 5 - DNA, 57 bp, Homo sapiens
  IL-22 leader sequence
atgggatggtcatgtatcatcctttttctagtagcaactgcaactggagtacattca

SEQ ID NO: 6 - protein, 19 aa, Homo sapiens
  IL-22 leader sequence
MGWSCIILFLVATATGVHS

SEQ ID NO: 7 - DNA, 1128 bp, Artificial Sequence
  Description of Artificial Sequence Synthetic polynucleotide
  IL-22 Fc fusion IgG4 (minus C-terminal Lys) N297G
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcggaagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 8 - protein, 376 aa, Artificial Sequence
  Description of Artificial Sequence Synthetic polypeptide
  IL-22 Fc fusion IgG4 (minus C-terminal Lys) N297G
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - DNA, 1128 bp, Artificial Sequence
  Description of Artificial Sequence Synthetic polynucleotide
  IL-22 Fc fusion IgG4 (minus C-terminal Lys) N297A
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcgctagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 10 - protein, 376 aa, Artificial Sequence
  Description of Artificial Sequence Synthetic polypeptide
  IL-22 Fc fusion IgG4 (minus C-terminal Lys) N297A
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - DNA, 1131 bp, Artificial Sequence
  Description of Artificial Sequence Synthetic polynucleotide
  IL-22 Fc fusion IgG1 (minus C-terminal Lys) N297G
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 12 - protein, 377 aa, Artificial Sequence
  Description of Artificial Sequence Synthetic polypeptide
  IL-22 Fc fusion IgG1 (minus C-terminal Lys) N297G
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - DNA, 1131 bp, Artificial Sequence
  Description of Artificial Sequence Synthetic polynucleotide
  IL-22 Fc fusion IgG1 (minus C-terminal Lys) N297A
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 14 - protein, 377 aa, Artificial Sequence
  Description of Artificial Sequence Synthetic polypeptide
  IL-22 Fc fusion IgG1 (minus C-terminal Lys) N297A
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - DNA, 1131 bp, Artificial Sequence
  Description of Artificial Sequence Synthetic polynucleotide
  IL-22 Fc fusion IgG4 (full) N297G
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcggaagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 16 - protein, 377 aa, Artificial Sequence
  Description of Artificial Sequence Synthetic polypeptide
  IL-22 Fc fusion IgG4 (full) N297G
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - DNA, 1131 bp, Artificial Sequence
  Description of Artificial Sequence Synthetic polynucleotide
  IL-22 Fc fusion IgG4 (full) N297A
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcgctagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 18 - protein, 377 aa, Artificial Sequence
  Description of Artificial Sequence Synthetic polypeptide
  IL-22 Fc fusion IgG4 (full) N297A
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 1134 bp, Artificial Sequence
  Description of Artificial Sequence Synthetic polynucleotide
  IL-22 Fc fusion IgG1 (full) N297G
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacggaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - protein, 378 aa, Artificial Sequence
  Description of Artificial Sequence Synthetic polypeptide
  IL-22 Fc fusion IgG1 (full) N297G
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1134 bp, Artificial Sequence
  Description of Artificial Sequence Synthetic polynucleotide
  IL-22 Fc fusion IgG1 (full) N297A
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgctagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 22 - protein, 378 aa, Artificial Sequence
  Description of Artificial Sequence Synthetic polypeptide
  IL-22 Fc fusion IgG1 (full) N297A
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1128 bp, Artificial Sequence
  Description of Artificial Sequence Synthetic polynucleotide
  IL-22 Fc fusion IgG4 (wt N297, minus Lys)
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 24 - protein, 376 aa, Artificial Sequence
  Description of Artificial Sequence Synthetic polypeptide
  IL-22 Fc fusion IgG4 (wt N297, minus Lys)
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 25 - DNA, 1131 bp, Artificial Sequence
  Description of Artificial Sequence Synthetic polynucleotide
  IL-22 Fc fusion IgG1 (wt N297, minus Lys)
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 26 - protein, 377 aa, Artificial Sequence
  Description of Artificial Sequence Synthetic polypeptide
  IL-22 Fc fusion IgG1 (wt N297, minus Lys)
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - DNA, 1131 bp, Artificial Sequence
  Description of Artificial Sequence Synthetic polynucleotide
  IL-22 Fc fusion IgG4 (N297 wt)
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattcgcgttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 28 - protein, 377 aa, Artificial Sequence
  Description of Artificial Sequence Synthetic polypeptide
  IL-22 Fc fusion IgG4 (N297 wt)
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - DNA, 1134 bp, Artificial Sequence
  Description of Artificial Sequence Synthetic polynucleotide
  IL-22 Fc fusion IgG1 (N297 wt)
gcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgagcccaaatctagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - protein, 378 aa, Artificial Sequence
  Description of Artificial Sequence Synthetic polypeptide
  IL-22 Fc fusion IgG1 (N297 wt)
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 5 aa, Artificial Sequence
  Description of Artificial Sequence Synthetic hinge peptide
CPPCP

SEQ ID NO: 32 - protein, 5 aa, Artificial Sequence
  Description of Artificial Sequence Synthetic linker peptide
DKTHT

SEQ ID NO: 33 - protein, 10 aa, Artificial Sequence
  Description of Artificial Sequence Synthetic linker peptide
EPKSCDKTHT

SEQ ID NO: 34 - protein, 11 aa, Artificial Sequence
  Description of Artificial Sequence Synthetic linker peptide
VEPKSCDKTHT

SEQ ID NO: 35 - protein, 12 aa, Artificial Sequence
  Description of Artificial Sequence Synthetic linker peptide
KVEPKSCDKTHT

SEQ ID NO: 36 - protein, 13 aa, Artificial Sequence
  Description of Artificial Sequence Synthetic linker peptide
KKVEPKSCDKTHT

SEQ ID NO: 37 - protein, 14 aa, Artificial Sequence
  Description of Artificial Sequence Synthetic linker peptide
DKKVEPKSCDKTHT

SEQ ID NO: 38 - protein, 15 aa, Artificial Sequence
  Description of Artificial Sequence Synthetic linker peptide
VDKKVEPKSCDKTHT

SEQ ID NO: 39 - protein, 16 aa, Artificial Sequence
  Description of Artificial Sequence Synthetic linker peptide
KVDKKVEPKSCDKTHT

SEQ ID NO: 40 - protein, 10 aa, Artificial Sequence
  Description of Artificial Sequence Synthetic linker peptide
EPKSSDKTHT

SEQ ID NO: 41 - protein, 8 aa, Artificial Sequence
  Description of Artificial Sequence Synthetic linker peptide
GGGDKTHT

SEQ ID NO: 42 - protein, 12 aa, Artificial Sequence
  Description of Artificial Sequence Synthetic linker (IgG3) peptide
ELKTPLGDTTHT

SEQ ID NO: 43 - protein, 6 aa, Artificial Sequence
  Description of Artificial Sequence Synthetic linker peptide
SKYGPP

SEQ ID NO: 44 - protein, 9 aa, Artificial Sequence
  Description of Artificial Sequence Synthetic linker peptide
RVESKYGPP

SEQ ID NO: 45 - protein, 3 aa, Artificial Sequence
  Description of Artificial Sequence Synthetic linker peptide
GGS

SEQ ID NO: 46 - protein, 4 aa, Artificial Sequence
  Description of Artificial Sequence Synthetic linker peptide
GGGS

SEQ ID NO: 47 - protein, 5 aa, Artificial Sequence
  Description of Artificial Sequence Synthetic linker peptide
GGGGS

SEQ ID NO: 48 - protein, 146 aa, Pan troglodytes
APISSHCRLDKSSFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGENGEIKAIGELDLLFMSLRNACI

SEQ ID NO: 49 - protein, 146 aa, Pongo abelii
APISSHCRLDKSNFQQPYITNRTFMLAKEASLADNNTDVRLIGEKLFRGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 50 - protein, 146 aa, Mus musculus
LPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 51 - protein, 146 aa, Canis familiaris
LPISSHCRLDKSNFQQPYITNRTFMLAKEASLADNNTDVRLIGEKLFHGVNMGERCYLMKEVLNFTLEEVLLPQSDRFQPYMQEVVPFLARLSNKLSQCHIENDDQHIQRNVQKLKDTVQKLGENGEIKAIGELDLLFMALRNACV

SEQ ID NO: 52 - DNA, 94 bp, Artificial Sequence
  Description of Artificial Sequence Synthetic IL-22 Fc fusion protein IgG1 forward primer
ttgaattccaccatgggatggtcatgtatcatcctttttctagtagcaactgcaactggagtacattcagcgcccatcagctcccactgcaggc

SEQ ID NO: 53 - DNA, 33 bp, Artificial Sequence
  Description of Artificial Sequence Synthetic IL-22 Fc fusion IgG1 reverse primer
aggtcgactcatttacccggagacagggagagg

SEQ ID NO: 54 - DNA, 94 bp, Artificial Sequence; identical to SEQ ID NO: 52
  Description of Artificial Sequence Synthetic IL-22 Fc fusion IgG4 forward primer

SEQ ID NO: 55 - DNA, 33 bp, Artificial Sequence
  Description of Artificial Sequence Synthetic IL-22 Fc fusion IgG4 reverse primer
aggtcgacttatttacccagagacagggagagg

SEQ ID NO: 56 - DNA, 35 bp, Artificial Sequence
  Description of Artificial Sequence Synthetic IgG1 N297G forward primer
gcgggaggagcagtacggaagcacgtaccgtgtgg

SEQ ID NO: 57 - DNA, 35 bp, Artificial Sequence
  Description of Artificial Sequence Synthetic IgG1 N297G reverse primer
ccacacggtacgtgcttccgtactgctcctcccgc

SEQ ID NO: 58 - DNA, 51 bp, Artificial Sequence
  Description of Artificial Sequence Synthetic IgG4 N297G forward primer
acaaagccgcgggaggagcagttcggaagcacgtaccgtgtggtcagcgtc

SEQ ID NO: 59 - DNA, 51 bp, Artificial Sequence
  Description of Artificial Sequence Synthetic IgG4 N297G reverse primer
gacgctgaccacacggtacgtgcttccgaactgctcctcccgcggctttgt

SEQ ID NO: 60 - protein, 411 aa, Artificial Sequence
  Description of Artificial Sequence Synthetic polypeptide
  IL-22 Fc fusion IgG2a
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACVARGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 61 - protein, 372 aa, Artificial Sequence
  Description of Artificial Sequence Synthetic polypeptide
  IL-22 IgG1 fusion knob (T366W) minus Lys
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 226 aa, Artificial Sequence
  Description of Artificial Sequence Synthetic polypeptide
  Monomeric Fc hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 63 - protein, 7 aa, Artificial Sequence
  Description of Artificial Sequence Synthetic linker peptide
GGGSTHT

SEQ ID NO: 64 - protein, 14 aa, Artificial Sequence
  Description of Artificial Sequence Synthetic linker peptide
DKKVEPKSSDKTHT

SEQ ID NO: 65 - protein, 16 aa, Artificial Sequence
  Description of Artificial Sequence Synthetic linker peptide
KVDKKVEPKSSDKTHT

SEQ ID NO: 66 - protein, 13 aa, Artificial Sequence
  Description of Artificial Sequence Synthetic linker peptide
KKVEPKSSDKTHT

SEQ ID NO: 67 - protein, 11 aa, Artificial Sequence
  Description of Artificial Sequence Synthetic linker peptide
VEPKSSDKTHT

SEQ ID NO: 68 - protein, 12 aa, Artificial Sequence
  Description of Artificial Sequence Synthetic linker peptide
KVEPKSSDKTHT

SEQ ID NO: 69 - protein, 15 aa, Artificial Sequence
  Description of Artificial Sequence Synthetic linker peptide
VDKKVEPKSSDKTHT

SEQ ID NO: 70 - DNA, 1152 bp, Homo sapiens
  CDS (58)..(597)
cttcagaacaggttctccttccccagtcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttttttaaccaaaaggaagatgggaagccaaactccatcatgatgggtggattccaaatgaacccctgcgttagttacaaaggaaaccaatgccacttttgtttataagaccagaaggtagactttctaagcatagatatttattgataacatttcattgtaactggtgttctatacacagaaaacaatttattttttaaataattgtctttttccataaaaaagattactttccattcctttaggggaaaaaacccctaaatagcttcatgtttccataatcagtactttatatttataaatgtatttattattattataagactgcattttatttatatcattttattaatatggatttatttatagaaacatcattcgatattgctacttgagtgtaaggctaatattgatatttatgacaataattatagagctataacatgtttatttgacctcaataaacacttggatatccc

SEQ ID NO: 71 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 72 - DNA, 1236 bp, Artificial Sequence
  Synthetic polypeptide
atggctgtcctgcagaaatctatgagtttttcccttatggggactttggccgccagctgcctgcttctcattgccctgtgggcccaggaggcaaatgcgctgcccgtcaacacccggtgcaagcttgaggtgtccaacttccagcagccatacatcgtcaaccgcacctttatgctggccaaggaggccagccttgcagataacaacacagatgtccggctcatcggggagaaactgttccgaggagtcagtgctaaggatcagtgctacctgatgaagcaggtgctcaacttcaccctggaagacgttctgctcccccagtcagacaggttccagccctacatgcaggaggtggtgcctttcctgaccaaactcagcaatcagctcagctcctgtcacatcagcggtgacgaccagaacatccagaagaatgtcagaaggctgaaggagacagtgaaaaagcttggagagagtggagagatcaaggcgattggggaactggacctgctgtttatgtctctgagaaatgcttgcgtcgctcgaggacccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctacgcgtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 73 - protein, 411 aa, Artificial Sequence; identical to SEQ ID NO: 60
  Synthetic polypeptide

SEQ ID NO: 74 - DNA, 20 bp, Artificial Sequence
  Synthetic primer
aggtccattcagatgctggt

SEQ ID NO: 75 - DNA, 20 bp, Artificial Sequence
  Synthetic primer
taggtgtggttgacgtggag

SEQ ID NO: 76 - DNA, 20 bp, Artificial Sequence
  Synthetic primer
ccaccccacactcacaccgg

SEQ ID NO: 77 - protein, 177 aa, Homo sapiens [sequence omitted]

SEQ ID NO: 78 - protein, 176 aa, Homo sapiens
MKASSLAFSLLSAAFYLLWTPSTGLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 79 - protein, 207 aa, Homo sapiens
MNFQQRLQSLWTLASRPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 80 - protein, 171 aa, Homo sapiens
MLVNFILRCGLLLVTLSLAIAKHKQSSFTKSCYPRGTLSQAVDALYIKAAWLKATIPEDRIKNIRLLKKKTKKQFMKNCQFQEQLLSFFMEDVFGQLQLQGCKKIRFVEDFHSLRQKLSHCISCASSAREMKSITRMKRIFYRIGNKGIYKAISELDILLSWIKKLLESSQ

SEQ ID NO: 81 - protein, 574 aa, Homo sapiens
MRTLLTILTVGSLAAHAPEDPSDLLQHVKFQSSNFENILTWDSGPEGTPDTVYSIEYKTYGERDWVAKKGCQRITRKSCNLTVETGNLTELYYARVTAVSAGGRSATKMTDRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRTWTYSFSGAFLFSMGFLVAVLCYLSYRYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSRPCSPSDQGPSPWGLLESLVCPKDEAKSPAPETSDLEQPTELDSLFRGLALTVQWES

SEQ ID NO: 82 - protein, 559 aa, Homo sapiens
HAPEDPSDLLQHVKFQSSNFENILTWDSGPEGTPDTVYSIEYKTYGERDWVAKKGCQRITRKSCNLTVETGNLTELYYARVTAVSAGGRSATKMTDRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRTWTYSFSGAFLFSMGFLVAVLCYLSYRYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSRPCSPSDQGPSPWGLLESLVCPKDEAKSPAPETSDLEQPTELDSLFRGLALTVQWES

SEQ ID NO: 83 - protein, 325 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 84 - protein, 306 aa, Homo sapiens
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

(19) United States
(12) Patent Application Publication
(10) Pub. No.: US 2020/0362003 A1
(43) Pub. Date: Nov. 19, 2020

(54) IL-22 Fc FUSION PROTEINS AND METHODS OF USE

(71) Applicant: Genentech, Inc., South San Francisco, CA (US)

(72) Inventors: Matthew KALO, South San Francisco, CA (US); Abigail Friederike Joyce PYNN, South San Francisco, CA (US); Lindsey Marie SILVA, South San Francisco, CA (US); Anjali SRIVASTAVA, South San Francisco, CA (US); Jayashree SUBRAMANIAN, South San Francisco, CA (US); Siddharth SUKUMARAN, South San Francisco, CA (US); Amy YOUNG, South San Francisco, CA (US); Tomasz BAGINSKI, South San Francisco, CA (US); Tracy Jane BENTLEY, South San Francisco, CA (US); Jeremy BESMER, South San Francisco, CA (US); Sherrie Patrice CURTIS, South San Francisco, CA (US); Peter William DAY, South San Francisco, CA (US)

(21) Appl. No.: 16/938,696

(22) Filed: Jul. 24, 2020

Related U.S. Application Data
(63) Continuation of application No. PCT/US2019/015277, filed on Jan. 25, 2019.
(60) Provisional application No. 62/622,767, filed on Jan. 26, 2018.

(51) Int. Cl. C07K 14/54; A61K 9/00; A61K 38/20; C12P 21/02; C07K 1/22; C07K 1/18; C07K 1/36; C07K 1/34

(52) U.S. Cl. CPC C07K 14/54; A61K 9/0019; A61K 38/20; C12P 21/02; C07K 2319/35; C07K 1/18; C07K 1/36; C07K 1/34; C07K 2319/30; C07K 1/22

(57) ABSTRACT

The invention relates to IL-22 Fc fusion proteins, composition comprising the same, methods of making and/or purifying the same, methods of selecting batches of IL-22 Fc fusion proteins or compositions thereof, and methods of using the composition for the treatment of diseases (e.g., IBD).

### SEQUENCE LISTING

The instant application contains a Sequence Listing which has been submitted electronically in ASCII format and is hereby incorporated by reference in its entirety. Said ASCII copy, created on Jul. 23, 2020, is named 50474-180002_Sequence_Listing_07.23.20_ST25 and is 121,869 bytes in size.

### FIELD OF THE INVENTION

The present invention relates to IL-22 Fc fusion proteins, compositions (e.g., pharmaceutical compositions) comprising the same, and methods of making, purifying, and using the same.

### BACKGROUND OF THE INVENTION

Interleukin (IL)-22 is a member of the IL-10 family of cytokines that is produced, e.g., by Th22 cells, NK cells, lymphoid tissue inducer (LTi) cells, dendritic cells, and Th17 cells. IL-22 binds to the IL-22R1/IL-10R2 receptor complex, which is expressed in innate cells (e.g., epithelial cells, hepatocytes, and keratinocytes) and in barrier epithelial tissues of several organs (e.g., dermis, pancreas, intestine, and the respiratory system).

IL-22 plays an important role in mucosal immunity, mediating early host defense against attaching and effacing bacterial pathogens. IL-22 promotes the production of anti-microbial peptides and pro-inflammatory cytokines from epithelial cells and stimulates proliferation and migration of colonic epithelial cells in the gut. Upon bacterial infection, IL-22 knock-out mice displayed impaired gut epithelial regeneration, high bacterial load, and increased mortality. Similarly, infection of IL-22 knock-out mice with influenza virus resulted in severe weight loss and impaired regeneration of tracheal and bronchial epithelial cells. Thus, IL-22 plays a pro-inflammatory role in suppressing microbial infection as well as an anti-inflammatory protective role in epithelial regeneration in inflammatory responses.

There remains a need for improved therapeutic agents and methods for treatment of inflammatory bowel disease (IBD), including ulcerative colitis and Crohn's disease, as well as other disorders including microbial infection, acute kidney injury, acute pancreatitis, wounds, cardiovascular conditions, metabolic syndrome, acute endotoxemia, graft-versus-host disease (GVHD), and sepsis. There also remains a need for improved methods for making and purifying such therapeutic agents.

### SUMMARY OF THE INVENTION

The present invention provides, inter alia, interleukin (IL)-22 Fc fusion proteins, compositions (e.g., pharmaceutical compositions) comprising the same, and methods of making, purifying, and using the same, e.g., for treatment of disorders including IBD, microbial infection, acute kidney injury, acute pancreatitis, wounds, cardiovascular conditions, metabolic syndrome, acute endotoxemia, GVHD, and sepsis, as well as methods of selecting a batch comprising IL-22 Fc fusion proteins for release. Also provided herein are methods of controlling sialic acid content of an IL-22 Fc fusion protein and methods of reducing in vivo clearance and/or increasing half-life by adjusting the sialic acid content of an IL-22 Fc fusion protein or a composition thereof.

In one aspect, the invention features a composition comprising an interleukin-22 (IL-22) Fc fusion protein, wherein the IL-22 Fc fusion protein comprises a glycosylated IL-22 polypeptide linked to an Fc region by a linker, and wherein the composition has an average sialic acid content in the range of 8 to 12 moles of sialic acid per mole of the IL-22 Fc fusion protein. In some embodiments, the IL-22 polypeptide is N-glycosylated. In some embodiments, the IL-22 polypeptide is glycosylated at one or more locations corresponding to amino acid residues Asn21, Asn35, Asn64, and/or Asn143 of SEQ ID NO: 4.

In another aspect, the invention features a composition comprising an IL-22 Fc fusion protein, wherein the IL-22 Fc fusion protein comprises a glycosylated IL-22 polypeptide linked to an Fc region by a linker, wherein the IL-22 polypeptide is glycosylated at one or more locations corresponding to amino acid residues Asn21, Asn35, Asn64, and/or Asn143 of SEQ ID NO: 4, and wherein: (a) the percent N-glycosylation site occupancy at residue Asn21 is in the range of 70 to 90; (b) the percent N-glycosylation site occupancy at residue Asn35 is in the range of 90 to 100; (c) the percent N-glycosylation site occupancy at residue Asn64 is in the range of 90 to 100; and/or (d) the percent N-glycosylation site occupancy at residue Asn143 is in the range of 25 to 35.

In some embodiments of any of the preceding aspects, the composition has an average sialic acid content in the range of 8 to 9 moles of sialic acid per mole of the IL-22 Fc fusion protein. In some embodiments, the composition has an average sialic acid content of 8 or 9 moles of sialic acid per mole of the IL-22 Fc fusion protein. In some embodiments, the composition has an average sialic acid content of 8 moles of sialic acid per mole of the IL-22 Fc fusion protein. In other embodiments, the composition has an average sialic acid content of 9 moles of sialic acid per mole of the IL-22 Fc fusion protein.

In some embodiments of any of the preceding aspects, the sialic acid glycosylation comprises N-acetylneuraminic acid (NANA).

In some embodiments of any of the preceding aspects, the composition has an average N-glycolylneuraminic acid (NGNA) content of less than 1 mole of NGNA per mole of the IL-22 Fc fusion protein.

In some embodiments of any of the preceding aspects, the composition is a liquid composition.

In some embodiments of any of the preceding aspects: (i) the IL-22 Fc fusion protein has a maximum observed concentration (Cₘₐₓ) of about 8,000 ng/mL to about 19,000 ng; (ii) the IL-22 Fc fusion protein has an area under the serum concentration-time curve from time 0 to the last measureable time point (AUCₗₐₛₜ) of about 7,000 day·ng/mL to about 25,000 day·ng/mL; and/or (iii) the IL-22 Fc fusion protein has a clearance (CL) of about 40 mL/kg/day to about 140 mL/kg/day. In some embodiments, the Cₘₐₓ, AUCₗₐₛₜ, and/or CL is assessed following intravenous administration of about 1,000 μg/kg of the IL-22 Fc fusion protein to a CD1 mouse.

In some embodiments of any of the preceding aspects, the IL-22 polypeptide comprises N-glycans having monoantennary, biantennary, triantennary, and/or tetraantennary structure. In some embodiments: (i) about 0.1% to about 2% of the N-glycans have monoantennary structure; (ii) about 10% to about 25% of the N-glycans have biantennary structure; (iii) about 25% to about 40% of the N-glycans have triantennary structure; and/or (iv) about 30% to about 51% of the N-glycans have tetraantennary structure. In some embodiments: (i) 0.1% to 2% of the N-glycans have monoantennary structure; (ii) 10% to 25% of the N-glycans have biantennary structure; (iii) 25% to 40% of the N-glycans have triantennary structure; and/or (iv) 30% to 51% of the N-glycans have tetraantennary structure.

In some embodiments of any of the preceding aspects, the IL-22 Fc fusion protein comprises N-glycans comprising zero, one, two, three, or four galactose moieties. In some embodiments: (i) about 9% to about 32% of the N-glycans comprise zero galactose moieties; (ii) about 10% to about 20% of the N-glycans comprise one galactose moiety; (iii) about 8% to about 25% of the N-glycans comprise two galactose moieties; (iv) about 12% to about 25% of the N-glycans comprise three galactose moieties; and/or (v) about 12% to about 30% of the N-glycans comprise four galactose moieties. In some embodiments: (i) 9% to 32% of the N-glycans comprise zero galactose moieties; (ii) 10% to 20% of the N-glycans comprise one galactose moiety; (iii) 8% to 25% of the N-glycans comprise two galactose moieties; (iv) 12% to 25% of the N-glycans comprise three galactose moieties; and/or (v) 12% to 30% of the N-glycans comprise four galactose moieties.

In some embodiments of any of the preceding aspects, the IL-22 Fc fusion protein comprises N-glycans comprising zero, one, two, three, or four sialic acid moieties. In some embodiments: (i) about 12% to about 35% of the N-glycans comprise zero sialic acid moieties; (ii) about 10% to about 30% of the N-glycans comprise one sialic acid moiety; (iii) about 10% to about 30% of the N-glycans comprise two sialic acid moieties; (iv) about 10% to about 30% of the N-glycans comprise three sialic acid moieties; and/or (v) about 1% to about 20% of the N-glycans comprise four sialic acid moieties. In some embodiments: (i) 12% to 35% of the N-glycans comprise zero sialic acid moieties; (ii) 10% to 30% of the N-glycans comprise one sialic acid moiety; (iii) 10% to 30% of the N-glycans comprise two sialic acid moieties; (iv) 10% to 30% of the N-glycans comprise three sialic acid moieties; and/or (v) 1% to 20% of the N-glycans comprise four sialic acid moieties.

In some embodiments of any of the preceding aspects, (i) the IL-22 polypeptide comprises about 0% to about 10% N-glycans comprising a terminal mannose moiety; and/or (ii) the IL-22 polypeptide comprises about 30% to about 55% N-glycans comprising a terminal N-acetylglucosamine (GlcNAc) moiety. In some embodiments, (i) the IL-22 polypeptide comprises 0% to 10% N-glycans comprising a terminal mannose moiety; and/or (ii) the IL-22 polypeptide comprises 30% to 55% N-glycans comprising a terminal GlcNAc moiety. In some embodiments, the IL-22 polypeptide comprises 0% to 10% N-glycans comprising a terminal mannose moiety. In some embodiments, the IL-22 polypeptide comprises 30% to 55% N-glycans comprising a terminal GlcNAc moiety.

In some embodiments of any of the preceding aspects, the N-glycans comprise one, two, three, or four terminal GlcNAc moieties. In some embodiments: (i) about 1% to about 20% of the N-glycans comprise one terminal GlcNAc moiety; (ii) about 1% to about 20% of the N-glycans comprise two terminal GlcNAc moieties; (iii) about 5% to about 25% of the N-glycans comprise three terminal GlcNAc moieties; and/or (iv) about 0% to about 15% of the N-glycans comprise four terminal GlcNAc moieties. In some embodiments: (i) 1% to 20% of the N-glycans comprise one terminal GlcNAc moiety; (ii) 1% to 20% of the N-glycans comprise two terminal GlcNAc moieties; (iii) 5% to 25% of the N-glycans comprise three terminal GlcNAc moieties; and/or (iv) 0% to 15% of the N-glycans comprise four terminal GlcNAc moieties.

In some embodiments of any of the preceding aspects, (i) the IL-22 polypeptide comprises about 20% to about 45% N-glycans comprising a terminal galactose (Gal) moiety; and/or (ii) the N-glycans comprise one, two, or three terminal Gal moieties. In some embodiments, (i) the IL-22 polypeptide comprises 20% to 45% N-glycans comprising a terminal Gal moiety; and/or (ii) the N-glycans comprise one, two, or three terminal Gal moieties.

In some embodiments of any of the preceding aspects: (i) about 15% to about 30% of the N-glycans comprise one terminal Gal moiety; (ii) about 1% to about 15% of the N-glycans comprise two terminal Gal moieties; and/or (iii) about 0.1% to about 6% of the N-glycans comprise three terminal Gal moieties. In some embodiments: (i) 15% to 30% of the N-glycans comprise one terminal Gal moiety; (ii) 1% to 15% of the N-glycans comprise two terminal Gal moieties; and/or (iii) 0.1% to 6% of the N-glycans comprise three terminal Gal moieties.

In some embodiments of any of the preceding aspects: (i) the IL-22 polypeptide comprises N-glycans comprising galactose N-acetylglucosamine (LacNAc) repeats; (ii) the IL-22 polypeptide comprises N-glycans comprising fucosylated N-glycans; and/or (iii) the IL-22 polypeptide comprises N-glycans comprising afucosylated N-glycans.

In another aspect, the invention provides a composition comprising an IL-22 Fc fusion protein having the N-glycan distribution shown in Table 12 or 13.

In some embodiments of any of the preceding aspects, the concentration of the IL-22 Fc fusion protein is about 0.5 mg/mL to about 20 mg/mL. In some embodiments, the concentration of the IL-22 Fc fusion protein is about 0.5 mg/mL to about 5 mg/mL. In some embodiments, the concentration of the IL-22 Fc fusion protein is about 1 mg/mL. In some embodiments, the concentration of the IL-22 Fc fusion protein is about 8 mg/mL to about 12 mg/mL. In some embodiments, the concentration of the IL-22 Fc fusion protein is about 10 mg/mL.

In some embodiments of any of the preceding aspects, the IL-22 Fc fusion protein has been produced from a production culture having a volume of at least about 500 L. In some embodiments of any of the preceding aspects, the IL-22 Fc fusion protein has been produced from a production culture having a volume of about 500 L to about 5,000 L. In some embodiments, the IL-22 Fc fusion protein has been produced from a production culture having a volume of about 1,000 L to about 3,000 L. In some embodiments the IL-22 Fc fusion protein has been produced from a production culture having a volume of about 1,500 L to about 2,500 L. In some embodiments, the IL-22 Fc fusion protein has been produced from a production culture having a volume of about 2000 L.

In some embodiments of any of the preceding aspects, the Fc region is not glycosylated. In some embodiments: (i) the amino acid residue at position 297 as in the EU index of the Fc region is Gly or Ala; and/or (ii) the amino acid residue at position 299 as in the EU index of the Fc region is Ala, Gly, or Val. In some embodiments, the amino acid residue at position 297 as in the EU index of the Fc region is Gly or Ala. In some embodiments, the amino acid residue at position 297 as in the EU index of the Fc region is Gly. In other embodiments, the amino acid residue at position 297 as in the EU index of the Fc region is Ala.

In some embodiments of any of the preceding aspects, the Fc region comprises the CH2 and CH3 domain of IgG1 or IgG4. In some embodiments, the Fc region comprises the CH2 and CH3 domain of IgG4.

In some embodiments of any of the preceding aspects, the IL-22 Fc fusion protein comprises an amino acid sequence having at least 95% (e.g., at least 95%, at least 96%, at least 97%, at least 98%, or at least 99%) sequence identity to the amino acid sequence of SEQ ID NO:8.

In some embodiments of any of the preceding aspects, the IL-22 Fc fusion protein comprises or consists of the amino acid sequence of SEQ ID NO:8, SEQ ID NO:10, or SEQ ID NO:16.

In some embodiments of any of the preceding aspects, the IL-22 polypeptide is a human IL-22 polypeptide. In some embodiments, the IL-22 polypeptide comprises the amino acid sequence of SEQ ID NO:4.

In some embodiments of any of the preceding aspects, the linker comprises or consists of the amino acid sequence RVESKYGPP (SEQ ID NO: 44).

In some embodiments of any of the preceding aspects, the IL-22 Fc fusion protein binds to IL-22 receptor. In some embodiments, the IL-22 receptor is human IL-22 receptor. In some embodiments, the human IL-22 receptor comprises a heterodimer consisting of an IL-22R1 polypeptide and an IL-10R2 polypeptide. In some embodiments, the IL-22R1 polypeptide comprises the amino acid sequence of SEQ ID NO:82 and the IL-10R2 polypeptide comprises the amino acid sequence of SEQ ID NO:84.

In some embodiments of any of the preceding aspects, the IL-22 Fc fusion protein consists of two single-chain units linked by two inter-chain disulfide bridges, wherein each single chain unit consists of a human IL-22 fusion protein comprising IL-22 fused with the Fc region of a human immunoglobulin IgG4.

In some embodiments of any of the preceding aspects, the composition is a pharmaceutical composition. In some embodiments, the composition is aqueous and/or sterile. In some embodiments, the composition further comprises an additional therapeutic agent. In some embodiments, the composition further comprises a gelling agent.

In another aspect, the invention features a method of treating inflammatory bowel disease (IBD) in a subject in need thereof, the method comprising administering to the subject any of the compositions described herein. In some embodiments, the IBD is ulcerative colitis or Crohn's disease. In some embodiments, the IBD is ulcerative colitis. In some embodiments, the ulcerative colitis is moderate to severe ulcerative colitis. In some embodiments, the IBD is Crohn's disease.

In another aspect, the invention features any of the compositions described herein for use as a medicament.

In another aspect, the invention features any of the compositions described herein for use in (i) treating inflammatory bowel disease (IBD), (ii) inhibiting microbial infection in the intestine, preserving goblet cells in the intestine during a microbial infection, enhancing epithelial cell integrity, epithelial cell proliferation, epithelial cell differentiation, epithelial cell migration or epithelial wound healing in the intestine, (iii) treating acute kidney injury or acute pancreatitis, (iv) accelerating or improving wound healing in a subject in need thereof, (v) preventing or treating a cardiovascular disease such as coronary artery disease, coronary microvascular disease, stroke, carotid artery disease, peripheral artery disease, or chronic kidney disease, (vi) treating metabolic syndrome, (vii) treating acute endotoxemia or sepsis, or (viii) treating GVHD.

In another aspect, the invention features any of the compositions described herein for the preparation of a medicament for use in (i) treating inflammatory bowel disease (IBD), (ii) inhibiting microbial infection in the intestine, preserving goblet cells in the intestine during a microbial infection, enhancing epithelial cell integrity, epithelial cell proliferation, epithelial cell differentiation, epithelial cell migration or epithelial wound healing in the intestine, (iii) treating acute kidney injury or acute pancreatitis, (iv) accelerating or improving wound healing in a subject in need thereof, (v) preventing or treating a cardiovascular disease such as coronary artery disease, coronary microvascular disease, stroke, carotid artery disease, peripheral artery disease, or chronic kidney disease, (vi) treating metabolic syndrome, (vii) treating acute endotoxemia or sepsis, or (viii) treating GVHD.

In another aspect, the invention features a method of inhibiting microbial infection in the intestine, preserving goblet cells in the intestine during a microbial infection, enhancing epithelial cell integrity, epithelial cell proliferation, epithelial cell differentiation, epithelial cell migration or epithelial wound healing in the intestine, of a subject in need thereof, the method comprising administering to the subject any of the compositions described herein.

In another aspect, the invention features a method of treating acute kidney injury or acute pancreatitis in a subject in need thereof, the method comprising administering to the subject any of the compositions described herein.

In another aspect, the invention features a method of accelerating or improving wound healing in a subject in need thereof, the method comprising administering to the subject any of the compositions described herein.

In another aspect, the invention features a method for preventing or treating a cardiovascular condition in a subject in need thereof, which condition includes a pathology of atherosclerotic plaque formation, the method comprising administering to the subject any of the compositions described herein.

In another aspect, the invention features a method for treating metabolic syndrome in a subject in need thereof, the method comprising administering to the subject any of the compositions described herein.

In another aspect, the invention features a method of treating acute endotoxemia, sepsis, or both, in a subject in need thereof, the method comprising administering to the subject any of the compositions described herein.

In another aspect, the invention features a method of treating GVHD in a subject in need thereof, the method comprising administering to the subject any of the compositions described herein.

In some embodiments of any of the preceding aspects, the composition is administered intravenously, subcutaneously, intraperitoneally, or topically.

In some embodiments of any of the preceding aspects, the subject is co-administered with at least one additional therapeutic agent.

In another aspect, the invention features a method of making a composition comprising an IL-22 Fc fusion protein, the method comprising the following steps: (a) providing a host cell comprising a nucleic acid encoding a IL-22 Fc fusion protein, the IL-22 Fc fusion protein comprising an IL-22 polypeptide linked to an Fc region by a linker; (b) culturing the host cell in a seed train medium under conditions suitable to form a seed train culture; (c) inoculating the seed train in an inoculum medium under conditions suitable to form an inoculum train culture; and (d) culturing the inoculum train in a production medium under conditions suitable to form a production culture, wherein the host cells of the production culture express the IL-22 Fc fusion protein, and wherein the duration of step (d) is at least 10 days, thereby making the composition comprising an IL-22 Fc fusion protein, wherein the IL-22 polypeptide is glycosylated, and wherein the composition has an average sialic acid content in the range of 6 to 12 moles of sialic acid per mole of the IL-22 Fc fusion protein. In some embodiments, the duration of step (d) is at least 11 days, at least 12 days, or at least 13 days. In some embodiments, the duration of step (d) is 12 days.

In some embodiments of any of the preceding aspects, the method further comprises the following step: (e) harvesting a cell culture fluid comprising the IL-22 Fc fusion protein from the production culture. In some embodiments, step (e) comprises: (i) cooling the production culture; (ii) removing the host cells from the production medium by centrifugation to form the cell culture fluid; and/or (iii) filtering the cell culture fluid.

In some embodiments of any of the preceding aspects, the method further comprises the following step: (f) purifying the IL-22 Fc fusion protein in the cell culture fluid. In some embodiments, step (f) comprises the following substeps: (i) contacting the cell culture fluid to an affinity chromatographic support, optionally washing the affinity chromatographic support with a wash buffer, eluting the IL-22 Fc fusion protein from the affinity chromatographic support with a first elution buffer to form an affinity pool, and optionally inactivating viruses in the affinity pool; (ii) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and (iii) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool. In some embodiments, step (f) further comprises one or more of the following substeps: (iv) concentrating the purified product pool to form a concentrated product pool; (v) ultrafiltering the purified product pool; (vi) exchanging the buffer of the concentrated product pool to form a ultrafiltration and diafiltration (UFDF) pool comprising the IL-22 Fc fusion protein; and/or (vii) conditioning the UFDF pool with a formulation buffer to form a conditioned UFDF pool comprising the IL-22 Fc fusion protein. In some embodiments, substep (i) further comprises inactivating viruses by adding a detergent to the cell culture fluid prior to contacting the cell culture fluid to the affinity column.

In another aspect, the invention features a method of making a composition comprising an IL-22 Fc fusion protein, the method comprising: culturing an inoculum train culture comprising a plurality of host cells in a production medium under conditions suitable to form a production culture for at least about 10 days, wherein the host cells comprise a nucleic acid encoding an IL-22 Fc fusion protein, the IL-22 Fc fusion protein comprising an IL-22 polypeptide linked to an Fc region by a linker, wherein the host cells express the IL-22 Fc fusion protein, thereby making the composition comprising an IL-22 Fc fusion protein, wherein the IL-22 polypeptide is glycosylated, and wherein the composition has an average sialic acid content in the range of 6 to 12 moles of sialic acid per mole of the IL-22 Fc fusion protein. In some embodiments, the duration of the culturing is at least 11 days, at least 12 days, or at least 13 days. In some embodiments, the duration of the culturing is 12 days.

In some embodiments of any of the preceding aspects, the method further comprises generating a seed train culture by culturing a host cell comprising a nucleic acid encoding the IL-22 Fc fusion protein in a seed train medium under conditions suitable to form the seed train culture prior to culturing the inoculum train culture in the production medium. In some embodiments, the method further comprises inoculating the seed train culture in an inoculum medium under conditions suitable to form an inoculum train culture prior to culturing the inoculum train culture in the production medium.

In some embodiments of any of the preceding aspects, the host cells are eukaryotic host cells. In some embodiments, the eukaryotic host cells are mammalian host cells. In some embodiments, the mammalian host cells are Chinese hamster ovary (CHO) cells. In some embodiments, harvesting the cell culture fluid comprises: (i) cooling the production culture; (ii) removing the host cells from the production medium by centrifugation to form the cell culture fluid; and/or (iii) filtering the cell culture fluid.

In some embodiments of any of the preceding aspects, the method further comprises purifying the IL-22 Fc fusion protein in the cell culture fluid. In some embodiments, purifying the IL-22 Fc fusion protein comprises the following substeps: (i) contacting the cell culture fluid to an affinity chromatographic support, optionally washing the affinity chromatographic support with a wash buffer, eluting the IL-22 Fc fusion protein from the affinity chromatographic support with a first elution buffer to form an affinity pool, and optionally inactivating viruses in the affinity pool; (ii) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and (iii) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool. In some embodiments, purifying the IL-22 Fc fusion protein further comprises one or more of the following substeps: (iv) concentrating the purified product pool to form a concentrated product pool; (v) ultrafiltering the purified product pool; (vi) exchanging the buffer of the concentrated product pool to form a ultrafiltration and diafiltration (UFDF) pool comprising the IL-22 Fc fusion protein; and/or (vii) conditioning the UFDF pool with a formulation buffer to form a conditioned UFDF pool comprising the IL-22 Fc fusion protein. In some embodiments, substep (i) further comprises inactivating viruses by adding a detergent to the cell culture fluid prior to contacting the cell culture fluid to the affinity column.

In some embodiments of any of the preceding aspects, the method further comprises enriching the sialic acid content of the composition. In some embodiments, the composition has an initial average sialic acid content in the range of 6 to 8 moles of sialic acid per mole of the IL-22 Fc fusion protein. In some embodiments, the composition has an initial average sialic acid content of 6, 7, or 8 moles of sialic acid per mole of the IL-22 Fc fusion protein. In some embodiments, the method further comprises enriching the average sialic acid content to the range of 8 to 12 moles of sialic acid per mole of the IL-22 Fc fusion protein. In some embodiments, the method further comprises enriching the average sialic acid content to the range of 8 to 9 moles of sialic acid per mole of the IL-22 Fc fusion protein.

In some embodiments of any of the preceding aspects, the affinity chromatographic support comprises a protein A resin, a protein G resin, or an IL-22 receptor resin. In some embodiments, the protein A resin is a MABSELECT SURE® resin.

In some embodiments of any of the preceding aspects, the anion-exchange chromatographic support comprises a strong anion exchanger with multimodal functionality resin. In some embodiments, the anion-exchange chromatographic support comprises a CAPTO™ adhere resin.

In some embodiments of any of the preceding aspects, the composition has an average sialic acid content of 8 to 12 moles of sialic acid per mole of the IL-22 Fc fusion protein.

In some embodiments of any of the preceding aspects, the composition has an average sialic acid content of 8 or 9 moles of sialic acid per mole of the IL-22 Fc fusion protein.

In another aspect, the invention features a composition produced by any of the methods described herein. In some embodiments, the composition is a pharmaceutical composition.

In some embodiments of any of the preceding aspects, the IL-22 Fc fusion protein consists of two single-chain units linked by two inter-chain disulfide bridges, wherein each single chain unit consists of a human IL-22 fusion protein comprising IL-22 fused with the Fc region of a human immunoglobulin IgG4.

In another aspect, the invention features a method of selecting a batch comprising an IL-22 Fc fusion protein for release, the method comprising the following steps: (a) providing a batch comprising IL-22 Fc fusion proteins; (b) assessing the levels of sialic acid in the batch; and (c) selecting the batch for release if the batch has an average sialic acid content in the range of 8 to 12 moles of sialic acid per mole of the IL-22 Fc fusion protein. In some embodiments, step (c) comprises selecting the batch for release if the batch has an average sialic acid content of 8 to 9 moles of sialic acid per mole of the IL-22 Fc fusion protein. In some embodiments, step (c) comprises selecting the batch for release if the batch has an average sialic acid content of 8 moles of sialic acid per mole of the IL-22 Fc fusion protein. In some embodiments, step (c) comprises selecting the batch for release if the batch has an average sialic acid content of 9 moles of sialic acid per mole of the IL-22 Fc fusion protein. In some embodiments, step (b) comprises using high-performance liquid chromatography (HPLC), ultra-high performance liquid chromatography (UHPLC), capillary electrophoresis, or a colorimetric assay to assess the levels of sialic acid in the batch. In some embodiments, step (b) comprises assessing the levels of sialic acid using HPLC.

In another aspect, the invention features a method for controlling sialic acid content of a composition comprising an IL-22 Fc fusion protein, the IL-22 Fc fusion protein comprising a glycosylated IL-22 polypeptide linked by a linker to an antibody Fc region, the method comprising: culturing an inoculum train culture comprising a plurality of host cells in a production medium under conditions suitable to form a production culture for at least 10 days, wherein the host cells comprise a nucleic acid encoding the IL-22 Fc fusion protein and express the IL-22 Fc fusion protein wherein the composition has an average sialic acid content in the range of 6 to 12 moles of sialic acid per mole of the IL-22 Fc fusion protein; and enriching the average sialic acid content of the composition to the range of 8 to 12 moles of sialic acid per mole of the IL-22 Fc fusion protein, thereby controlling the sialic acid content of the composition. In some embodiments, the method comprises enriching the average sialic acid content of the composition to the range of 8 to 9 moles of sialic acid per mole of the IL-22 Fc fusion protein.

In another aspect, the invention features a method for controlling sialic acid content of a composition comprising an IL-22 Fc fusion protein, the IL-22 Fc fusion protein comprising a glycosylated IL-22 polypeptide linked by a linker to an antibody Fc region, the method comprising: culturing an inoculum train culture comprising a plurality of host cells in a production medium under conditions suitable to form a production culture for at least 10 days, wherein the host cells comprise a nucleic acid encoding the IL-22 Fc fusion protein and express the IL-22 Fc fusion protein, wherein the composition has an average sialic acid content in the range of 6 to 12 moles of sialic acid per mole of the IL-22 Fc fusion protein; and enriching the average sialic acid content of the composition to the range of 8 to 12 moles of sialic acid per mole of the IL-22 Fc fusion protein, thereby controlling the sialic acid content of the composition.

In some embodiments of any of the preceding aspects, the method comprises enriching the average sialic acid content of the composition to the range of 8 to 9 moles of sialic acid per mole of the IL-22 Fc fusion protein.

In some embodiments of any of the preceding aspects, enriching the average sialic acid content comprises harvesting a cell culture fluid comprising the IL-22 Fc fusion protein from the production culture. In some embodiments, harvesting the cell culture fluid comprises: (i) cooling the production culture; (ii) removing the host cells from the production medium by centrifugation to form the cell culture fluid; and/or (iii) filtering the cell culture fluid.

In some embodiments of any of the preceding aspects, enriching the average sialic acid content of the composition further comprises purifying the IL-22 Fc fusion protein in the cell culture fluid. In some embodiments, purifying the IL-22 Fc fusion protein comprises the following substeps: (i) contacting the cell culture fluid to an affinity chromatographic support, optionally washing the affinity chromatographic support with a wash buffer, eluting the IL-22 Fc fusion protein from the affinity chromatographic support with a first elution buffer to form an affinity pool, and optionally inactivating viruses in the affinity pool; (ii) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and (iii) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool. In some embodiments, purifying the IL-22 Fc fusion protein further comprises one or more of the following substeps: (iv) concentrating the purified product pool to form a concentrated product pool; (v) ultrafiltering the purified product pool; (vi) exchanging the buffer of the concentrated product pool to form a ultrafiltration and diafiltration (UFDF) pool comprising the IL-22 Fc fusion protein; and/or (vii) conditioning the UFDF pool with a formulation buffer to form a conditioned UFDF pool comprising the IL-22 Fc fusion protein. In some embodiments, substep (i) further comprises inactivating viruses by adding a detergent to the cell culture fluid prior to contacting the cell culture fluid to the affinity column. In some embodiments, the affinity chromatographic support comprises a protein A resin, a protein G resin, or an IL-22 receptor resin. In some embodiments, the protein A resin is a MABSELECT SURE® resin. In some embodiments, the anion-exchange chromatographic support comprises a strong anion exchanger with multimodal functionality resin. In some embodiments, the anion-exchange chromatographic support comprises a CAPTO™ adhere resin.

In one aspect, the invention features an IL-22 Fc fusion protein comprising an IL-22 polypeptide linked to an Fc region by a linker, wherein the IL-22 polypeptide is glycosylated, and wherein the IL-22 Fc fusion protein has a sialic acid content in the range of from 8 to 12 moles of sialic acid per mole of the IL-22 Fc fusion protein. In certain aspects, 8 to 12 moles of sialic acid per mole of the IL-22 Fc fusion protein means that 8 to 12 sialic acid moieties are comprised in one mole of the IL-22 fusion protein. In some embodiments, the IL-22 Fc fusion protein has a sialic acid content in the range of 8 to 9 moles of sialic acid per mole of the IL-22 Fc fusion protein.

In another aspect, the invention features an IL-22 Fc fusion protein comprising an IL-22 polypeptide linked to an Fc region by a linker, wherein the IL-22 polypeptide is glycosylated, and wherein the IL-22 Fc fusion protein has a potency of about 40% to about 130% relative to a reference IL-22 Fc fusion protein having a sialic acid content of about 8 moles of sialic acid per mole of the IL-22 Fc fusion protein. In some embodiments, the IL-22 Fc fusion protein has a potency of about 80% to about 120% relative to a reference IL-22 Fc fusion protein having a sialic acid content of about 8 moles of sialic acid per mole of the IL-22 Fc fusion protein. In some embodiments, the IL-22 Fc fusion protein has a potency of about 60% to about 110% relative to a reference IL-22 Fc fusion protein having a sialic acid content of about 8 moles of sialic acid per mole of the IL-22 Fc fusion protein. In some embodiments, the IL-22 Fc fusion protein has a potency of about 80% to about 100% relative to a reference IL-22 Fc fusion protein having a sialic acid content of about 8 moles of sialic acid per mole of the IL-22 Fc fusion protein. In some embodiments, potency is assessed in a receptor binding assay or a cell-based binding assay. In some embodiments, the reference IL-22 Fc fusion protein has the N-glycan distribution shown in Table 12 and/or Table 13.

In some embodiments of any of the preceding aspects, the IL-22 Fc fusion protein has a sialic acid content of from about 8 to about 12 moles of sialic acid per mole of the IL-22 Fc fusion protein. In some embodiments, the IL-22 Fc fusion protein has a sialic acid content of from about 8 to about 11 moles of sialic acid per mole of the IL-22 Fc fusion protein. In some embodiments, the IL-22 Fc fusion protein has a sialic acid content of from about 8 to about 10 moles of sialic acid per mole of the IL-22 Fc fusion protein. In some embodiments, the IL-22 Fc fusion protein has a sialic acid content of from about 8 to about 9 moles of sialic acid per mole of the IL-22 Fc fusion protein. In some embodiments, the IL-22 Fc fusion protein has a sialic acid content of about 8 moles of sialic acid per mole of the IL-22 Fc fusion protein. In some embodiments, the IL-22 Fc fusion protein has a sialic acid content of 8 moles of sialic acid per mole of the IL-22 Fc fusion protein.

In some embodiments of any of the preceding aspects, the IL-22 Fc fusion protein has a sialic acid content of about 9 moles of sialic acid per mole of the IL-22 Fc fusion protein. In some embodiments, the IL-22 Fc fusion protein has a sialic acid content of 9 moles of sialic acid per mole of the IL-22 Fc fusion protein. In some embodiments, the sialic acid is N-acetylneuraminic acid (NANA). In some embodiments, the IL-22 Fc fusion protein has a maximum observed concentration (Cₘₐₓ) of about 9,000 ng/mL to about 18,000 ng/ml. In some embodiments, the Cₘₐₓ is assessed following intravenous administration of about 1,000 μg/kg of the IL-22 Fc fusion protein to a CD1 mouse. In some embodiments, the IL-22 Fc fusion protein has an area under the serum concentration-time curve from time 0 to the last measureable time point (AUCₗₐₛₜ) of about 7,000 day·ng/mL to about 25,000 day·ng/mL. In some embodiments, the AUCₗₐₛₜ is assessed following intravenous administration of about 1,000 μg/kg of the IL-22 Fc fusion protein to a CD1 mouse. In some embodiments, the IL-22 Fc fusion protein has a clearance (CL) of about 40 mL/kg/day to about 140 mL/kg/day. In some embodiments, the CL is assessed following intravenous administration of about 1,000 μg/kg of the IL-22 Fc fusion protein to a CD1 mouse.

In some embodiments of any of the preceding aspects, the IL-22 polypeptide is N-glycosylated.

In some embodiments of any of the preceding aspects, the IL-22 polypeptide comprises N-glycans having monoantennary, biantennary, triantennary, and/or tetraantennary structure. In some embodiments, about 0.1% to about 2% of the N-glycans have monoantennary structure. In some embodiments, about 0.5% to about 1.5% of the N-glycans have monoantennary structure. In some embodiments, about 1% of the N-glycans have monoantennary structure. In some embodiments, about 10% to about 25% of the N-glycans have biantennary structure. In some embodiments, about 12% to about 21% of the N-glycans have biantennary structure. In some embodiments, about 17% of the N-glycans have biantennary structure. In some embodiments, about 25% to about 40% of the N-glycans have triantennary structure. In some embodiments, about 28% to about 35% of the N-glycans have triantennary structure. In some embodiments, about 31% of the N-glycans have triantennary structure. In some embodiments, about 30% to about 51% of the N-glycans have tetraantennary structure. In some embodiments, about 35% to about 48% of the N-glycans have tetraantennary structure. In some embodiments, about 42% of the N-glycans have tetraantennary structure.

In some embodiments of any of the preceding aspects, the IL-22 Fc fusion protein comprises N-glycans comprising zero, one, two, three, or four galactose moieties. In some embodiments, about 9% to about 32% of the N-glycans comprise zero galactose moieties. In some embodiments, about 15% to about 25% of the N-glycans comprise zero galactose moieties. In some embodiments, about 21% of the N-glycans comprise zero galactose moieties. In some embodiments, about 10% to about 20% of the N-glycans comprise one galactose moiety. In some embodiments, about 12% to about 16% of the N-glycans comprise one galactose moiety. In some embodiments, about 14% of the N-glycans comprise one galactose moiety. In some embodiments, about 8% to about 25% of the N-glycans comprise two galactose moieties. In some embodiments, about 10% to about 16% of the N-glycans comprise two galactose moieties. In some embodiments, about 13% of the N-glycans comprise two galactose moieties. In some embodiments, about 12% to about 25% of the N-glycans comprise three galactose moieties. In some embodiments, about 15% to about 22% of the N-glycans comprise three galactose moieties. In some embodiments, about 19% of the N-glycans comprise three galactose moieties. In some embodiments, about 12% to about 30% of the N-glycans comprise four galactose moieties. In some embodiments, about 15% to about 25% of the N-glycans comprise four galactose moieties. In some embodiments, about 24% of the N-glycans comprise four galactose moieties.

In some embodiments of any of the preceding aspects, the IL-22 Fc fusion protein comprises N-glycans comprising zero, one, two, three, or four sialic acid moieties. In some embodiments, about 12% to about 35% of the N-glycans comprise zero sialic acid moieties. In some embodiments, about 20% to about 30% of the N-glycans comprise zero sialic acid moieties. In some embodiments, about 24% of the N-glycans comprise zero sialic acid moieties. In some embodiments, about 10% to about 30% of the N-glycans comprise one sialic acid moiety. In some embodiments, about 15% to about 25% of the N-glycans comprise one sialic acid moiety. In some embodiments, about 20% of the N-glycans comprise one sialic acid moiety. In some embodiments, about 10% to about 30% of the N-glycans comprise two sialic acid moieties. In some embodiments, about 15% to about 25% of the N-glycans comprise two sialic acid moieties. In some embodiments, about 21% of the N-glycans comprise two sialic acid moieties. In some embodiments, about 10% to about 30% of the N-glycans comprise three sialic acid moieties. In some embodiments, about 12% to about 24% of the N-glycans comprise three sialic acid moieties. In some embodiments, about 17% of the N-glycans comprise three sialic acid moieties. In some embodiments, about 1% to about 20% of the N-glycans comprise four sialic acid moieties. In some embodiments, about 5% to about 15% of the N-glycans comprise four sialic acid moieties. In some embodiments, about 9% of the N-glycans comprise four sialic acid moieties.

In some embodiments of any of the preceding aspects, the IL-22 polypeptide comprises about 0% to about 10% N-glycans comprising a terminal mannose moiety. In some embodiments, about 1% to about 4% of the N-glycans comprise a terminal mannose moiety. In some embodiments, about 2% of the N-glycans comprise a terminal mannose moiety.

In some embodiments of any of the preceding aspects, the IL-22 polypeptide comprises about 30% to about 55% N-glycans comprising a terminal N-acetylglucosamine (GlcNAc) moiety. In some embodiments, about 35% to about 50% of the N-glycans comprise a terminal GlcNAc moiety. In some embodiments, about 42% of the N-glycans comprise a terminal GlcNAc moiety.

In some embodiments of any of the preceding aspects, the N-glycans comprise one, two, three, or four terminal GlcNAc moieties. In some embodiments, about 1% to about 20% of the N-glycans comprise one terminal GlcNAc moiety. In some embodiments, about 5% to about 15% of the N-glycans comprise one terminal GlcNAc moiety. In some embodiments, about 10% of the N-glycans comprise one terminal GlcNAc moiety. In some embodiments, about 1% to about 20% of the N-glycans comprise two terminal GlcNAc moieties. In some embodiments, about 5% to about 15% of the N-glycans comprise two terminal GlcNAc moieties. In some embodiments, about 10% of the N-glycans comprise two terminal GlcNAc moieties. In some embodiments, about 5% to about 25% of the N-glycans comprise three terminal GlcNAc moieties. In some embodiments, about 10% to about 20% of the N-glycans comprise three terminal GlcNAc moieties. In some embodiments, about 14% of the N-glycans comprise three terminal GlcNAc moieties. In some embodiments, about 0% to about 15% of the N-glycans comprise four terminal GlcNAc moieties. In some embodiments, about 4% to about 12% of the N-glycans comprise four terminal GlcNAc moieties. In some embodiments, about 7% of the N-glycans comprise four terminal GlcNAc moieties.

In some embodiments of any of the preceding aspects, the IL-22 polypeptide comprises about 20% to about 45% N-glycans comprising a terminal galactose (Gal) moiety. In some embodiments, about 25% to about 35% of the N-glycans comprise a terminal Gal moiety. In some embodiments, about 32% of the N-glycans comprise a terminal Gal moiety.

In some embodiments of any of the preceding aspects, the N-glycans comprise one, two, or three terminal Gal moieties. In some embodiments, about 15% to about 30% of the N-glycans comprise one terminal Gal moiety. In some embodiments, about 20% to about 25% of the N-glycans comprise one terminal Gal moiety. In some embodiments, about 23% of the N-glycans comprise one terminal Gal moiety. In some embodiments, about 1% to about 15% of the N-glycans comprise two terminal Gal moieties. In some embodiments, about 2% to about 12% of the N-glycans comprise two terminal Gal moieties. In some embodiments, about 7% of the N-glycans comprise two terminal Gal moieties. In some embodiments, about 0.1% to about 6% of the N-glycans comprise three terminal Gal moieties. In some embodiments, about 1% to about 3% of the N-glycans comprise three terminal Gal moieties. In some embodiments, about 2% of the N-glycans comprise three terminal Gal moieties.

In some embodiments of any of the preceding aspects, the IL-22 polypeptide comprises N-glycans comprising galactose N-acetylglucosamine (LacNAc) repeats. In some embodiments, about 1% to about 10% of the N-glycans comprise LacNAc repeats. In some embodiments, about 3% to about 6% of the N-glycans comprise LacNAc repeats. In some embodiments, about 5% of the N-glycans comprise LacNAc repeats.

In some embodiments of any of the preceding aspects, the IL-22 polypeptide comprises N-glycans comprising fucosylated N-glycans. In some embodiments, about 60% to about 80% of the N-glycans are fucosylated. In some embodiments, about 65% to about 75% of the N-glycans are fucosylated. In some embodiments, about 70% of the N-glycans are fucosylated.

In some embodiments of any of the preceding aspects, the IL-22 polypeptide comprises N-glycans comprising afucosylated N-glycans. In some embodiments, about 10% to about 30% of the N-glycans are afucosylated. In some embodiments, about 15% to about 25% of the N-glycans are afucosylated. In some embodiments, about 20% of the N-glycans are afucosylated.

In some embodiments of any of the preceding aspects, the IL-22 polypeptide is glycosylated on amino acid residues Asn21, Asn35, Asn64, and/or Asn143 of SEQ ID NO:4. In some embodiments, the IL-22 polypeptide is glycosylated on amino acid residues Asn21, Asn35, Asn64, and Asn143 of SEQ ID NO:4. In some embodiments, the glycosylation occupancy on amino acid residue Asn21 of SEQ ID NO:4 is about 70% to about 90%. In some embodiments, the glycosylation occupancy on amino acid residue Asn21 of SEQ ID NO:4 is about 75% to about 85%. In some embodiments, the glycosylation occupancy on amino acid residue Asn21 of SEQ ID NO:4 is about 81% to about 84%. In some embodiments, the glycosylation occupancy on amino acid residue Asn21 of SEQ ID NO:4 is about 82%. In some embodiments, the glycosylation occupancy on amino acid residue Asn35 of SEQ ID NO:4 is about 90% to about 100%. In some embodiments, the glycosylation occupancy on amino acid residue Asn35 of SEQ ID NO:4 is about 95% to about 100%. In some embodiments, the glycosylation occupancy on amino acid residue Asn35 of SEQ ID NO:4 is about 100%. In some embodiments, the glycosylation occupancy on amino acid residue Asn64 of SEQ ID NO:4 is about 90% to about 100%. In some embodiments, the glycosylation occupancy on amino acid residue Asn64 of SEQ ID NO:4 is about 95% to about 100%. In some embodiments, the glycosylation occupancy on amino acid residue Asn64 of SEQ ID NO:4 is about 100%. In some embodiments, the glycosylation occupancy on amino acid residue Asn143 of SEQ ID NO:4 is about 15% to about 45%. In some embodiments, the glycosylation occupancy on amino acid residue Asn143 of SEQ ID NO:4 is about 25% to about 35%. In some embodiments, the glycosylation occupancy on amino acid residue Asn143 of SEQ ID NO:4 is about 32% to about 35%. In some embodiments, the glycosylation occupancy on amino acid residue Asn143 of SEQ ID NO:4 is about 33%.

In some embodiments, the IL-22 polypeptide is glycosylated on amino acid residues Asn21, Asn35, Asn64, and Asn143 of SEQ ID NO:4, wherein the glycosylation occupancy on amino acid residue Asn21 of SEQ ID NO:4 is about 81% to about 84%, the glycosylation occupancy on amino acid residue Asn35 of SEQ ID NO:4 is about 100%, the glycosylation occupancy on amino acid residue Asn64 of SEQ ID NO:4 is about 100% and the glycosylation occupancy on amino acid residue Asn143 of SEQ ID NO:4 is about 32% to about 35%. In some embodiments, the IL-22 polypeptide is glycosylated on amino acid residues Asn21, Asn35, Asn64, and Asn143 of SEQ ID NO:4, wherein the glycosylation occupancy on amino acid residue Asn21 of SEQ ID NO:4 is 81% to 84%, the glycosylation occupancy on amino acid residue Asn35 of SEQ ID NO:4 is 100%, the glycosylation occupancy on amino acid residue Asn64 of SEQ ID NO:4 is 100% and the glycosylation occupancy on amino acid residue Asn143 of SEQ ID NO:4 is 32% to 35%.

In another aspect, the invention an IL-22 Fc fusion protein having the N-glycan distribution shown in Table 12 or 13.

In some embodiments of any of the preceding aspects, the Fc region is not glycosylated. In some embodiments, the amino acid residue at position 297 as in the EU index of the Fc region is glycine (Gly). In some embodiments, the amino acid residue at position 297 as in the EU index of the Fc region is alanine (Ala). In some embodiments, the amino acid residue at position 299 as in the EU index of the Fc region is Ala, Gly, or valine (Val). In some embodiments, the Fc region comprises the CH2 and CH3 domain of IgG1 or IgG4. In some embodiments, the Fc region comprises the CH2 and CH3 domain of IgG4.

In some embodiments of any of the preceding aspects, the IL-22 Fc fusion protein comprises an amino acid sequence having at least 95% sequence identity to the amino acid sequence of SEQ ID NO:8. In some embodiments, the IL-22 Fc fusion protein comprises an amino acid sequence having at least 96% sequence identity to the amino acid sequence of SEQ ID NO:8. In some embodiments, the IL-22 Fc fusion protein comprises an amino acid sequence having at least 97% sequence identity to the amino acid sequence of SEQ ID NO:8. In some embodiments, the IL-22 Fc fusion protein comprises an amino acid sequence having at least 98% sequence identity to the amino acid sequence of SEQ ID NO:8. In some embodiments, the IL-22 Fc fusion protein comprises an amino acid sequence having at least 99% sequence identity to the amino acid of SEQ ID NO:8. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8. In some embodiments, the IL-22 Fc fusion protein comprises the amino acid sequence of SEQ ID NO:10 In some embodiments, the IL-22 Fc fusion protein consists of the amino acid sequence of SEQ ID NO:10. In some embodiments, the IL-22 Fc fusion protein comprises the amino acid sequence of SEQ ID NO:16. In some embodiments, the IL-22 Fc fusion protein consists of the amino acid sequence of SEQ ID NO:16. In some embodiments, the Fc region is not N-glycosylated.

In some embodiments of any of the preceding aspects, the IL-22 Fc fusion protein is a dimeric IL-22 Fc fusion protein. In other embodiments of any of the preceding aspects, the IL-22 Fc fusion protein is a monomeric IL-22 Fc fusion protein. In some embodiments, the IL-22 polypeptide is a human IL-22 polypeptide. In some embodiments, the IL-22 polypeptide comprises the amino acid sequence of SEQ ID NO:4.

In some embodiments of any of the preceding aspects, the linker comprises the amino acid sequence RVESKYGPP (SEQ ID NO: 44). In some embodiments, the linker consists of the amino acid sequence RVESKYGPP (SEQ ID NO: 44).

In some embodiments of any of the preceding aspects, the IL-22 Fc fusion protein binds to IL-22 receptor. In some embodiments, the IL-22 receptor is human IL-22 receptor. In some embodiments, the IL-22 Fc fusion protein binds to IL-22RA1 and/or IL-10R2. In some embodiments, the IL-22 Fc fusion protein binds to IL-22RA1.

In some embodiments of any of the preceding aspects, the IL-22 Fc fusion protein is produced by the method comprising the step of culturing a host cell capable of expressing the IL-22 Fc fusion protein under conditions suitable for expression of the IL-22 Fc fusion protein. In some embodiments, the method further comprises the step of obtaining the IL-22 Fc fusion protein from the cell culture or culture medium. In some embodiments, the host cell is a CHO cell.

In some embodiments of any of the preceding aspects, the IL-22 Fc fusion protein has an N-glycolylneuraminic acid (also known as Neu5Gc or NGNA) content of less than about 5 moles of NGNA per mole of the IL-22 Fc fusion protein. In some embodiments, the IL-22 Fc fusion protein has an NGNA content of less than 1 mole of NGNA per mole of the IL-22 Fc fusion protein.

In another aspect, the invention features a pharmaceutical composition comprising any of the IL-22 Fc fusion proteins described herein and at least one pharmaceutically acceptable carrier. In some embodiments, the IL-22 Fc fusion protein has a sialic acid content in the range of 8 to 12 moles of sialic acid per mole of the IL-22 Fc fusion protein. In some embodiments, the IL-22 Fc fusion protein has a sialic acid content in the range of 8 to 10 moles of sialic acid per mole of the IL-22 Fc fusion protein. In some embodiments, the IL-22 Fc fusion protein has a sialic acid content in the range of 8 to 9 moles of sialic acid per mole of the IL-22 Fc fusion protein. In some embodiments, the IL-22 Fc fusion protein has a sialic acid content of about 8 moles of sialic acid per mole of the IL-22 Fc fusion protein. In some embodiments, the IL-22 Fc fusion protein has a sialic acid content of 8 moles of sialic acid per mole of the IL-22 Fc fusion protein. In some embodiments, the IL-22 Fc fusion protein has a sialic acid content of about 9 moles of sialic acid per mole of the IL-22 Fc fusion protein. In some embodiments, the IL-22 Fc fusion protein has a sialic acid content of 9 moles of sialic acid per mole of the IL-22 Fc fusion protein. In some embodiments, the sialic acid is N-acetylneuraminic acid (NANA). In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein comprises the amino acid sequence of SEQ ID NO:16.

In some embodiments of any of the preceding aspects, the pharmaceutical composition further comprises an additional therapeutic agent. In some embodiments, the pharmaceutical composition further comprises a gelling agent. In some embodiments, the gelling agent is a polysaccharide. In some embodiments, the gelling agent is a cellulosic agent. In some embodiments, the gelling agent is methylcellulose, hydroxyethyl cellulose, carboxymethyl cellulose, hydroxypropyl cellulose, POE-POP block polymers, alginate, hyaluronic acid, polyacrylic acid, hydroxyethyl methylcellulose or hydroxypropyl methylcellulose. In some embodiments, the gelling agent is a hydroxypropyl methylcellulose. In some embodiments, the pharmaceutical composition is for topical administration.

In another aspect, the invention features a method of treating inflammatory bowel disease (IBD) in a subject in need thereof, the method comprising administering to the any of the IL-22 Fc fusion proteins described herein or any of the pharmaceutical compositions described herein. In some embodiments, the IBD is ulcerative colitis or Crohn's disease. In some embodiments, the IBD is ulcerative colitis. In some embodiments, the ulcerative colitis is moderate to severe ulcerative colitis. In some embodiments, the IBD is Crohn's disease.

In another aspect, the invention features a method of inhibiting microbial infection in the intestine, preserving goblet cells in the intestine during a microbial infection, enhancing epithelial cell integrity, epithelial cell proliferation, epithelial cell differentiation, epithelial cell migration or epithelial wound healing in the intestine, of a subject in need thereof, the method comprising administering to the subject any of the IL-22 Fc fusion proteins described herein or any of the pharmaceutical compositions described herein. In some embodiments, the epithelial cell is an intestinal epithelial cell.

In another aspect, the invention features a method of treating acute kidney injury or acute pancreatitis in a subject in need thereof, the method comprising administering to the subject any of the IL-22 Fc fusion proteins described herein or any of the pharmaceutical compositions described herein.

In another aspect, the invention features a method of accelerating or improving wound healing in a subject in need thereof, the method comprising administering to the subject any of the IL-22 Fc fusion proteins described herein or any of the pharmaceutical compositions described herein. In some embodiments, the wound is a chronic wound or an infected wound. In some embodiments, the subject is diabetic. In some embodiments, the diabetic subject has type II diabetes. In some embodiments, the wound is a diabetic foot ulcer. In some embodiments, the IL-22 Fc fusion protein or the pharmaceutical composition is administered until there is complete wound closure.

In another aspect, the invention features a method for preventing or treating a cardiovascular condition in a subject in need thereof, which condition includes a pathology of atherosclerotic plaque formation, the method comprising administering to the subject any of the IL-22 Fc fusion proteins described herein or any of the pharmaceutical compositions described herein. In some embodiments, the cardiovascular disease is coronary artery disease, coronary microvascular disease, stroke, carotid artery disease, peripheral artery disease, or chronic kidney disease. In some embodiments, the method further comprises slowing down the progression of atherosclerotic plaque formation or preventing indicia of atherosclerosis. In some embodiments, the indicia of atherosclerosis include plaque accumulation and/or vascular inflammation.

In another aspect, the invention features a method for treating metabolic syndrome in a subject in need thereof, the method comprising administering to the subject any of the IL-22 Fc fusion proteins described herein or any of the pharmaceutical compositions described herein. In some embodiments, the method further comprises reducing one or more risk factors associated with metabolic syndrome, including one or more of abdominal obesity, hyperglycemia, dyslipidemia, and hypertension. In some embodiments, the method further comprises reducing the level of bacterial lipopolysaccharide in the subject.

In another aspect, the invention features a method of treating acute endotoxemia, sepsis, or both, in a subject in need thereof, the method comprising administering the subject any of the IL-22 Fc fusion proteins described herein or any of the pharmaceutical compositions described herein. In some embodiments, the subject is in need of a change in HDL/LDL lipid profile.

In another aspect, the invention features a method of treating GVHD in a subject in need thereof, the method comprising administering the subject any of the IL-22 Fc fusion proteins described herein or any of the pharmaceutical compositions described herein.

In a further aspect, the invention features a composition comprising any of the IL-22 Fc fusion proteins described herein or the pharmaceutical compositions described herein for use as medicament.

In another aspect, the invention features a composition comprising any of the IL-22 Fc fusion proteins described herein or the pharmaceutical compositions described herein for use in (i) treating inflammatory bowel disease (IBD), (ii) inhibiting microbial infection in the intestine, preserving goblet cells in the intestine during a microbial infection, enhancing epithelial cell integrity, epithelial cell proliferation, epithelial cell differentiation, epithelial cell migration or epithelial wound healing in the intestine, (iii) treating acute kidney injury or acute pancreatitis, (iv) accelerating or improving wound healing in a subject in need thereof, (v) preventing or treating a cardiovascular disease such as coronary artery disease, coronary microvascular disease, stroke, carotid artery disease, peripheral artery disease, or chronic kidney disease, (vi) treating metabolic syndrome, (vii) treating acute endotoxemia or sepsis, or (viii) treating GVHD.

In yet another aspect, the invention features the use of a composition comprising any of the IL-22 Fc fusion proteins described herein or the pharmaceutical compositions described herein for the preparation of a medicament for use in (i) treating inflammatory bowel disease (IBD), (ii) inhibiting microbial infection in the intestine, preserving goblet cells in the intestine during a microbial infection, enhancing epithelial cell integrity, epithelial cell proliferation, epithelial cell differentiation, epithelial cell migration or epithelial wound healing in the intestine, (iii) treating acute kidney injury or acute pancreatitis, (iv) accelerating or improving wound healing in a subject in need thereof, (v) preventing or treating a cardiovascular disease such as coronary artery disease, coronary microvascular disease, stroke, carotid artery disease, peripheral artery disease, or chronic kidney disease, (vi) treating metabolic syndrome, (vii) treating acute endotoxemia or sepsis, or (viii) treating GVHD.

In some embodiments of any of the preceding aspects, the IL-22 Fc fusion protein has a sialic acid content of from about 8 to about 12 moles of sialic acid per mole of the IL-22 Fc fusion protein. In some embodiments, the IL-22 Fc fusion protein has a sialic acid content of from about 8 to about 10 moles of sialic acid per mole of the IL-22 Fc fusion protein. In some embodiments, the IL-22 Fc fusion protein has a sialic acid content of from about 8 to about 9 moles of sialic acid per mole of the IL-22 Fc fusion protein. In some embodiments, the IL-22 Fc fusion protein has a sialic acid content of about 8 moles of sialic acid per mole of the IL-22 Fc fusion protein. In some embodiments, the IL-22 Fc fusion protein has a sialic acid content of about 9 moles of sialic acid per mole of the IL-22 Fc fusion protein. In some embodiments, the sialic acid is N-acetylneuraminic acid (NANA). In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein comprises the amino acid sequence of SEQ ID NO:16.

In some embodiments of any of the preceding aspects, the IL-22 Fc fusion protein or the pharmaceutical composition is administered intravenously, subcutaneously, intraperitoneally, or topically. In some embodiments, the IL-22 Fc fusion protein or the pharmaceutical composition is administered intravenously. In some embodiments, the IL-22 Fc fusion protein or the pharmaceutical composition is administered subcutaneously.

In some embodiments of any of the preceding aspects, the subject is co-administered with at least one additional therapeutic agent.

In some embodiments of any of the preceding aspects, the subject is a human.

In another aspect, the invention features a method of making any of the IL-22 Fc fusion proteins described herein, the method comprising the following steps: (a) providing a host cell comprising a nucleic acid encoding any of the IL-22 Fc fusion proteins described herein; (b) culturing the host cell in a seed train medium under conditions suitable to form a seed train; (c) inoculating the seed train into an inoculum medium and culturing under conditions suitable to form an inoculum train; and (d) culturing the inoculum train in a production medium under conditions suitable to form a production culture, wherein the host cells of the production culture express the IL-22 Fc fusion protein, thereby making the IL-22 Fc fusion protein.

In another aspect, the invention features a method of making an IL-22 Fc fusion protein, the method comprising the following steps: (a) providing a host cell comprising a nucleic acid encoding a IL-22 Fc fusion protein, the IL-22 Fc fusion protein comprising an IL-22 polypeptide linked to an Fc region by a linker; (b) culturing the host cell in a seed train medium under conditions suitable to form a seed train; (c) inoculating the seed train in an inoculum medium under conditions suitable to form an inoculum train; and (d) culturing the inoculum train in a production medium under conditions time suitable to form a production culture, wherein the host cells of the production culture express the IL-22 Fc fusion protein, thereby making the IL-22 Fc fusion protein, wherein the IL-22 polypeptide is glycosylated, and wherein the IL-22 Fc fusion protein has a sialic acid content of from about 8 to about 12 moles of sialic acid per mole of the IL-22 Fc fusion protein. In some embodiments, the IL-22 Fc fusion protein has a sialic acid content in the range of 8 to 9 moles of sialic acid per mole of the IL-22 Fc fusion protein.

In some embodiments of any of the preceding aspects, the host cell is a frozen host cell, and step (a) further comprises thawing the frozen host cell in a seed train medium.

In some embodiments of any of the preceding aspects, the method further comprises passaging the inoculum train about 1 to about 10 times prior to step (d). In some embodiments, the inoculum train is passaged about 2 to about 6 times prior to step (d). In some embodiments, the inoculum train is passaged about 5 times prior to step (d).

In some embodiments of any of the preceding aspects, the seed train medium comprises a selection agent capable of selecting for the host cell. In some embodiments, the selection agent is methionine sulfoximine, methotrexate, or an antibiotic. In some embodiments, the selection agent is methionine sulfoximine. In some embodiments, the selection agent is an antibiotic. In some embodiments, the antibiotic is selected from blasticidin, geneticin, hygromycin B, puromycin, mycophenolic acid, or zeocin.

In some embodiments of any of the preceding aspects, the seed train medium, the inoculum medium, and/or the production medium comprises an antifoaming agent. In some embodiments, the antifoaming agent is simethicone emulsion, antifoam 204, antifoam A, antifoam B, antifoam C, antifoam Y-30, or antifoam SE-15. In some embodiments, the antifoaming agent is simethicone emulsion.

In some embodiments of any of the preceding aspects, the seed train medium, the inoculum medium, and/or the production medium includes a buffering agent, a cell protective agent, a polysaccharide, and/or an osmolality adjustment agent.

In some embodiments of any of the preceding aspects, step (b) is performed at a temperature of about 25° C. to about 40° C. In some embodiments, step (b) is performed at a temperature of about 35° C. to about 39° C. In some embodiments, step (b) is performed at a temperature of about 37° C.

In some embodiments of any of the preceding aspects, step (b) is performed in a spinner, a spin tube, a shake flask, a single-use bioreactor (e.g., a WAVE BIOREACTOR™ or an AMBR® bioreactor (e.g., an AMBR® 15 bioreactor)), or a seed train bioreactor. In some embodiments, step (b) is performed in a seed train spinner or a shake flask. In other embodiments, step (b) is performed in a single-use bioreactor (e.g., a WAVE BIOREACTOR™ or an AMBR® bioreactor (e.g., an AMBR® 15 bioreactor or an AMBR® 250 bioreactor)). In some embodiments, step (b) has a duration of about 1 day to about 12 days per passage. In some embodiments, step (b) has a duration of about 2 days to about 7 days per passage. In some embodiments, step (b) is performed in a seed train bioreactor.

In some embodiments of any of the preceding aspects, the pH of the seed train medium is about 6 to about 8. In some embodiments, the pH of the seed train medium is about 6.5 to about 7.5. In some embodiments, the pH of the seed train medium is about 7.15.

In some embodiments of any of the preceding aspects, the dissolved oxygen of the seed train medium is about 15% to about 50%. In some embodiments, the dissolved oxygen of the seed train medium is about 20% to about 40%. In some embodiments, the dissolved oxygen of the seed train medium is about 30%.

In some embodiments of any of the preceding aspects, step (b) has a duration of about 1 day to about 10 days. In some embodiments, step (b) has a duration of about 2 days to about 5 days.

In some embodiments of any of the preceding aspects, step (c) is performed at a temperature of about 25° C. to about 40° C. In some embodiments, step (c) is performed at a temperature of about 35° C. to about 39° C. In some embodiments, step (c) is performed at a temperature of about 37° C.

In some embodiments of any of the preceding aspects, step (c) is performed in one or more bioreactors. In some embodiments, step (c) is performed in three or four bioreactors.

In some embodiments of any of the preceding aspects, the pH of the inoculum medium is about 6 to about 8. In some embodiments, the pH of the inoculum medium is about 6.5 to about 7.5. In some embodiments, the pH of the inoculum medium is about 7.1.

In some embodiments of any of the preceding aspects, the dissolved oxygen of the inoculum medium is about 15% to about 50%. In some embodiments, the dissolved oxygen of the inoculum medium is about 20% to about 40%. In some embodiments, the dissolved oxygen of the inoculum medium is about 30%.

In some embodiments of any of the preceding aspects, step (c) has a duration of about 1 day to about 5 days. In some embodiments, step (c) has a duration of about 2 days to about 3 days.

In some embodiments of any of the preceding aspects, step (d) includes a temperature shift from an initial temperature to a post-shift temperature. In some embodiments, the initial temperature is about 25° C. to about 40° C. In some embodiments, the initial temperature is about 35° C. to about 39° C. In some embodiments, the initial temperature is about 37° C. In some embodiments, the post-shift temperature is about 25° C. to about 40° C. In some embodiments, the post-shift temperature is about 30° C. to about 35° C. In some embodiments, the post-shift temperature is about 33° C. In some embodiments, the temperature shift occurs over a period of about 12 h to about 120 h. In some embodiments, the temperature shift occurs over a period of about 48 h to about 96 h. In some embodiments, the temperature shift occurs over a period of about 72 h.

In some embodiments of any of the preceding aspects, the pH of the production medium is about 6 to about 8. In some embodiments, the pH of the production medium is about 6.5 to about 7.5. In some embodiments, the pH of the production medium is about 7.0. In some embodiments, step (d) is performed in a production bioreactor. In some embodiments, the dissolved oxygen of the production medium is about 15% to about 50%. In some embodiments, the dissolved oxygen of the production medium is about 20% to about 40%. In some embodiments, the dissolved oxygen of the production medium is about 30%.

In some embodiments of any of the preceding aspects, step (d) has a duration of about 5 days to about 25 days. In some embodiments, step (d) has a duration of about 7 days to about 16 days. In some embodiments, step (d) has a duration of about 8 days to about 16 days. In some embodiments, step (d) has a duration of about 12 days. In some embodiments, step (d) further comprises adding nutrients to the production medium by a nutrient feed.

In some embodiments of any of the preceding aspects, the host cell is a prokaryotic cell or a eukaryotic cell. In some embodiments, the host cell is a eukaryotic cell. In some embodiments, the eukaryotic cell is a mammalian cell. In some embodiments, the mammalian cell is a Chinese hamster ovary (CHO) cell. In some embodiments, the CHO cell is a suspension-adapted CHO cell.

In some embodiments of any of the preceding aspects, the method comprises the following step: (e) harvesting a cell culture fluid comprising the IL-22 Fc fusion protein from the production culture. In some embodiments, step (e) comprises cooling the production culture. In some embodiments, step (e) comprises cooling the production culture to about 2° C. to about 8° C. In some embodiments, step (e) comprises removing the host cells from the production medium by centrifugation to form the cell culture fluid. In some embodiments, step (e) further comprises filtering the cell culture fluid.

In some embodiments of any of the preceding aspects, the method comprises the following step: (f) purifying the IL-22 Fc fusion protein in the cell culture fluid. In some embodiments, step (f) comprises the following substeps: (i) contacting the cell culture fluid to an affinity chromatographic support, optionally washing the affinity chromatographic support with a wash buffer, eluting the IL-22 Fc fusion protein from the affinity chromatographic support with a first elution buffer to form an affinity pool, and optionally inactivating viruses in the affinity pool; (ii) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and (iii) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool. In some embodiments, step (f) further comprises the following substep: (iv) concentrating the purified product pool to form a concentrated product pool. In some embodiments, step (f) further comprises the following substep: (v) ultrafiltering the purified product pool. In some embodiments, ultrafiltering comprises filtering the purified product pool with a 10-kDa composite regenerated cellulose ultrafiltration membrane. In some embodiments, step (f) further comprises the following substep: (vi) exchanging the buffer of the concentrated product pool to form a ultrafiltration and diafiltration (UFDF) pool comprising the IL-22 Fc fusion protein. In some embodiments, the buffer of the concentrated product pool is exchanged with a diafiltration buffer comprising 0.01 M sodium phosphate, pH 7.2, final concentration. In some embodiments, step (f) further comprises the following substep: (vii) conditioning the UFDF pool with a formulation buffer to form a conditioned UFDF pool comprising the IL-22 Fc fusion protein. In some embodiments, substep (i) further comprises inactivating viruses by adding a detergent to the cell culture fluid prior to contacting the cell culture fluid to the affinity column. In some embodiments, substep (i) comprises inactivating viruses by adding a detergent to the affinity pool. In some embodiments, the detergent is TRITON® X-100 or TRITON® CG110. In some embodiments, the final concentration of the detergent is about 0.01% to about 2% (v/v). In some embodiments, the final concentration of the detergent is about 0.1% to about 1% (v/v). In some embodiments, the final concentration of the detergent is about 0.3% to about 0.5% (v/v). In some embodiments, the final concentration of the detergent is about 0.5%. In some embodiments, the virus inactivation is performed at about 12° to about 25° C. In some embodiments, inactivating viruses has a duration of greater than about 0.5 h.

In another aspect, the invention features a method of purifying an IL-22 Fc fusion protein, the method comprising: (a) providing a cell culture fluid comprising an IL-22 Fc fusion protein and optionally inactivating viruses in the cell culture fluid; (b) contacting the cell culture fluid to an affinity chromatographic support, optionally washing the affinity chromatographic support with a wash buffer, and eluting the IL-22 Fc fusion protein from the affinity chromatographic support with a first elution buffer to form an affinity pool, and optionally inactivating viruses in the affinity pool; (c) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and (d) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool. In some embodiments, the IL-22 polypeptide is glycosylated, and wherein the IL-22 Fc fusion protein has a sialic acid content of from about 8 to about 12 moles of sialic acid per mole of the IL-22 Fc fusion protein.

In some embodiments of any of the preceding aspects, the affinity chromatographic support comprises a protein A resin, a protein G resin, or an IL-22 receptor resin. In some embodiments, the protein A resin is a MABSELECT SURE® resin. In some embodiments, the wash buffer comprises 0.4 M potassium phosphate, pH 7.0, final concentration.

In some embodiments of any of the preceding aspects, the first elution buffer comprises 0.3 M L-arginine hydrochloride, 0.013 M sodium phosphate, pH 3.8, final concentration.

In some embodiments of any of the preceding aspects, the anion-exchange chromatographic support comprises a strong anion exchanger with multimodal functionality resin. In some embodiments, the anion-exchange chromatographic support comprises a CAPTO™ adhere resin. In some embodiments, the first equilibration buffer comprises 0.04 M sodium acetate, pH 5.8, final concentration.

In some embodiments, the second elution buffer is a gradient elution buffer. In some embodiments, the gradient elution buffer comprises 0.04 M sodium acetate, pH 5.8 as Buffer A of the gradient elution buffer and 0.04 M sodium acetate, 0.3M sodium sulfate pH 5.8 as Buffer B of the gradient, wherein the gradient starts at 10% of Buffer B. In some embodiments, the second equilibration buffer comprises 0.025 M MOPS, 0.3 M sodium sulfate, pH 7.0, final concentration.

In another aspect, any of the IL-22 Fc fusion proteins described herein or any of the pharmaceutical compositions described herein can be used in a method of treating IBD in a subject in need thereof. In some embodiments, the IBD is ulcerative colitis (UC) or Crohn's disease. In some embodiments, the IBD is ulcerative colitis (UC). In some embodiments, the ulcerative colitis is moderate to severe ulcerative colitis. In some embodiments, the IBD is Crohn's disease.

In another aspect, any of the IL-22 Fc fusion proteins described herein or any of the pharmaceutical compositions described herein can be used in a method of inhibiting microbial infection in the intestine, preserving goblet cells in the intestine during a microbial infection, enhancing epithelial cell integrity, epithelial cell proliferation, epithelial cell differentiation, epithelial cell migration or epithelial wound healing in the intestine, of a subject in need thereof. In some embodiments, the epithelial cell is an intestinal epithelial cell.

In another aspect, any of the IL-22 Fc fusion proteins described herein or any of the pharmaceutical compositions described herein can be used in a method of treating acute kidney injury or acute pancreatitis in a subject in need thereof.

In another aspect, any of the IL-22 Fc fusion proteins described herein or any of the pharmaceutical compositions described herein can be used in a method of accelerating or improving wound healing in a subject in need thereof. In some embodiments, the wound is a chronic wound or an infected wound. In some embodiments, the subject is diabetic. In some embodiments, the diabetic subject has type II diabetes. In some embodiments, the wound is a diabetic foot ulcer. In some embodiments, the IL-22 Fc fusion protein or the pharmaceutical composition is administered until there is complete wound closure.

In another aspect, any of the IL-22 Fc fusion proteins described herein or any of the pharmaceutical compositions described herein can be used in a method for preventing or treating a cardiovascular condition in a subject in need thereof, which condition includes a pathology of atherosclerotic plaque formation. In some embodiments, the cardiovascular disease is coronary artery disease, coronary microvascular disease, stroke, carotid artery disease, peripheral artery disease, or chronic kidney disease. In some embodiments, the method comprises slowing down the progression of atherosclerotic plaque formation or preventing indicia of atherosclerosis. In some embodiments, the indicia of atherosclerosis include plaque accumulation and/or vascular inflammation.

In another aspect, any of the IL-22 Fc fusion proteins described herein or any of the pharmaceutical compositions described herein can be used in a method for treating metabolic syndrome in a subject in need thereof. In some embodiments, the method further comprises reducing one or more risk factors associated with metabolic syndrome, including one or more of abdominal obesity, hyperglycemia, dyslipidemia, and hypertension. In some embodiments, the method further comprises reducing the level of bacterial lipopolysaccharide in the subject.

In another aspect, any of the IL-22 Fc fusion proteins described herein or any of the pharmaceutical compositions described herein can be used in a method of treating acute endotoxemia, sepsis, or both, in a subject in need thereof.

In some embodiments of any of the preceding aspects, the subject is in need of a change in HDL/LDL lipid profile.

In some embodiments of any of the preceding aspects, the IL-22 Fc fusion protein or the pharmaceutical composition is to be administered intravenously, subcutaneously, intraperitoneally, or topically. In some embodiments, the IL-22 Fc fusion protein or the pharmaceutical composition is to be administered intravenously. In some embodiments, the IL-22 Fc fusion protein or the pharmaceutical composition is to be administered subcutaneously.

In some embodiments of any of the preceding aspects, the subject is to be co-administered with at least one additional therapeutic agent. In some embodiments of any of the preceding aspects, the subject is a human.

Each and every embodiment can be combined unless the context clearly suggests otherwise. Each and every embodiment can be applied to each and every aspect of the invention unless the context clearly suggests otherwise.

Specific embodiments of the present invention will become evident from the following more detailed description of certain preferred embodiments and the claims.

### BRIEF DESCRIPTION OF THE DRAWINGS

FIG. 1A is a schematic diagram showing a schematic design configuration of an exemplary dimeric IL-22 Fc fusion protein having two human interleukin-22 (IL-22) polypeptides each fused to a human immunoglobulin G4 (IgG4) Fc region. The two Fc regions are connected by two inter-chain disulfide linkages. Also depicted is the presence of four N-glycans on each IL-22 polypeptide.

FIG. 1B is an annotated amino acid sequence of the human interleukin-22 (IL-22) cytokine region of the IL-22 Fc fusion protein. IL-22 receptor binding regions are shown in bold. The glycosylation sites at Asn²¹, Asn³⁵, Asn⁶⁴, and Asn¹⁴³ are shown as N.

FIG. 1C is an annotated amino acid sequence of the human immunoglobulin G4 (IgG4) Fc region of the IL-22 Fc fusion protein. The Fc mutation of N81 G to remove the N-glycan, minimizing the potential for Fc effector function, is denoted by G.

FIG. 2A is a chromatogram showing the mass spectrometry profile of intact, deglycosylated IL-22 Fc fusion protein Reference Standard Batch, confirming the molecular mass predicted for the intact molecule. The species at 85,265 Da and 85,393 Da are IL-22 Fc fusion protein with one C-terminal lysine residue and two C-terminal lysine residues, respectively.

FIG. 2B is a chromatogram showing the mass spectrometry profile of reduced, deglycosylated IL-22 Fc fusion protein Reference Standard Batch, confirming the molecular mass predicted for the reduced molecule. The species at 42,706 Da is IL-22 Fc fusion protein with one C-terminal lysine residue.

FIGS. 3A-3B are a series of chromatograms showing an expanded view of the chromatographic profile of the tryptic digested IL-22 Fc fusion protein Reference Standard Batch between 0 and 50 minutes (3A) and 50-110 minutes (3B).

FIGS. 3C-3D are a series of chromatograms showing an expanded view of the comparison of the chromatographic profiles of the tryptic digested IL-22 Fc fusion protein Reference Standard Batch and Clinical Batches 1, 2, and 3 between 0 and 50 minutes (3C) and 50-110 minutes (3D), verifying the primary structure and demonstrating batch-to-batch consistency of peptide pattern.

FIGS. 4A-4B are a series of chromatograms showing the full-scale view (4A) and expanded view (4B) of the size exclusion high performance liquid chromatography (SE-HPLC) profile of the IL-22 Fc fusion protein Reference Standard Batch and Clinical Batches 1, 2, and 3 providing quantitative information about the molecular size heterogeneity of the IL-22 Fc fusion protein. Differences observed in the apex of the main peak are attributed to glycosylation.

FIGS. 5A-5B are a series of chromatograms showing the full-scale view (5A) and expanded view (5B) of the capillary electrophoresis sodium dodecyl sulfate, non-gel sieving (CE-SDS-NGS) analysis of the non-reduced, fluorescently labeled IL-22 Fc fusion protein Reference Standard Batch and Clinical Batches 1, 2, and 3, demonstrating the presence of one major peak with consistent peak patterns and percent corrected peak areas (CPA). Differences in the shape of the main peak are attributed to glycosylation.

FIGS. 5C-5D are a series of chromatograms showing the full-scale view (5C) and expanded view (5D) of the CE-SDS-NGS analysis of the reduced, fluorescently labeled IL-22 Fc fusion protein Reference Standard Batch and Clinical Batches 1, 2, and 3, demonstrating the presence of one major peak with consistent peak patterns and percent corrected peak areas (CPA). Differences in the shape of the main peak are attributed to glycosylation. IRS=incompletely reduced species.

FIGS. 6A-6B show the SYPRO® Ruby-stained sodium dodecyl sulfate-polyacrylamide gel electrophoresis (SDS-PAGE) of reduced (6A) and non-reduced (6B) samples of IL-22 Fc fusion protein Reference Standard Batch and Clinical Batches 1, 2, and 3, demonstrating consistent banding patterns across all batches. Lane 1: Precision plus unstained protein standard (Biorad), Lane 2: 8 ng bovine serum albumin (BSA), Lane 3: 2 ng BSA, Lane 4: IL-22 Fc fusion protein Reference Standard Batch, Lane 5: IL-22 Fc fusion protein Clinical Batch 1, Lane 6: IL-22 Fc fusion protein Clinical Batch 2, and Lane 7: IL-22 Fc fusion protein Clinical Batch 3.

FIGS. 7A-7B are a series of chromatograms showing the full-scale view (7A) and expanded view (7B) of the imaged capillary isoelectric focusing (ICIEF) of native IL-22 Fc fusion protein Reference Standard Batch and Clinical Batches 1, 2, and 3.

FIGS. 7C-7D are a series of chromatograms showing the full-scale view (7C) and expanded view (7D) of the ICIEF of carboxypeptidase B (CpB)-treated IL-22 Fc fusion protein Reference Standard Batch and Clinical Batches 1, 2, and 3 heterogeneity following the removal of C-terminal lysines. Minor differences in the pl of the profiles are instrument related and have no effect on percent peak area.

FIG. 7E is a chromatogram showing the ICIEF profile of native and CpB-treated IL-22 Fc fusion protein Reference Standard Batch.

FIGS. 8A-8B are a series of chromatograms showing the relative N-glycan distribution of the IL-22 Fc fusion protein Reference Standard Batch and Clinical Batches 1, 2, and 3 by 2-aminobenzoic acid hydrophilic interaction liquid chromatography-ultra-high-performance liquid chromatography (2-AA HILIC-UHPLC) from 0-40 minutes (8A) and 40-75 minutes (8B).

FIGS. 8C-8D are a series of graphs showing the relative N-glycan distribution, represented as peak area percentage (%), of the IL-22 Fc fusion protein Reference Standard Batch and Clinical Batches 1, 2, and 3 (8C) and Reference Standard Batch and Clinical Batches 2, 3, 4, 5, and 6 (8D) by 2-AA HILIC-UHPLC.

FIG. 9 is a graph showing the relative N-glycan distribution, represented as peak area %, of the IL-22 Fc fusion protein Reference Standard Batch and Clinical Batches 1, 2, and 3 at site Asn21 by Lys-C peptide mapping and LC-MS.

FIG. 10 is a circular dichroism (CD) spectra of the IL-22 Fc fusion protein Reference Standard Batch and Clinical Batches 1, 2, and 3, showing that there are no discernable differences in higher order structural characteristics between the batches.

FIG. 11 is a schematic overview of the cell-based IL-22 Fc fusion protein binding potency assay using the human colon cancer cell line Colo 205, which endogenously express IL-22 receptor and stably express the STAT3 luciferase reporter gene.

FIG. 12A is a graph demonstrating the relationship between sialic acid content and potency in an in vitro assay as compared to the cell-based IL-22 Fc fusion protein binding potency assay.

FIG. 12B is a graph comparing the potency of the IL-22 Fc fusion protein Reference Standard Batch and Clinical Batches 2, 4, 5, and 6 before and after desialylation with sialidase. For all desialylated samples and Reference Standard Batches, error bars represent % difference of n=2. For potency at release values, error bars are the standard deviation of n=3. Asterisks (*) indicate estimate of potency, result outside of the validated assay range.

FIG. 13 is a series of graphs examining the potency of the IL-22 Fc fusion protein Reference Standard Batch and Clinical Batches 2, 4, 5, and 6 following deglycosylation with PNGase F enzyme. The process control was exposed to same incubations as samples, but no PNGase F was added.

FIG. 14 is a graph comparing the serum IL-22 Fc fusion protein concentration over time in mice for sialic acid variants of IL-22 Fc fusion protein following a single intravenous (IV) administration.

FIG. 15 is a graph showing the opposing effects of the impact of sialic acid levels on in vitro potency of the IL-22 Fc fusion protein and on its exposure in mice following a single IV administration of the indicated IL-22 Fc fusion protein sialic acid variants.

FIG. 16A is a graph showing the impact of REG3β response to sialic acid variants of the IL-22 Fc fusion protein following a single IV administration in mice, presented as serum REG3β concentration (ng/mL) over time.

FIG. 16B is a graph showing the relationship between IL-22 Fc fusion protein exposure and serum REG3β response to IL-22 Fc fusion protein sialic acid variants following a single IV administration in mice, presented as REG3β AUC (day×ng/mL) versus IL-22Fc fusion protein AUC (day×ng/mL).

FIG. 17 is a cell culture process flow chart showing the in-process controls, process stage, and media for the production of IL-22 Fc fusion protein.

FIG. 18 is a purification process flow chart showing the process stage and in-process controls for the purification of IL-22 Fc fusion protein.

FIG. 19 shows an amino acid sequence alignment of mature IL-22 from different mammalian species: human (GenBank Accession No.Q9GZX6, SEQ ID NO:4, chimpanzee (GenBank Accession No.XP_003313906, SEQ ID NO:48), orangutan (GenBank Accession No. XP_002823544, SEQ ID NO:49), mouse (GenBank Accession No. Q9JJY9, SEQ ID NO:50) and dog (GenBank Accession No. XP_538274, SEQ ID NO:51).

FIG. 20 is a graph showing the change in sialic acid levels over the course of cell culture. Each line plot shows a different production run. A reverse phase high performance liquid chromatography (RP-HPLC) assay was used to determine the sialic acid levels. Sialic levels per mole of IL-22 Fc protein (shown in the y-axis) decrease with increasing cell culture duration (shown in the x-axis).

### DETAILED DESCRIPTION OF EMBODIMENTS OF THE INVENTION

### I. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, reference to “an isolated peptide” means one or more isolated peptides.

Throughout this specification and claims, the word “comprise,” or variations such as “comprises” or “comprising” will be understood to imply the inclusion of a stated integer or group of integers but not the exclusion of any other integer or group of integers.

The term “IL-22 Fc fusion protein” or “IL-22 fusion protein” or “IL-22 Ig fusion protein” as used herein refers to a fusion protein in which IL-22 protein or polypeptide is linked, directly or indirectly, to an IgG Fc region. In some embodiments, the IL-22 protein or polypeptide is glycosylated. In particular embodiments, the IL-22 protein or polypeptide is sialylated. In certain preferred embodiments, the IL-22 Fc fusion protein comprises a human IL-22 protein or polypeptide linked to a human IgG Fc region. In certain preferred embodiments, the IL-22 Fc fusion protein comprises two human interleukin-22 (IL-22) polypeptides each fused to a human immunoglobulin G4 (IgG4) Fc region, wherein the two Fc regions are connected by two inter-chain disulfide linkages. In certain embodiments, the human IL-22 protein comprises the amino acid sequence of SEQ ID NO:4. However, it is understood that minor sequence variations such as insertions, deletions, substitutions, especially conservative amino acid substitutions of IL-22 or Fc that do not affect the function and/or activity of IL-22 or IL-22 Fc fusion protein are also contemplated by the invention. The IL-22 Fc fusion protein of the invention can bind to IL-22 receptor, which can lead to IL-22 receptor downstream signaling. In certain embodiments, the IL-22 Fc fusion protein is capable of binding to IL-22 receptor, and/or is capable of leading to IL-22 receptor downstream signaling. The functions and/or activities of the IL-22 Fc fusion protein can be assayed by methods known in the art, including without limitation, ELISA, ligand-receptor binding assay and Stat3 luciferase assay. In certain embodiments, the invention provides an IL-22 Fc fusion protein that binds to IL-22 receptor, in which the binding can lead to IL-22 receptor downstream signaling, the IL-22 Fc fusion protein comprising an amino acid sequence having at least 95% sequence identity to the amino acid sequence selected from the group consisting of SEQ ID NO:8, SEQ ID NO:10, SEQ ID NO:12, SEQ ID NO:14, and SEQ ID NO:16, and wherein the Fc region is not glycosylated. In certain particular embodiments, the Fc region of the IL-22 fusion protein does not possess effector activities (e.g., does not bind to FcγIIIR) or exhibits substantially lower effector activity than a whole (e.g., wild-type) IgG antibody. In certain other embodiments, the Fc region of the IL-22 Fc fusion protein does not trigger cytotoxicity such as antibody-dependent cellular cytotoxicity (ADCC) or complement dependent cytotoxicity (CDC). Unless otherwise specified, “IL-22 fusion protein,” “IL-22 Fc fusion,” “IL-22 Ig fusion protein,” “IL-22 Fc fusion protein,” or “IL-22 Fc” are used interchangeably throughout this application.

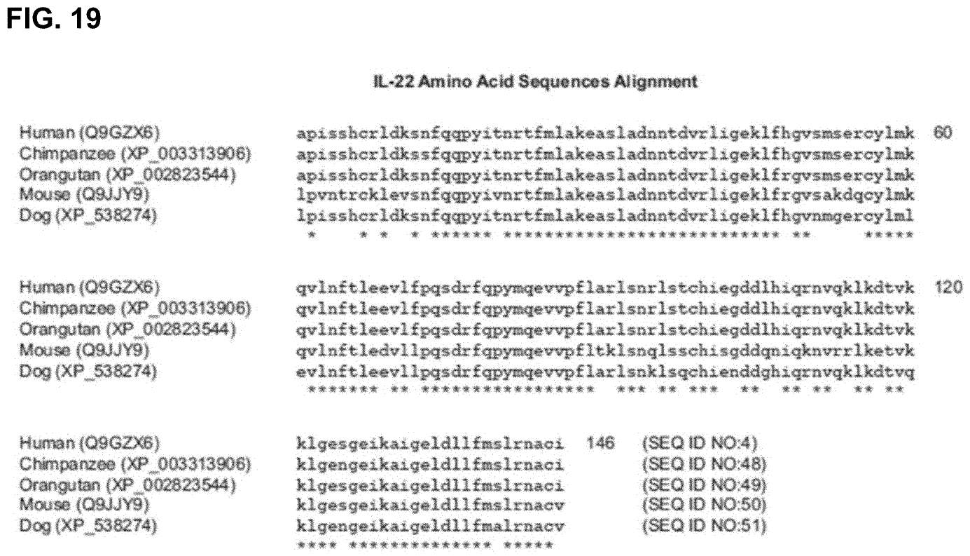

The term “IL-22” or “IL-22 polypeptide” or “IL-22 protein” as used herein, broadly refers to any native IL-22 from any mammalian source, including primates (e.g. humans) and rodents (e.g., mice and rats), unless otherwise indicated. The term encompasses “full-length,” unprocessed IL-22 as well as any forms of IL-22 that result from processing in the cell. For example, both full-length IL-22 containing the N-terminal leader sequence and the mature form IL-22 are encompassed by the current invention. The leader sequence (or signal peptide) can be the endogenous IL-22 leader sequence or an exogenous leader sequence of another mammalian secretary protein. In certain embodiments, the leader sequence can be from a eukaryotic or prokaryotic secretary protein. The term also encompasses naturally occurring variants of IL-22, e.g., splice variants or allelic variants. The amino acid sequence of an exemplary human IL-22 is shown in SEQ ID NO:4 (mature form, without a signal peptide). In certain embodiments, the amino acid sequence of full-length IL-22 protein with the endogenous leader sequence is provided in SEQ ID NO:71; while in other embodiments, the amino acid sequence of mature IL-22 protein with an exogenous leader sequence is provided in SEQ ID NO:2. Minor sequence variations, especially conservative amino acid substitutions of IL-22 that do not affect the IL-22's function and/or activity (e.g., binding to IL-22 receptor), are also contemplated by the invention. FIG. 19 shows an amino acid sequence alignment of mature IL-22 from several exemplary mammalian species. The asterisks indicate highly conserved amino acid residues across species that are likely important for the functions and/or activities of IL-22. Accordingly, in certain embodiments, the IL-22 Fc fusion protein comprises an IL-22 polypeptide comprising an amino acid sequence having at least 95%, at least 96%, at least 97%, at least 98%, or at least 99% sequence identity to SEQ ID NO:4. In certain other embodiments, the IL-22 protein has 95% or more sequence identity to SEQ ID NO:71, 96% or more sequence identity to SEQ ID NO:71, 97% or more sequence identity to SEQ ID NO:71; 98% or more sequence identity to SEQ ID NO:71; or 99% or more sequence identity to SEQ ID NO:71. The IL-22 polypeptides described herein can be isolated from a variety of sources, such as from human tissue or from another source, or prepared by recombinant or synthetic methods.

The term “IL-22 receptor” or “IL-22R” refers to a heterodimer consisting of IL-22R1 and IL-10R2 or naturally occurring allelic variants thereof. See, e.g., Ouyang et al., 2011, Annu. Rev. Immunol. 29:159-63. IL-10R2 is ubiquitously expressed by many cell types, and IL-22R1 is expressed only in innate cells such as epithelial cells, hepatocytes and keratinocytes. IL-22R1 is also known as IL-22Rα1 or IL-22Rα1. IL-22R1 may be paired with other polypeptides to form heterodimeric receptors for other IL-10 family members, for example IL-20 or IL-24. See, e.g., Ouyang et al., 2011, supra. The full-length amino acid sequence of an exemplary IL-22R1 polypeptide is shown in SEQ ID NO:81. This full-length sequence of IL-22R1 includes an N-terminal signal sequence (amino acids 1-15) which is cleaved in the final functional molecule (an exemplary amino acid sequence of which is shown in SEQ ID NO:82). The full-length amino acid sequence of an exemplary IL10R2 polypeptide is shown in SEQ ID NO:83. This full-length sequence of IL10R2 includes an N-terminal signal sequence (amino acids 1-19) which is cleaved in the final functional molecule (an exemplary amino acid sequence of which is shown in SEQ ID NO:84).

A “native sequence IL-22 polypeptide” or a “native sequence IL-22R polypeptide” refers to a polypeptide comprising the same amino acid sequence as a corresponding IL-22 or IL-22R polypeptide derived from nature. Such native sequence IL-22 or IL-22R polypeptides can be isolated from nature or can be produced by recombinant or synthetic means. The terms specifically encompass naturally-occurring truncated or secreted forms of the specific IL-22 or IL-22R polypeptide (e.g., an IL-22 lacking its associated signal peptide), naturally-occurring variant forms (e.g., alternatively spliced forms), and naturally-occurring allelic variants of the polypeptide. In various embodiments of the invention, the native sequence IL-22 or IL-22R polypeptides disclosed herein are mature or full-length native sequence polypeptides. An exemplary full length native human IL-22 is shown in SEQ ID NO:70 (DNA) and SEQ ID NO:71 (protein). While the IL-22 and IL-22R polypeptide sequences are shown to begin with methionine residues designated herein as amino acid position 1, it is conceivable and possible that other methionine residues located either upstream or downstream from the amino acid position 1 can be employed as the starting amino acid residue for the IL-22 or IL-22R polypeptides.

An “IL-22 variant,” an “IL-22R variant,” an “IL-22 variant polypeptide,” or an “IL-22R variant polypeptide” means an active IL-22 or IL-22R polypeptide as defined above having at least about 80% amino acid sequence identity with a full-length native sequence IL-22 or IL-22R polypeptide sequence. Ordinarily, an IL-22 or IL-22R polypeptide variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity, and alternatively at least about 99% amino acid sequence identity to a full-length or mature native sequence IL-22 or IL-22R polypeptide sequence.

The term “Fc region,” “Fc domain,” or “Fc” refers to a C-terminal non-antigen binding region of an immunoglobulin heavy chain that contains at least a portion of the constant region. The term includes native Fc regions and variant Fc regions. In certain embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, Md., 1991.

In certain embodiments, Fc region refers to an immunoglobulin IgG heavy chain constant region comprising a hinge region (starting at Cys226), an IgG CH2 domain, and CH3 domain. The term “hinge region” or “hinge sequence” as used herein refers to the amino acid sequence located between the linker and the CH2 domain. In certain embodiments, the hinge region comprises the amino acid sequence CPPCP (SEQ ID NO:31). In certain embodiments, the hinge region for IL-22 IgG4 Fc fusion protein comprises the CPPCP sequence (SEQ ID NO:31), a sequence found in the native IgG1 hinge region, to facilitate dimerization. In certain other embodiments, the Fc region starts at the hinge region and extends to the C-terminus of the IgG heavy chain. In certain particular embodiments, the Fc region comprises the Fc region of human IgG1, IgG2, IgG3 or IgG4. In certain particular embodiments, the Fc region comprises the CH2 and CH3 domain of IgG4. In certain other particular embodiments, the Fc region comprises the CH2 and CH3 domain of IgG1.

In certain embodiments, the IgG CH2 domain starts at Ala 231. In certain other embodiments, the CH3 domain starts at Gly 341. It is understood that the C-terminus Lys residue of human IgG can be optionally absent. It is also understood that conservative amino acid substitutions of the Fc region without affecting the desired structure and/or stability of Fc is contemplated within the scope of the invention.

In certain embodiments, the IL-22 is linked to the Fc region via a linker. In certain particular embodiments, the linker is a peptide that connects the C-terminus of IL-22 to the Fc region as described herein. In certain embodiments, native IgG sequences are present in the linker and/or hinge region to minimize and/or avoid the risk of immunogenicity. In other embodiments, minor sequence variations can be introduced to the native sequences to facilitate manufacturing. IL-22 Fc fusion constructs comprising exogenous linker or hinge sequences that exhibit high activity (as measured, e.g., by a luciferase assay) are also within the scope of the invention. In certain embodiments, the linker comprises an amino acid sequence that is 8-20 amino acids, 8-16, 8-15, 8-14, 8-13, 8-12, 8-11, 8-10, 8-9, 10-11, 10-12, 10-13, 10-14, 10-15, 10-16, 11-16, 8, 9, 10, 11, 12, 13, 14, 15, or 16 amino acids long. In certain other embodiments, the linker comprises the amino acid sequence DKTHT (SEQ ID NO:32). In certain particular embodiments, the linker does not comprise the sequence Gly-Gly-Ser (SEQ ID NO:45), Gly-Gly-Gly-Ser (SEQ ID NO:46), or Gly-Gly-Gly-Gly-Ser (SEQ ID NO:47).

In certain embodiments, the IL-22 Fc fusion protein comprises an IL-22 polypeptide linked to an Fc region by a linker. The term “linked to” or “fused to” refers to a covalent bond, e.g., a peptide bond, formed between two moieties.

The terms “glycosylation” and “glycosylated” as used herein refers to the presence of a carbohydrate (e.g., an oligosaccharide or a polysaccharide, also referred to as a “glycan”) attached to biological molecule (e.g., a protein or a lipid). In particular embodiments, glycosylation refers to the presence of a glycan (e.g., an N-glycan) attached to a protein (e.g., an IL-22 Fc fusion protein) or a portion of a protein of interest (e.g., an IL-22 polypeptide moiety of an IL-22 Fc fusion protein). N-linked glycosylation refers to the attachment of the carbohydrate moiety to the side-chain of an asparagine residue. The tripeptide sequences, asparagine-X-serine and asparagine-X-threonine, wherein X is any amino acid except proline, are recognition sequences for enzymatic attachment of the carbohydrate moiety to the asparagine side chain. O-linked glycosylation refers to the attachment of one of the sugars N-acetylgalactosamine, galactose, or xylose to a hydroxyamino acid, most commonly serine or threonine, although 5-hydroxyproline or 5-hydroxylysine can also be involved in O-linked glycosylation. For a review of glycosylation, see, e.g., Varki et al., Essentials of Glycobiology, 3^{rd} Edition, Cold Spring Harbor Laboratory Press, 2015-2017.

The terms “aglycosylated” and “not glycosylated,” as used interchangeably herein, refer to a protein or a portion of a protein of interest (e.g., the Fc region of an IL-22 Fc fusion protein) that is not glycosylated (e.g., not N-glycosylated). It is to be understood that in some embodiments, a portion of a protein of interest (e.g., an IL-22 Fc fusion protein) is glycosylated (e.g., the IL-22 polypeptide portion of an IL-22 Fc fusion protein), while another portion of the protein of interest is not glycosylated (e.g., the Fc region of the IL-22 Fc fusion protein).

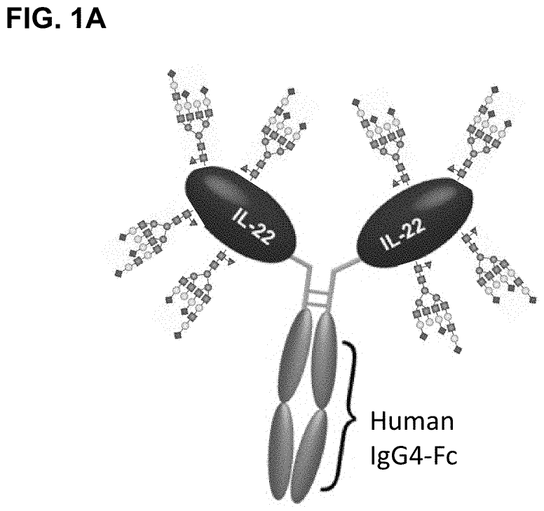

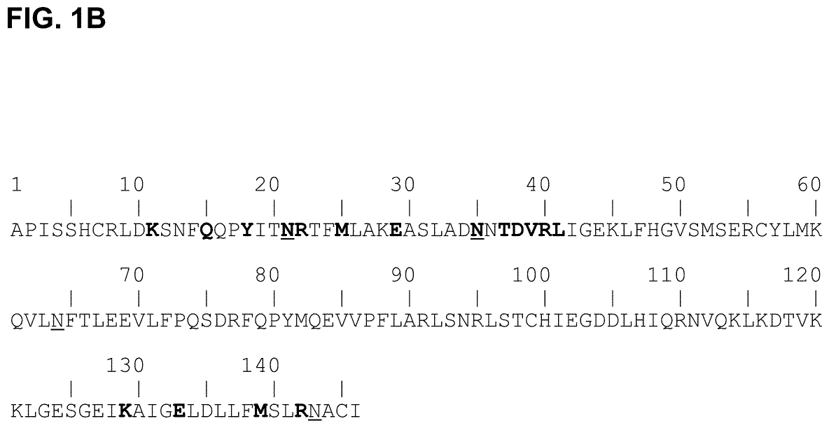

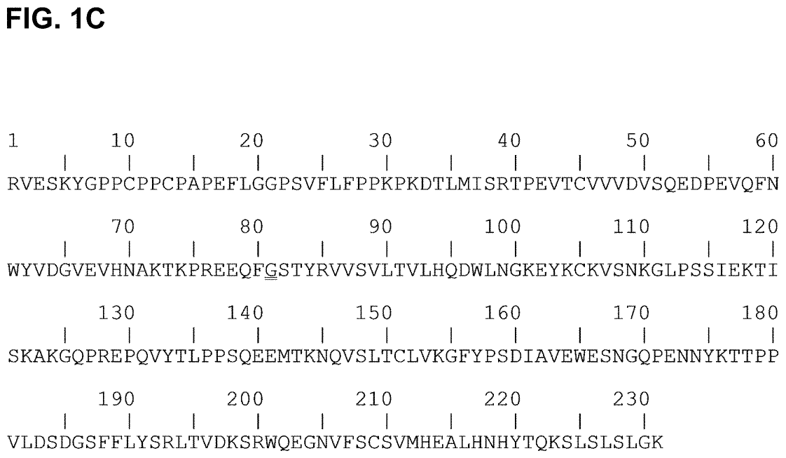

In some embodiments, provided herein are IL-22 Fc fusion proteins in which the Fc region or CH2 domain is not glycosylated. In certain embodiments, the N-glycosylation site in the CH2 domain is mutated to prevent glycosylation. For example, an IL-22 Fc fusion protein with an aglycosylated Fc region can be made by mutagenizing the amino acid residue at position 297 as in the EU index in the CH2 domain of the Fc region (e.g., N297) (also referred to as residue N81, see, e.g., FIG. 1C). In certain embodiments, the glycosylation in the CH2 domain of the Fc region can be eliminated by altering the glycosylation consensus site, i.e., Asn at position 297 followed by any amino acid residue (in the case of human IgG, Ser) and Thr. The glycosylation site can be altered by amino acid insertions, deletions, and/or substitutions. For example, one or more amino acid residues can be inserted between Asn and Ser or between Ser and Thr to alter the original glycosylation site, wherein the insertions do not regenerate an N-glycosylation site. In certain particular embodiments, the amino acid residue at position 297 as in the EU index (e.g., the N-glycosylated site in Fc) within the CH2 domain of human IgG Fc is mutated to abolish the glycosylation site. In certain particular embodiments, the amino acid residue at position 297 as in the EU index (e.g., N297) is changed to Gly, Ala, Gln, Asp, or Glu. In some particular embodiments, the amino acid residue at position 297 as in the EU index (e.g., N297) is changed to Gly or Ala. In other particular embodiments, the amino acid residue at position 297 as in the EU index (e.g., N297) is changed to Gly. In certain other embodiments, the amino acid residue at position 299 as in the EU index can be substituted with another amino acid, for example, Ala, Val, or Gly. In certain particular embodiments, the mutations that result in an aglycosylated Fc do not affect the structure and/or stability of the IL-22 Fc fusion protein.

In certain embodiments, the IL-22 Fc fusion protein comprises an Fc region in which the amino acid residue at position 297 as in the EU index in the CH2 domain is mutated. In certain embodiments, the amino acid residue at position 297 as in the EU index is changed to Gly or Ala, preferably to Gly. In certain other embodiments, the amino acid residue at position 297 as in the EU index is deleted. In certain embodiments, the IL-22 Fc fusion protein comprising an Fc having an amino acid substitution at the amino acid residue at position 297 as in the EU index is aglycosylated or not glycosylated.

In other embodiments, the N-glycan attached to the wild type amino acid residue at position 297 as in the EU index (e.g., N297) can be removed enzymatically, e.g., by deglycosylation. Suitable glycolytic enzymes include without limitation, peptide-N-glycosidase (PNGase).

The term “glycosylation occupancy” as used herein refers to the probability that a protein is glycosylated at a particular glycosylation site (e.g., an Asn residue of a consensus glycosylation site) or the percentage of proteins in a population of proteins that are glycosylated at a particular glycosylation site. For example, an IL-22 polypeptide may be glycosylated on amino acid residues Asn21, Asn35, Asn64, and/or Asn143 of SEQ ID NO: 4. In a further specific example, (a) the percent N-glycosylation site occupancy at residue Asn21 may be in the range of 70 to 90; (b) the percent N-glycosylation site occupancy at residue Asn35 may be in the range of 90 to 100; (c) the percent N-glycosylation site occupancy at residue Asn64 may be in the range of 90 to 100; and/or (d) the percent N-glycosylation site occupancy at residue Asn143 may be in the range of 25 to 35.

The terms “sialylation” and “sialylated” refers to the presence of sialic acid on a protein or a portion of a protein of interest, particularly as a component of a glycan (e.g., N-glycan) chain attached to a protein. Sialic acid (also referred to herein as a “sialic acid moiety”) refers generally to N- or O-substituted derivatives of neuraminic acid. N-acetylneuraminic acid (5-acetamido-2-keto-3,5-dideoxy-D-glycero-D-galactonononic acid; also known as NANA or Neu5Ac) is the most common sialic acid in mammals. Other exemplary sialic acids include, without limitation, 2-keto-3-deoxy-D-glycero-D-galactonononic acid (also known as Kdn), N-glycolylneuraminic acid (also known as Neu5Gc or NGNA), neuraminic acid (also known as Neu), and 2-deoxy-2,3-didehydro-Neu5Ac (also known as Neu2en5Ac). Free sialic acid (Sia) can be used for glycan synthesis after activation onto the nucleotide donor CMP-Sia. Transfer of Sia from CMP-Sias onto newly synthesized glycoconjugates (e.g., glycoproteins) in the Golgi system of eukaryotes is catalyzed by a family of linkage-specific sialyl-transferases (STs). Sialic acids are typically the terminating residues of glycan (e.g., N-glycan) branches. In some embodiments, sialic acids can occupy internal positions within glycans, most commonly when one sialic acid residue is attached to another. For a review of sialylation and sialic acid, see, e.g., Chapter 15 of Varki et al., Essentials of Glycobiology, 3rd Edition, Cold Spring Harbor Laboratory Press, 2015-2017.

The term “sialic acid content” refers to the level or amount of sialylation of a glycosylated protein (e.g., an IL-22 Fc fusion protein) or a portion of a protein of interest. In some embodiments, an IL-22 Fc fusion protein has a sialic acid content of from about 4 to about 16 moles (e.g., about 4, about 5, about 6, about 7, about 8, about 9, about 10, about 11, about 12, about 13, about 14, about 15, or about 16 moles) of sialic acid per mole of the IL-22 Fc fusion protein. In some embodiments, an IL-22 Fc fusion protein has a sialic acid content of about 8, 9, 10, 11, or 12 moles of sialic acid per mole of the IL-22 Fc fusion protein.

The term “average sialic acid content” with respect to a composition containing an IL-22 Fc fusion protein (e.g., a pharmaceutical composition or a batch) according to the invention refers to the total number of moles of sialic acid in the composition per mole of IL-22 Fc fusion protein in the composition. Thus, for example, such a composition may contain a heterogeneous pool of IL-22 Fc fusion proteins with individual IL-22 Fc fusion proteins within the composition having varying levels of sialylation (e.g., in the range of 0-25 moles of sialic acid per mole of IL-22 Fc fusion protein). Unless indicated otherwise, all values for sialic acid content, including average sialic acid content, described herein refer to dimeric IL-22 Fc fusion proteins.

The term “batch” as used herein refers to the product of a run of a production process, including, for example, IL-22 Fc fusion proteins or compositions thereof. For example, the methods described herein can be used to produce batches of IL-22 Fc fusion proteins or compositions thereof. The batches can be selected for release (i.e., for distribution or sale) according to the methods described herein, for example, by assessing the average sialic acid content of the batch.

The term “afucosylation,” “afucosylated,” “defucosylation,” or “defucosylated” refers to the absence or removal of core-fucose from an N-glycan, e.g., an N-glycan attached to a protein (e.g., an IL-22 polypeptide) or a portion of a protein (e.g., the CH2 domain of Fc).

The term “dimeric IL-22 Fc fusion protein” refers to a dimer in which each monomer comprises an IL-22 Fc fusion protein. The term “monomeric IL-22 Fc fusion protein” refers to a dimer in which one monomer comprises an IL-22 Fc fusion protein (the IL-22 Fc arm), while the other monomer comprises an Fc region without the IL-22 polypeptide (the Fc arm). Accordingly, the dimeric IL-22 Fc fusion protein is bivalent with respect to IL-22R binding, whereas the monomeric IL-22 Fc fusion protein is monovalent with respect to IL-22R binding. The heterodimerization of the monomeric IL-22 Fc fusion protein can be facilitated by methods known in the art, including without limitation, heterodimerization by the knob-into-hole technology. The structure and assembly method of the knob-into-hole technology can be found in, e.g., U.S. Pat. Nos. 5,821,333, 7,642,228, US 2011/0287009, and PCT/US2012/059810, hereby incorporated by reference in their entireties. This technology was developed by introducing a “knob” (or a protuberance) by replacing a small amino acid residue with a large one in the CH3 domain of one Fc, and introducing a “hole” (or a cavity) in the CH3 domain of the other Fc by replacing one or more large amino acid residues with smaller ones. In certain embodiments, the IL-22 Fc fusion arm comprises a knob, and the Fc only arm comprises a hole.

The preferred residues for the formation of a knob are generally naturally occurring amino acid residues and are preferably selected from arginine (R), phenylalanine (F), tyrosine (Y), and tryptophan (W). Most preferred are tryptophan and tyrosine. In one embodiment, the original residue for the formation of the knob has a small side chain volume, such as alanine, asparagine, aspartic acid, glycine, serine, threonine or valine. Exemplary amino acid substitutions in the CH3 domain for forming the knob include without limitation the T366W, T366Y, or F405W substitution.

The preferred residues for the formation of a hole are usually naturally occurring amino acid residues and are preferably selected from alanine (A), serine (S), threonine (T), and valine (V). In one embodiment, the original residue for the formation of the hole has a large side chain volume, such as tyrosine, arginine, phenylalanine, or tryptophan. Exemplary amino acid substitutions in the CH3 domain for generating the hole include without limitation the T366S, L368A, F405A, Y407A, Y407T, and Y407V substitutions. In certain embodiments, the knob comprises T366W substitution, and the hole comprises the T366S/L368A/Y407V substitutions. In certain particular embodiments, the Fc region of the monomeric IL-22 Fc fusion protein comprises an IgG1 Fc region. In certain particular embodiments, the monomeric IL-22 IgG1 Fc fusion comprises an IL-22 Fc knob arm and an Fc hole arm. In certain embodiments, the IL-22 Fc knob arm comprises a T366W substitution (SEQ ID NO:61), and the Fc hole arm comprises T366S, L368A, and Y407V (SEQ ID NO:62). In certain other embodiments, the Fc region of both arms further comprises an N297G or N297A mutation. In certain embodiments, the monomeric IL-22 Fc fusion protein is expressed in E. coli cells. It is understood that other modifications to the Fc region known in the art that facilitate heterodimerization are also contemplated and encompassed by the instant application.

The term “wound” refers to an injury, especially one in which the skin or another external surface is torn, pierced, cut, or otherwise broken.

The term “ulcer” is a site of damage to the skin or mucous membrane that is often characterized by the formation of pus, death of tissue, and is frequently accompanied by an inflammatory reaction.

The terms “intestine” or “gut” as used interchangeably herein broadly encompasses the small intestine and large intestine.

The term “accelerating wound healing” or “acceleration of wound healing” refers to the increase in the rate of healing, e.g., a reduction in time until complete wound closure occurs or a reduction in time until a percent (%) reduction in wound area occurs.

A “diabetic wound” is a wound that associated with diabetes.

A “diabetic ulcer” is an ulcer that is associated with diabetes.

A “chronic wound” refers to a wound that does not heal. See, e.g., Lazarus et al., Definitions and guidelines for assessment of wounds and evaluation of healing, Arch. Dermatol. 130:489-93 (1994). Chronic wounds include, but are not limited to, e.g., arterial ulcers, diabetic ulcers, pressure ulcers or bed sores, venous ulcers, and the like. An acute wound can develop into a chronic wound. Acute wounds include, but are not limited to, wounds caused by, e.g., thermal injury (e.g., burn), trauma, surgery, excision of extensive skin cancer, deep fungal and bacterial infections, vasculitis, scleroderma, pemphigus, toxic epidermal necrolysis, and the like. Thus, in certain embodiments, a chronic wound is an infected wound. A “normal wound” refers to a wound that undergoes normal wound healing repair. “Affinity” refers to the strength of the sum total of non-covalent interactions between a single binding site of a molecule (e.g., a ligand or an antibody) and its binding partner (e.g., a receptor or an antigen). Unless indicated otherwise, as used herein, “binding affinity” refers to intrinsic binding affinity which reflects a 1:1 interaction between members of a binding pair (e.g., IL-22 Fc fusion protein and IL-22 receptor). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

The term “potency,” as used herein with respect to an IL-22 Fc fusion protein, refers to the ability of an IL-22 Fc fusion protein to bind to an IL-22R (e.g., IL-22-R1a, or a portion thereof, e.g., the extracellular domain) and/or to activate downstream IL-22R signaling (e.g., STAT3 signaling). In some embodiments, potency is assessed in a receptor binding assay or a cell-based binding assay, for example, as described in Example 2. In some embodiments, potency is assessed using in vivo assays, e.g., as described in Example 2. In some embodiments, potency is compared to a reference IL-22 Fc fusion protein, for example, an IL-22 Fc fusion protein having the N-glycan distribution shown in Table 12 and/or Table 13.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g., scFv), and multispecific antibodies formed from antibody fragments.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Effector functions” or “effector activities” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation. In certain embodiments, the IL-22 Fc fusion protein does not exhibit any effector function or any detectable effector function. In certain other embodiments, the IL-22 Fc fusion protein exhibits substantially reduced effector function, e.g., about 50%, 60%, 70% 80%, or 90% reduced effector function.

An “effective amount” or “therapeutically effective amount” of an agent, e.g., a pharmaceutical formulation, refers to an amount effective, at dosages and for periods of time necessary, to achieve the desired therapeutic or prophylactic result.

For example, in the case of a cardiovascular disease or condition, the therapeutically effective amount of the IL-22 Fc fusion protein can reduce the degree of atherosclerotic plaque formation; reduce the size of the atherosclerotic plaque(s); inhibit (i.e., slow to some extent and preferably stop) atherosclerotic plaque; inhibit (i.e., slow to some extent and preferably stop) thrombosis or rupture of an atherosclerotic plaque; and/or relieve to some extent one or more of the symptoms associated with the disease or condition.

By “reduce or inhibit” is meant the ability to cause an overall decrease preferably of 20% or greater, more preferably of 50% or greater, and most preferably of 75%, 85%, 90%, 95%, or greater. Reduce or inhibit can refer to the symptoms of the disorder being treated, the presence or size of atherosclerotic plaques, or the number of atherosclerotic plaque(s).

A “suboptimal amount” refers to the amount less than the optimal amount of a therapeutic agent typically used for a certain treatment. When two therapeutic agents are given to a subject, either concurrently or sequentially, each therapeutic agent can be given at a suboptimal amount as compared to the treatment when each therapeutic agent is given alone. For example, in certain embodiments, the subject in need of IBD treatment is administered with the pharmaceutical composition comprising the IL-22 Fc fusion protein of the invention and a dexamethasone at a suboptimal amount.

The terms “full-length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. The transformed cell includes transiently or stably transformed cell. Progeny may not be completely identical in nucleic acid content to a parent cell, but may contain mutations. Mutant progeny that have the same function or biological activity as screened or selected for in the originally transformed cell are included herein. In certain embodiments, the host cell is transiently transfected with the exogenous nucleic acid. In certain other embodiments, the host cell is stably transfected with the exogenous nucleic acid.

An “immunoconjugate” is an antibody or a fragment of an antibody conjugated to one or more heterologous molecule(s), including but not limited to a cytotoxic agent.

An “individual,” “subject,” or “patient” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual, subject or patient is a human.

An “isolated” IL-22 Fc fusion protein is one which has been separated from the environment of a host cell that recombinantly produces the fusion protein. In some embodiments, an IL-22 Fc fusion protein is purified to greater than 80%, 85%, 90%, 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC) approaches.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “isolated nucleic acid encoding an IL-22 Fc fusion protein” refers to one or more nucleic acid molecules encoding an IL-22 Fc fusion protein, including such nucleic acid molecule(s) in a single vector or separate vectors, such nucleic acid molecule(s) transiently or stably transfected into a host cell, and such nucleic acid molecule(s) present at one or more locations in a host cell.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

A “native sequence Fc region” comprises an amino acid sequence identical to the amino acid sequence of an Fc region found in nature. Native sequence human Fc regions include, without limitation, a native sequence human IgG1 Fc region (non-A and A allotypes); native sequence human IgG2 Fc region; native sequence human IgG3 Fc region; and native sequence human IgG4 Fc region, as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith. In certain embodiments, the variant Fc region is not glycosylated.

A “disorder,” a “disease,” or a “condition,” as used interchangeably herein, is any condition that would benefit from treatment with a composition (e.g., a pharmaceutical composition) described herein, e.g., a composition (e.g., a pharmaceutical composition) that includes an IL-22 Fc fusion protein. This includes chronic and acute disorders or diseases including those pathological conditions which predispose the mammal to the disorder in question. In some embodiments, the disorder an IL-22 associated disorder. Exemplary disorders include, but are not limited to, IBD (e.g., UC or Crohn's disease), microbial infection, acute kidney injury, acute pancreatitis, wounds, cardiovascular conditions, metabolic syndrome, acute endotoxemia, and sepsis.

The terms “inflammatory bowel disorder,” “inflammatory bowel disease,” and “IBD,” as used interchangeably herein, are used herein in the broadest sense and includes all diseases and pathological conditions the pathogenesis of which involves recurrent inflammation in the intestine, including small intestine and colon. IBD includes, e.g., ulcerative colitis and Crohn's disease. IBD is not limited to UC and CD. The manifestations of the disease include but not limited to inflammation and a decrease in epithelial integrity in the intestine.

The terms “cardiovascular disease” or “cardiovascular disorder” are used herein in the broadest sense and includes all diseases and pathological conditions the pathogenesis of which involves abnormalities of the blood vessels, such as, for example, atherosclerotic plaque formation (including stable or unstable/vulnerable plaques), atherosclerosis, arteriosclerosis, arteriolosclerosis, and elevated systemic lipopolysaccharide (LPS) exposure. The term additionally includes diseases and pathological conditions that benefit from the inhibition of the formation of atherosclerotic plaques. Cardiovascular diseases include, without limitation, coronary artery atherosclerosis, coronary microvascular disease, stroke, carotid artery disease, peripheral arterial disease, ischemia, coronary artery disease (CAD), acute coronary syndrome (ACS), coronary heart disease (CHD), conditions associated with CAD and CHD, cerebrovascular disease, peripheral vascular disease, aneurysm, vasculitis, venous thrombosis, diabetes mellitus, metabolic syndromechronic kidney disease, remote tissue injury after ischemia and reperfusion, and cardiopulmonary bypass. Specifically included within this group are all cardiovascular diseases associated with the occurrence, development, or progression of which can be controlled by the inhibition of the atherosclerotic plaque formation.

The term “cardiovascular condition” is used herein in the broadest sense and includes all cardiovascular conditions and diseases the pathology of which involves atherosclerotic plaque formation (including stable or unstable/vulnerable plaques), atherosclerosis, arteriosclerosis, arteriolosclerosis, and elevated systemic lipopolysaccharide (LPS) exposure. Specifically included within this group are all cardiovascular conditions and diseases associated with the atherosclerotic plaque formation, the occurrence, development, or progression of which can be controlled by the inhibition of the atherosclerotic plaque formation. The term specifically includes diseases and pathological conditions that benefit from the inhibition of the formation of atherosclerotic plaques. Cardiovascular conditions include, without limitation, coronary artery atherosclerosis, coronary microvascular disease, stroke, carotid artery disease, peripheral arterial disease, ischemia, coronary artery disease (CAD), coronary heart disease (CHD), conditions associated with CAD and CHD, cerebrovascular disease and conditions associated with cerebrovascular disease, peripheral vascular disease and conditions associated with peripheral vascular disease, aneurysm, vasculitis, venous thrombosis, diabetes mellitus, metabolic syndromechronic kidney disease, remote tissue injury after ischemia and reperfusion, and cardiopulmonary bypass. “Conditions associated with cerebrovascular disease” as used herein include, for example, transient ischemic attack (TIA) and stroke. “Conditions associated with peripheral vascular disease” as used herein include, for example, claudication. Specifically included within this group are all cardiovascular diseases and conditions associated with the occurrence, development, or progression of which can be controlled by the inhibition of the atherosclerotic plaque formation.

Atherosclerotic plaque formation can occur as a result of an innate immune response to metabolic endotoxemia, which is characterized by elevated levels of systemic lipopolysaccharides (LPS) that originate from gut microbiota and a loss of functional integrity in the gut mucosal barrier. The innate immune response to endotoxemia results in the low-grade chronic inflammation that is responsible for plaque formation.

The term “metabolic syndrome” is used herein in the broadest sense. Metabolic syndrome includes the co-occurrence in an adult subject of several metabolic risk factors, including at least three of the following five traits: abdominal obesity, which can be, for example, a waist circumference in men of greater than or equal to 90 cm and in women greater than or equal to 80 cm; elevated serum triglycerides, which can be, for example, greater than or equal to 150 mg/dL, or drug treatment for elevated triglycerides; reduced serum HDL cholesterol level, which can be, for example, below 40 mg/dL in men and below 50 mg/dL in women, or drug treatment for low HDL cholesterol; hypertension, which can be, for example, systolic blood pressure greater than 130 mmHg and diastolic blood pressure greater than 85 mmHg, or drug treatment for hypertension; and elevated fasting plasma glucose, which can be, for example, greater than or equal to 100 mg/dL, drug treatment for elevated glucose, or previously diagnosed type 2 diabetes.

For children over 16 years old, the above criteria for adults can be used. For children between 10-16 year old, metabolic syndrome includes the co-occurrence in a subject of several metabolic risk factors, including at least three of the following five traits: abdominal obesity, which can be, for example, a waist circumference greater than 90th percentile; elevated serum triglycerides, which can be, for example, greater than or equal to 110 mg/dL, greater than 95th percentile, or drug treatment for elevated triglycerides; reduced serum HDL cholesterol level, which can be, for example, below 40 mg/dL, less than 5th percentile, or drug treatment for low HDL cholesterol; hypertension, which can be, for example, systolic blood pressure greater than 130 mmHg and diastolic blood pressure greater than 85 mmHg, greater than 90th percentile, or drug treatment for hypertension; and elevated fasting plasma glucose, which can be, for example, greater than or equal to 100 mg/dL, impaired glucose tolerance, drug treatment for elevated glucose, or previously diagnosed type 2 diabetes.

Generally speaking, the risk factors that co-occur in metabolic syndrome include obesity (such as abdominal obesity), hyperglycemia, dyslipidemia, insulin resistance, and/or hypertension. All these risk factors promote the development of atherosclerotic cardiovascular disease, diabetes, or both. Metabolic syndrome can also feature chronic adipose tissue inflammation.

Metabolic syndrome can be recognized as a proinflammatory, prothrombic state, and can be associated with elevated levels of one or more of C-reactive protein, IL-6, LPS, and plasminogen activator inhibitor 1; such markers can be associated with an increased risk for subsequent development of atherosclerotic cardiovascular disease, diabetes, or both.

Metabolic syndrome can be associated with several obesity-related disorders, including one or more of fatty liver disease with steatosis, fibrosis, and cirrhosis, hepatocellular and intrahepatic cholangiocarcinoma, chronic kidney disease, polycystic ovary syndrome, sleep disordered breathing, including obstructive sleep apnea, and hyperuricemia and gout.

The term “insulin-related disorder” encompasses diseases or conditions characterized by impaired glucose tolerance. In one embodiment, the insulin-related disorder is diabetes mellitus including, without limitation, Type I (insulin-dependent diabetes mellitus or IDDM), Type II (non-insulin dependent diabetes mellitus or NIDDM) diabetes, gestational diabetes, and any other disorder that would be benefited by agents that stimulate insulin secretion. In another embodiment, the insulin-related disorder is characterized by insulin resistance.

The term “sepsis” is used in its broadest sense and can encompass a systemic inflammatory state caused by severe infection. Sepsis can caused by the immune system's response to a serious infection, most commonly bacteria, but also fungi, viruses, and parasites in the blood, urinary tract, lungs, skin, or other tissues.

The term “acute endotoxemia” is used in its broadest sense and can encompass the condition of increased plasma bacterial lipopolysaccharide (LPS). Acute endotoxemia in turn could result in sepsis. Increased LPS in systemic circulation will induce low grade chronic inflammation, activating the endogenous protective host response to elevate plasma lipids that, in the chronic condition contributes to diet induced obesity, insulin resistance and atherosclerosis, and eventual CVD events.

The term “graft-versus-host disease (GVHD)” refers to a complication of allogeneic stem cell transplantation. In GVHD, donor hematopoietic stem cells recognize the transplant recipient as foreign and attack the patient's tissues and organs, which can impair the tissue or organ's function or cause it to fail. As used herein, GVHD includes, for example, acute GVHD or chronic GVHD. Further, non-limiting examples include intestinal GVHD.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

For example, with regard to IBD, “treatment” can refer to a decrease in the likelihood of developing IBD, a decrease in the rate of developing IBD, and a decrease in the severity of the disease. As another example, with regard to atherosclerotic plaque formation, “treatment” can refer to a decrease in the likelihood of developing atherosclerotic plaque deposits, a decrease in the rate of development of deposits, a decrease in the number or size of existing deposits, or improved plaque stability. Those in need of treatment include those already with the disorder as well as those in which the disorder is to be prevented. Desirable effects of treatment include, but are not limited to, preventing occurrence or recurrence of disease, alleviating symptoms, diminishing any direct or indirect pathological consequences of the disease, preventing the disease, decreasing the rate of disease progression, ameliorating or palliating the disease state, and causing remission or improved prognosis. In some embodiments, IL-22 Fc fusion protein of the invention are used to delay development of a disease or to slow the progression of a disease.

In certain embodiments, a “subject in need thereof” in the context of preventing or treating a cardiovascular condition refers to a subject diagnosed with a cardiovascular disease or cardiovascular condition (CVD) or metabolic syndrome or exhibiting one or more conditions associated with CVD or metabolic syndrome, a subject who has been diagnosed with or exhibited one or more conditions associated with CVD or metabolic syndrome in the past, or a subject who has been deemed at risk of developing CVD or metabolic syndrome or one or more conditions associated with CVD or metabolic syndrome in the future due to hereditary or environmental factors. Therefore, in certain embodiments, a subject in need thereof can be a subject exhibiting a CVD or metabolic syndrome or a condition associated with a CVD or metabolic syndrome or a subject that has exhibited a CVD or metabolic syndrome or a condition associated with a CVD or metabolic syndrome in the past or has been deemed at risk for developing a CVD or metabolic syndrome or a condition associated with a CVD or metabolic syndrome in the future.

In treatment of a cardiovascular disease or condition, a therapeutic agent can directly alter the magnitude of response of a component of the immune response, or render the disease more susceptible to treatment by other therapeutic agents, e.g., antibiotics, antifungals, anti-inflammatory agents, chemotherapeutics, etc. In treatment of an arterial disease, treatment might, for example, prevent or slow down the progression of a disease. Thus, treatment of an arterial disease specifically includes the prevention, inhibition, or slowing down of the development of the condition, or of the progression from one stage of the condition to another, more advanced stage, or into a more severe, related condition.

The “pathology” of a disease or condition includes all phenomena that compromise the well-being of the subject. In the case of a cardiovascular disease or condition, this includes, without limitation, atherosclerotic plaque formation (including stable or unstable/vulnerable plaques), atherosclerosis, arteriosclerosis, arteriolosclerosis, and elevated systemic lipopolysaccharide (LPS) exposure.

“Alleviation,” “alleviating,” or equivalents thereof, refer to both therapeutic treatment and prophylactic or preventative measures, wherein the object is to ameliorate, prevent, slow down (lessen), decrease or inhibit a disease or condition, e.g., the formation of atherosclerotic plaques. Those in need of treatment include those already with the disease or condition as well as those prone to having the disease or condition or those in whom the disease or condition is to be prevented.

“Chronic” administration refers to administration of an agent(s) in a continuous mode as opposed to an acute mode, so as to maintain the initial therapeutic effect for an extended period of time.

“Intermittent” administration is treatment that is not consecutively done without interruption, but rather is cyclic in nature.

The term “package insert” is used to refer to instructions customarily included in commercial packages of therapeutic products, that contain information about the indications, usage, dosage, administration, combination therapy, contraindications, and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Below are examples of how to calculate the % amino acid sequence identity of the amino acid sequence designated “Comparison Protein” or “Reference Protein” to the amino acid sequence designated “IL-22,” wherein “IL-22” represents the amino acid sequence of an IL-22 polypeptide of interest, “Comparison Protein” represents the amino acid sequence of a polypeptide against which the “IL-22” polypeptide of interest is being compared, and “X,” “Y,” and “Z” each represent different amino acid residues.

| | | |
|---|---|---|
| IL-22 | XXXXXXXXXXXXXXX | (Length = 15 amino acids) |
| Reference Protein | XXXXXYYYYYYY | (Length = 12 amino acids) |

% amino acid sequence identity =
(the number of identically matching amino acid residues between the two polypeptide sequences)
divided by (the total number of amino acid residues of the IL-22 polypeptide) =
5 divided by 15 = 33.3%

| | | |
|---|---|---|
| IL-22 | XXXXXXXXXX | (Length = 10 amino acids) |
| Reference Protein | XXXXXYYYYYYZZYZ | (Length = 15 amino acids) |

% amino acid sequence identity =
(the number of identically matching amino acid residues between the two polypeptide
sequences) divided by (the total number of amino acid residues of the IL-22 polypeptide) =
5 divided by 10 = 50%

The term “agonist” is used in the broadest sense and includes any molecule that partially or fully mimics a biological activity of an IL-22 polypeptide. Also encompassed by “agonist” are molecules that stimulate the transcription or translation of mRNA encoding the polypeptide.

Suitable agonist molecules include, e.g., agonist antibodies or antibody fragments; a native polypeptide; fragments or amino acid sequence variants of a native polypeptide; peptides; antisense oligonucleotides; small organic molecules; and nucleic acids that encode polypeptides agonists or antibodies. Reference to “an” agonist encompasses a single agonist or a combination of two or more different agonists.

The term “IL-22 agonist” is used in the broadest sense, and includes any molecule that mimics a qualitative biological activity (as hereinabove defined) of a native sequence IL-22 polypeptide. IL-22 agonists specifically include IL-22-Fc or IL-22 Ig polypeptides (immunoadhesins), but also small molecules mimicking at least one IL-22 biological activity. Preferably, the biological activity is binding of the IL-22 receptor, interacting with IL-22BP, facilitating an innate immune response pathway, or in the case of a cardiovascular disease or condition, to affect the formation of atherosclerotic plaques, in particular to inhibit formation of atherosclerotic plaque formation. Inhibition of plaque formation can be assessed by any suitable imaging method known to those of ordinary skill in the art.

IL-22R1 pairs with other proteins to form heterodimers as the receptors for certain IL-10 family members. See Ouyang et al., 2011, supra. Thus, in certain embodiments, IL-22 agonists may include an IL-22 receptor agonist, including a cytokine (or a fusion protein or agonist thereof) that binds to and triggers downstream signaling of the IL-22R1. In certain embodiments, the IL-22 agonists include an IL-22R1 agonist, including without limitation an anti-IL-22R1 agonist antibody; an IL-20 agonist, including without limitation IL-20 polypeptide or IL-20 Fc fusion protein; and an IL-24 agonist, including without limitation IL-24 polypeptide or IL-24 fusion protein. In certain other embodiments, the IL-22R1 agonists include an IL-19 agonist, including without limitation IL-19 polypeptide or IL-19 Fc fusion protein; and an IL-26 agonist, including without limitation IL-26 polypeptide or IL-26 Fc fusion protein. Exemplary sequences for IL-19 (GenBank Accession No. AAG16755.1, SEQ ID NO:77), IL-20 (GenBank Accession No. AAH69311.1, SEQ ID NO:78), IL-24 (GenBank Accession No. AAH09681.1, SEQ ID NO:79) and IL-26 (GenBank Accession No. NP_060872.1, SEQ ID NO:80) are provided herein. In certain embodiments, an IL-19 polypeptide comprises the amino acid sequence of SEQ ID NO:77 or the mature protein without the signal peptide. In certain other embodiments, an IL-20 polypeptide comprises the amino acid sequence of SEQ ID NO:78 or the mature protein without the signal peptide. In yet other embodiments, an IL-24 polypeptide comprises the amino acid sequence of SEQ ID NO:79 or the mature protein without the signal peptide. In certain other embodiments, an IL-26 polypeptide comprises the amino acid sequence of SEQ ID NO:80 or the mature protein without the signal peptide.

A “small molecule” is defined herein to have a molecular weight below about 600, preferably below about 1000 daltons.

An “agonist antibody,” as used herein, is an antibody which partially or fully mimics a biological activity of an IL-22 polypeptide.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, diluent, stabilizer, or preservative.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

Within this application, unless otherwise stated, the techniques utilized may be found in any of several well-known references such as: Molecular Cloning: A Laboratory Manual (Sambrook, et al., 1989, Cold Spring Harbor Laboratory Press), PCR Protocols: A Guide to Methods and Applications (Innis, et al. 1990. Academic Press, San Diego, Calif.), and Harlow and Lane (1988) Antibodies: A Laboratory Manual ch. 14 (Cold Spring Harbor Laboratory, Cold Spring Harbor, N.Y.).

As appropriate, procedures involving the use of commercially available kits and reagents are generally carried out in accordance with manufacturer defined protocols and/or parameters unless otherwise noted. Before the present methods and uses therefore are described, it is to be understood that this invention is not limited to the particular methodology, protocols, cell lines, animal species or genera, constructs, and reagents described as such can, of course, vary. It is also to be understood that the terminology used herein is for the purpose of describing particular embodiments only, and is not intended to limit the scope of the present invention which will be limited only by the appended claims.

### II. Compositions and Methods

The invention provides IL-22 Fc fusion proteins, compositions thereof (e.g., pharmaceutical compositions), and uses thereof, for example, for the treatment of IL-22 associated diseases such as IBD (e.g., ulcerative colitis (UC) and Crohn's disease), cardiovascular conditions, metabolic syndrome, GVHD, and for accelerating wound healing (e.g., diabetic wound healing). Also provided herein are methods of making and methods of purifying IL-22 Fc fusion proteins. The invention is based, at least in part, on the discovery that the IL-22 polypeptide moiety of IL-22 Fc fusion proteins is sialylated, and that the sialylation content is associated with both the potency and pharmacokinetic properties of the IL-22 Fc fusion proteins provided herein. This discovery was made in part in connection with identifying certain properties of the molecule that are affected by the manufacturing process and that impact the activity and PK/PD properties of the molecule. For example, it is presently discovered that IL-22 Fc-containing compositions having overall low glycosylation (including, but not limited to, e.g., IL-22 Fc fusion proteins and compositions thereof with an average sialic acid content of less than about 8 moles of sialic acid per mole of IL-22 Fc fusion protein) as described herein have undesirably fast clearance in vivo, and further, that high glycosylation of those compositions (including, but not limited to, e.g., IL-22 Fc fusion proteins and compositions thereof having greater than about 12 moles of sialic acid per mole of IL-22 Fc fusion protein) have undesirable binding properties to the IL-22 receptor. Thus, in certain aspects, a solution to the identified problems was to identify a range of average sialic acid content for the IL-22 Fc fusion proteins and compositions thereof which have both suitable clearance rates as well as suitable binding activity, as described herein. More particularly, it is presently discovered that the desired ranges are ranges which are less than full sialylation, which otherwise is typically what the skilled artisan would select, e.g., for ease of manufacture. In a specific embodiment, a particularly preferred range of average sialic acid content for the IL-22 Fc fusion proteins and compositions thereof is 8 to 9 moles of sialic acid per mole of IL-22 Fc fusion protein.

A. IL-22 Fc Fusion Proteins and Compositions

The invention provides IL-22 Fc fusion proteins and compositions thereof. In general, the IL-22 Fc fusion proteins include an IL-22 polypeptide linked to an Fc region by a linker. In some embodiments, the IL-22 polypeptide is glycosylated (e.g., N-glycosylated). In particular embodiments, the IL-22 polypeptide is sialylated. In some embodiments, the Fc region is not glycosylated, and thus, is also not sialylated.

In some embodiments, the sialic acid content of the IL-22 Fc fusion protein is more than about 3 moles of sialic acid per mole of the IL-22 Fc fusion protein. In some embodiments, the sialic acid content of the IL-22 Fc fusion protein is more than about 4 moles of sialic acid per mole of the IL-22 Fc fusion protein. In some embodiments, the sialic acid content of the IL-22 Fc fusion protein is more than about 5 moles of sialic acid per mole of the IL-22 Fc fusion protein. In some embodiments, the sialic acid content is more than about 6 moles of sialic acid per mole of the IL-22 Fc fusion protein. In some embodiments, the sialic acid content is more than about 7 moles of sialic acid per mole of the IL-22 Fc fusion protein. In some embodiments, the sialic acid content is more than about 8 moles of sialic acid per mole of the IL-22 Fc fusion protein. In some embodiments, the sialic acid content is more than about 9 moles of sialic acid per mole of the IL-22 Fc fusion protein. In some embodiments, the sialic acid content is more than about 10 moles of sialic acid per mole of the IL-22 Fc fusion protein. In some embodiments, the sialic acid content is more than about 11 moles of sialic acid per mole of the IL-22 Fc fusion protein. In some embodiments, the sialic acid content is more than about 12 moles of sialic acid per mole of the IL-22 Fc fusion protein. In some embodiments, the sialic acid content is more than about 13 moles of sialic acid per mole of the IL-22 Fc fusion protein. In some embodiments, the sialic acid content is more than about 14 moles of sialic acid per mole of the IL-22 Fc fusion protein. In some embodiments, the sialic acid content is more than about 15 moles of sialic acid per mole of the IL-22 Fc fusion protein. In some embodiments, the sialic acid content is more than about 16 moles of sialic acid per mole of the IL-22 Fc fusion protein. In some embodiments, the sialic acid content is more than about 17 moles of sialic acid per mole of the IL-22 Fc fusion protein. In some embodiments, the sialic acid content is more than about 18 moles of sialic acid per mole of the IL-22 Fc fusion protein. In some embodiments, the sialic acid content is more than about 19 moles of sialic acid per mole of the IL-22 Fc fusion protein. In some embodiments, the sialic acid content is more than about 20 moles of sialic acid per mole of the IL-22 Fc fusion protein.

In some embodiments, the sialic acid content is less than about 20 moles of sialic acid per mole of the IL-22 Fc fusion protein. In some embodiments, the sialic acid content is less than about 19 moles of sialic acid per mole of the IL-22 Fc fusion protein. In some embodiments, the sialic acid content is less than about 18 moles of sialic acid per mole of the IL-22 Fc fusion protein. In some embodiments, the sialic acid content is less than about 17 moles of sialic acid per mole of the IL-22 Fc fusion protein. In some embodiments, the sialic acid content is less than about 16 moles of sialic acid per mole of the IL-22 Fc fusion protein. In some embodiments, the sialic acid content is less than about 15 moles of sialic acid per mole of the IL-22 Fc fusion protein. In some embodiments, the sialic acid content is less than about 14 moles of sialic acid per mole of the IL-22 Fc fusion protein. In some embodiments, the sialic acid content is less than about 13 moles of sialic acid per mole of the IL-22 Fc fusion protein. In some embodiments, the sialic acid content is less than about 12 moles of sialic acid per mole of the IL-22 Fc fusion protein. In some embodiments, the sialic acid content is less than about 11 moles of sialic acid per mole of the IL-22 Fc fusion protein. In some embodiments, the sialic acid content is less than about 10 moles of sialic acid per mole of the IL-22 Fc fusion protein. In some embodiments, the sialic acid content is less than about 9 moles of sialic acid per mole of the IL-22 Fc fusion protein. In some embodiments, the sialic acid content is less than about 8 moles of sialic acid per mole of the IL-22 Fc fusion protein. In some embodiments, the sialic acid content is less than about 7 moles of sialic acid per mole of the IL-22 Fc fusion protein. In some embodiments, the sialic acid content is less than about 6 moles of sialic acid per mole of the IL-22 Fc fusion protein. In some embodiments, the sialic acid content is less than about 5 moles of sialic acid per mole of the IL-22 Fc fusion protein.

For example, in one aspect, the invention provides an IL-22 Fc fusion proteins that includes an IL-22 polypeptide linked to an Fc region by a linker, wherein the IL-22 polypeptide is glycosylated, and wherein the IL-22 Fc fusion protein has a sialic acid content of from about 4 to about 20 moles (e.g., about 4, about 5, about 6, about 7, about 8, about 9, about 10, about 11, about 12, about 13, about 14, or about 15 moles, about 16 moles, about 17 moles, about 18 moles, about 19 moles, or about 20 moles) of sialic acid per mole of the IL-22 Fc fusion protein.

In another aspect, the invention provides an IL-22 Fc fusion protein comprising an IL-22 polypeptide linked to an Fc region by a linker, wherein the IL-22 polypeptide is glycosylated, and wherein the IL-22 Fc fusion protein has a potency of about 20% to about 180% (e.g., about 20%, about 30%, about 40%, about 50%, about 60%, about 70%, about 80%, about 90%, about 100%, about 110%, about 120%, about 130%, about 140%, about 150%, about 160%, about 170%, or about 180%), for example, relative to a reference IL-22 Fc fusion protein having a sialic acid content of about 8 moles of sialic acid per mole of the IL-22 Fc fusion protein. In some embodiments, the IL-22 Fc fusion protein has a potency of about 40% to about 130%, for example, relative to a reference IL-22 Fc fusion protein having a sialic acid content of about 8 moles of sialic acid per mole of the IL-22 Fc fusion protein. In some embodiments, the IL-22 Fc fusion protein has a potency of about 80% to about 120%, for example, relative to a reference IL-22 Fc fusion protein having a sialic acid content of about 8 moles to about 12 moles (e.g., about 8, about 9, about 10, about 11, or about 12 moles) of sialic acid per mole of the IL-22 Fc fusion protein. In some embodiments, the IL-22 Fc fusion protein has a potency of about 60% to about 110%, for example, relative to a reference IL-22 Fc fusion protein having a sialic acid content of about 8 moles to about 12 moles (e.g., about 8, about 9, about 10, about 11, or about 12 moles) of sialic acid per mole of the IL-22 Fc fusion protein. In some embodiments, the IL-22 Fc fusion protein has a potency of about 80% to about 10%, for example, relative to a reference IL-22 Fc fusion protein having a sialic acid content of about 8 moles to about 12 moles (e.g., about 8, about 9, about 10, about 11, or about 12 moles) of sialic acid per mole of the IL-22 Fc fusion protein. In some embodiments, the IL-22 Fc fusion protein has a potency of about 40% to about 130%, for example, relative to a reference IL-22 Fc fusion protein having a sialic acid content of about 8 moles of sialic acid per mole of the IL-22 Fc fusion protein. In some embodiments, the IL-22 Fc fusion protein has a potency of about 60% to about 110%, for example, relative to a reference IL-22 Fc fusion protein having a sialic acid content of about 8 moles of sialic acid per mole of the IL-22 Fc fusion protein. In some embodiments, the IL-22 Fc fusion protein has a potency of about 80% to about 10%, for example, relative to a reference IL-22 Fc fusion protein having a sialic acid content of about 8 of sialic acid per mole of the IL-22 Fc fusion protein. In some embodiments, potency is assessed in a receptor binding assay or a cell-based binding assay, as described herein (e.g., in Example 2). In some embodiments, the reference IL-22 Fc fusion protein has the N-glycan distribution shown in Table 12 and/or Table 13.

For example, in some embodiments of any of the preceding aspects, the sialic acid content is from about 5 to about 16 moles of sialic acid per mole of the IL-22 Fc fusion protein, about 5 to about 15 moles of sialic acid per mole of the IL-22 Fc fusion protein, from about 5 to about 14 moles of sialic acid per mole of the IL-22 Fc fusion protein, from about 5 to about 13 moles of sialic acid per mole of the IL-22 Fc fusion protein, from about 5 to about 12 moles of sialic acid per mole of the IL-22 Fc fusion protein, from about 5 to about 11 moles of sialic acid per mole of the IL-22 Fc fusion protein, from about 5 to about 10 moles of sialic acid per mole of the IL-22 Fc fusion protein, from about 5 to about 9 moles of sialic acid per mole of the IL-22 Fc fusion protein, from about 5 to about 8 moles of sialic acid per mole of the IL-22 Fc fusion protein, from about 5 to about 7 moles of sialic acid per mole of the IL-22 Fc fusion protein, from about 5 to about 6 moles of sialic acid per mole of the IL-22 Fc fusion protein, from about 6 to about 16 moles of sialic acid per mole of the IL-22 Fc fusion protein, from about 6 to about 15 moles of sialic acid per mole of the IL-22 Fc fusion protein, from about 6 to about 14 moles of sialic acid per mole of the IL-22 Fc fusion protein, from about 6 to about 13 moles of sialic acid per mole of the IL-22 Fc fusion protein, from about 6 to about 12 moles of sialic acid per mole of the IL-22 Fc fusion protein, from about 6 to about 11 moles of sialic acid per mole of the IL-22 Fc fusion protein, from about 6 to about 10 moles of sialic acid per mole of the IL-22 Fc fusion protein, from about 6 to about 9 moles of sialic acid per mole of the IL-22 Fc fusion protein, from about 6 to about 8 moles of sialic acid per mole of the IL-22 Fc fusion protein, from about 6 to about 7 moles of sialic acid per mole of the IL-22 Fc fusion protein, from about 7 to about 16 moles of sialic acid per mole of the IL-22 Fc fusion protein, from about 7 to about 15 moles of sialic acid per mole of the IL-22 Fc fusion protein, from about 7 to about 14 moles of sialic acid per mole of the IL-22 Fc fusion protein, from about 7 to about 13 moles of sialic acid per mole of the IL-22 Fc fusion protein, from about 7 to about 12 moles of sialic acid per mole of the IL-22 Fc fusion protein, from about 7 to about 11 moles of sialic acid per mole of the IL-22 Fc fusion protein, from about 7 to about 10 moles of sialic acid per mole of the IL-22 Fc fusion protein, from about 7 to about 9 moles of sialic acid per mole of the IL-22 Fc fusion protein, from about 7 to about 8 moles of sialic acid per mole of the IL-22 Fc fusion protein, from about 8 to about 16 moles of sialic acid per mole of the IL-22 Fc fusion protein, from about 8 to about 15 moles of sialic acid per mole of the IL-22 Fc fusion protein, from about 8 to about 14 moles of sialic acid per mole of the IL-22 Fc fusion protein, from about 8 to about 13 moles of sialic acid per mole of the IL-22 Fc fusion protein, from about 8 to about 12 moles of sialic acid per mole of the IL-22 Fc fusion protein, from about 8 to about 11 moles of sialic acid per mole of the IL-22 Fc fusion protein, from about 8 to about 10 moles of sialic acid per mole of the IL-22 Fc fusion protein, from about 9 to about 16 moles of sialic acid per mole of the IL-22 Fc fusion protein, from about 9 to about 15 moles of sialic acid per mole of the IL-22 Fc fusion protein, from about 9 to about 14 moles of sialic acid per mole of the IL-22 Fc fusion protein, from about 9 to about 13 moles of sialic acid per mole of the IL-22 Fc fusion protein, from about 9 to about 12 moles of sialic acid per mole of the IL-22 Fc fusion protein, from about 9 to about 11 moles of sialic acid per mole of the IL-22 Fc fusion protein, from about 9 to about 10 moles of sialic acid per mole of the IL-22 Fc fusion protein, from about 10 to about 16 moles of sialic acid per mole of the IL-22 Fc fusion protein, from about 10 to about 15 moles of sialic acid per mole of the IL-22 Fc fusion protein, from about 10 to about 14 moles of sialic acid per mole of the IL-22 Fc fusion protein, from about 10 to about 13 moles of sialic acid per mole of the IL-22 Fc fusion protein, from about 10 to about 12 moles of sialic acid per mole of the IL-22 Fc fusion protein, from about 10 to about 11 moles of sialic acid per mole of the IL-22 Fc fusion protein, from about 11 to about 16 moles of sialic acid per mole of the IL-22 Fc fusion protein, from about 11 to about 15 moles of sialic acid per mole of the IL-22 Fc fusion protein, from about 11 to about 14 moles of sialic acid per mole of the IL-22 Fc fusion protein, from about 11 to about 13 moles of sialic acid per mole of the IL-22 Fc fusion protein, from about 11 to about 12 moles of sialic acid per mole of the IL-22 Fc fusion protein, from about 12 to about 16 moles of sialic acid per mole of the IL-22 Fc fusion protein, from about 12 to about 15 moles of sialic acid per mole of the IL-22 Fc fusion protein, from about 12 to about 14 moles of sialic acid per mole of the IL-22 Fc fusion protein, from about 12 to about 13 moles of sialic acid per mole of the IL-22 Fc fusion protein, from about 13 to about 16 moles of sialic acid per mole of the IL-22 Fc fusion protein, from about 13 to about 15 moles of sialic acid per mole of the IL-22 Fc fusion protein, from about 13 to about 14 moles of sialic acid per mole of the IL-22 Fc fusion protein, from about 14 to about 16 moles of sialic acid per mole of the IL-22 Fc fusion protein, from about 14 to about 15 moles of sialic acid per mole of the IL-22 Fc fusion protein, or from about 15 to about 16 moles of sialic acid per mole of the IL-22 Fc fusion protein.

In some embodiments, the sialic acid content is from about 8 to about 12 moles (e.g., about 8, about 9, about 10, about 11, or about 12 moles) per mole of the IL-22 Fc fusion protein. For example, in particular embodiments, the sialic acid content is about 8 moles of sialic acid per mole of the IL-22 Fc fusion protein. In other particular embodiments, the sialic acid content is about 9 moles of sialic acid per mole of the IL-22 Fc fusion protein.

The sialic acid may be any suitable sialic acid known in the art or any suitable combination thereof. For example, in some embodiments, the sialic acid is N-acetylneuraminic acid (NANA), Kdn, NGNA, Neu, Neu2en5Ac, or a combination thereof. In some embodiments, the predominant sialic acid is NANA. In some embodiments, substantially all of the sialic acid is NANA.

Any of the preceding IL-22 Fc fusion proteins can have a maximum observed concentration (Cₘₐₓ) of about 6,000 ng/mL to about 25,000 ng, e.g., about 6,000 ng/mL, about 7,000 ng/mL, about 8,000 ng/mL, about 9,000 ng/mL, about 10,000 ng/mL, about 11,000 ng/mL, about 12,000 ng/mL, about 13,000 ng/mL, about 14,000 ng/mL, about 15,000 ng/mL, about 16,000 ng/mL, about 17,000 ng/mL, about 18,000 ng/mL, about 19,000 ng/mL, about 20,000 ng/mL, about 21,000 ng/mL, about 22,000 ng/mL, about 23,000 ng/mL, about 24,000 ng/mL, or about 25,000 ng/mL. In some embodiments, the IL-22 Fc fusion protein has a Cₘₐₓ of about 9,000 ng/mL to about 18,000 ng, e.g., about 9,000 ng/mL, about 10,000 ng/mL, about 11,000 ng/mL, about 12,000 ng/mL, about 13,000 ng/mL, about 14,000 ng/mL, about 15,000 ng/mL, about 16,000 ng/mL, about 17,000 ng/mL, or about 18,000 ng/mL. In some embodiments, the IL-22 Fc fusion protein has a Cₘₐₓ of about 8,000 ng/mL to about 19,000 ng. In some embodiments, the Cₘₐₓ is assessed following intravenous administration of about 1,000 μg/kg of the IL-22 Fc fusion protein to a CD1 mouse, or is an equivalent human Cₘₐₓ value.

Any of the preceding IL-22 Fc fusion proteins can have an area under the serum concentration-time curve from time 0 to the last measureable time point (AUCₗₐₛₜ) of about 2,000 day·ng/mL to about 42,000 day·ng/mL, e.g., about 2,000 day·ng/mL, about 4,000 day·ng/mL, about 6,000 day·ng/mL, about 7,000 day·ng/mL, about 7,500 day·ng/mL, about 8,000 day·ng/mL, about 8,500 day·ng/mL, about 9,000 day·ng/mL, about 9,500 day·ng/mL, about 10,000 day·ng/mL, about 12,000 day·ng/mL, about 16,000 day·ng/mL, about 20,000 day·ng/mL, about 24,000 day·ng/mL, about 30,000 day·ng/mL, about 36,000 day·ng/mL, or about 42,000 day·ng/mL. For example, in some embodiments, the IL-22 Fc fusion protein has an AUCₗₐₛₜ of about 7,000 day·ng/mL to about 25,000 day·ng/mL. In some embodiments, the AUCₗₐₛₜ is assessed following intravenous administration of about 1,000 μg/kg of the IL-22 Fc fusion protein to a CD1 mouse, or is an equivalent human AUCₗₐₛₜ value.

Any of the preceding IL-22 Fc fusion proteins can have a clearance (CL) of about 25 mL/kg/day to about 400 mL/kg/day, e.g., about 25 mL/kg/day, about 50 mL/kg/day, about 75 mL/kg/day, about 100 mL/kg/day, about 125 mL/kg/day, about 150 mL/kg/day, about 175 mL/kg/day, about 200 mL/kg/day, about 225 mL/kg/day, about 250 mL/kg/day, about 275 mL/kg/day, about 300 mL/kg/day, about 325 mL/kg/day, about 350 mL/kg/day, about 375 mL/kg/day, or about 400 mL/kg/day. In some embodiments, the CL is about 40 mL/kg/day to about 140 mL/kg/day. In some embodiments, the CL is assessed following intravenous administration of about 1,000 μg/kg of the IL-22 Fc fusion protein to a CD1 mouse, or is an equivalent human CL value.

In some embodiments, the NGNA content is less than about 5 moles of NGNA per mole of the IL-22 Fc fusion protein. In some embodiments, the NGNA content is less than about 4 moles of NGNA per mole of the IL-22 Fc fusion protein. In some embodiments, the NGNA content is less than about 3 moles of NGNA per mole of the IL-22 Fc fusion protein. In some embodiments, the NGNA content is less than about 2 moles of NGNA per mole of the IL-22 Fc fusion protein. In some embodiments, the NGNA content is less than about 1 moles of NGNA per mole of the IL-22 Fc fusion protein. In some embodiments, the NGNA content is less than about 0.5 moles of NGNA per mole of the IL-22 Fc fusion protein. In some embodiments, the NGNA content is less than about 0.2 moles of NGNA per mole of the IL-22 Fc fusion protein. In some embodiments, the NGNA content is less than about 0.1 moles of NGNA per mole of the IL-22 Fc fusion protein. In some embodiments, the NGNA content is less than about 0.08 moles of NGNA per mole of the IL-22 Fc fusion protein. In some embodiments, the NGNA content is less than about 0.05 moles of NGNA per mole of the IL-22 Fc fusion protein. In some embodiments, the NGNA content is less than about 0.01 moles of NGNA per mole of the IL-22 Fc fusion protein. In some embodiments, the NGNA content is less than about 0.001 moles of NGNA per mole of the IL-22 Fc fusion protein. In some embodiments, the NGNA content is between about 0.001 moles to about 5 mole of NGNA per mole of the IL-22-Fc fusion protein, between about 0.001 moles to about 1 mole of NGNA per mole of the IL-22-Fc fusion protein, between about 0.01 moles to about 1 mole of NGNA per mole of the IL-22-Fc fusion protein, between about 0.1 moles to about 1 mole of NGNA per mole of the IL-22-Fc fusion protein, or between about 0.5 moles to about 1 mole of NGNA per mole of the IL-22-Fc fusion protein.

In any of the preceding aspects, the IL-22 polypeptide may be N-glycosylated. Any of the preceding IL-22 Fc fusion proteins can include N-glycans having monoantennary, biantennary, triantennary, and/or tetrantennary structure.

For example, in some embodiments, about 0.01% to about 5% (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%) of the N-glycans have monoantennary structure. In some embodiments, about 0.1% to about 2% of the N-glycans have monoantennary structure. In some embodiments, about 0.5% to about 1.5% of the N-glycans have monoantennary structure. In some embodiments, about 0.6% to about 1.5% of the N-glycans have monoantennary structure. In some embodiments, about 0.3% to about 1.7% of the N-glycans have monoantennary structure. In some embodiments, about 1% of the N-glycans have monoantennary structure.

For example, in some embodiments, about 5% to about 40% (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%) of the N-glycans have biantennary structure. In some embodiments, about 10% to about 25% of the N-glycans have biantennary structure. In some embodiments, about 10% to about 20% of the N-glycans have biantennary structure. In some embodiments, about 13.1% to about 20.4% of the N-glycans have biantennary structure. In some embodiments, about 10.6% to about 22.8% of the N-glycans have biantennary structure. In some embodiments, about 17% of the N-glycans have biantennary structure.

In some embodiments, about 10% to about 50% (e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%) of the N-glycans have triantennary structure. In some embodiments, about 20% to about 40% of the N-glycans have triantennary structure. In some embodiments, about 25% to about 35% of the N-glycans have triantennary structure. In some embodiments, about 28.2% to about 33.5% of the N-glycans have triantennary structure. In some embodiments, about 26.5% to about 35.3% of the N-glycans have triantennary structure. In some embodiments, about 31% of the N-glycans have triantennary structure.

In some embodiments, about 20% to about 60% (e.g., about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, or about 55%, about 56%, about 57%, about 58%, about 59%, or about 60%) of the N-glycans have tetraantennary structure. In some embodiments, about 30% to about 50% of the N-glycans have tetraantennary structure. In some embodiments, about 35% to about 45% of the N-glycans have tetraantennary structure. In some embodiments, about 35.9% to about 47% of the N-glycans have tetraantennary structure. In some embodiments, about 26.5% to about 35.3% of the N-glycans have tetraantennary structure. In some embodiments, about 42% of the N-glycans have tetraantennary structure.

Any of the preceding IL-22 Fc fusion proteins can comprise N-glycans comprising zero, one, two, three, or four galactose moieties.

For example, in some embodiments, about 5% to about 40% (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%) of the N-glycans comprise zero galactose moieties. In some embodiments, about 10% to about 30% of the N-glycans comprise zero galactose moieties. In some embodiments, about 15% to about 25% of the N-glycans comprise zero galactose moieties. In some embodiments, about 13.7% to about 27.5% of the N-glycans comprise zero galactose moieties. In some embodiments, about 9.1% to about 32.1% of the N-glycans comprise zero galactose moieties. In some embodiments, about 21% of the N-glycans comprise zero galactose moieties.

In another example, in some embodiments, about 1% to about 35% (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35%) of the N-glycans comprise one galactose moiety. In some embodiments, about 10% to about 30% of the N-glycans comprise one galactose moiety. In some embodiments, about 10% to about 20% of the N-glycans comprise one galactose moiety. In some embodiments, about 12% to about 16% of the N-glycans comprise one galactose moiety. In some embodiments, about 12.3% to about 15.6% of the N-glycans comprise one galactose moiety. In some embodiments, about 11.2% to about 16.7% of the N-glycans comprise one galactose moiety. In some embodiments, about 14% of the N-glycans comprise one galactose moiety.

In yet another example, in some embodiments, about 1% to about 35% (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35%) of the N-glycans comprise two galactose moieties. In some embodiments, about 5% to about 25% of the N-glycans comprise two galactose moieties. In some embodiments, about 8% to about 25% of the N-glycans comprise two galactose moieties. In some embodiments, about 10% to about 16% of the N-glycans comprise two galactose moieties. In some embodiments, about 10% to about 20% of the N-glycans comprise two galactose moieties. In some embodiments, about 10.9% to about 15.7% of the N-glycans comprise two galactose moieties. In some embodiments, about 9.3% to about 17.4% of the N-glycans comprise two galactose moieties. In some embodiments, about 13% of the N-glycans comprise two galactose moieties.

In a still further example, in some embodiments, about 5% to about 40% (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%) of the N-glycans comprise three galactose moieties. In some embodiments, about 10% to about 30% of the N-glycans comprise three galactose moieties. In some embodiments, about 12% to about 25% of the N-glycans comprise three galactose moieties. In some embodiments, about 16.4% to about 20.6% of the N-glycans comprise three galactose moieties. In some embodiments, about 15% to about 22% of the N-glycans comprise three galactose moieties. In some embodiments, about 19% of the N-glycans comprise three galactose moieties.

In another example, in some embodiments, about 5% to about 45% (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45%) of the N-glycans comprise four galactose moieties. In some embodiments, about 10% to about 30% of the N-glycans comprise four galactose moieties. In some embodiments, about 15% to about 25% of the N-glycans comprise four galactose moieties. In some embodiments, about 20.8% to about 26.4% of the N-glycans comprise four galactose moieties. In some embodiments, about 18.9% to about 28.3% of the N-glycans comprise four galactose moieties. In some embodiments, about 24% of the N-glycans comprise four galactose moieties.

Any of the preceding IL-22 Fc fusion proteins can comprise N-glycans comprising zero, one, two, three, or four sialic acid moieties.

For example, in some embodiments, about 10% to about 50% (e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%) of the N-glycans comprise zero sialic acid moieties. In some embodiments, about 15% to about 35% of the N-glycans comprise zero sialic acid moieties. In some embodiments, about 20% to about 30% of the N-glycans comprise zero sialic acid moieties. In some embodiments, about 17.3% to about 30% of the N-glycans comprise zero sialic acid moieties. In some embodiments, about 13.1% to about 34.3% of the N-glycans comprise zero sialic acid moieties. In some embodiments, about 24% of the N-glycans comprise zero sialic acid moieties.

In another example, in some embodiments, about 5% to about 45% (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45%) of the N-glycans comprise one sialic acid moiety. In some embodiments, about 10% to about 30% of the N-glycans comprise one sialic acid moiety. In some embodiments, about 15% to about 25% of the N-glycans comprise one sialic acid moiety. In some embodiments, about 17.6% to about 22.3% of the N-glycans comprise one sialic acid moiety. In some embodiments, about 16% to about 23.9% of the N-glycans comprise one sialic acid moiety. In some embodiments, about 20% of the N-glycans comprise one sialic acid moiety.

In yet another example, in some embodiments, about 5% to about 45% (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45%) of the N-glycans comprise two sialic acid moieties. In some embodiments, about 10% to about 30% of the N-glycans comprise two sialic acid moieties. In some embodiments, about 15% to about 25% of the N-glycans comprise two sialic acid moieties. In some embodiments, about 17.5% to about 23.7% of the N-glycans comprise two sialic acid moieties. In some embodiments, about 15.5% to about 25.8% of the N-glycans comprise two sialic acid moieties. In some embodiments, about 21% of the N-glycans comprise two sialic acid moieties.

In another example, in some embodiments, about 5% to about 40% (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%) of the N-glycans comprise three sialic acid moieties. In some embodiments, about 10% to about 30% of the N-glycans comprise three sialic acid moieties. In some embodiments, about 12% to about 24% of the N-glycans comprise three sialic acid moieties. In some embodiments, about 14.2% to about 19.1% of the N-glycans comprise three sialic acid moieties. In some embodiments, about 12.5% to about 20.7% of the N-glycans comprise three sialic acid moieties. In some embodiments, about 17% of the N-glycans comprise three sialic acid moieties.

For example, in some embodiments, about 1% to about 30% (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) of the N-glycans comprise four sialic acid moieties. In some embodiments, about 1% to about 20% of the N-glycans comprise four sialic acid moieties. In some embodiments, about 5% to about 15% of the N-glycans comprise four sialic acid moieties. In some embodiments, about 6.4% to about 12% of the N-glycans comprise four sialic acid moieties. In some embodiments, about 4.5% to about 13.9% of the N-glycans comprise four sialic acid moieties. In some embodiments, about 9% of the N-glycans comprise four sialic acid moieties.

In any of the preceding IL-22 Fc fusion proteins, the IL-22 polypeptide can include about 0% to about 20% (e.g., about 0%, about 0.1, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%) N-glycans that include a terminal mannose moiety. In some embodiments, about 0.1% to about 5% of the N-glycans comprise a terminal mannose moiety. In some embodiments, about 1% to about 4% of the N-glycans comprise a terminal mannose moiety. In some embodiments, about 1.6% to about 2.9% of the N-glycans comprise a terminal mannose moiety. In some embodiments, about 1.2% to about 3.3% of the N-glycans comprise a terminal mannose moiety. For example, in some embodiments, about 2% of the N-glycans comprise a terminal mannose moiety.

In any of the preceding IL-22 Fc fusion proteins, the IL-22 polypeptide can include about 10% to about 70% (e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 55%, about 56%, about 57%, about 58%, about 59%, about 60%, about 61%, about 62%, about 63%, about 64%, about 65%, about 66%, about 67%, about 68%, about 69%, or about 70%) N-glycans that include a terminal N-acetylglucosamine (GlcNAc) moiety. For example, in some embodiments, about 30% to about 50% of the N-glycans comprise a terminal GlcNAc moiety. In some embodiments, about 35% to about 45% of the N-glycans comprise a terminal GlcNAc moiety. In some embodiments, about 35.1% to about 49.2% of the N-glycans comprise a terminal GlcNAc moiety. In some embodiments, about 30.4% to about 53.8% of the N-glycans comprise a terminal GlcNAc moiety. In some embodiments, about 42% of the N-glycans comprise a terminal GlcNAc moiety.

In some embodiments of any of the preceding IL-22 Fc fusion proteins, about 1% to about 35% (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35%) of the N-glycans comprise one terminal GlcNAc moiety. In some embodiments, about 1% to about 20% of the N-glycans comprise one terminal GlcNAc moiety. In some embodiments, about 5% to about 15% of the N-glycans comprise one terminal GlcNAc moiety. In some embodiments, about 8.4% to about 12.5% of the N-glycans comprise one terminal GlcNAc moiety. In some embodiments, about 7% to about 13.8% of the N-glycans comprise one terminal GlcNAc moiety. In some embodiments, about 10% of the N-glycans comprise one terminal GlcNAc moiety.

In some embodiments of any of the preceding IL-22 Fc fusion proteins, about 1% to about 35% (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35%) of the N-glycans comprise two terminal GlcNAc moieties. In some embodiments, about 1% to about 20% of the N-glycans comprise two terminal GlcNAc moieties. In some embodiments, about 5% to about 15% of the N-glycans comprise two terminal GlcNAc moieties. In some embodiments, about 8.1% to about 12.5% of the N-glycans comprise two terminal GlcNAc moieties. In some embodiments, about 6.7% to about 14% of the N-glycans comprise two terminal GlcNAc moieties. In some embodiments, about 10% of the N-glycans comprise two terminal GlcNAc moieties.

In some embodiments of any of the preceding IL-22 Fc fusion proteins, about 1% to about 40% (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%) of the N-glycans comprise three terminal GlcNAc moieties. In some embodiments, about 5% to about 25% of the N-glycans comprise three terminal GlcNAc moieties. In some embodiments, about 10% to about 20% of the N-glycans comprise three terminal GlcNAc moieties. In some embodiments, about 10.1% to about 18.6% of the N-glycans comprise three terminal GlcNAc moieties. In some embodiments, about 7.2% to about 21.5% of the N-glycans comprise three terminal GlcNAc moieties. In some embodiments, about 14% of the N-glycans comprise three terminal GlcNAc moieties.

In some embodiments of any of the preceding IL-22 Fc fusion proteins, about 0.1% to about 25% (e.g., about 0.1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% of the N-glycans comprise four terminal GlcNAc moieties. In some embodiments, about 1% to about 15% of the N-glycans comprise four terminal GlcNAc moieties. In some embodiments, about 4% to about 24% of the N-glycans comprise four terminal GlcNAc moieties. In some embodiments, about 2.3% to about 11.8% of the N-glycans comprise four terminal GlcNAc moieties. In some embodiments, about 0.1% to about 15% of the N-glycans comprise four terminal GlcNAc moieties. In some embodiments, about 7% of the N-glycans comprise four terminal GlcNAc moieties.

Any of the preceding IL-22 Fc fusion proteins can include about 10% to about 70% (e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 55%, about 56%, about 57%, about 58%, about 59%, about 60%, about 61%, about 62%, about 63%, about 64%, about 65%, about 66%, about 67%, about 68%, about 69%, or about 70%) N-glycans that include a terminal galactose moiety. For example, in some embodiments, about 20% to about 50% of the N-glycans include a terminal Gal moiety. In some embodiments, about 25% to about 35% of the N-glycans include a terminal Gal moiety. In some embodiments, about 26.1% to about 38.3% of the N-glycans include a terminal Gal moiety. In some embodiments, about 22.1% to about 42.3% of the N-glycans include a terminal Gal moiety. In some embodiments, about 32% of the N-glycans include a terminal Gal moiety.

Any of the preceding IL-22 Fc fusion proteins can include one, two, or three terminal Gal moieties.

For example, in some embodiments of any of the preceding IL-22 Fc fusion proteins, about 5% to about 50% (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%) of the N-glycans comprise one terminal Gal moiety. In some embodiments, about 10% to about 30% of the N-glycans comprise one terminal Gal moiety. In some embodiments, about 15% to about 25% of the N-glycans comprise one terminal Gal moiety. In some embodiments, about 19.8% to about 27.1% of the N-glycans comprise one terminal Gal moiety. In some embodiments, about 17.4% to about 29.5% of the N-glycans comprise one terminal Gal moiety. In some embodiments, about 23% of the N-glycans comprise one terminal Gal moiety.

In some embodiments of any of the preceding IL-22 Fc fusion proteins, about 0% to about 25% (e.g., about 0%, about 0.1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%) of the N-glycans comprise two terminal Gal moieties. In some embodiments, about 1% to about 15% of the N-glycans comprise two terminal Gal moieties. In some embodiments, about 2% to about 12% of the N-glycans comprise two terminal Gal moieties. In some embodiments, about 4.6% to about 9.2% of the N-glycans comprise two terminal Gal moieties. In some embodiments, about 3% to about 10.8% of the N-glycans comprise two terminal Gal moieties. In some embodiments, about 7% of the N-glycans comprise two terminal Gal moieties.

In some embodiments of any of the preceding IL-22 Fc fusion proteins, about 0% to about 15% (e.g., about 0%, about 0.1%, about 1%, about 2%, about 3%, about 4%, about 5%, about 6%, about 7%, about 8%, about 9%, about 10%, about 11%, about 12%, about 13%, about 14%, or about 15%) of the N-glycans comprise three terminal Gal moieties. In some embodiments, about 0.1% to about 10% of the N-glycans comprise three terminal Gal moieties. In some embodiments, about 1% to about 5% of the N-glycans comprise three terminal Gal moieties. In some embodiments, about 1.1% to about 2.6% of the N-glycans comprise three terminal Gal moieties. In some embodiments, about 0.7% to about 3% of the N-glycans comprise three terminal Gal moieties. In some embodiments, about 2% of the N-glycans comprise three terminal Gal moieties.

In any of the preceding IL-22 Fc fusion proteins, the IL-22 polypeptide can include N-glycans that include galactose N-acetylglucosamine (LacNAc) repeats. In some embodiments, about 0% to about 20% (e.g., e.g., about 0%, about 0.1%, about 1%, about 2%, about 3%, about 4%, about 5%, about 6%, about 7%, about 8%, about 9%, about 10%, about 11%, about 12%, about 13%, about 14%, or about 15%, about 16%, about 17%, about 18%, about 19%, or about 20%) of the N-glycans include LacNAc repeats. For example, in some embodiments, about 1% to about 10% of the N-glycans comprise LacNAc repeats. In some embodiments, about 2% to about 8% of the N-glycans comprise LacNAc repeats. In some embodiments, about 3.7% to about 5.2% of the N-glycans comprise LacNAc repeats. In some embodiments, about 3.2% to about 5.7% of the N-glycans comprise LacNAc repeats. In some embodiments, about 5% of the N-glycans comprise LacNAc repeats.

In any of the preceding IL-22 Fc fusion proteins, the IL-22 polypeptide can include N-glycans that include fucosylated N-glycans. In some embodiments, about 50% to about 100% (e.g., about 50%, about 55%, about 56%, about 57%, about 58%, about 59%, about 60%, about 61%, about 62%, about 63%, about 64%, about 65%, about 66%, about 67%, about 68%, about 69%, about 70%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%) of the N-glycans are fucosylated. For example, in some embodiments, about 60% to about 80% of the N-glycans are fucosylated. In some embodiments, about 65% to about 75% of the N-glycans are fucosylated. In some embodiments, about 65.1% to about 75% of the N-glycans are fucosylated. In some embodiments, about 61.7% to about 78.3% of the N-glycan are fucosylated. In some embodiments, about 70% of the N-glycans are fucosylated.

In any of the preceding IL-22 Fc fusion proteins, the IL-22 polypeptide can include N-glycans that include afucosylated N-glycans. In some embodiments, about 5% to about 50% (e.g., about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%) of the N-glycans are afucosylated. For example, in some embodiments, about 10% to about 30% of the N-glycans are afucosylated. In some embodiments, about 15% to about 25% of the N-glycans are afucosylated. In some embodiments, about 16.4% to about 23.7% of the N-glycans are afucosylated. In some embodiments, about 14% to about 16.1% of the N-glycans are afucosylated. In some embodiments, about 20% of the N-glycans are afucosylated.

Any of the preceding IL-22 polypeptides can be glycosylated on amino acid residues Asn21, Asn35, Asn64, and/or Asn143 of SEQ ID NO:4. For example, in some embodiments, the IL-22 polypeptide is glycosylated on amino acid residues Asn21, Asn35, Asn64, and Asn143 of SEQ ID NO:4.

For example, in any of the preceding IL-22 Fc fusion proteins, the glycosylation occupancy on amino acid residue Asn21 of SEQ ID NO:4 can be about 50% to about 100% (e.g., about 50%, about 55%, about 56%, about 57%, about 58%, about 59%, about 60%, about 61%, about 62%, about 63%, about 64%, about 65%, about 66%, about 67%, about 68%, about 69%, about 70%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%). In some embodiments, the glycosylation occupancy on amino acid residue Asn21 of SEQ ID NO:4 is about 70% to about 90%. In some embodiments, the glycosylation occupancy on amino acid residue Asn21 of SEQ ID NO:4 is about 75% to about 85%. In some embodiments, the glycosylation occupancy on amino acid residue Asn21 of SEQ ID NO:4 is about 82%.

In any of the preceding IL-22 Fc fusion proteins, in some embodiments, the glycosylation occupancy on amino acid residue Asn35 of SEQ ID NO:4 can be about 60% to about 100% (e.g., about 60%, about 61%, about 62%, about 63%, about 64%, about 65%, about 66%, about 67%, about 68%, about 69%, about 70%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%). In some embodiments, the glycosylation occupancy on amino acid residue Asn35 of SEQ ID NO:4 is about 90% to about 100%. In some embodiments, the glycosylation occupancy on amino acid residue Asn35 of SEQ ID NO:4 is about 95% to about 100%. In some embodiments, the glycosylation occupancy on amino acid residue Asn35 of SEQ ID NO:4 is about 100%.

In any of the preceding IL-22 Fc fusion proteins, in some embodiments, the glycosylation occupancy on amino acid residue Asn64 of SEQ ID NO:4 can be about 60% to about 100% (e.g., about 60%, about 61%, about 62%, about 63%, about 64%, about 65%, about 66%, about 67%, about 68%, about 69%, about 70%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%). In some embodiments, the glycosylation occupancy on amino acid residue Asn64 of SEQ ID NO:4 is about 90% to about 100%. In some embodiments, the glycosylation occupancy on amino acid residue Asn64 of SEQ ID NO:4 is about 95% to about 100%. In some embodiments, the glycosylation occupancy on amino acid residue Asn64 of SEQ ID NO:4 is about 100%.

In any of the preceding IL-22 Fc fusion proteins, in some embodiments, the glycosylation occupancy on amino acid residue Asn143 of SEQ ID NO:4 can be about 1% to about 60% (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%). In some embodiments, the glycosylation occupancy on amino acid residue Asn143 of SEQ ID NO:4 is about 15% to about 45%. In some embodiments, the glycosylation occupancy on amino acid residue Asn143 of SEQ ID NO:4 is about 25% to about 35%. In some embodiments, the glycosylation occupancy on amino acid residue Asn143 of SEQ ID NO:4 is about 33%.

In some embodiments of any of the preceding IL-22 Fc fusion proteins, the Fc region is not glycosylated. In some embodiments, the amino acid residue at position 297 as in the EU index of the Fc region is Gly. In some embodiments, the amino acid residue at position 297 as in the EU index of the Fc region is Ala. In some embodiments, the amino acid residue at position 299 as in the EU index of the Fc region is Ala, Gly, or Val. In some embodiments, the Fc region comprises the CH2 and CH3 domain of IgG1 or IgG4. In some embodiments, the Fc region comprises the CH2 and CH3 domain of IgG4.

In some embodiments of any of the preceding IL-22 Fc fusion proteins, the IL-22 Fc fusion protein comprises an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence selected from the group consisting of SEQ ID NO:8, SEQ ID NO:10, SEQ ID NO:12, SEQ ID NO:14, and SEQ ID NO:16. In some embodiments, the IL-22 Fc fusion protein comprises an amino acid sequence having at least 96% sequence identity to the amino acid sequence of SEQ ID NO:8. In some embodiments, the IL-22 Fc fusion protein comprises an amino acid sequence having at least 97% sequence identity to the amino acid sequence of SEQ ID NO:8. In some embodiments, the IL-22 Fc fusion protein comprises an amino acid sequence having at least 98% sequence identity to the amino acid sequence of SEQ ID NO:8. In some embodiments, the IL-22 Fc fusion protein comprises an amino acid sequence having at least 99% sequence identity to the amino acid sequence of SEQ ID NO:8. In some embodiments, the IL-22 Fc fusion protein comprises the amino acid sequence of SEQ ID NO:8, SEQ ID NO:10, or SEQ ID NO:16. In some embodiments, the IL-22 Fc fusion protein comprises the amino acid sequence of SEQ ID NO:8. In some embodiments, the IL-22 Fc fusion protein consists of the amino acid sequence of SEQ ID NO:8. In some embodiments, the IL-22 Fc fusion protein comprises the amino acid sequence of SEQ ID NO:10. In some embodiments, the IL-22 Fc fusion protein consists of the amino acid sequence of SEQ ID NO:10. In some embodiments, the IL-22 Fc fusion protein comprises the amino acid sequence of SEQ ID NO:16. In some embodiments, the IL-22 Fc fusion protein consists of the amino acid sequence of SEQ ID NO:16. In some embodiments, the Fc region is not N-glycosylated.

Any of the preceding IL-22 Fc fusion proteins can be a dimeric IL-22 Fc fusion protein. In other embodiments, any of the preceding IL-22 Fc fusion proteins can be a monomeric IL-22 Fc fusion protein.

Any of the preceding IL-22 Fc fusion proteins can include a human IL-22 polypeptide. In some embodiments, the amino acid sequence of SEQ ID NO:4.

Any suitable linker can be used in the IL-22 Fc fusion proteins described herein. In some embodiments, the linker comprises the amino acid sequence RVESKYGPP (SEQ ID NO: 44). In some embodiments, the linker consists of the amino acid sequence RVESKYGPP (SEQ ID NO: 44).

In some embodiments, any of the IL-22 Fc fusion proteins described herein binds to IL-22 receptor. In some embodiments, the IL-22 receptor is human IL-22 receptor. In some embodiments, the IL-22 Fc fusion protein binds to IL-22RA1 and/or IL-10R2. In some embodiments, the IL-22 Fc fusion protein binds to IL-22RA1.

In some embodiments, any of the preceding IL-22 Fc fusion proteins is produced by the method comprising the step of culturing a host cell capable of expressing the IL-22 Fc fusion protein under conditions suitable for expression of the IL-22 Fc fusion protein. In some embodiments, the method further comprises the step of obtaining the IL-22 Fc fusion protein from the cell culture or culture medium. In some embodiments, the host cell is a CHO cell.

Any of the IL-22 Fc fusion proteins described herein (e.g., described above) can be included in a composition (e.g., a pharmaceutical composition). For example, any of the values described above with respect to an IL-22 Fc fusion protein may be the average value for a composition of IL-22 Fc proteins.

For example, provided herein is a composition including an interleukin (IL)-22 Fc fusion protein, wherein the IL-22 Fc fusion protein includes an IL-22 polypeptide linked to an Fc region by a linker, wherein the IL-22 polypeptide is glycosylated, and wherein the composition has an average sialic acid content in the range of 8 to 12 moles of sialic acid per mole of the IL-22 Fc fusion protein. In some embodiments, the IL-22 polypeptide is N-glycosylated.

In another example, provided herein is a composition including an IL-22 Fc fusion protein, wherein the IL-22 Fc fusion protein includes an IL-22 polypeptide linked to an Fc region by a linker, wherein the IL-22 polypeptide is glycosylated on amino acid residues Asn21, Asn35, Asn64, and/or Asn143 of SEQ ID NO: 4, and wherein: (a) the percent N-glycosylation site occupancy at residue Asn21 is in the range of 70 to 90; (b) the percent N-glycosylation site occupancy at residue Asn35 is in the range of 90 to 100; (c) the percent N-glycosylation site occupancy at residue Asn64 is in the range of 90 to 100; and/or (d) the percent N-glycosylation site occupancy at residue Asn143 is in the range of 25 to 35.

Any of the compositions may have an average sialic acid content in the range of 8 to 9 moles of sialic acid per mole of the IL-22 Fc fusion protein. In some embodiments, the composition has an average sialic acid content of 8 or 9 moles of sialic acid per mole of the IL-22 Fc fusion protein. In some embodiments, the composition has an average sialic acid content of 8 moles of sialic acid per mole of the IL-22 Fc fusion protein. In other embodiments, the composition has an average sialic acid content of 9 moles of sialic acid per mole of the IL-22 Fc fusion protein.

In any of the compositions described herein, the sialic acid may be N-acetylneuraminic acid (NANA).

Any of the compositions may have an average NGNA content of less than 1 mole of NGNA per mole of the IL-22 Fc fusion protein.

In some embodiments: (i) the IL-22 Fc fusion protein may have a maximum observed concentration (Cₘₐₓ) of about 8,000 ng/mL to about 19,000 ng; (ii) the IL-22 Fc fusion protein may have an area under the serum concentration-time curve from time 0 to the last measureable time point (AUCₗₐₛₜ) of about 7,000 day·ng/mL to about 25,000 day·ng/mL; and/or (iii) the IL-22 Fc fusion protein may have a clearance (CL) of about 40 mL/kg/day to about 140 mL/kg/day. In some embodiments, the Cₘₐₓ, AUCₗₐₛₜ, and/or CL is assessed following intravenous administration of about 1,000 μg/kg of the IL-22 Fc fusion protein to a CD1 mouse.

In any of the compositions, the IL-22 polypeptide may include N-glycans having monoantennary, biantennary, triantennary, and/or tetraantennary structure. In some embodiments: (i) about 0.1% to about 2% of the N-glycans have monoantennary structure; (ii) about 10% to about 25% of the N-glycans have biantennary structure; (iii) about 25% to about 40% of the N-glycans have triantennary structure; and/or (iv) about 30% to about 51% of the N-glycans have tetraantennary structure. In some embodiments: (i) 0.1% to 2% of the N-glycans have monoantennary structure; (ii) 10% to 25% of the N-glycans have biantennary structure; (iii) 25% to 40% of the N-glycans have triantennary structure; and/or (iv) 30% to 51% of the N-glycans have tetraantennary structure.

In any of the compositions, the IL-22 Fc fusion protein may include N-glycans including zero, one, two, three, or four galactose moieties. In some embodiments: (i) about 9% to about 32% of the N-glycans include zero galactose moieties; (ii) about 10% to about 20% of the N-glycans include one galactose moiety; (iii) about 8% to about 25% of the N-glycans include two galactose moieties; (iv) about 12% to about 25% of the N-glycans include three galactose moieties; and/or (v) about 12% to about 30% of the N-glycans include four galactose moieties. In some embodiments: (i) 9% to 32% of the N-glycans include zero galactose moieties; (ii) 10% to 20% of the N-glycans include one galactose moiety; (iii) 8% to 25% of the N-glycans include two galactose moieties; (iv) 12% to 25% of the N-glycans include three galactose moieties; and/or (v) 12% to 30% of the N-glycans include four galactose moieties.

In any of the compositions, the IL-22 Fc fusion protein may include N-glycans including zero, one, two, three, or four sialic acid moieties. In some embodiments: (i) about 12% to about 35% of the N-glycans include zero sialic acid moieties; (ii) about 10% to about 30% of the N-glycans include one sialic acid moiety; (iii) about 10% to about 30% of the N-glycans include two sialic acid moieties; (iv) about 10% to about 30% of the N-glycans include three sialic acid moieties; and/or (v) about 1% to about 20% of the N-glycans include four sialic acid moieties. In some embodiments: (i) 12% to 35% of the N-glycans include zero sialic acid moieties; (ii) 10% to 30% of the N-glycans include one sialic acid moiety; (iii) 10% to 30% of the N-glycans include two sialic acid moieties; (iv) 10% to 30% of the N-glycans include three sialic acid moieties; and/or (v) 1% to 20% of the N-glycans include four sialic acid moieties.

In any of the compositions, (i) the IL-22 polypeptide may include about 0% to about 10% N-glycans including a terminal mannose moiety; and/or (ii) the IL-22 polypeptide includes about 30% to about 55% N-glycans including a terminal N-acetylglucosamine (GlcNAc) moiety. In some embodiments, (i) the IL-22 polypeptide includes 0% to 10% N-glycans including a terminal mannose moiety; and/or (ii) the IL-22 polypeptide includes 30% to 55% N-glycans including a terminal GlcNAc moiety. In some embodiments, the IL-22 polypeptide includes 0% to 10% N-glycans including a terminal mannose moiety. In some embodiments, the IL-22 polypeptide includes 30% to 55% N-glycans including a terminal GlcNAc moiety.

In any of the compositions, the N-glycans may include one, two, three, or four terminal GlcNAc moieties. In some embodiments: (i) about 1% to about 20% of the N-glycans include one terminal GlcNAc moiety; (ii) about 1% to about 20% of the N-glycans include two terminal GlcNAc moieties; (iii) about 5% to about 25% of the N-glycans include three terminal GlcNAc moieties; and/or (iv) about 0% to about 15% of the N-glycans include four terminal GlcNAc moieties. In some embodiments: (i) 1% to 20% of the N-glycans include one terminal GlcNAc moiety; (ii) 1% to 20% of the N-glycans include two terminal GlcNAc moieties; (iii) 5% to 25% of the N-glycans include three terminal GlcNAc moieties; and/or (iv) 0% to 15% of the N-glycans include four terminal GlcNAc moieties.

In any of the compositions, (i) the IL-22 polypeptide may include about 20% to about 45% N-glycans including a terminal galactose (Gal) moiety; and/or (ii) the N-glycans include one, two, or three terminal Gal moieties. In some embodiments, (i) the IL-22 polypeptide includes 20% to 45% N-glycans including a terminal Gal moiety; and/or (ii) the N-glycans include one, two, or three terminal Gal moieties.

In any of the compositions: (i) about 15% to about 30% of the N-glycans may include one terminal Gal moiety; (ii) about 1% to about 15% of the N-glycans may include two terminal Gal moieties; and/or (iii) about 0.1% to about 6% of the N-glycans may include three terminal Gal moieties. In some embodiments: (i) 15% to 30% of the N-glycans include one terminal Gal moiety; (ii) 1% to 15% of the N-glycans include two terminal Gal moieties; and/or (iii) 0.1% to 6% of the N-glycans include three terminal Gal moieties.

In any of the compositions: (i) the IL-22 polypeptide may include N-glycans including galactose N-acetylglucosamine (LacNAc) repeats; (ii) the IL-22 polypeptide may include N-glycans including fucosylated N-glycans; and/or (iii) the IL-22 polypeptide may include N-glycans including afucosylated N-glycans.

In any of the compositions, the Fc region of the IL-22 Fc fusion protein may be not glycosylated. In some embodiments: (i) the amino acid residue at position 297 as in the EU index of the Fc region is Gly or Ala; and/or (ii) the amino acid residue at position 299 as in the EU index of the Fc region is Ala, Gly, or Val. In some embodiments, the amino acid residue at position 297 as in the EU index of the Fc region is Gly or Ala. In some embodiments, the amino acid residue at position 297 as in the EU index of the Fc region is Gly. In other embodiments, the amino acid residue at position 297 as in the EU index of the Fc region is Ala.

In any of the compositions, the Fc region of the IL-22 Fc fusion protein may include the CH2 and CH3 domain of IgG1 or IgG4. In some embodiments, the Fc region includes the CH2 and CH3 domain of IgG4.

In any of the compositions, the IL-22 Fc fusion protein may include an amino acid sequence having at least 95% (e.g., at least 95%, at least 96%, at least 97%, at least 98%, or at least 99%) sequence identity to the amino acid sequence of SEQ ID NO:8.

In any of the compositions, the IL-22 Fc fusion protein may include or consist of the amino acid sequence of SEQ ID NO:8, SEQ ID NO:10, or SEQ ID NO:16.

In any of the compositions, the IL-22 polypeptide may be a human IL-22 polypeptide. In some embodiments, the IL-22 polypeptide includes the amino acid sequence of SEQ ID NO:4.

In any of the compositions, the linker of the IL-22 Fc fusion protein may include or consist of the amino acid sequence RVESKYGPP (SEQ ID NO: 44).

In any of the compositions, the IL-22 Fc fusion protein may bind to IL-22 receptor. In some embodiments, the IL-22 receptor is human IL-22 receptor.

Any suitable concentration of the IL-22 Fc fusion protein may be used. For example, in some embodiments, the concentration of the IL-22 Fc fusion protein may be about 0.5 mg/mL to about 20 mg/mL. In some embodiments, the concentration of the IL-22 Fc fusion protein is about 0.5 mg/mL to about 5 mg/mL. In some embodiments, the concentration of the IL-22 Fc fusion protein is about 1 mg/mL. In some embodiments, the concentration of the IL-22 Fc fusion protein is about 8 mg/mL to about 12 mg/mL. In some embodiments, the concentration of the IL-22 Fc fusion protein is about 10 mg/mL.

The IL-22 Fc fusion proteins described herein may be produced from a production culture having a volume of at least about 500 L. In some embodiments of any of the preceding aspects, the IL-22 Fc fusion protein has been produced from a production culture having a volume of about 500 L to about 5,000 L. In some embodiments, the IL-22 Fc fusion protein has been produced from a production culture having a volume of about 1,000 L to about 3,000 L. In some embodiments the IL-22 Fc fusion protein has been produced from a production culture having a volume of about 1,500 L to about 2,500 L. In some embodiments, the IL-22 Fc fusion protein has been produced from a production culture having a volume of about 2000 L.

Any of the compositions may be a pharmaceutical composition. In some embodiments, the composition further includes an additional therapeutic agent. In some embodiments, the composition further includes a gelling agent.

1. Exemplary IL-22 Polypeptides

Any suitable IL-22 polypeptide can be included in the IL-22 Fc fusion proteins provided herein. For example, in any of the IL-22 Fc fusion proteins described herein, the IL-22 polypeptide can include a polypeptide comprising an amino acid sequence comprising SEQ ID NO:71 (human IL-22 with the endogenous IL-22 leader sequence), or a polypeptide comprising an amino acid sequence that has at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity) with SEQ ID NO:71. In certain embodiments, the IL-22 polypeptide comprises an amino acid sequence comprising SEQ ID NO:4 (human IL-22 without a leader sequence) or a polypeptide comprising an amino acid sequence that has at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) sequence identity with SEQ ID NO:4. In certain embodiments, the IL-22 polypeptide comprises an amino acid sequence comprising SEQ ID NO:4.

The preparation of native IL-22 molecules, along with their nucleic acid and polypeptide sequences, can be achieved through methods known to those of ordinary skill in the art. For example, IL-22 polypeptides can be produced by culturing cells transformed or transfected with a vector containing IL-22 nucleic acid. It is, of course, contemplated that alternative methods, which are well known in the art, can be employed to prepare IL-22. For instance, the IL-22 sequence, or portions thereof, can be produced by direct peptide synthesis using solid-phase techniques (see, e.g., Stewart et al., 1969, Solid-Phase Peptide Synthesis, W.H. Freeman Co., San Francisco, Calif. (1969); Merrifield, J. Am. Chem. Soc., 1963, 85:2149-2154). In vitro protein synthesis can be performed using manual techniques or by automation. Automated synthesis can be accomplished, for instance, using an Applied Biosystems Peptide Synthesizer (Foster City, Calif.) using manufacturer's instructions. Various portions of IL-22 can be chemically synthesized separately and combined using chemical or enzymatic methods to produce the full-length IL-22.

IL-22 variants can be prepared by introducing appropriate nucleotide changes into the DNA encoding a native sequence IL-22 polypeptide, or by synthesis of the desired IL-22 polypeptide. Those skilled in the art will appreciate that amino acid changes can alter post-translational processes of IL-22, such as changing the number or position of glycosylation sites or altering the membrane anchoring characteristics.

Variations in the native sequence IL-22 polypeptides described herein can be made, for example, using any of the techniques and guidelines for conservative and non-conservative mutations set forth, for instance, in U.S. Pat. No. 5,364,934. Variations can be a substitution, deletion, or insertion of one or more codons encoding a native sequence or variant IL-22 that results in a change in its amino acid sequence as compared with a corresponding native sequence or variant IL-22. Optionally the variation is by substitution of at least one amino acid with any other amino acid in one or more of the domains of a native sequence IL-22 polypeptide. Guidance in determining which amino acid residue can be inserted, substituted or deleted without adversely affecting the desired activity can be found by comparing the sequence of the IL-22 with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions can optionally be in the range of 1 to 5 amino acids. The variation allowed can be determined by systematically making insertions, deletions or substitutions of amino acids in the sequence and testing the resulting variants for activity, for example, in the in vitro assay described in the Examples below.

In particular embodiments, conservative substitutions of interest are shown in Table A under the heading of preferred substitutions. If such substitutions result in a change in biological activity, then more substantial changes, denominated exemplary substitutions in Table A, or as further described below in reference to amino acid classes, are introduced and the products screened.

Another type of covalent modification of the IL-22 polypeptides included within the scope of this invention comprises altering the native glycosylation pattern of the polypeptides. “Altering the native glycosylation pattern” is intended for purposes herein to mean deleting one or more carbohydrate moieties found in native sequence IL-22, and/or adding one or more glycosylation sites that are not present in the native sequence IL-22, and/or alteration of the ratio and/or composition of the sugar residues attached to the glycosylation site(s).

Glycosylation of polypeptides is typically either N-linked or O-linked. Addition of glycosylation sites to the IL-22 polypeptide can be accomplished by altering the amino acid sequence. The alteration can be made, for example, by the addition of, or substitution by, one or more serine or threonine residues to the native sequence IL-22 (for N-linked glycosylation sites), or the addition of a recognition sequence for O-linked glycosylation. The IL-22 amino acid sequence can optionally be altered through changes at the DNA level, particularly by mutating the DNA encoding the IL-22 polypeptide at preselected bases such that codons are generated that will translate into the desired amino acids.

Another means of increasing the number of carbohydrate moieties on the IL-22 polypeptide is by chemical or enzymatic coupling of glycosides to the polypeptide. Such methods are described in the art, e.g., in WO 87/05330 and in Aplin et al., CRC Crit. Rev. Biochem., pp. 259-306 (1981).

Removal of carbohydrate moieties present on an IL-22 polypeptide can be accomplished chemically or enzymatically or by mutational substitution of codons encoding for amino acid residues that serve as targets for glycosylation. Chemical deglycosylation techniques are known in the art and described, for instance, by Hakimuddin et al., Arch. Biochem. Biophys. 259:52 (1987) and by Edge et al., Anal. Biochem. 118:131 (1981). Enzymatic cleavage of carbohydrate moieties on polypeptides can be achieved by the use of a variety of endo- and exo-glycosidases as described by Thotakura et al., Meth. Enzymol. 138:350 (1987).

The variations can be made using methods known in the art such as oligonucleotide-mediated (site-directed) mutagenesis, alanine scanning, and PCR mutagenesis. Site-directed mutagenesis (Carter et al., 1986, Nucl. Acids Res. 13:4331; Zoller et al., 1987, Nucl. Acids Res. 10:6487), cassette mutagenesis (Wells et al., 1985, Gene 34:315), restriction selection mutagenesis (Wells et al., 1986, Philos. Trans. R. Soc. London A 317:415), or other known techniques can be performed on the cloned DNA to produce the IL-22 variant DNA.

Fragments of an IL-22 polypeptide are also provided herein. Such fragments can be truncated at the N-terminus or C-terminus, or can lack internal residues, for example, when compared with a full length native protein. Certain fragments lack amino acid residues that are not essential for a desired biological activity of an IL-22 polypeptide of the present invention. Accordingly, in certain embodiments, a fragment of an IL-22 polypeptide is biologically active. In certain embodiments, a fragment of full length IL-22 lacks the N-terminal signal peptide sequence.

Covalent modifications of native sequence and variant IL-22 polypeptides are included within the scope of this invention. One type of covalent modification includes reacting targeted amino acid residues of IL-22 with an organic derivatizing agent that is capable of reacting with selected side chains or the N- or C-terminal residues of the IL-22 polypeptide. Derivatization with bifunctional agents is useful, for instance, for crosslinking IL-22 to a water-insoluble support matrix or surface, for example, for use in the method for purifying anti-IL-22 antibodies. Commonly used crosslinking agents include, e.g., 1,1-bis(diazo-acetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl-propionate), bifunctional maleimides such as bis-N-maleimido-1,8-octane, and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, 1983, Proteins: Structure and Molecular Properties, W. H. Freeman & Co., San Francisco, pp. 79-86i), acetylation of the N-terminal amine, and amidation of any C-terminal carboxyl group.

Another type of covalent modification of IL-22 comprises linking the IL-22 polypeptide to one of a variety of nonproteinaceous polymers, e.g., polyethylene glycol, polypropylene glycol, or polyoxyalkylenes, for example in the manner set forth in U.S. Pat. Nos. 4,640,835; 4,496,689; 4,301,144; 4,670,417; 4,791,192; or 4,179,337. The native sequence and variant IL-22 can also be modified in a way to form a chimeric molecule comprising IL-22, including fragments of IL-22, fused to another, heterologous polypeptide or amino acid sequence.

In one embodiment, such a chimeric molecule comprises a fusion of IL-22 with a tag polypeptide which provides an epitope to which an anti-tag antibody can selectively bind. The epitope tag is generally placed at the amino- or carboxyl-terminus of the IL-22 polypeptide. The presence of such epitope-tagged forms of the IL-22 polypeptide can be detected using an antibody against the tag polypeptide. Also, provision of the epitope tag enables the IL-22 polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., 1988, Mol. Cell. Biol., 8:2159-2165); the c-myc tag and the 8F9, 3C7, 6E10, G4, and 9E10 antibodies thereto (Evan et al., 1985, Mol. Cell. Biol. 5:3610-3616); and the Herpes Simplex virus glycoprotein D (gD) tag and its antibody (Paborsky et al., 1990, Protein Engineering 3(6):547-553). Other tag polypeptides include the Flag-peptide (Hopp et al., 1988, BioTechnology 6:1204-1210); the KT3 epitope peptide (Martin et al., 1992, Science 255:192-194); a tubulin epitope peptide (Skinner et al., 1991, J. Biol. Chem. 266:15163-15166); and the T7 gene 10 protein peptide tag (Lutz-Freyermuth et al., 1990, Proc. Natl. Acad. Sci. USA, 87:6393-6397).

In another embodiment, the chimeric molecule can comprise a fusion of the IL-22 polypeptide or a fragment thereof with an immunoglobulin or a particular region of an immunoglobulin. For a bivalent form of the chimeric molecule, such a fusion can be to the Fc region of an IgG molecule. These fusion polypeptides are antibody-like molecules which combine the binding specificity of a heterologous protein (an “adhesin”) with the effector functions of immunoglobulin constant domains, and are often referred to as immunoadhesins. Structurally, the immunoadhesins comprise a fusion of an amino acid sequence of IL-22, or a variant thereof, and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin can be obtained from any immunoglobulin, such as IgG1, IgG2, IgG3, or IgG4 subtypes, IgA (including IgA1 and IgA2), IgE, IgD, or IgM. In certain embodiments, the IL-22 Fc fusion protein exhibits modified effector activities.

The IL-22 polypeptide, or a fragment thereof, can be fused, for example, to an immunoglobulin heavy chain constant region sequence to produce an IL-22-Ig fusion protein (e.g., IL-22 Fc fusion protein). The IL-22 polypeptide can be human or murine IL-22. The immunoglobulin heavy chain constant region sequence can be human or murine immunoglobulin heavy chain constant region sequence.

2. Exemplary IL-22 Fc Fusion Proteins

In certain embodiments, any of the IL-22 Fc fusion proteins described herein binds to and induces IL-22 receptor activity or signaling and/or is an agonist of IL-22 receptor activity.

In another aspect, an IL-22 Fc fusion protein provided herein comprises a polypeptide having at least 90%, 91%, 92%, 93%, 94%, 95%, 96%, 97%, 98%, 99%, or 100% sequence identity to the amino acid sequence of SEQ ID NO:4. In other embodiments, the IL-22 Fc fusion protein comprises a polypeptide having at least 90%, 91%, 92%, 93%, 94%, 95%, 96%, 97%, 98%, or 99% sequence identity contains substitutions (e.g., conservative substitutions), insertions, or deletions relative to the reference sequence, but an IL-22 Fc fusion protein comprising that sequence retains the ability to bind to IL-22 receptor. In certain embodiments, a total of 1 to 10 amino acids have been substituted, inserted, and/or deleted in SEQ ID NOs:8, 10, 12, 14, 16, 24, or 26. In certain embodiments, substitutions, insertions, or deletions occur in regions outside the IL-22 (i.e., in the Fc). In some embodiments, the substitutions, insertions, or deletions can be in the linker, the hinge, the CH2 domain, the CH3 domain of the IL-22 Fc fusion protein. In certain particular embodiments, the C-terminus Lys residue of Fc is deleted. In certain other embodiments, the C-terminus Gly and Lys residues of Fc are both deleted.

In some embodiments, the linker has at least 90%, 91%, 92%, 93%, 94%, 95%, 96%, 97%, 98%, 99%, or 100% sequence identity to DKTHT (SEQ ID NO:32), EPKSCDKTHT (SEQ ID NO:33), VEPKSCDKTHT (SEQ ID NO:34), KVEPKSCDKTHT (SEQ ID NO:35), KKVEPKSCDKTHT (SEQ ID NO:36), DKKVEPKSCDKTHT (SEQ ID NO:37), VDKKVEPKSCDKTHT (SEQ ID NO:38), KVDKKVEPKSCDKTHT (SEQ ID NO:39), EPKSSDKTHT (SEQ ID NO:40), GGGDKTHT (SEQ ID NO:41), ELKTPLGDTTHT (SEQ ID NO:42), SKYGPP (SEQ ID NO:43), RVESKYGPP (SEQ ID NO:44), GGGSTHT (SEQ ID NO:63), DKKVEPKSSDKTHT (SEQ ID NO:64), KVDKKVEPKSSDKTHT (SEQ ID NO:65), or KKVEPKSSDKTHT (SEQ ID NO:66). See, e.g., Table 2 of U.S. Pat. No. 9,815,880, which is incorporated herein by reference in its entirety.

In certain embodiments, IL-22 Fc fusion proteins variants having one or more amino acid substitutions are provided. Conservative substitutions are shown in Table A under the heading of “preferred substitutions.” More substantial changes are provided in Table A under the heading of “exemplary substitutions,” and as further described below in reference to amino acid side chain classes. Amino acid substitutions may be introduced into the IL-22 Fc fusion protein and the products screened for a desired activity, e.g., retained/improved IL-22 receptor binding, decreased immunogenicity, or improved IL-22 receptor signaling.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

A useful method for identification of residues or regions of a protein that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the protein with its binding partner is affected. Further substitutions may be introduced at the amino acid locations demonstrating functional sensitivity to the initial substitutions. Alternatively, or additionally, a crystal structure of a protein complex (e.g., a cytokine-receptor complex) can be used to identify contact points between a protein and its binding partner. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues.

Provided herein are nucleic acids encoding IL-22 Fc fusion proteins. In some embodiments, the nucleic acid encodes the IL-22 Fc fusion protein comprising the amino acid sequence of SEQ ID NO:8, SEQ ID NO:10, SEQ ID NO:12, SEQ ID NO:14, SEQ ID NO:24 or SEQ ID NO:26, preferably SEQ ID NO:8, SEQ ID NO:10, or SEQ ID NO:16, more preferably SEQ ID NO:8. In certain other embodiments, the nucleic acid comprises the polynucleotide sequence of SEQ ID NO:7, SEQ ID NO:9, SEQ ID NO:11, SEQ ID NO:13, SEQ ID NO:23 or SEQ ID NO:25. In certain particular embodiments, the nucleic acid comprises the polynucleotide sequence of SEQ ID NO:7 or SEQ ID NO:11, preferably SEQ ID NO:7. In certain embodiments, the isolated nucleic acid comprises a polynucleotide sequence that is at least 80%, 85%, 86%, 87%, 88%, 89%, 90%, 91%, 92%, 93%, 94%, 95%, 96%, 97%, 98%, 99% or 100% sequence identity to the polynucleotide sequence of SEQ ID NO:7, SEQ ID NO:9, SEQ ID NO:11, SEQ ID NO:13; SEQ ID NO:23 or SEQ ID NO:25. In certain embodiments, the isolated nucleic acid comprises a polynucleotide sequence that is at least 80%, 85%, 86%, 87%, 88%, 89%, 90%, 91%, 92%, 93%, 94%, 95%, 96%, 97%, 98%, 99% or 100% sequence identity to the polynucleotide sequence of SEQ ID NO:7, SEQ ID NO:9, SEQ ID NO:11, SEQ ID NO:13; SEQ ID NO:23 or SEQ ID NO:25, wherein the isolated nucleic acid is capable of encoding an IL-22 Fc fusion protein that is capable of binding to IL-22R and/or triggering IL-22R activity and wherein the Fc region of the IL-22 Fc fusion protein is not glycosylated. In certain embodiments, the isolated nucleic acid comprises a polynucleotide sequence that is at least 80%, 85%, 86%, 87%, 88%, 89%, 90%, 91%, 92%, 93%, 94%, 95%, 96%, 97%, 98%, 99% or 100% sequence identity to the polynucleotide sequence of SEQ ID NO:7, SEQ ID NO:9, SEQ ID NO:11, SEQ ID NO:13; SEQ ID NO:23 or SEQ ID NO:25, wherein the isolated nucleic acid is capable of encoding an IL-22 Fc fusion protein comprising the amino acid sequence of SEQ ID NO:8, 10, 12, or 14. In related aspects, the invention provides vectors comprising the nucleic acid described above, and a host cell comprising the vector. In certain embodiments, the host cell is a prokaryotic cell or eukaryotic cell. In certain particular embodiments, the host cell is a prokaryotic cell, including without limitation, an E. coli cell. In certain other embodiments, the host cell is a eukaryotic cell, including without limitation, a CHO cell. In certain embodiments, the host cell comprises a vector comprising a nucleic acid encoding the IL-22 Fc fusion protein comprising the amino acid sequence of SEQ ID NO:8.

### a) Glycosylation Variants

In certain embodiments, an IL-22 Fc fusion protein provided herein is altered to increase or decrease the extent to which the Fc portion of the fusion protein is glycosylated. Addition or deletion of glycosylation sites to a protein may be conveniently accomplished by altering the amino acid sequence such that one or more glycosylation sites is created or removed.

Where the fusion protein comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody or the Fc region of an antibody may be made in order to create Fc variants with certain improved properties.

The amount of fucose attached to the CH2 domain of the Fc region can be determined by calculating the average amount of fucose within the sugar chain at Asn297, relative to the sum of all glycostructures attached to Asn 297 or N297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108; US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1; and WO 2004/056312 A1, especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### b) Fc Region Variants

In certain embodiments, one or more amino acid modifications may be introduced into the Fc region of an Fc fusion protein provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an Fc variant that possesses some but not all effector functions, which make it a desirable candidate for applications in which the half-life of the antibody or a fusion protein comprising an Fc region in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody or Fc lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch et al., Annu. Rev. Immunol. 9:457-492 (1991). Non-limiting examples of in vitro assays to assess ADCC activity of a molecule of interest is described in U.S. Pat. No. 5,500,362 (see, e.g. Hellstrom et al., Proc. Nat'l Acad. Sci. USA 83:7059-7063 (1986) and Hellstrom et al., Proc. Nat'l Acad. Sci. USA 82:1499-1502 (1985); U.S. Pat. No. 5,821,337 (see Bruggemann et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CYTOTOX 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody or Fc is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg et al., Blood 101:1045-1052 (2003); and Cragg et al., Blood 103:2738-2743 (2004)). FcRn binding and in vivo clearance/half-life determinations can also be performed using methods known in the art (see, e.g., Petkova et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody or Fc variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an IL-22 Fc fusion protein comprises an Fc variant with one or more amino acid substitutions which reduce ADCC, e.g., substitution at position 297 of the Fc region to remove the N-glycosylation site and yet retain FcRn binding activity (EU numbering of residues).

In some embodiments, alterations are made in the Fc region that result in diminished C1g binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### c) Cysteine Engineered Variants

In certain embodiments, it may be desirable to create cysteine engineered Fc fusion protein, in which one or more residues of the Fc region of an antibody are substituted with cysteine residues. In particular embodiments, the substituted residues occur at accessible sites of the Fc. By substituting those residues with cysteine, reactive thiol groups are thereby positioned at accessible sites of the Fc and may be used to conjugate the Fc to other moieties, such as drug moieties or linker-drug moieties, to create an immunoconjugate, as described further herein. For example, S400 (EU numbering) of the heavy chain Fc region can be substituted with Cysteine. See e.g., U.S. Pat. No. 7,521,541.

B. Methods of Making and/or Purifying IL-22 Fc Fusion Proteins

The IL-22 Fc fusion proteins provided herein can be prepared by any suitable method, e.g., culturing cells transformed or transfected with a vector containing a nucleic acid encoding an IL-22 Fc fusion protein, a fragment, or a variant thereof. Host cells comprising any such vector are also provided. Any suitable host cell can be used, e.g., mammalian cells (e.g., CHO cells), E. coli, or yeast. Processes for producing any of the herein described IL-22 Fc fusion proteins are further provided and, in general, involve culturing host cells under conditions suitable for expression of the desired IL-22 Fc fusion protein and recovering, and optionally purifying, the desired IL-22 Fc fusion protein from the cell culture. Also provided herein are methods of selecting batches that include IL-22 Fc fusion proteins.

For example, provided herein is a method of making any of the IL-22 Fc fusion proteins described herein that includes one, two, three, or all four of the following steps: (a) providing a host cell comprising a nucleic acid encoding any of the IL-22 Fc fusion proteins described herein (e.g., an IL-22 Fc fusion protein that includes an IL-22 polypeptide linked to an Fc region by a linker); (b) culturing the host cell in a seed train medium under conditions suitable to form a seed train culture; (c) inoculating the seed train culture into an inoculum medium and culturing under conditions suitable to form an inoculum train culture; and/or (d) culturing the inoculum train culture in a production medium under conditions suitable to form a production culture, wherein the host cells of the production culture express the IL-22 Fc fusion protein, thereby making the IL-22 Fc fusion protein. In some embodiments, the IL-22 polypeptide is glycosylated. In some embodiments, the IL-22 Fc fusion protein has a sialic acid content of from about 6 to about 16 moles of sialic acid (e.g., about 6, about 7, about 8, about 9, about 10, about 11, about 12, about 13, about 14, about 15, or about 16 moles of sialic acid) per mole of the IL-22 Fc fusion protein.

In any of the preceding methods, the host cell can be a frozen host cell, and step (a) further includes thawing the frozen host cell in a seed train medium. The host cell can be frozen at any suitable temperature, e.g., about 0° C., about −10° C., about −20° C., about −30° C., about −40° C., about −50° C., about −60° C., about −70° C., about −80° C., about −90° C., about −100° C., or lower. The frozen host cell can be thawed for any suitable amount of time and at any suitable temperature(s). In other examples, a rolling seed train can be used for production of IL-22 Fc fusion protein. In this example, the seed train is grown continuously (up to a certain cell age) to inoculate the inoculum train rather than using frozen host cells.

In some embodiments of any of the preceding methods, the seed train medium or the seed train culture has a volume of about 1 L to about 100 L, e.g., about 1 L, about 2 L, about 3 L, about 4 L, about 5 L, about 10 L, about 15 L, about 20 L, about 25 L, about 30 L, about 35 L, about 40 L, about 45 L, about 50 L, about 55 L, about 60 L, about 70 L, about 75 L, about 80 L, about 85 L, about 90 L, about 95 L, or about 100 L. In some embodiments, the seed train medium or the seed train culture has a volume of about 5 L to about 50 L. In some embodiments, the seed train medium or the seed train culture has a volume of about 10 L to about 40 L. In some embodiments, the seed train medium or the seed train culture has a volume of about 15 L to about 25 L. In some embodiments, the seed train medium or the seed train culture has a volume of about 20 L.

The inoculum train medium or the inoculum train culture may have any suitable volume. In some embodiments of any of the preceding methods, the inoculum train medium or the inoculum train culture has a volume of about 10 L to about 4,000 L, e.g., about 10 L, about 15 L, about 20 L, about 25 L, about 30 L, about 35 L, about 40 L, about 45 L, about 50 L, about 55 L, about 60 L, about 70 L, about 75 L, about 80 L, about 85 L, about 90 L, about 95 L, about 100 L, about 105 L, about 110 L, about 115 L, about 120 L, about 125 L, about 130 L, about 135 L, about 140 L, about 145 L, about 150 L, about 155 L, about 160 L, about 165 L, about 170 L, about 175 L, about 180 L, about 185 L, about 190 L, about 195 L, about 200 L, about 300 L, about 400 L, about 500 L, about 600 L, about 700 L, about 800 L, about 900 L, about 1000 L, about 1,500 L, about 2,000 L, about 2,500 L, about 3,000 L, about 3,500 L, or about 4,000 L. In some embodiments, the inoculum train medium or the inoculum train culture has a volume of about 50 L to about 100 L. In some embodiments, the inoculum train medium or the inoculum train culture has a volume of about 75 L to about 90 L. In some embodiments, the inoculum train medium or the inoculum train culture has a volume of about 80 L. In other embodiments, the inoculum train medium or the inoculum train culture has a volume of about 300 L to about 500 L (e.g., about 300 L, about 320 L, about 340 L, about 360 L, about 380 L, about 400 L, about 420 L, about 440 L, about 460 L, about 480 L, or about 500 L). In some embodiments, the inoculum train medium or the inoculum train culture has a volume of about 350 L to about 450 L. In some embodiments, the inoculum train medium or the inoculum train culture has a volume of about 400 L.

The production medium or the production culture may have any suitable volume. In some embodiments of any of the preceding methods, the production medium or the production culture has a volume of about 100 L to about 30,000 L, e.g., about 100 L, about 200 L, about 300 L, about 400 L, about 500 L, about 600 L, about 700 L, about 800 L, about 900 L, about 1000 L, about 1,500 L, about 2,000 L, about 2,500 L, about 3,000 L, about 3,500 L, about 4,000 L, about 4,500 L, about 5,000 L, about 5,500 L, about 6,000 L, about 6,500 L, about 7,000 L, about 7,500 L, about 8,000 L, about 8,500 L, about 9,000 L, about 9,500 L, about 10,000 L, about 12,000 L, about 15,000 L, about 20,000 L, about 25,000 L, or about 30,000 L. In some embodiments, the production medium or the production culture has a volume of about 500 L to about 5,000 L. In some embodiments, the production medium or the production culture has a volume of about 1,000 L to about 3,000 L. In some embodiments, the production medium or the production culture has a volume of about 1,500 L to about 2,500 L. In some embodiments, the production medium or the production culture has a volume of about 2000 L.

In some embodiments of any of the preceding methods, the method further comprises passaging the inoculum train culture about 1 to about 20 times prior to step (d), e.g., about 1, about 2, about 3, about 4, about 5, about 6, about 7, about 8, about 9, about 10, about 11, about 12, about 13, about 14, about 15, about 16, about 17, about 18, about 19, or about 20 times. In some embodiments, the inoculum train culture is passaged about 1 to about 10 times prior to step (d). In some embodiments, the inoculum train culture is passaged about 2 to about 6 times prior to step (d). In some embodiments, the inoculum train culture is passaged about 2 to about 3 times prior to step (d). In some embodiments, the inoculum train culture is passaged about 5 times prior to step (d). In some embodiments, the inoculum train culture is passaged about 2 times prior to step (d). In some embodiments, the inoculum train culture is passaged about 3 times prior to step (d). In some embodiments, the inoculum train culture is passaged about 4 times prior to step (d).

In some embodiments of any of the preceding methods, the seed train medium, the inoculum train medium, and/or the production medium includes a selection agent capable of selecting for the host cell. In some embodiments, the seed train medium includes a selection agent. Any suitable selection agent can be used. In some embodiments, the selection agent is methionine sulfoximine, methotrexate, or an antibiotic (e.g., blasticidin, geneticin, hygromycin B, puromycin, mycophenolic acid, or zeocin). In particular embodiments, the selection agent is methionine sulfoximine.

In any of the preceding methods, the seed train medium, the inoculum medium, and/or the production medium can include an antifoaming agent. Any suitable antifoaming agent can be used. In some embodiments, the antifoaming agent is simethicone emulsion, antifoam 204, antifoam A, antifoam B, antifoam C, antifoam Y-30, or antifoam SE-15. In particular embodiments, the antifoaming agent is simethicone emulsion. In some embodiments, the concentration of the antifoaming agent is about 10% to about 50%, e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% (e.g., w/v). In some embodiments, the concentration of the antifoaming agent is about 30% (w/v). In some embodiments, 30% simethicone is used to make 1% or 10% antifoam solutions which are added to the culture (e.g., the seed train culture, the inoculum culture, and/or the production culture) as needed to minimize foam.

In any of the preceding methods, the seed train medium, the inoculum medium, and/or the production medium can include a buffering agent, a cell protective agent, a polysaccharide, and/or an osmolality adjustment agent.

In any of the preceding methods, step (b) can be performed at any suitable temperature, for example, a temperature of about 20° C. to about 45° C., e.g., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C. In some embodiments, step (b) is performed at a temperature of about 25° C. to about 40° C. In some embodiments, step (b) is performed at a temperature of about 35° C. to about 39° C. In some embodiments, step (b) is performed at a temperature of about 36° C. to about 38° C. In some embodiments, step (b) is performed at a temperature of about 37° C.

In any of the preceding methods, step (b) can be performed in any suitable culture vessel, for example, a spinner, a shake flask, or a seed train bioreactor (e.g., a stainless steel bioreactor or a single-use bioreactor (e.g., a WAVE BIOREACTOR™ or an AMBR® bioreactor (e.g., an AMBR® 15 or an AMBR® 250 bioreactor))). In some embodiments, step (b) is performed in a speed train spinner or a shake flask. In other embodiments, step (b) is performed in a single-use bioreactor (e.g., a WAVE BIOREACTOR™ or an AMBR® bioreactor (e.g., an AMBR® 15 bioreactor or an AMBR® 250 bioreactor)). In other embodiments, step (b) is performed in a speed train bioreactor.

In any of the preceding methods, step (b) can have a duration of about 1 day to about 20 days per passage, e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, or about 20 days per passage. In some embodiments, step (b) has a duration of about 1 day to about 12 days per passage. In some embodiments, step (b) has a duration of about 2 days to about 7 days per passage. In some embodiments, step (b) has a duration of about 2 days to about 6 days per passage. In some embodiments, step (b) has a duration of about 2 days to about 5 days per passage. In some embodiments, step (b) has a duration of about 2 days to about 4 days per passage. In some embodiments, step (b) has a duration of about 2 days to about 3 days per passage.

In any of the preceding methods, the seed train medium or the seed train culture can have any suitable pH. For example, in some embodiments, the pH of the seed train medium or the seed train culture is about 5 to about 9, e.g., about 5, about 5.5, about 6, about 6.5, about 6.6, about 6.7, about 6.8, about 6.9, about 7.0, about 7.1, about 7.15, about 7.2, about 7.3, about 7.4, about 7.5, about 8.0, about 8.5, or about 9. In some embodiments, the pH of the seed train medium or the seed train culture is about 6.5 to about 7.5. In some embodiments, the pH of the seed train medium or the seed train culture is about 7.0 to about 7.5, e.g., about 7.0, about 7.05, about 7.1, about 7.15, about 7.2, about 7.25, about 7.3, about 7.35, about 7.4, about 7.45, or about 7.5. In some embodiments, the pH of the seed train medium or the seed train culture is about 7.15. In some embodiments, the pH of the seed train culture is about 7.15.

In any of the preceding methods, the seed train medium or the seed train culture can have any suitable dissolved oxygen (e.g., percent of dissolved oxygen, where 100% indicates that the medium is saturated), e.g., about 10% to about 60% (e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%. In some embodiments, the dissolved oxygen of the seed train medium or the seed train culture is about 15% to about 50%. In some embodiments, the dissolved oxygen of the seed train medium or the seed train culture is about 20% to about 40%. In some embodiments, the dissolved oxygen of the seed train medium or the seed train culture is about 25% to about 35%. In some embodiments, the dissolved oxygen of the seed train medium or the seed train culture is about 30%. In some embodiments, the dissolved oxygen of the seed train culture is about 30%.

In any of the preceding methods, step (b) can have any suitable duration, for example, about 6 hours to about 20 days, e.g., about 6 h, about 7 h, about 8 h, about 9 h, about 10 h, about 11 h, about 12 h, about 13 h, about 14 h, about 15 h, about 16 h, about 18 h, about 19 h, about 20 h, about 21 h, about 22 h, about 23 h, about 1 day, about 1.5 days, about 2 days, about 2.5 days, about 3 days, around 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, about 10 days, about 10.5 days, about 11 days, about 11.5 days, about 12 days, about 12.5 days, about 13 days, about 13.5 days, about 14 days, about 14.5 days, about 15 days, about 15.5 days, about 16 days, about 16.5 days, about 17 days, about 17.5 days, about 18 days, about 18.5 days, about 19 days, about 19.5 days, or about 20 days. In some embodiments, step (b) has a duration of about 1 day to about 10 days. In some embodiments, step (b) has a duration of about 2 days to about 8 days. In some embodiments, step (b) has a duration of about 2 days to about 7 days. In some embodiments, step (b) has a duration of about 2 days to about 6 days. In some embodiments, step (b) has a duration of about 2 days to about 5 days. In some embodiments, step (b) has a duration of about 2 days to about 4 days. In some embodiments, step (b) has a duration of about 2 days to about 3 days.

In any of the preceding methods, step (c) can be performed at any suitable temperature, for example, a temperature of about 20° C. to about 45° C., e.g., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C. In some embodiments, step (c) is performed at a temperature of about 25° C. to about 40° C. In some embodiments, step (c) is performed at a temperature of about 35° C. to about 39° C. In some embodiments, step (c) is performed at a temperature of about 36° C. to about 38° C. In some embodiments, step (c) is performed at a temperature of about 37° C.

In any of the preceding methods, step (c) can be performed in one or more bioreactors, e.g., 1, 2, 3, 4, 5, 6, 7, 8, 9, 10, 20, or more bioreactors (e.g., a stainless steel bioreactor or a single-use bioreactor (e.g., a WAVE BIOREACTOR™)). In some embodiments, step (c) is performed in 3 bioreactors or 4 bioreactors. In some embodiments, step (c) is performed in 3 bioreactors.

In any of the preceding methods, the inoculum medium or the inoculum culture can have any suitable pH. For example, in some embodiments, the pH of the inoculum medium or the inoculum culture is about 5 to about 9, e.g., about 5, about 5.5, about 6, about 6.5, about 6.6, about 6.7, about 6.8, about 6.9, about 7.0, about 7.1, about 7.15, about 7.2, about 7.3, about 7.4, about 7.5, about 8.0, about 8.5, or about 9. In some embodiments, the pH of the inoculum medium or the inoculum culture is about 6.5 to about 7.5. In some embodiments, the pH of the inoculum medium or the inoculum culture is about 7.0 to about 7.5, e.g., about 7.0, about 7.05, about 7.1, about 7.15, about 7.2, about 7.25, about 7.3, about 7.35, about 7.4, about 7.45, or about 7.5. In some embodiments, the pH of the inoculum medium or the inoculum culture is about 7.1. In some embodiments, the pH of the inoculum culture is about 7.1.

In any of the preceding methods, the inoculum medium or the inoculum culture can have any suitable dissolved oxygen, e.g., about 10% to about 60% (e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%. In some embodiments, the dissolved oxygen of the inoculum medium or the inoculum culture is about 15% to about 50%. In some embodiments, the dissolved oxygen of the inoculum medium or the inoculum culture is about 20% to about 40%. In some embodiments, the dissolved oxygen of the inoculum medium or the inoculum culture is about 25% to about 35%. In some embodiments, the dissolved oxygen of the inoculum medium or the inoculum culture is about 30%. In some embodiments, the dissolved oxygen of the inoculum culture is about 30%.

In any of the preceding methods, step (c) can have any suitable duration, for example, about 6 hours to about 20 days, e.g., about 6 hours, about 7 hours, about 8 hours, about 9 hours, about 10 hours, about 11 hours, about 12 hours, about 13 hours, about 14 hours, about 15 hours, about 16 hours, about 18 hours, about 19 hours, about 20 hours, about 21 hours, about 22 hours, about 23 hours, about 1 day, about 1.5 days, about 2 days, about 2.5 days, about 3 days, around 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, about 10 days, about 10.5 days, about 11 days, about 11.5 days, about 12 days, about 12.5 days, about 13 days, about 13.5 days, about 14 days, about 14.5 days, about 15 days, about 15.5 days, about 16 days, about 16.5 days, about 17 days, about 17.5 days, about 18 days, about 18.5 days, about 19 days, about 19.5 days, or about 20 days. In some embodiments, step (c) has a duration of about 1 day to about 10 days. In some embodiments, step (c) has a duration of about 2 days to about 8 days. In some embodiments, step (c) has a duration of about 2 days to about 7 days. In some embodiments, step (c) has a duration of about 2 days to about 6 days. In some embodiments, step (c) has a duration of about 2 days to about 5 days. In some embodiments, step (c) has a duration of about 2 days to about 4 days. In some embodiments, step (c) has a duration of about 2 days to about 3 days.

In any of the preceding methods, step (d) can include a temperature shift from an initial temperature to a post-shift temperature. In some embodiments, the initial temperature is about 20° C. to about 45° C., e.g., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C. In some embodiments, the initial temperature is about 25° C. to about 40° C. In some embodiments, the initial temperature is about 35° C. to about 39° C. In some embodiments the initial temperature is about 36° C. to about 38° C. In some embodiments, the initial temperature is about 37° C.

In any of the preceding methods, the post-shift temperature can be below or above the initial temperature. In some embodiments, the post-shift is about 20° C. to about 45° C., e.g., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C. In some embodiments, the post-shift is about 25° C. to about 35° C. In some embodiments, the initial temperature is about 30° C. to about 35° C. In some embodiments the initial temperature is about 32° C. to about 34° C. In some embodiments, the initial temperature is about 33° C.

In any of the preceding methods, the temperature shift can occur over a period of about 1 h to about 140 h, e.g., about 1 h, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 18 h, about 19 h, about 20 h, about 21 h, about 22 h, about 23 h, about 24 h, about 25 h, about 30 h, about 35 h, about 40 h, about 45 h, about 50 h, about 55 h, about 56 h, about 57 h, about 58 h, about 59 h, about 60 h, about 61 h, about 62 h, about 63 h, about 64 h, about 65 h, about 66 h, about 67 h, about 68 h, about 69 h, about 70 h, about 71 h, about 72 h, about 73, about 74 h, about 75 h, about 76 h, about 77 h, about 78 h, about 79 h, about 80 h, about 85 h, about 90 h, about 95 h, about 100 h, about 105 h, about 110 h, about 115 h, about 120 h, about 125 h, about 130 h, about 135 h, or about 140 h. For example, in some embodiments, the temperature shift occurs over a period of about 12 h to about 120 h. In some embodiments, the temperature shift occurs over a period of about 24 h to about 96 h. In some embodiments, the temperature shift occurs over a period of about 48 h to about 96 h. In some embodiments, the temperature shift occurs over a period of about 60 h to about 80 h. In some embodiments, the temperature shift occurs over a period of about 72 h.

In any of the preceding methods, the production medium or the production culture can have any suitable pH. For example, in some embodiments, the pH of the production medium or the production culture is about 5 to about 9, e.g., about 5, about 5.5, about 6, about 6.5, about 6.6, about 6.7, about 6.8, about 6.9, about 7.0, about 7.1, about 7.15, about 7.2, about 7.3, about 7.4, about 7.5, about 8.0, about 8.5, or about 9. In some embodiments, the pH of the production medium or the production culture is about 6.5 to about 7.5. In some embodiments, the pH of the production medium or the production culture is about 7.0 to about 7.5, e.g., about 7.0, about 7.05, about 7.1, about 7.15, about 7.2, about 7.25, about 7.3, about 7.35, about 7.4, about 7.45, or about 7.5. In some embodiments, the pH of the production medium or the production culture is about 7.0. In some embodiments, the pH of the production culture is about 7.0.

In any of the preceding methods, step (d) can be performed in any suitable culture vessel, e.g., a production bioreactor (e.g., a stainless steel bioreactor or a single-use bioreactor (e.g., a WAVE BIOREACTOR™)).

In any of the preceding methods, the production medium or the production culture can have any suitable dissolved oxygen, e.g., about 10% to about 60% (e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%. In some embodiments, the dissolved oxygen of the production medium or the production culture is about 15% to about 50%. In some embodiments, the dissolved oxygen of the production medium or the production culture is about 20% to about 40%. In some embodiments, the dissolved oxygen of the production medium or the production culture is about 25% to about 35%. In some embodiments, the dissolved oxygen of the production medium or the production culture is about 30%. In some embodiments, the dissolved oxygen of the production culture is about 30%.

In any of the preceding methods, step (d) can have any suitable duration, for example, about 6 hours to about 30 days, e.g., about 6 hours, about 7 hours, about 8 hours, about 9 hours, about 10 hours, about 11 hours, about 12 hours, about 13 hours, about 14 hours, about 15 hours, about 16 hours, about 18 hours, about 19 hours, about 20 hours, about 21 hours, about 22 hours, about 23 hours, about 1 day, about 1.5 days, about 2 days, about 2.5 days, about 3 days, around 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, about 10 days, about 10.5 days, about 11 days, about 11.5 days, about 12 days, about 12.5 days, about 13 days, about 13.5 days, about 14 days, about 14.5 days, about 15 days, about 15.5 days, about 16 days, about 16.5 days, about 17 days, about 17.5 days, about 18 days, about 18.5 days, about 19 days, about 19.5 days, about 20 days, about 20.5 days, about 21 days, about 21.5 days, about 22 days, about 22.5 days, about 23 days, about 23.5 days, about 24 days, about 24.5 days, about 25 days, about 25.5 days, about 26 days, about 26.5 days, about 27 days, about 27.5 days, about 28 days, about 28.5 days, about 29 days, about 29.5 days, or about 30 days. In some embodiments, step (c) has a duration of about 1 day to about 10 days. In some embodiments, step (d) has a duration of about 2 days to about 25 days. In some embodiments, step (d) has a duration of about 5 days to about 25 days. In some embodiments, step (d) has a duration of about 7 days to about 14 days. In some embodiments, step (d) has a duration of about 8 days to about 16 days. In some embodiments, step (c) has a duration of about 10 days to about 14 days. In some embodiments, step (d) has a duration of about 11 days to about 13 days. In some embodiments, step (d) has a duration of about 12 days.

In another aspect, provided herein is a method of making a composition comprising an IL-22 Fc fusion protein, the method comprising the following steps: (a) providing a host cell comprising a nucleic acid encoding a IL-22 Fc fusion protein, the IL-22 Fc fusion protein comprising an IL-22 polypeptide linked to an Fc region by a linker; (b) culturing the host cell in a seed train medium under conditions suitable to form a seed train culture; (c) inoculating the seed train in an inoculum medium under conditions suitable to form an inoculum train culture; and (d) culturing the inoculum train in a production medium under conditions suitable to form a production culture, wherein the host cells of the production culture express the IL-22 Fc fusion protein, and wherein the duration of step (d) is at least 10 days, thereby making the composition comprising an IL-22 Fc fusion protein, wherein the IL-22 polypeptide is glycosylated, and wherein the composition has an average sialic acid content in the range of 6 to 12 moles of sialic acid per mole of the IL-22 Fc fusion protein. In some embodiments, the duration of step (d) is at least 11 days, at least 12 days, or at least 13 days. In some embodiments, the duration of step (d) is 12 days.

In any of the preceding methods, step (d) can further include adding nutrients to the production medium or the production culture by a nutrient feed.

In any of the preceding methods, any suitable host cell can be used. In some embodiments, the host cell is a prokaryotic cell. In other embodiments, the host cell is a eukaryotic cell. In some embodiments, the eukaryotic cell is a mammalian cell (e.g., a CHO cell, such as a suspension-adapted CHO cell). Additional suitable host cells are known in the art and described below, for example, insect cells or plant cells.

Any of the preceding methods can further include the following step: (e) harvesting a cell culture fluid comprising the IL-22 Fc fusion protein from the production culture. In some embodiments, step (e) comprises cooling the production culture (e.g., to about 1° C. to about 10° C. (e.g., about 1° C., about 2° C., about 3° C., about 4° C., about 5° C., about 6° C., about 8° C., about 9° C., or about 10° C.), e.g., 2° C. to about 8° C.). In some embodiments, step (e) comprises removing the host cells from the production medium by centrifugation to form the cell culture fluid. In some embodiments, step (e) further comprises filtering the cell culture fluid.

Any of the preceding methods can further include the following step: (f) purifying the IL-22 Fc fusion protein in the cell culture fluid. In some embodiments, step (f) includes one, two, three, or all four of the following substeps: (i) contacting the cell culture fluid to an affinity chromatographic support, optionally washing the affinity chromatographic support with a wash buffer, eluting the IL-22 Fc fusion protein from the affinity chromatographic support with a first elution buffer to form an affinity pool, and optionally inactivating viruses in the affinity pool; (ii) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and (iii) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool.

In another aspect, the invention provides a method of purifying an IL-22 Fc fusion protein that includes one, two, three, or all four of the following steps: (a) providing a cell culture fluid comprising an IL-22 Fc fusion protein and optionally inactivating viruses in the cell culture fluid; (b) contacting the cell culture fluid to an affinity chromatographic support, optionally washing the affinity chromatographic support with a wash buffer, eluting the IL-22 Fc fusion protein from the affinity chromatographic support with a first elution buffer to form an affinity pool, and optionally inactivating viruses in the affinity pool; (c) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and (d) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool. In some embodiments, the IL-22 polypeptide is glycosylated. In some embodiments, the IL-22 Fc fusion protein has a sialic acid content of from about 6 to about 16 moles of sialic acid (e.g., about 6, about 7, about 8, about 9, about 10, about 11, about 12, about 13, about 14, about 15, or about 16 moles of sialic acid) per mole of the IL-22 Fc fusion protein.

Any of the preceding methods can include concentrating the purified product pool to form a concentrated product pool. Any of the preceding methods can include ultrafiltering the purified product pool. In some embodiments of any of the preceding methods, ultrafiltering comprises filtering the purified product pool with a regenerated cellulose ultrafiltration membrane, e.g., a 10 kDa composite regenerated cellulose ultrafiltration membrane. Any of the preceding methods can include exchanging the buffer of the concentrated product pool to form an ultrafiltration and diafiltration (UFDF) pool comprising the IL-22 Fc fusion protein. In some embodiments, the buffer of the concentrated product pool is exchanged with a diafiltration buffer comprising 0.01 M sodium phosphate, pH 7.2, final concentration. Any of the preceding methods can include conditioning the UFDF pool with a formulation buffer to form a conditioned UFDF pool comprising the IL-22 Fc fusion protein.

Any of the preceding methods can include one or more virus inactivation steps. For example, in some embodiments of any of the preceding methods, inactivating viruses includes adding a detergent to the cell culture fluid, the affinity pool, the anion-exchange pool, and/or the purified product pool. In some embodiments, inactivating viruses includes adding a detergent to the cell culture fluid. For example, in some embodiments, substep (i) further comprises inactivating viruses adding a detergent to the cell culture fluid prior to contacting the cell culture fluid to the affinity column. In some embodiments, inactivating viruses includes adding a detergent to the affinity pool. In some embodiments, substep (i) comprises inactivating viruses by adding a detergent to the affinity pool.

Any suitable detergent can be used for inactivating viruses, for example, TRITON® X-100 or TRITON® CG110. In some embodiments, the final concentration of the detergent in the cell culture fluid is about 0.001% to about 5% (e.g., v/v), e.g., about 0.001%, about 0.01%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 2%, about 3%, about 4%, or about 5%. In some embodiments, the final concentration of the detergent in the cell culture fluid is about 0.01% to about 2%. In some embodiments, the final concentration of the detergent is about 0.1% to about 1%. In some embodiments, the final concentration of the detergent is about 0.3% to about 0.5%. In some embodiments, the final concentration of the detergent is about 0.5%. The virus inactivation can be performed at any suitable temperature, e.g., about 4° C. to about 40° C., e.g., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In some embodiments, the virus inactivation is performed at about 2012° to about 25° C. In some embodiments, the virus inactivation has a duration of greater than about 0.25 h, e.g., greater than about 0.25 h, about 0.5 h, about 1 h, about 1.5 h, about 2 h, about 2.5 h, about 3 h, about 3.5 h, about 4 h, about 4.5 h, about 5 h, about 5.5 h, about 6 h, or longer. In some embodiments, the virus inactivation has a duration of greater than about 0.5 h, e.g., about 5 h to 48 h, about 5 h to about 24 h, or any other suitable duration.

In another example, the invention provides a method of making a composition comprising an IL-22 Fc fusion protein that includes culturing an inoculum train culture comprising a plurality of host cells in a production medium under conditions suitable to form a production culture, wherein the host cells comprise a nucleic acid encoding an IL-22 Fc fusion protein, the IL-22 Fc fusion protein comprising an IL-22 polypeptide linked to an Fc region by a linker, wherein the host cells express the IL-22 Fc fusion protein, and wherein the duration of the culturing is at least 10 days, thereby making the composition comprising an IL-22 Fc fusion protein, wherein the IL-22 polypeptide is glycosylated, and wherein the composition has an average sialic acid content in the range of 6 to 12 moles of sialic acid per mole of the IL-22 Fc fusion protein. In some embodiments, the duration of the culturing is at least 11 days, at least 12 days, or at least 13 days. In some embodiments, the duration of the culturing is 12 days.

In some embodiments of any of the preceding aspects, the method further includes generating a seed train culture by culturing a host cell comprising a nucleic acid encoding the IL-22 Fc fusion protein in a seed train medium under conditions suitable to form the seed train culture prior to culturing the inoculum train culture in the production medium. In some embodiments, the method further includes inoculating the seed train culture in an inoculum medium under conditions suitable to form an inoculum train culture prior to culturing the inoculum train culture in the production medium.

Any suitable host cell may be used. In any of the methods, the host cells may be eukaryotic host cells or prokaryotic host cells. In some embodiments, the eukaryotic host cells are mammalian host cells. In some embodiments, the mammalian host cells are Chinese hamster ovary (CHO) cells. In some embodiments, harvesting the cell culture fluid comprises: (i) cooling the production culture; (ii) removing the host cells from the production medium by centrifugation to form the cell culture fluid; and/or (iii) filtering the cell culture fluid.

Any of the methods may further include purifying the IL-22 Fc fusion protein in the cell culture fluid. In some embodiments, purifying the IL-22 Fc fusion protein comprises the following substeps: (i) contacting the cell culture fluid to an affinity chromatographic support, optionally washing the affinity chromatographic support with a wash buffer, eluting the IL-22 Fc fusion protein from the affinity chromatographic support with a first elution buffer to form an affinity pool, and optionally inactivating viruses in the affinity pool; (ii) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and (iii) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool. In some embodiments, purifying the IL-22 Fc fusion protein further comprises one or more of the following substeps: (iv) concentrating the purified product pool to form a concentrated product pool; (v) ultrafiltering the purified product pool; (vi) exchanging the buffer of the concentrated product pool to form a ultrafiltration and diafiltration (UFDF) pool comprising the IL-22 Fc fusion protein; and/or (vii) conditioning the UFDF pool with a formulation buffer to form a conditioned UFDF pool comprising the IL-22 Fc fusion protein. In some embodiments, substep (i) further comprises inactivating viruses by adding a detergent to the cell culture fluid prior to contacting the cell culture fluid to the affinity column. In another example, the invention provides a method for controlling sialic acid content of a composition comprising an IL-22 Fc fusion protein, the IL-22 Fc fusion protein comprising a glycosylated IL-22 polypeptide linked by a linker to an antibody Fc region, the method comprising: culturing an inoculum train culture comprising a plurality of host cells in a production medium under conditions suitable to form a production culture for at least 10 days, wherein the host cells comprise a nucleic acid encoding the IL-22 Fc fusion protein and express the IL-22 Fc fusion protein, wherein the composition has an average sialic acid content in the range of 6 to 12 moles of sialic acid per mole of the IL-22 Fc fusion protein; and enriching the average sialic acid content of the composition to the range of 8 to 12 moles of sialic acid per mole of the IL-22 Fc fusion protein, thereby controlling the sialic acid content of the composition. In some embodiments, the method comprises enriching the average sialic acid content of the composition to the range of 8 to 9 moles of sialic acid per mole of the IL-22 Fc fusion protein.

Any of the methods described herein may include enriching the sialic acid content of the composition. Enrichment may be performed using any suitable approach, for example, by purifying the IL-22 Fc fusion protein as described herein. For example, in some embodiments, enriching the average sialic acid content comprises harvesting a cell culture fluid comprising the IL-22 Fc fusion protein from the production culture. In some embodiments, harvesting the cell culture fluid comprises: (i) cooling the production culture; (ii) removing the host cells from the production medium by centrifugation to form the cell culture fluid; and/or (iii) filtering the cell culture fluid. Enriching the average sialic acid content of the composition may include purifying the IL-22 Fc fusion protein in a cell culture fluid. In some embodiments, purifying the IL-22 Fc fusion protein comprises the following substeps: (i) contacting the cell culture fluid to an affinity chromatographic support, optionally washing the affinity chromatographic support with a wash buffer, eluting the IL-22 Fc fusion protein from the affinity chromatographic support with a first elution buffer to form an affinity pool, and optionally inactivating viruses in the affinity pool; (ii) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and (iii) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool. In some embodiments, purifying the IL-22 Fc fusion protein further comprises one or more of the following substeps: (iv) concentrating the purified product pool to form a concentrated product pool; (v) ultrafiltering the purified product pool; (vi) exchanging the buffer of the concentrated product pool to form a ultrafiltration and diafiltration (UFDF) pool comprising the IL-22 Fc fusion protein; and/or (vii) conditioning the UFDF pool with a formulation buffer to form a conditioned UFDF pool comprising the IL-22 Fc fusion protein. In some embodiments, substep (i) further comprises inactivating viruses by adding a detergent to the cell culture fluid prior to contacting the cell culture fluid to the affinity column. In some embodiments, the affinity chromatographic support comprises a protein A resin, a protein G resin, or an IL-22 receptor resin. In some embodiments, the protein A resin is a MABSELECT SURE® resin. In some embodiments, the anion-exchange chromatographic support comprises a strong anion exchanger with multimodal functionality resin. In some embodiments, the anion-exchange chromatographic support comprises a CAPTO™ adhere resin.

In some embodiments, the composition has an initial average sialic acid content in the range of about 1 to about 8 moles (e.g., about 1, about 2, about 3, about 4, about 5, about 6, about 7, or about 8 moles) of sialic acid per mole of the IL-22 Fc fusion protein. In some embodiments, the composition has an initial average sialic acid content of about 6, about 7, or about 8 moles of sialic acid per mole of the IL-22 Fc fusion protein. In some embodiments, the composition has an initial average sialic acid content of 6 moles of sialic acid per mole of the IL-22 Fc fusion protein. In other embodiments, the composition has an initial average sialic acid content of 7 moles of sialic acid per mole of the IL-22 Fc fusion protein. In still other embodiments, the composition has an initial average sialic acid content of 8 moles of sialic acid per mole of the IL-22 Fc fusion protein. In some embodiments, the method further includes enriching the average sialic acid content to the range of 8 to 12 moles of sialic acid per mole of the IL-22 Fc fusion protein. In some embodiments, the method further includes enriching the average sialic acid content to the range of 8 to 9 moles of sialic acid per mole of the IL-22 Fc fusion protein.

For example, in some embodiments, the method further includes enriching the average sialic acid content from an initial average sialic acid content in the range of about 1 to about 8 moles of sialic acid per mole of the IL-22 Fc fusion protein (e.g., about 1, about 2, about 3, about 4, about 5, about 6, about 7 moles, or about 8 moles of sialic acid per mole of the IL-22 Fc fusion protein) to the range of about 8 to about 12 moles of sialic acid per mole of the IL-22 Fc fusion protein. In some embodiments, the method further includes enriching the average sialic acid content from an initial average sialic acid content of about 3 moles of sialic acid per mole of the IL-22 Fc fusion protein to the range of about 8 to about 12 moles of sialic acid per mole of the IL-22 Fc fusion protein. In some embodiments, the method further includes enriching the average sialic acid content from an initial average sialic acid content of about 4 moles of sialic acid per mole of the IL-22 Fc fusion protein to the range of about 8 to about 12 moles of sialic acid per mole of the IL-22 Fc fusion protein. In some embodiments, the method further includes enriching the average sialic acid content from an initial average sialic acid content of about 5 moles of sialic acid per mole of the IL-22 Fc fusion protein to the range of about 8 to about 12 moles of sialic acid per mole of the IL-22 Fc fusion protein. In some embodiments, the method further includes enriching the average sialic acid content from an initial average sialic acid content of about 6 moles of sialic acid per mole of the IL-22 Fc fusion protein to the range of about 8 to about 12 moles of sialic acid per mole of the IL-22 Fc fusion protein. In some embodiments, the method further includes enriching the average sialic acid content from an initial average sialic acid content of about 7 moles of sialic acid per mole of the IL-22 Fc fusion protein to the range of about 8 to about 12 moles of sialic acid per mole of the IL-22 Fc fusion protein. In some embodiments, the method further includes enriching the average sialic acid content from an initial average sialic acid content of about 8 moles of sialic acid per mole of the IL-22 Fc fusion protein to the range of about 8 to about 12 moles of sialic acid per mole of the IL-22 Fc fusion protein.

In other examples, in some embodiments, the method further includes enriching the average sialic acid content from an initial average sialic acid content in the range of 1 to 8 moles of sialic acid per mole of the IL-22 Fc fusion protein (e.g., 1, 2, 3, 4, 5, 6, 7, or 8 moles of sialic acid per mole of the IL-22 Fc fusion protein) to the range of 8 to 12 moles of sialic acid per mole of the IL-22 Fc fusion protein. In some embodiments, the method further includes enriching the average sialic acid content from an initial average sialic acid content of 3 moles of sialic acid per mole of the IL-22 Fc fusion protein to the range of 8 to 12 moles of sialic acid per mole of the IL-22 Fc fusion protein. In some embodiments, the method further includes enriching the average sialic acid content from an initial average sialic acid content of 4 moles of sialic acid per mole of the IL-22 Fc fusion protein to the range of 8 to 12 moles of sialic acid per mole of the IL-22 Fc fusion protein. In some embodiments, the method further includes enriching the average sialic acid content from an initial average sialic acid content of 5 moles of sialic acid per mole of the IL-22 Fc fusion protein to the range of 8 to 12 moles of sialic acid per mole of the IL-22 Fc fusion protein. In some embodiments, the method further includes enriching the average sialic acid content from an initial average sialic acid content of 6 moles of sialic acid per mole of the IL-22 Fc fusion protein to the range of 8 to 12 moles of sialic acid per mole of the IL-22 Fc fusion protein. In some embodiments, the method further includes enriching the average sialic acid content from an initial average sialic acid content of 7 moles of sialic acid per mole of the IL-22 Fc fusion protein to the range of 8 to 12 moles of sialic acid per mole of the IL-22 Fc fusion protein. In some embodiments, the method further includes enriching the average sialic acid content from an initial average sialic acid content of 8 moles of sialic acid per mole of the IL-22 Fc fusion protein to the range of 8 to 12 moles of sialic acid per mole of the IL-22 Fc fusion protein.

In other embodiments, the method further includes enriching the average sialic acid content from an initial average sialic acid content of about 1 to about 8 moles of sialic acid per mole of the IL-22 Fc fusion protein (e.g., about 1, about 2, about 3, about 4, about 5, about 6, about 7 moles, or about 8 moles of sialic acid per mole of the IL-22 Fc fusion protein) to the range of about 8 to about 9 moles of sialic acid per mole of the IL-22 Fc fusion protein. For example, in some embodiments, the method further includes enriching the average sialic acid content from an initial average sialic acid content of about 3 moles of sialic acid per mole of the IL-22 Fc fusion protein to the range of about 8 to about 9 moles of sialic acid per mole of the IL-22 Fc fusion protein. In some embodiments, the method further includes enriching the average sialic acid content from an initial average sialic acid content of about 3 moles of sialic acid per mole of the IL-22 Fc fusion protein to the range of about 8 to about 9 moles of sialic acid per mole of the IL-22 Fc fusion protein. In some embodiments, the method further includes enriching the average sialic acid content from an initial average sialic acid content of about 4 moles of sialic acid per mole of the IL-22 Fc fusion protein to the range of about 8 to about 9 moles of sialic acid per mole of the IL-22 Fc fusion protein. In some embodiments, the method further includes enriching the average sialic acid content from an initial average sialic acid content of about 5 moles of sialic acid per mole of the IL-22 Fc fusion protein to the range of about 8 to about 9 moles of sialic acid per mole of the IL-22 Fc fusion protein. In some embodiments, the method further includes enriching the average sialic acid content from an initial average sialic acid content of about 6 moles of sialic acid per mole of the IL-22 Fc fusion protein to the range of about 8 to about 9 moles of sialic acid per mole of the IL-22 Fc fusion protein. In some embodiments, the method further includes enriching the average sialic acid content from an initial average sialic acid content of about 7 moles of sialic acid per mole of the IL-22 Fc fusion protein to the range of about 8 to about 9 moles of sialic acid per mole of the IL-22 Fc fusion protein. In some embodiments, the method further includes enriching the average sialic acid content from an initial average sialic acid content of about 8 moles of sialic acid per mole of the IL-22 Fc fusion protein to the range of about 8 to about 9 moles of sialic acid per mole of the IL-22 Fc fusion protein.

In still other embodiments, the method further includes enriching the average sialic acid content from an initial average sialic acid content of 1 to 8 moles of sialic acid per mole of the IL-22 Fc fusion protein (e.g., 1, 2, 3, 4, 5, 6, 7, or 8 moles of sialic acid per mole of the IL-22 Fc fusion protein) to the range of 8 to 9 moles of sialic acid per mole of the IL-22 Fc fusion protein. For example, in some embodiments, the method further includes enriching the average sialic acid content from an initial average sialic acid content of 3 moles of sialic acid per mole of the IL-22 Fc fusion protein to the range of 8 to 9 moles of sialic acid per mole of the IL-22 Fc fusion protein. In some embodiments, the method further includes enriching the average sialic acid content from an initial average sialic acid content of 3 moles of sialic acid per mole of the IL-22 Fc fusion protein to the range of 8 to 9 moles of sialic acid per mole of the IL-22 Fc fusion protein. In some embodiments, the method further includes enriching the average sialic acid content from an initial average sialic acid content of 4 moles of sialic acid per mole of the IL-22 Fc fusion protein to the range of 8 to 9 moles of sialic acid per mole of the IL-22 Fc fusion protein. In some embodiments, the method further includes enriching the average sialic acid content from an initial average sialic acid content of 5 moles of sialic acid per mole of the IL-22 Fc fusion protein to the range of 8 to 9 moles of sialic acid per mole of the IL-22 Fc fusion protein. In some embodiments, the method further includes enriching the average sialic acid content from an initial average sialic acid content of 6 moles of sialic acid per mole of the IL-22 Fc fusion protein to the range of 8 to 9 moles of sialic acid per mole of the IL-22 Fc fusion protein. In some embodiments, the method further includes enriching the average sialic acid content from an initial average sialic acid content of 7 moles of sialic acid per mole of the IL-22 Fc fusion protein to the range of 8 to 9 moles of sialic acid per mole of the IL-22 Fc fusion protein. In some embodiments, the method further includes enriching the average sialic acid content from an initial average sialic acid content of 8 moles of sialic acid per mole of the IL-22 Fc fusion protein to the range of 8 to 9 moles of sialic acid per mole of the IL-22 Fc fusion protein.

In some embodiments of any of the preceding methods, the affinity chromatographic support comprises a protein A resin, a protein G resin, or an IL-22 receptor resin. In some embodiments of any of the preceding methods, the affinity chromatographic support comprises a protein A resin. In some embodiments, the protein A resin is a MABSELECT SURE® resin. In some embodiments, the wash buffer comprises 0.4 M potassium phosphate, pH 7.0, final concentration. In some embodiments, the first elution buffer comprises 0.3 M L-arginine hydrochloride, 0.013 M sodium phosphate, pH 3.8, final concentration.

In some embodiments of any of the preceding methods, the anion-exchange chromatographic support comprises a strong anion exchanger with multimodal functionality resin. In some embodiments, the anion-exchange chromatographic support comprises a CAPTO™ adhere resin. In some embodiments, the first equilibration buffer comprises 0.04 M sodium acetate, pH 5.8, final concentration.

In some embodiments of any of the preceding methods, the second elution buffer is a gradient elution buffer. In some embodiments, the gradient elution buffer comprises 0.04 M sodium acetate, pH 5.8 to 0.04 M sodium acetate, 0.3M sodium sulfate pH 5.8.

In some embodiments of any of the preceding methods, the second equilibration buffer comprises 0.025 M MOPS, 0.3 M sodium sulfate, pH 7.0, final concentration.

The invention also provides a method of selecting a batch that includes an IL-22 Fc fusion protein for release, the method including one, two, or all three of the following steps: (a) providing a batch comprising an IL-22 Fc fusion protein; (b) assessing the levels of sialic acid in the batch; and (c) selecting the batch for release if the batch has an average sialic acid content in the range of 8 to 12 moles of sialic acid per mole of the IL-22 Fc fusion protein. In some embodiments, step (c) includes selecting the batch for release if the batch has an average sialic acid content of 8 to 9 moles of sialic acid per mole of the IL-22 Fc fusion protein. In some embodiments, step (c) includes selecting the batch for release if the batch has an average sialic acid content of 8 moles of sialic acid per mole of the IL-22 Fc fusion protein. In some embodiments, step (c) includes selecting the batch for release if the batch has an average sialic acid content of 9 moles of sialic acid per mole of the IL-22 Fc fusion protein. In some embodiments, step (b) includes using high-performance liquid chromatography (HPLC, including reverse phase HPLC (RP-HPLC)), ultra-high performance liquid chromatography (UHPLC), capillary electrophoresis, or a colorimetric assay to assess the levels of sialic acid in the batch. In some embodiments, step (b) includes using HPLC (e.g., RP-HPLC).

Any of the methods described herein can be used in a method of controlling sialic acid content of an IL-22 Fc fusion protein or a composition thereof. Any of the methods described herein can be used in a method of reducing in vivo clearance/increasing half-life of an IL-22 Fc fusion protein or a composition thereof by adjusting the sialic acid content of an IL-22 Fc fusion protein or a composition thereof.

Host cells are transfected or transformed with expression or cloning vectors described herein for IL-22 polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook et al., supra.

Methods of transfection are known to the ordinarily skilled artisan, for example, by CaPO₄ and electroporation, or lipofection (e.g., using LIPOFECTAMINE®). Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transformations have been described in U.S. Pat. No. 4,399,216. Transformations into yeast are typically carried out according to the method of Van Solingen et al., J. Bact, 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, can also be used. For various techniques for transforming mammalian cells, see Keown et al., Methods in Enzymology, 185:527-537 (1990) and Mansour et al., Nature, 336:348-352 (1988).

Recombinantly expressed polypeptides of the present invention can be recovered from culture medium or from host cell lysates. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of a polypeptide of the present invention. Various methods of protein purification can be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular polypeptide produced.

Alternative methods, which are well known in the art, can be employed to prepare a polypeptide of the present invention. For example, a sequence encoding a polypeptide or portion thereof, can be produced by direct peptide synthesis using solid-phase techniques (see, e.g., Stewart et al., 1969, Solid-Phase Peptide Synthesis, W.H. Freeman Co., San Francisco, Calif.; Merrifield, J. 1963, Am. Chem. Soc., 85:2149-2154. In vitro protein synthesis can be performed using manual techniques or by automation. Automated synthesis can be accomplished, for instance, using an Applied Biosystems Peptide Synthesizer (Foster City, Calif.) using manufacturer's instructions. Various portions of a polypeptide of the present invention or portion thereof can be chemically synthesized separately and combined using chemical or enzymatic methods to produce the full-length polypeptide or portion thereof.

In other embodiments, the invention provides chimeric molecules comprising any of the herein described polypeptides fused to a heterologous polypeptide or amino acid sequence. Examples of such chimeric molecules include, but are not limited to, any of the herein described polypeptides fused to an epitope tag sequence or an Fc region of an immunoglobulin.

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635).

In addition to prokaryotes, eukaryotic microbes such as filamentous fungi or yeast are suitable cloning or expression hosts for IL-22-encoding vectors. Saccharomyces cerevisiae is a commonly used lower eukaryotic host microorganism.

Suitable host cells for the expression of glycosylated-IL-22 are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 cells transformed by SV40 (COS-7, ATCC CRL 1651); human embryonic kidney cells (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor cells (MMT 060562, ATCC CCL51). The selection of the appropriate host cell is deemed to be within the skill in the art.

The nucleic acid (e.g., cDNA or genomic DNA) encoding IL-22 can be inserted into a replicable vector for cloning (amplification of the DNA) or for expression. Various vectors are publicly available. The vector can, for example, be in the form of a plasmid, cosmid, viral particle, or phage. The appropriate nucleic acid sequence can be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The IL-22 polypeptides can be produced recombinantly not only directly, but also as a fusion polypeptide with a heterologous polypeptide, which can be a signal sequence or other polypeptide having a specific cleavage site at the N-terminus of the mature protein or polypeptide, as well as an IL-22 Fc fusion protein. In general, the signal sequence can be a component of the vector, or it can be a part of the IL-22 DNA that is inserted into the vector. The signal sequence can be a prokaryotic signal sequence selected, for example, from the group of the alkaline phosphatase, penicillinase, 1 pp, or heat-stable enterotoxin II leaders. For yeast secretion the signal sequence can be, e.g., the yeast invertase leader, alpha factor leader (including Saccharomyces and Kluyveromyces”—factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences can be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2: plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells is one that enables the identification of cells competent to take up the IL-22 nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [see, e.g., Stinchcomb et al., Nature, 282:39(1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the IL-22 nucleic acid sequence to direct mRNA synthesis. Promoters recognized by a variety of potential host cells are well known. Promoters suitable for use with prokaryotic hosts include the quadrature-lactamase and lactose promoter systems (see, e.g., Chang et al., Nature, 275:615 (1978); Goeddel et al., Nature, 281:544 (1979)), alkaline phosphatase, a tryptophan (trp) promoter system (see, e.g., Goeddel, Nucleic Acids Res., 8:4057 (1980); EP 36,776), and hybrid promoters such as the tac promoter (see, e.g., deBoer et al., Proc. Natl. Acad. Sci. USA, 80:21-25 (1983)). Promoters for use in bacterial systems also will contain a Shine-Dalgarno (S.D.) sequence operably linked to the DNA encoding IL-22.

Examples of suitable promoter sequences for use with yeast hosts include the promoters for 3-phosphoglycerate kinase (see, e.g., Hitzeman et al., J. Biol. Chem, 255:2073 (1980)) or other glycolytic enzymes (see, e.g., Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

IL-22 transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the IL-22 polypeptides by higher eukaryotes can be increased by inserting an enhancer sequence into the vector. Enhancers are cis-acting elements of DNA, usually about from 10 to 300 bp, which act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer can be spliced into the vector at a position 5′ or 3′ to the IL-22 coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding IL-22.

Still other methods, vectors, and host cells suitable for adaptation to the synthesis of IL-22 in recombinant vertebrate cell culture are described in Gething et al., Nature, 293:620-625 (1981); Mantei et al., Nature, 281:4046 (1979); EP 117,060; and EP 117,058.

Gene amplification and/or expression can be measured in a sample directly, for example, by conventional Southern blotting, Northern blotting to quantitate the transcription of mRNA (see, e.g., Thomas, Proc. Natl. Acad. Sci. USA, 77:5201-5205 (1980)), dot blotting (DNA analysis), or in situ hybridization, using an appropriately labeled probe, based on the sequences provided herein. Alternatively, antibodies can be employed that can recognize specific duplexes, including DNA duplexes, RNA duplexes, and DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining of cells or tissue sections and assay of cell culture or body fluids, to quantitate directly the expression of gene product. Antibodies useful for immunohistochemical staining and/or assay of sample fluids can be either monoclonal or polyclonal, and can be prepared in any mammal. Conveniently, the antibodies can be prepared against a native sequence IL-22 polypeptide or against a synthetic peptide based on the DNA sequences provided herein or against exogenous sequence fused to IL-22 DNA and encoding a specific antibody epitope.

Forms of IL-22 can be recovered from culture medium or from host cell lysates. If membrane-bound, it can be released from the membrane using a suitable detergent solution (e.g. TRITON® X-100) or by enzymatic cleavage. Cells employed in expression of IL-22 can be disrupted by various physical or chemical means, such as freeze-thaw cycling, sonication, mechanical disruption, or cell lysing agents.

It may be desired to purify IL-22 from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the IL-22 polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular IL-22 produced. The above-described general methods can be applied to the preparation of IL-2 Fc fusion protein as well.

Similarly, IL-22 Fc fusion proteins may be produced using recombinant methods and compositions, as described in, e.g., Molecular Cloning: A Laboratory Manual (Sambrook, et al., 1989, Cold Spring Harbor Laboratory Press) and PCR Protocols: A Guide to Methods and Applications (Innis, et al. 1990. Academic Press, San Diego, Calif.). In one embodiment, isolated nucleic acid encoding IL-22 Fc fusion proteins described herein is provided. In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with) a vector comprising a nucleic acid that encodes an amino acid sequence comprising the IL-22 Fc fusion protein. In certain embodiment, the vector is an expression vector. In one embodiment, the host cell is eukaryotic, e.g. a Chinese Hamster Ovary (CHO) cell or lymphoid cell (e.g., Y0, NS0, Sp20 cell). In one embodiment, a method of making an IL-22 Fc fusion protein is provided, wherein the method comprises culturing a host cell comprising a nucleic acid encoding the IL-22 Fc fusion protein, as provided above, under conditions suitable for expression of the Fc fusion protein, and optionally recovering the Fc fusion protein from the host cell (or host cell culture medium).

For recombinant production of an IL-22 Fc fusion protein, nucleic acid encoding an Fc fusion protein, e.g., as described herein, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the fusion protein). In certain embodiments, when preparing the IL-22 Fc fusion proteins, nucleic acid encoding the IL-22 polypeptide or a fragment thereof can be ligated to nucleic acid encoding an immunoglobulin constant domain sequence at specified location on the constant domain to result in an Fc fusion at the C-terminus of IL-22; however N-terminal fusions are also possible.

As an example of constructing an IL-22 Fc fusion protein, the DNA encoding IL-22 is cleaved by a restriction enzyme at or proximal to the 3′ end of the DNA encoding IL-22 and at a point at or near the DNA encoding the N-terminal end of the mature polypeptide (where use of a different leader is contemplated) or at or proximal to the N-terminal coding region for IL-22 full-length protein (where a native signal is employed). This DNA fragment then is readily inserted into DNA encoding an immunoglobulin light or heavy chain constant region and, if necessary, tailored by deletional mutagenesis. Preferably, this is a human immunoglobulin when the fusion protein is intended for in vivo therapy for humans.

In some embodiments, the IL-22-immunoglobulin chimeras are assembled as monomers, hetero- or homo-multimer, or as dimers or tetramers. Generally, these assembled immunoglobulins will have known unit structures as represented by the following diagrams. A basic four chain structural unit is the form in which IgG, IgD, and IgE exist. A four chain unit is repeated in the higher molecular weight immunoglobulins; IgM generally exists as a pentamer of, basic four-chain units held together by disulfide bonds. IgA globulin, and occasionally IgG globulin, may also exist in a multimeric form in serum. In the case of multimers, each four chain unit may be the same or different. See also Capon et al. U.S. Pat. No. 5,116,964, incorporated herein by reference in its entirety.

DNA encoding immunoglobulin light or heavy chain constant regions is known or readily available from cDNA libraries or is synthesized. See for example, Adams et al., Biochemistry 19:2711-2719 (1980); Gough et al., Biochemistry 19:2702-2710 (1980); Dolby et al; P.N.A.S. USA, 77:6027-6031 (1980); Rice et al P.N.A.S USA 79:7862-7865 (1982); Falkner et al; Nature 298:286-288 (1982); and Morrison et al; Ann. Rev. Immunol. 2:239-256 (1984). DNA sequence encoding human IL-22 with the endogenous leader sequence is provided herein (SEQ ID NO:70). DNA sequences encoding other desired binding partners which are known or readily available from cDNA libraries are suitable in the practice of this invention.

DNA encoding an IL-22 Fc fusion protein of this invention is transfected into a host cell for expression. If multimers are desired then the host cell is transformed with DNA encoding each chain that will make up the multimer, with the host cell optimally being selected to be capable of assembling the chains of the multimers in the desired fashion. If the host cell is producing an immunoglobulin prior to transfection then one needs only transfect with the binding partner fused to light or to heavy chain to produce a heteroantibody. The aforementioned immunoglobulins having one or more arms bearing the binding partner domain and one or more arms bearing companion variable regions result in dual specificity for the binding partner ligand and for an antigen or therapeutic moiety. Multiply cotransformed cells are used with the above-described recombinant methods to produce polypeptides having multiple specificities such as the heterotetrameric immunoglobulins discussed above.

Although the presence of an immunoglobulin light chain is not required in the immunoadhesins of the present invention, an immunoglobulin light chain might be present either covalently associated to an IL-22-immunoglobulin heavy chain fusion polypeptide. In this case, DNA encoding an immunoglobulin light chain is typically co-expressed with the DNA encoding the IL-22-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567 issued Mar. 28, 1989.Suitable host cells for cloning or expression of target protein-encoding vectors include prokaryotic or eukaryotic cells described herein. For example, IL-22 Fc fusion protein may be produced in bacteria, in particular when glycosylation and Fc effector function are not needed or are detrimental. For expression of polypeptides in bacteria, see, e.g., U.S. Pat. Nos. 5,648,237, 5,789,199, and 5,840,523. See also Charlton, Methods in Molecular Biology, Vol. 248 (B. K. C. Lo, ed., Humana Press, Totowa, N.J., 2003), pp. 245-254, describing expression of antibody fragments in E. coli. After expression, the Fc fusion protein may be isolated from the bacterial cell paste in a soluble fraction and can be further purified. As exemplified in the example section, further purification methods include without limitation purification using a Protein A column.

In addition to prokaryotes, eukaryotic microbes such as filamentous fungi or yeast are suitable cloning or expression hosts, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177; 6,040,498; 6,420,548; 7,125,978; and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

C. Assays

IL-22 Fc fusion protein provided herein may be identified, screened for, or characterized for their physical/chemical properties and/or biological activities by various assays known in the art.

1. Binding Assays and Other Assays

In one aspect, an IL-22 Fc fusion protein of the invention is tested for its receptor binding activity, e.g., by known methods such as ELISA, western blotting analysis, cell surface binding by Scatchard, surface plasmon resonance. In another aspect, competition assays may be used to identify an antibody that competes with the IL-22 Fc fusion protein for binding to the IL-22 receptor. In a further aspect, an IL-22 Fc fusion protein of the invention can be used for detecting the presence or amount of IL-22 receptor or IL22-Binding Protein (soluble receptor) present in a biological sample. In a further aspect, an IL-22 Fc fusion protein of the invention can be used for detecting the presence or amount of IL-22 receptor present in a biological sample. In certain embodiments, the biological sample is first blocked with a non-specific isotype control antibody to saturate any Fc receptors in the sample.

2. Activity Assays

In one aspect, assays are provided for identifying biological activity of IL-22 Fc fusion protein. Biological activity of an IL-22 polypeptide or IL-22 Fc fusion protein may include, e.g., binding to IL-22 receptor, stimulating IL-22 signaling, and inducing STAT3, RegIII and/or PancrePAP expression. In some embodiments, the assay is a potency assay as described in Example 2 (e.g., a receptor binding assay, a cell-based potency assay, or an in vivo assay). In some embodiments, potency is compared to a reference IL-22 Fc fusion protein, for example, an IL-22 Fc fusion protein having the N-glycan distribution shown in Table 12 and/or Table 13. Further, in the case of a cardiovascular disease or condition, the biological activity may include affecting the formation of atherosclerotic plaques, in particular to inhibit formation of atherosclerotic plaque formation. Inhibition of plaque formation can be assessed by any suitable imaging method known to those of ordinary skill in the art.

D. Conjugates

The invention also provides conjugates comprising an IL-22 Fc fusion protein described herein conjugated to one or more agents for detection, formulation, half-life extension, mitigating immunogenicity or tissue penetration. Exemplary conjugation includes without limitation PEGylation and attaching to radioactive isotopes.

In another embodiment, a conjugate comprises an IL-22 Fc fusion protein as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

E. Pharmaceutical Formulations

The compositions (e.g., pharmaceutical compositions comprising IL-22 Fc fusion proteins) herein will be formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners. In one embodiment, the composition can be used for increasing the duration of survival of a human subject susceptible to or diagnosed with the disease or condition disease. Duration of survival is defined as the time from first administration of the drug to death.

Pharmaceutical formulations are prepared using standard methods known in the art by mixing the active ingredient having the desired degree of purity with one or more optional pharmaceutically acceptable carriers (Remington's Pharmaceutical Sciences 16th edition, Osol, A. Ed. (1980) and Remington's Pharmaceutical Sciences 20th edition, ed. A. Gennaro, 2000, Lippincott, Williams & Wilkins, Philadelphia, Pa.), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Optionally, the formulation contains a pharmaceutically acceptable salt, preferably sodium chloride, and preferably at about physiological concentrations.

Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, benzalkonium chloride and propylparaben are preferred preservatives. Optionally, the formulations of the invention can include a pharmaceutically acceptable surfactant at a concentration of 0.005 to 0.02%.

The formulation herein can also contain more than one active compound as necessary for the particular indication being treated, preferably those with complementary activities that do not adversely affect each other. Such molecules are suitably present in combination in amounts that are effective for the purpose intended.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide a steroid, TNF antagonist or other anti-inflammatory therapeutics. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the IL-22 Fc fusion protein, which matrices are in the form of shaped articles, e.g., films or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

A pharmaceutical composition for topical administration can be formulated, for example, in the form of a topical gel. See e.g., U.S. Pat. Nos. 4,717,717; 5,130,298; 5,427,778; 5,457,093; 5,705,485; 6,331,309; and WO2006/138,468. In certain embodiments, the composition can be formulated in the presence of cellulose derivatives. In certain other embodiments, the topical formulation can be reconstituted from lyophilized formulation with sufficient buffer or diluent before administration. In certain embodiments, IL-22 polypeptide or IL-22 Fc fusion protein is formulated for topical administration to a subject having a defect in epithelial wound healing. In certain particular embodiments, the epithelial wound healing occurs in the skin. In certain other particular embodiments, the subject is a human having a defect in wound healing. In certain other embodiments, the topical formulation comprising an IL-22 Fc fusion protein of the invention can be used to improve wound healing after internal or external surgical incisions.

In one embodiment of the invention, an IL-22 polypeptide or IL-22 Fc fusion protein for use in accelerating, promoting or improving wound healing is in a formulation of a topical gel, e.g., in a pre-filled syringe or container, or alternatively, the compound of the invention can be mixed with a gel matrix right before topical administration to a patient. In certain embodiments, an additional therapeutic agent is also administered topically, either concurrently or sequentially. Other routes of administration can also be optionally used, e.g., administered by any suitable means, including but not limited to, parenteral, subcutaneous, intraperitoneal, intrapulmonary, intracerobrospinal, subcutaneous, intra-articular, intrasynovial, intrathecal, oral, and intranasal administration. Parenteral infusions include intramuscular, intravenous, intraarterial, intraperitoneal, or subcutaneous administration.

Typically for wound healing, an IL-22 Fc fusion protein is formulated for site-specific delivery. When applied topically, the IL-22 Fc fusion protein is suitably combined with other ingredients, such as carriers and/or adjuvants. There are no limitations on the nature of such other ingredients, except that they must be pharmaceutically acceptable and efficacious for their intended administration, and cannot degrade the activity of the active ingredients of the composition. Examples of suitable vehicles include ointments, creams, gels, sprays, or suspensions, with or without purified collagen. The compositions also may be impregnated into sterile dressings, transdermal patches, plasters, and bandages, optionally in liquid or semi-liquid form. An oxidized regenerated cellulose/collagen matrices can also be used, e.g., PROMOGRAN Matrix Wound Dressing or PROMOGRAN PRISMA MATRIX.

For obtaining a gel formulation, the IL-22 polypeptide or IL-22 Fc fusion protein formulated in a liquid composition may be mixed with an effective amount of a water-soluble polysaccharide or synthetic polymer to form a gel (e.g., a gelling agent) such as polyethylene glycol to form a formulation of the proper viscosity to be applied topically. The polysaccharide or gelling agent that may be used includes, for example, cellulose derivatives such as etherified cellulose derivatives, including alkyl celluloses, hydroxyalkyl celluloses, and alkylhydroxyalkyl celluloses, for example, methylcellulose, hydroxyethyl cellulose, carboxymethyl cellulose, hydroxypropyl methylcellulose, and hydroxypropyl cellulose; Sodium carboxymethyl cellulose; POE-POP block polymers: poloxamer USP in various grades; Hyaluronic acid; Polyacrylic acid such as carbopol 940; starch and fractionated starch; agar; alginic acid and alginates; gum Arabic; pullullan; agarose; carrageenan; dextrans; dextrin; fructans; inulin; mannans; xylans;

arabinans; chitosans; glycogens; glucans; and synthetic biopolymers; as well as gums such as xanthan gum; guar gum; locust bean gum; gum Arabic; tragacanth gum; and karaya gum; and derivatives, combinations and mixtures thereof. In one embodiment of the invention, the gelling agent herein is one that is, e.g., inert to biological systems, nontoxic, simple to prepare, and/or not too runny or viscous, and will not destabilize the IL-22 polypeptide or IL-22 Fc fusion held within it.

In certain embodiments of the invention, the polysaccharide is an etherified cellulose derivative, in another embodiment one that is well defined, purified, and listed in USP, e.g., methylcellulose and the hydroxyalkyl cellulose derivatives, such as hydroxypropyl cellulose, hydroxyethyl cellulose, and hydroxypropyl methylcellulose (all referred to as cellulosic agents). In some embodiments, the polysaccharide is hydroxyethyl methylcellulose or hydroxypropyl methylcellulose.

The polyethylene glycol useful for gelling is typically a mixture of low and high molecular weight polyethylene glycols to obtain the proper viscosity. For example, a mixture of a polyethylene glycol of molecular weight 400-600 with one of molecular weight 1500 would be effective for this purpose when mixed in the proper ratio to obtain a paste.

The term “water soluble” as applied to the polysaccharides and polyethylene glycols is meant to include colloidal solutions and dispersions. In general, the solubility of the cellulose derivatives is determined by the degree of substitution of ether groups, and the stabilizing derivatives useful herein should have a sufficient quantity of such ether groups per anhydroglucose unit in the cellulose chain to render the derivatives water soluble. A degree of ether substitution of at least 0.35 ether groups per anhydroglucose unit is generally sufficient. Additionally, the cellulose derivatives may be in the form of alkali metal salts, for example, the Li, Na, K, or Cs salts.

In certain embodiments, methylcellulose is employed in the gel, for example, it comprises about 1-5%, or about 1%, about 2%, about 3%, about 4% or about 5%, of the gel and the IL-22 Fc fusion protein is present in an amount of about 50-2000 μg, 100-2000 μg, or 100-1000 μg per ml of gel. In certain embodiments, the effective amount of IL-22 Fc fusion protein for wound healing by topical administration can be about 25 μg to about 500 μg, about 50 μg to about 300 μg, about 100 μg to about 250 μg, about 50 μg to about 250 μg, about 50 μg to about 150 μg, about 75 μg, about 100 μg, about 125 μg, about 150 μg, about 175 μg, about 200 μg, about 225 μg, about 250 μg, about 300 μg, or about 350 μg, per cm² wound area.

The formulations to be used for in vivo administration are generally sterile. Sterility may be readily accomplished, e.g., by filtration through sterile filtration membranes.

The present invention provides dosages for the IL-22 Fc fusion protein-based therapeutics. For example, depending on the type and severity of the disease, about 1 μg/kg to 15 mg/kg (e.g. 0.1-20 mg/kg) of polypeptide is an initial candidate dosage for administration to the subject, whether, for example, by one or more separate administrations, or by continuous infusion. A typical daily dosage might range from about 1μ·g/kg to 100 mg/kg or more, depending on the factors mentioned above. For repeated administrations over several days or longer, depending on the condition, the treatment is sustained until a desired suppression of disease symptoms occurs. However, other dosage regimens can be useful. The progress of this therapy is easily monitored by conventional techniques and assays.

For the prevention or treatment of disease, the appropriate dosage of a polypeptide of the invention (when used alone or in combination with one or more other additional therapeutic agents) will depend on the type of disease to be treated, the type of polypeptide, the severity and course of the disease, whether the polypeptide is administered for preventive or therapeutic purposes, previous therapy, the subjects clinical history and response to the polypeptide, and the discretion of the attending physician. The polypeptide is suitably administered to the subject at one time or over a series of treatments. Depending on the type and severity of the disease, about 1 μg/kg to 20 mg/kg (e.g. 0.1 mg/kg-15 mg/kg) of the polypeptide can be an initial candidate dosage for administration to the subject, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the polypeptide would be in the range from about 0.05 mg/kg to about 20 mg/kg. Thus, one or more doses of about 0.5 mg/kg, 2.0 mg/kg, 4.0 mg/kg, 10 mg/kg, 12 mg/kg, 15 mg/kg, or 20 mg/kg (or any combination thereof) may be administered to the subject. In certain embodiments, about 0.5 mg/kg, 1.0 mg·kg, 2.0 mg/kg, 3.0 mg/kg, 4.0 mg/kg, 5.0 mg/kg, 6.0 mg/kg, 7.0 mg/kg, 8.0 mg/kg, 9.0 mg/kg, 10 mg/kg, 12 mg/kg, 15 mg/kg, or 20 mg/kg (or any combination thereof) may be administered to the subject. Such doses may be administered intermittently, e.g. every week, every two weeks, or every three weeks (e.g. such that the subject receives from about two to about twenty, or e.g. about six doses of the polypeptide). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

The compounds of the invention for prevention or treatment of a cardiovascular disease or condition, metabolic syndrome, acute endotoxemia or sepsis, GVHD, or diabetes are typically administered by intravenous injection.

Other methods of administration can also be used, which includes but is not limited to, topical, parenteral, as intravenous, subcutaneous, intraperitoneal, intrapulmonary, intranasal, ocular, intraocular, intravitreal, intralesional, intracerobrospinal, intra-articular, intrasynovial, intrathecal, oral, or inhalation administration. Parenteral infusions include intramuscular, intravenous, intraarterial, intraperitoneal, or subcutaneous administration. In addition, the compounds described herein are administered to a human subject, in accord with known methods, such as intravenous administration as a bolus or by continuous infusion over a period of time.

F. Therapeutic Methods and Compositions

Any of the IL-22 Fc fusion proteins and compositions thereof (e.g., pharmaceutical compositions) provided herein may be used in therapeutic methods.

a) Inflammatory Bowel Disease

In one aspect, an IL-22 Fc fusion protein for use as a medicament is provided. In further aspects, an IL-22 Fc fusion protein for use in treating IBD, including UC and CD, is provided. In certain embodiments, an IL-22 Fc fusion protein for use in a method of treatment is provided. In certain embodiments, the invention provides an IL-22 Fc fusion protein for use in a method of treating an individual having UC or CD comprising administering to the individual an effective amount of the IL-22 Fc fusion protein. In one such embodiment, the method further comprises administering to the individual an effective amount of at least one additional therapeutic agent, e.g., as described below. In further embodiments, the invention provides an IL-22 Fc fusion protein for use in enhancing epithelial proliferation, differentiation and/or migration. In certain particular embodiments, the epithelial tissue is intestinal epithelial tissue. In certain embodiments, the invention provides an IL-22 Fc fusion protein for use in a method of enhancing epithelial proliferation, differentiation and/or migration in an individual comprising administering to the individual an effective amount of the IL-22 Fc fusion protein to enhance epithelial proliferation, differentiation and/or migration. In yet other embodiments, the invention provides an IL-22 Fc fusion protein for use in treating diabetes, especially type II diabetes, diabetic wound healing, metabolic syndromes and atherosclerosis. In certain embodiments, the invention provides an IL-22 Fc fusion protein for use in a method of treating diabetes, especially type II diabetes, diabetic wound healing, metabolic syndromes and atherosclerosis in an individual comprising administering to the individual an effective amount of the IL-22 Fc fusion protein. See International Patent Application Publication No. WO 2014/145016, which is incorporated herein by reference in its entirety. An “individual” or “subject” or “patient” according to any of the above embodiments is preferably a human.

In a further aspect, the invention provides for the use of an IL-22 polypeptide or IL-22 Fc fusion protein in the manufacture or preparation of a medicament. In one embodiment, the medicament is for treatment of IBD and wound healing. In a further embodiment, the medicament is for use in a method of treating IBD and wound healing comprising administering to an individual having IBD an effective amount of the medicament. In one such embodiment, the method further comprises administering to the individual an effective amount of at least one additional therapeutic agent, e.g., as described below. In a further embodiment, the medicament is for suppressing inflammatory response in the gut epithelial cells. In a further embodiment, the medicament is for use in a method of enhancing epithelial proliferation, differentiation and/or migration in an individual comprising administering to the individual an amount effective of the medicament to enhance epithelial proliferation, differentiation and/or migration. An “individual” according to any of the above embodiments may be a human.

In a further aspect, the invention provides a method for treating IBD, including UC and CD. In one embodiment, the method comprises administering to an individual having IBD an effective amount of an IL-22 polypeptide or an IL-22 Fc fusion protein. In one such embodiment, the method further comprises administering to the individual an effective amount of at least one additional therapeutic agent, as described below. An “individual” according to any of the above embodiments may be a human.

In a further aspect, the invention provides a method for enhancing epithelial proliferation, differentiation and/or migration in an individual. In one embodiment, the method comprises administering to the individual an effective amount of an IL-22 polypeptide or IL-22 Fc fusion protein to enhance epithelial proliferation, differentiation and/or migration. In one embodiment, an “individual” is a human.

b) Other Therapeutic Indications

The present invention provides IL-22 Fc fusion protein-based therapeutic agents for cardiovascular diseases and conditions, metabolic syndrome, acute endotoxemia and sepsis, graft-versus-host disease (GVHD), and diabetes. For the prevention, treatment or reduction in the severity of a given disease or condition, the appropriate dosage of a compound of the invention will depend on the type of disease or condition to be treated, as defined above, the severity and course of the disease or condition, whether the agent is administered for preventive or therapeutic purposes, previous therapy, the subject's clinical history and response to the compound, and the discretion of the attending physician. The compound is suitably administered to the subject at one time or over a series of treatments. Preferably, it is desirable to determine the dose-response curve and the pharmaceutical composition of the invention first in vitro, and then in useful animal models prior to testing in humans.

In one aspect, the present invention provides methods of treatment for a cardiovascular disease or disorder, metabolic syndrome, acute endotoxemia and sepsis, GVHD, and an insulin-related disorder. In one embodiment, the method comprises administering to a subject in need a therapeutically effective amount of an IL-22 Fc fusion protein. In another aspect, the invention provides a method for the delaying or slowing down of the progression of a cardiovascular disease or disorder, metabolic syndrome, GVHD, and an insulin-related disorder. In one embodiment, the method comprises administering to subject diagnosed with the disease, condition, or disorder, an effective amount of an IL-22 Fc fusion protein. In another aspect, the invention provides a method for preventing indicia of a cardiovascular disease or disorder, GVHD, and an insulin-related disorder. In one embodiment, the method comprises administering an effective amount of an IL-22 Fc fusion protein to a subject at risk of the disease, condition, or disorder, wherein the IL-22 Fc fusion protein is effective against the development of indicia of the disease, condition, or disorder. In one aspect, the present invention provides methods of treatment for GVHD. In another aspect, the invention provides a method for the delaying or slowing down of the progression of GVHD. In one embodiment, the method comprises administering to subject diagnosed with the disease, condition, or disorder, an effective amount of an IL-22 Fc fusion protein.

Cardiovascular Diseases and Conditions

In one aspect, the IL-22 Fc fusion proteins provide a therapeutic, preventative, or prophylactic effect against the development of, or the progression of, clinical and/or histological and/or biochemical and/or pathological indicia (including both symptoms and signs) of cardiovascular diseases or conditions in a subject. In one embodiment, the disease or condition is atherosclerosis. In one embodiment, the indicia include atherosclerotic plaque formation and/or vascular inflammation. In another embodiment, the subject is at risk for cardiovascular disease. In general, a subject at risk will previously have had a cardiovascular disease or condition as described herein, or will have a genetic predisposition for a cardiovascular disease or condition.

The efficacy of the treatment of cardiovascular diseases and conditions can be measured by various assessments commonly used in evaluating cardiovascular diseases. For example, cardiovascular health can be assessed. Cardiovascular health can be evaluated by, but not limited to, e.g., blood tests (e.g., total cholesterol, LDL-C, HDL-C, triglyceride, C-reactive protein, fibrinogen, homocysteine, fasting insulin, ferritin, lipoprotein, and LPS), blood pressure, auscultation, electrocardiogram, cardiac stress testing, cardiac imaging (e.g., coronary catheterization, echocardiogram, intravascular ultrasound, positron emission tomography, computed tomography angiography, and magnetic resonance imaging).

Metabolic Syndrome

In one aspect, the IL-22 Fc fusion proteins provide a therapeutic, preventative, or prophylactic effect against the development of, or the progression of, clinical and/or histological and/or biochemical and/or pathological indicia (including both symptoms and signs) of metabolic syndrome (or metabolic disorder or disease) in a subject. In one or more embodiment, the subject is at risk for metabolic syndrome.

The efficacy of the treatment of metabolic syndrome can be measured by various assessments commonly used in evaluating metabolic syndrome. For example, obesity can be measured. As a further example, hyperglycemia, dyslipidemia, insulin resistance, chronic adipose tissue inflammation, and/or hypertension can be measured. Reduction in in levels of one or more of C-reactive protein, IL-6, LPS, and plasminogen activator inhibitor 1 can be measured. These measurements can be performed by any methods well known in the art.

Insulin-Related Disorders For insulin-related disorders, the term “treatment” refers to both therapeutic treatment and prophylactic or preventative measures for the disorder, wherein the object is to prevent or slow down (lessen) the targeted pathologic condition or disorder. Those in need of treatment include those already with an insulin-related disorder as well as those prone to have such a disorder or those in whom the disorder is to be prevented.

In one aspect, the IL-22 Fc fusion proteins provide a preventative or prophylactic effect against the development of, or the progression of, clinical and/or histological and/or biochemical and/or pathological indicia (including both symptoms and signs) of an insulin-related disorder in a subject. In one embodiment, the disorder is Type I diabetes, Type II diabetes, or gestational diabetes. In one embodiment, the pathology or pathological indicia include one or more of: little or no insulin production by the pancreas (e.g., islet cells), insulin resistance, and hyperglycemia. In another embodiment, the subject is at risk for an insulin-related disorder. In general, a subject at risk has a genetic predisposition for an insulin-related disorder, has been exposed to a virus that triggers autoimmune destruction of islet cells (e.g., Epstein-Barr virus, coxsackievirus, mumps virus or cytomegalovirus), is obese, is pre-diabetic (higher than normal blood sugar levels), or has gestational diabetes.

The efficacy of the treatment of an insulin-related disorder can be measured by various assessments commonly used in evaluating such disorders. For example, both Type I and Type II diabetes can be evaluated with one or more of the following: a glycated hemoglobin test (A1C), a regular blood sugar test, and a fasting blood sugar test. Type I can also be evaluated by testing for autoantibodies in the blood and/or ketones in the urine. Type II can also be evaluated by testing for oral glucose tolerance.

Acute Endotoxemia and Sepsis

In one aspect, the IL-22 Fc fusion proteins provide a therapeutic, preventative or prophylactic effect against the development of, or the progression of, clinical and/or histological and/or biochemical and/or pathological indicia (including both symptoms and signs) of acute endotoxemia, sepsis, or both, in a subject. In one or more embodiment, the subject is at risk for acute endotoxemia, sepsis, or both.

The efficacy of the treatment of acute endotoxemia, sepsis, or both can be measured by various assessments commonly used in evaluating acute endotoxemia, sepsis, or both. For example, reduction in in levels of LPS or inflammatory markers can be measured. These measurements can be performed by any methods well known in the art.

Wound Healing

There are a variety of ways to measure wound healing. Often images are taken to calculate linear dimensions, perimeter and area. The NIH has a free program, Image J, which allows measurement of wound areas from an image. The final healing prognosis can be extrapolated from initial healing rates based on the migration of the periphery towards the center. This is done using a number of mathematical equations, the most common of which is a modified Gilman's equation. In addition to visual inspection, wound healing measurement can also be aided by spectroscopic methods or MRI. See e.g., Dargaville et al., Biosensors Bioelectronics, 2013, 41:30-42, Tan et al., 2007, British J. Radiol. 80:939-48. If healing is slow/inadequate, biopsies of the wound edges may be taken to rule out or determine infection and malignancy. In certain embodiments, the acceleration or improvement of wound healing can be assessed by comparing wound closure in IL-22-treated and control wounds. In certain embodiments, the acceleration or improvement of wound healing is at least 20%, 30%, 40%, 50%, 60%, 70%, 80% or 90% faster or better than the control.

In certain aspect, the invention provides methods for promoting/accelerating/improving healing of a wound with or without active infection, microbial contamination or colonization in the wound. The IL-22 Fc fusion proteins can be used for treating infected wounds or promoting/accelerating/improving infected wound healing. In certain embodiments, the IL-22 Fc fusion proteins can be used for treating wounds, or promoting/accelerating/improving wound healing, in the presence of infection. In some embodiments, the IL-22 Fc fusion proteins can be used for treating wounds or promoting/accelerating/improving wound healing in the presence of microbial contamination or colonization with risk for infection. In further embodiments, the patient in need of wound healing treatment can be a diabetic patient. Accordingly, in some embodiments, the wound is a diabetic wound, for example, diabetic foot ulcer. In some further embodiments, the wound is an infected diabetic wound, for example, infected diabetic foot ulcer.

An IL-22 Fc fusion protein of the invention can be used either alone or in combination with other agents in a therapy. For instance, an IL-22 Fc fusion protein of the invention may be co-administered with at least one additional therapeutic agent. In certain embodiments, an additional therapeutic agent is an immune suppressant that reduces the inflammatory response, including, without limitation, methotrexate, a TNF inhibitor, a TNF antagonist, mesalazine, steroid, dexamethasone, azathioprine, and a combination thereof. Suitable additional therapeutic agents that reduce an inflammatory response include, without limitation, 5-aminosalicylic acid (5-ASA), mercaptopurine (also called 6-mercaptopurine or 6-MP), or combination thereof. In certain embodiments, the IL-22 Fc fusion may be co-administered with one or more additional therapeutic agents that reduce an inflammatory response (for example, 5-ASA, 6-MP, or a TNF antagonist) for the treatment of IBD. In certain other embodiments, the IL-22 Fc fusion may be co-administered with an integrin antagonist such as etrolizumab for the treatment of IBD. In one embodiment, the IL-22 Fc fusion protein is used in combination with an IL-22 agonist.

For accelerating chronic wound healing, such as for the treatment of diabetic foot ulcer, the administration of an IL-22 Fc fusion protein can be combined with one or more additional wound healing agents. Suitable additional wound healing agents include, without limitation, growth factors (e.g., EGF, FGF, IGF, PDGF, TGF, and VEGF), nerve growth factor (NGF), angiogenesis factors (e.g., HGF, TNF-α, angiogenin, IL-8, angiopoietins 1 and 2, Tie-2, integrin α5, matrix metalloproteinases, nitric oxide, and COX-2), members of the platelet derived growth factor (PDGF) family (e.g., PDGF-A, PDGF-B, PDGF-C, and PDGF-D), members of the insulin growth factor (IGF) family (e.g., IGF-I and IGF-II), members of the transforming growth factor (TGF) family (e.g., TGF-α and TGF-β), and anabolic oxygen (vacuum therapy). In certain embodiments, the IL-22 Fc fusion can be co-administered with one or more additional wound healing agents described herein and/or one or more antibacterial agents or antibiotics suitable for use in topical administration. See WO 2006/138468, which is incorporated herein by reference in its entirety. In such embodiments, the antibiotic can be a sulfur antibiotic, including, without limitation, silver sulfadiazine, i.e., silvadeen. The co-administered one or more additional agents can be administered concurrently, alternatively, or sequentially with the IL-22 Fc fusion protein.

In further exemplary embodiments, if the target is prevention or treatment of cardiovascular diseases or conditions or metabolic syndrome, the administration of an IL-22 Fc fusion protein can be combined with or supplement the administration of the cholesterol-lowering agents such as statins (e.g., lovastatin, rosuvastatin, fluvastatin, atorvastatin, pravastatin, and simvastatin), bile acid binding resins (colestipol, cholestyramine sucrose, and colesevelam), ezetimibe, or a ezetimibe-simvastatin combination; anti-platelet agents such as cyclooxygenase inhibitors (e.g., aspirin), adenosine diphosphate (ADP) receptor inhibitors (e.g., clopidogrel, prasugrel, ticagrelor, and ticlopidine), phosphodiesterase inhibitors (e.g., cilostazol), glycoprotein IIB/IIIA inhibitors (e.g., abciximab, eptifibatide, and tirofiban), adenosine reuptake inhibitors (e.g., dipyridamole), thromboxane inhibitors (e.g., thromboxane synthase inhibitors, thromboxane receptor antagonists, and terutroban); beta blockers such as alprenolol, bucindolol, carteolol, carvedilol, labetalol, nadolol, oxprenolol, penbutolol, pindolol, propranolol, sotalol, timolol, eucommia bark, acebutolol, atenolol, betaxolol, bisoprolol, celiprolol, esmolol, metoprolol, nebivolol, butaxamine, ICI-118,551, and SR 59230A; angiotensin-converting enzyme (ACE) inhibitors such as captopril, zofenopril, dicarboxylate-containing agents (e.g., enalapril, ramipril, quinapril, perindopril, lisinopril, benazepril, imidapril, and zofenopril), phosphonate-containing agents (e.g., fosinopril), casokinins, lactokinins, lactotripeptides (e.g., Val-Pro-Pro, and Ile-Pro-Pro produced by the probiotic Lactobacillus helveticus or derived from casein); calcium channel blockers such as dihydropyridines (e.g., amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, and pranidipine), phenylalkylamine (e.g., verapamil), benzothiazepines (e.g., diltiazem), mibefradil, bepridil, fluspirilene, and fendiline; diuretics such as high ceiling loop diuretics (e.g., furosemide, ethacrynic acid, torsemide and bumetanide), thiazides (e.g., hydrochlorothiazide acid), carbonic anhydrase inhibitors (e.g., acetazolamide and methazolamide), potassium-sparing diuretics (e.g., aldosterone antagonists: spironolactone, and epithelial sodium channel blockers: amiloride and triamterene), and calcium-sparing diuretics, and pharmaceutically acceptable salts, acids or derivatives of any of the above.

For insulin-related disorders or metabolic syndrome, the administration of an IL-22 Fc fusion protein can be combined with or supplement the administration of various therapeutic agents. In the case of Type I diabetes (insulin-dependent diabetes mellitus or IDDM), the IL-22 Fc fusion protein described herein can be combined with one or more of regular insulin replacement therapy (including rapid-acting and long-acting insulin), immunosuppression treatment, islet transplantation and stem cell therapy. In one embodiment, the regular insulin replacement therapy includes, without limitation, regular insulin (e.g., HUMULIN R®, NOVOLIN R®), insulin isophane (e.g., HUMULIN N®, NOVOLIN N®), insulin lispro (e.g., HUMALOG®), insulin aspart (e.g., NOVOLOG®), insulin glargine (e.g., LANTUS®), and insulin detemir (e.g., LEVEMIR®). In other embodiments, the insulin replacement therapy further includes pramlintide (SYMLIN®).

In the case of Type II diabetes (non-insulin dependent diabetes mellitus or NIDDM) or metabolic syndrome, the IL-22 Fc fusion protein described herein can be combined with one or more of insulin replacement therapy (as discussed above), an agent to lower glucose production by the liver, an agent to stimulate pancreatic production and release of insulin, an agent that blocks enzymatic break down of carbohydrates, or an agent that increases insulin sensitivity. In one embodiment, the agent to lower glucose production is metformin (e.g., GLUCOPHAGE® and GLUMETZA®). In another embodiment, the agent to stimulate pancreatic production and release of insulin is glipizide (e.g., GLUCOTROL® and GLUCOTROL XL®), glyburide (e.g., DIABETA® and GLYNASE®) or glimepiride (e.g., AMARYL®). In one other embodiment, the agent that blocks enzymatic break down of carbohydrates or increases insulin sensitivity is pioglitazone (e.g., Actos). In another embodiment, the IL-22 Fc fusion protein can be combined with one of the following replacements for metformin: sitagliptin (e.g., JANUVIA®), saxagliptin (e.g., ONGLYZA®), repaglinide (e.g., PRANDIN®) and nateglinide (e.g., STARLIX®), exenatide (e.g., BYETTA®) and liraglutide (e.g., VICTOZA®). In another embodiment, the IL-22 Fc fusion protein can be combined with an oral hypoglycemic agent, e.g., sulfonylureas.

In the case of gestational diabetes or metabolic syndrome, the IL-22 Fc fusion protein described herein can be combined with an oral blood sugar control medication. In one embodiment, the medication is glyburide.

GVHD

In one aspect, the IL-22 Fc fusion proteins may provide a prophylactic effect against the development of, or a therapeutic effect against the progression of, clinical and/or histological and/or biochemical and/or pathological indicia (including both symptoms and signs) of GVHD. For example, the method provides a method for treating GVHD that includes administering to a subject in need thereof an effective amount of an IL-22 Fc fusion protein or composition thereof (including a pharmaceutical composition) as described herein. Administration of an IL-22 Fc fusion protein or composition thereof as described herein may reduce one or more symptoms of GVHD, including pain, rashes, skin thickness, yellow skin or eyes, mouth dryness or ulcers, taste abnormalities, dry eyes, infections, or weight loss. The IL-22 Fc fusion proteins or compositions thereof can be administered in combination with additional GVHD therapy, including, for example, immunosuppressive agents (e.g., cyclosporine, mycophenolate mofetil (MMF), or tacrolimus), mTOR inhibitors (e.g., sirolimus or everolimus)), chemotherapy agents (e.g., imatinib, pentostatin, methotrexate, or thalidomide), TNF antagonists (e.g., etanercept), steroids (e.g., prednisolone, methylprednisolone, topical steroids, or steroid eye drops), light treatment (e.g., extracorporeal photopheresis), hydroxychloroquine, anti-fibrotic agents (e.g., halofuginone), monoclonal antibodies (e.g., alemtuzumab, infliximab, or rituximab), or combinations thereof.

The combination therapy can provide “synergy” and prove “synergistic,” i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g. by different injections in separate syringes. In general, during alternation therapy, an effective dosage of each active ingredient is administered sequentially, i.e., serially, whereas in combination therapy, effective dosages of two or more active ingredients are administered together.

Such combination therapies noted above encompass combined administration (where two or more therapeutic agents are included in the same or separate formulations), and separate administration, in which case, administration of the IL-22 Fc fusion protein of the invention can occur prior to, simultaneously, and/or following, administration of the additional therapeutic agent or agents. In one embodiment, administration of the IL-22 Fc fusion protein and administration of an additional therapeutic agent occur within about one month, or within about one, two or three weeks, or within about one, two, three, four, five, or six days, of each other.

An IL-22 Fc fusion protein of the invention (and any additional therapeutic agent) can be administered by any suitable means, including parenteral, intrapulmonary, topical and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

An IL-22 Fc fusion protein of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The IL-22 Fc fusion protein need not be, but is optionally formulated with one or more agents currently used to prevent or treat the disorder in question. The effective amount of such other agents depends on the amount of the fusion protein present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an IL-22 Fc fusion protein of the invention (when used alone or in combination with one or more other additional therapeutic agents) will depend on the type of disease to be treated, the type of Fc region, the severity and course of the disease, whether the fusion protein is administered for preventive or therapeutic purposes, previous therapy, the patients clinical history and response to the IL-22 Fc fusion protein, and the discretion of the attending physician. The IL-22 Fc fusion protein is suitably administered to the patient at one time or over a series of treatments. Depending on the type and severity of the disease, about 1 μg/kg to 15 mg/kg (e.g., 0.1 mg/kg-10 mg/kg) or about 0.1 μg/kg to 1.5 mg/kg (e.g., 0.01 mg/kg-1 mg/kg) of the IL-22 Fc fusion protein can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the IL-22 Fc fusion protein would be in the range from about 0.05 mg/kg to about 10 mg/kg. Certain other dosages include the range from about 0.01 mg/kg to about 10 mg/kg, about 0.02 mg/kg to about 10 mg/kg, and about 0.05 mg/kg to about 10 mg/kg. Thus, one or more doses of about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 2.0 mg/kg, 3.0 mg/kg, 4.0 mg/kg, 5 mg/kg, 6 mg/kg, 7 mg/kg, 8 mg/kg, 9 mg/kg or 10 mg/kg (or any combination thereof) may be administered to the patient. For topical wound healing, one or more doses of about 0.001 mg/cm² to about 10 mg/cm² wound area, about 0.05 mg/cm² to about 5 mg/cm² wound area, about 0.01 mg/cm² to about 1 mg/cm² wound area, about 0.05 mg/cm² to about 0.5 mg/cm² wound area, about 0.01 mg/cm² to about 0.5 mg/cm² wound area, about 0.05 mg/cm² to about 0.2 mg/cm² wound area, or about 0.1 mg/cm² to about 0.5 mg/cm² wound area (or any combination thereof) may be administered to the patient. In certain embodiments, one or more doses of about 0.01 mg/cm², 0.02 mg/cm², 0.03 mg/cm², 0.04 mg/cm², 0.05 mg/cm², 0.06 mg/cm², 0.07 mg/cm², 0.08 mg/cm², 0.09 mg/cm², 0.1 mg/cm², 0.15 mg/cm², 0.2 mg/cm², 0.25 mg/cm², 0.3 mg/cm², 0.4 mg/cm², or 0.5 mg/cm² wound area may be administered to the patient. Such doses may be administered intermittently, e.g., every week or every three weeks (e.g., such that the patient receives from about two to about twenty, or e.g., about six doses of the IL-22 Fc fusion protein). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using conjugate of the invention in place of or in addition to an IL-22 Fc fusion protein.

G. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an IL-22 Fc fusion protein provided herein. The label or package insert indicates that the composition is used for treating the condition of choice. Moreover, the article of manufacture may comprise (a) a first container with a composition contained therein, wherein the composition comprises an IL-22 Fc fusion protein of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include a conjugate of the invention in place of or in addition to an IL-22 Fc fusion protein.

### EXAMPLES

The following are examples of methods and compositions of the invention. It is understood that various other embodiments may be practiced, given the general description provided above, and the examples are not intended to limit the scope of the claims.

### Example 1: Structural and Molecular Characterization of the IL-22 Fc Fusion Protein

### Primary Structure of Exemplary IL-22 Fc Fusion Proteins

The exemplary IL-22 Fc fusion proteins of the invention consist of two single-chain units linked by two inter-chain disulfide bridges. Each single chain consists of a human interleukin-22 (IL-22) fusion protein composed of the cytokine IL-22 fused with the Fc region of a human immunoglobulin G4 (IgG4).

The Fc region improves the cytokine's pharmacokinetic characteristics. The Fc region of the fusion protein incorporates an N81G mutation (this corresponds to an N227G mutation when numbered from the N-terminus of the entire fusion polypeptide of the cytokine and Fc, and to an N297G mutation with respect to the numbering of the Fc region according to the EU index), which removes glycosylation, minimizing the potential for Fc effector function. Additionally, a modified hinge region generated by substituting Ser to Pro, e.g., as shown in the bolded and underlined Pro residue in the amino acid sequence of CPPCP (SEQ ID NO:31) via a site-directed mutation was performed to increase stability of the molecule. The IL-22 Fc fusion protein was produced by Chinese hamster ovary (CHO) cells and has a predicted molecular mass of approximately 85,131 Da (intact, peptide chains only, without the C-terminal lysine residue on the Fc). The calculated molecular mass of the IL-22 cytokine without carbohydrates is 16,749.4 Da (cysteine residues are in the reduced form). The calculated molecular mass of an IgG4 Fc without C-terminal lysine residue is 25,844.3 Da (cysteine residues are in the reduced form). The structure of the IL-22 Fc fusion protein is shown in FIG. 1A. The IL-22 cytokine and IgG4 Fc region amino acid sequences of the IL-22 Fc fusion protein are shown in FIG. 1B and FIG. 1C, respectively.

### Characterization Test Methods

The structural and molecular properties of the IL-22 Fc fusion protein were investigated with emphasis on the following physiochemical attributes: primary structure, size and charge heterogeneity, isoelectric point, extinction coefficient, N-glycan distribution and sialic acid content, higher order structure, and biological activity. The test methods used for characterization are listed in Table 1 and described herein.

Characterization studies were performed on the IL-22 Fc fusion protein Reference Standard Batch and Clinical Batches 1, 2, and 3. All batches were formulated in 10 mM sodium phosphate, 240 mM sucrose, 5 mM methionine, and 0.02% (w/v) polysorbate 20, pH 7.1, at a final nominal concentration of 10 mg/mL IL-22 Fc fusion protein.

TABLE 1
Characterization Test Methods

| Test Method | Purpose |
|---|---|
| Electrospray Ionization- | To confirm molecular masses predicted for |
| Mass Spectrometry | the intact and reduced molecule |
| Tryptic Peptide Map | To verify the primary IL-22 Fc Fusion Protein |
| Analysis | structure and demonstrate batch-to-batch |
| | consistency of peptide pattern |
| SE-HPLC and | To provide quantitative information about the |
| CE-SDS-NGS (Non- | molecular size heterogeneity of IL-22 Fc |
| reduced and reduced) | Fusion Protein |
| Sodium Dodecyl | To confirm overall product purity and |
| Sulfate-Polyacrylamide | qualitatively assess the relative abundance |
| Gel Electrophoresis | of protein impurities |
| Analysis with SYPRO | |
| Ruby Staining | |
| ICIEF | To provide quantitative information on the |
| | charge heterogeneity of IL-22 Fc Fusion |
| | Protein with and without C-terminal lysine |
| | heterogeneity |
| Isoelectric Point by | To determine the pH at which IL-22 Fc |
| ICIEF | Fusion Protein has no net charge |
| Extinction Coefficient | To determine the extinction coefficient to be |
| Determination | used for the determination of IL-22 Fc Fusion |
| | Protein protein concentrations |
| N-glycosylation Site | To determine the extent of N-glycosylation |
| Occupancy by Lys-C | site occupancy of Asn21, Asn35, Asn64, and |
| LC-MS Peptide | Asn143 |
| Mapping | |
| N-linked Glycan | To determine the relative distribution of N- |
| Analysis by 2-AA | linked glycans |
| HILIC-UHPLC | |
| Site-specific | To determine the relative distribution of N- |
| N-glycosylation | linked glycans at Asn21 |
| Analysis by Lys-C | |
| LC-MS Peptide | |
| Mapping | |
| Sialic Acid (SA) | To provide quantitative information about the |
| Determination by | amount of Sialic Acid |
| RP-HPLC | |
| Potency Binding Assay | To provide quantitative information on the |
| | ability of IL-22 Fc Fusion Protein to bind to |
| | the IL22-RA1 extracellular domain |
| Circular Dichroism | To look for the presence of higher order |
| | structural elements in IL-22 Fc Fusion Protein |

Abbreviation: 2-AA HILIC-UHPLC = 2-aminobenzoic acid hydrophilic interaction liquid chromatography - ultra-high-performance liquid chromatography;
CE-SDS-NGS = capillary electrophoresis sodium dodecyl sulfate, non-gel sieving;
CPA = corrected peak area;
HMW = high molecular weight;
HPLC = high-performance liquid chromatography;
ICIEF = imaged capillary isoelectric focusing;
RP = reversed phase;
SE = size-exclusion.

### Physiochemical Properties

Mass Spectrometry

The IL-22 Fc fusion protein samples were analyzed by electrospray ionization-mass spectrometry (ESI-MS) in the intact state after deglycosylation with PNGase F, and after deglycosylation and reduction of the disulfide bonds with tris(2-carboxyethyl)phosphine hydrochloride (TCEP). ESI-MS analysis was performed after desalting of the samples by reversed-phase high-performance liquid chromatography (RP-HPLC) for direct online MS analysis.

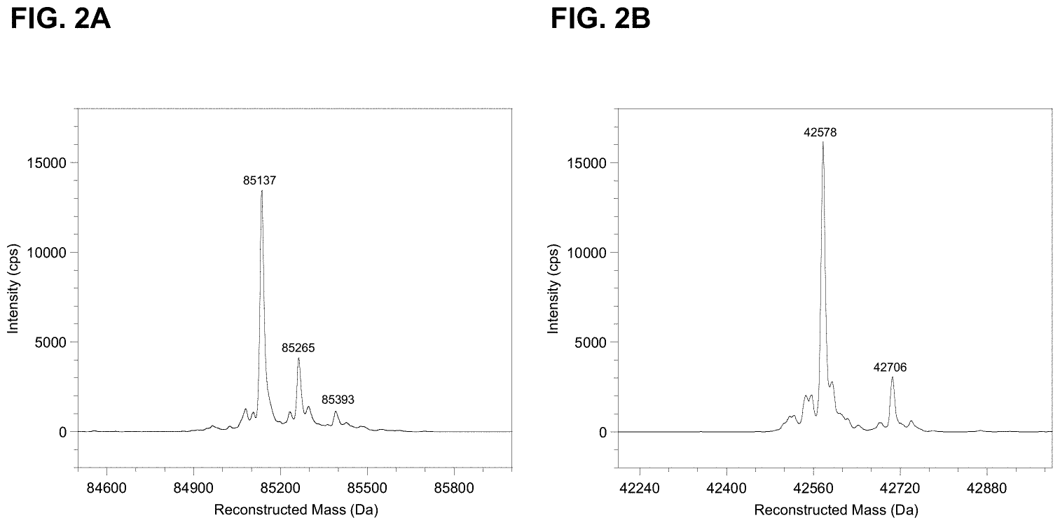

Analysis of non-reduced, deglycosylated IL-22 Fc fusion protein was used to obtain the masses of the main species of intact IL-22 Fc fusion protein (FIG. 2A), while analysis of reduced, deglycosylated IL-22 Fc fusion protein was used to obtain the masses of the single chain molecule (FIG. 2B). The observed masses of IL-22 Fc fusion protein correspond to the predicted masses deduced from the amino acid sequence (Table 2). The major masses obtained for the intact molecule correspond to the predicted masses for the IL-22 Fc fusion protein without the carboxy-terminal lysine residue and without N-linked glycans.

MS analysis confirms that the molecular masses are in accordance with predicted masses deduced from the amino acid sequence of IL-22 Fc fusion protein.

TABLE 2
Electrospray Ionization-Mass Spectrometry of Deglycosylated,
Intact, and Reduced IL-22 Fc Fusion Protein

| | Observed Mass (Da)^{a} | |
|---|---|---|
| | | Reduced |
| | Intact | Deglycosylated |
| | Deglycosylated | Single Chain |
| | Molecule | Molecule |
| Predicted Mass (Da)^{b} | 85139 | 42580 |
| Referene Standard | 85137 | 42578 |
| Batch | | |
| Clinical Batch 1 | 85139 | 42578 |
| Clinical Batch 2 | 85139 | 42578 |
| Clinical Batch 3 | 85139 | 42578 |

^{a}Observed mass does not include C-terminal lysine.
^{b}Average masses.

Tryptic Peptide Map

Peptide map analysis by high-resolution liquid chromatography-tandem mass spectrometry (LC-MS-MS) analysis was used to verify the predicted primary structure and demonstrate batch-to-batch consistency of the peptide pattern. In addition, post-translational modifications, as well as chemical modifications to the amino acid side chains caused by processing or storage of the recombinant protein were detected.

To generate the IL-22 Fc fusion protein peptide maps, the protein was digested with trypsin after subjecting the protein to denaturing conditions with guanidinium hydrochloride, reduction with dithiothreitol, and carboxymethylation of cysteines with iodoacetic acid. The resulting peptides were separated by RP-HPLC coupled to an MS-MS capable mass spectrometer, and the elution of peptides was monitored at 214 nm. Masses of the tryptic peptides were determined by LC-MS analysis of the separated digest mixture.

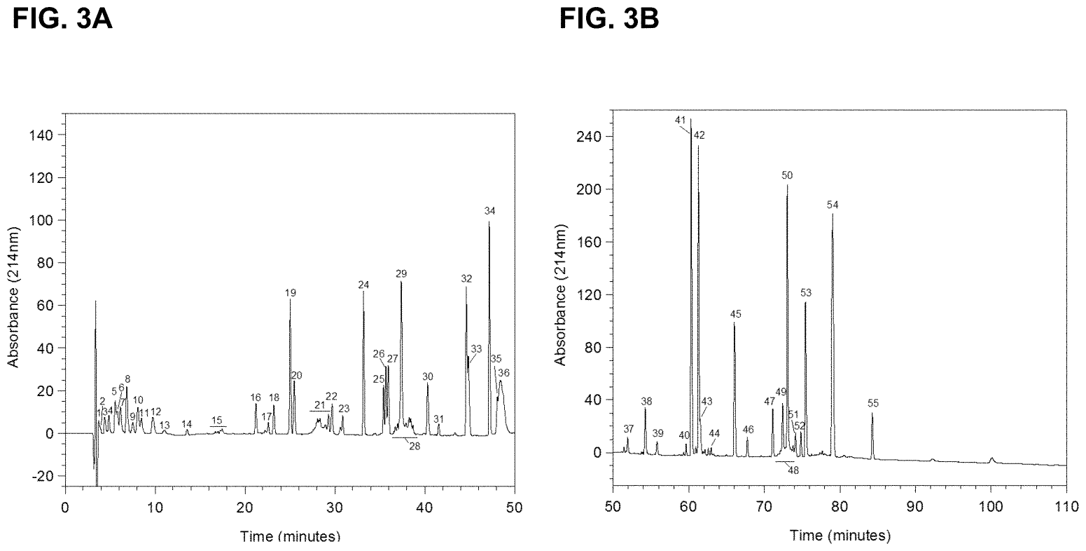

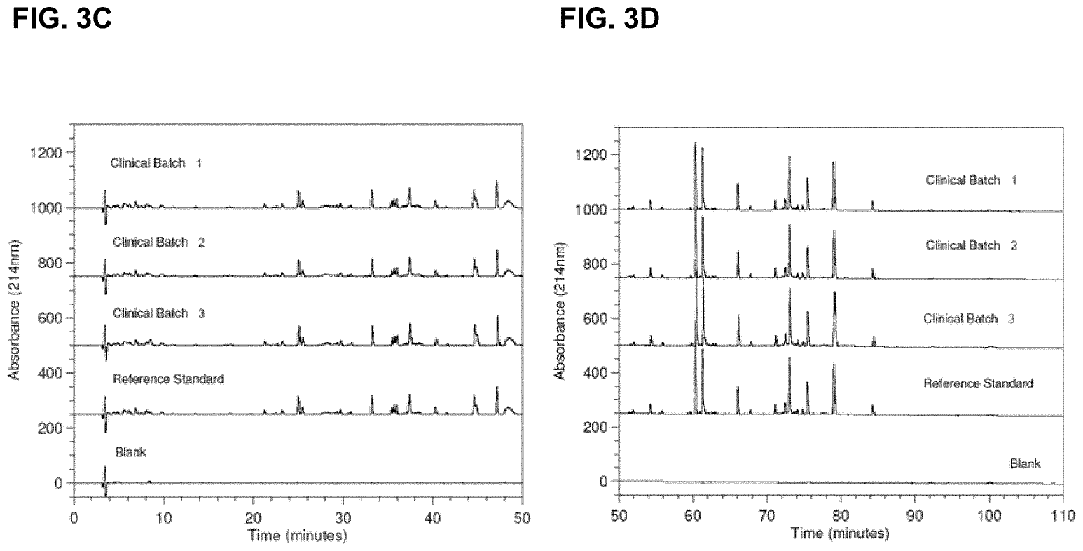

Peptide assignments were based on the observed masses of the intact peptides (FIG. 3A and FIG. 3B). The tryptic peptides associated with N-linked carbohydrates are presented as grouped peaks. The sequence of the peptides and their predicted and observed masses are provided for the Reference Standard Batch in Table 3 and Table 4. All of the observed peptides were consistent with the peptides expected from the tryptic digestion of a protein with the sequence of the IL-22 Fc fusion protein, including common post-translational modifications. No sequence variants were observed.

TABLE 3
Tryptic Peptides from the Human IL-22 Cytokine of the IL-22 Fc Fusion Protein

| | | | Observed | Predicted | |
|---|---|---|---|---|---|
| | | Peak | Mass | Mass | |
| Peptide^{a} | Residues | No.^{b} | (Da)^{c} | (Da)^{c} | Peptide Sequence^{d} |
| T1C | 1-8 | 19 | 928.43 | 928.43 | APISSHCR |
| T2C | 9-11 | 9 | 375.22 | 375.22 | LDK |
| T3C | 12-22 | 31 | 1367.67 | 1367.67 | SNFQQPYITNR |
| T3C | 12-22 | 28 | Multiple | Multiple | SNFQQPYITNR + N-linked carbohydrates |
| T3C^{e} | 12-18 | 26 | 883.40 | 883.40 | SNFQQPY |
| T3C^{e} | 19-22 | 11 | 503.29 | 503.29 | ITNR |
| T4C | 23-28 | 33 | 710.39 | 710.39 | TFMLAK |
| T5C | 29-40 | 21 | Multiple | Multiple | EASLADNNTDVR + N-linked carbohydrates |
| T6C | 41-45 | 20 | 559.35 | 559.35 | LIGEK |
| T7C | 46-55 | 32 | 1162.57 | 1162.57 | LFHGVSMSER |
| T8C | 56-60 | 29 | 715.32 | 715.32 | CYLMK |
| T9C | 61-77 | 48 | Multiple | Multiple | QVLNFTLEEVLFPQSDR + N-linked |
| | | | | | carbohydrates |
| T10C | 78-91 | 49 | 1724.89 | 1724.88 | FQPYMQEVVPFLAR |
| T10C^{e} | 78-81 | 27 | 554.26 | 554.26 | FQPY |
| T10C^{e} | 82-88 | 39 | 849.42 | 849.42 | MQEVVPF |
| T10C^{e} | 78-88 | 46 | 1384.66 | 1384.66 | FQPYMQEVVPF |
| T10C^{e} | 82-91 | 43 | 1189.64 | 1189.64 | MQEVVPFLAR |
| T10C^{e} | 89-91 | 13 | 359.24 | 359.24 | LAR |
| T11C | 92-95 | 10 | 489.28 | 489.28 | LSNR |
| T12C | 96-110 | 34 | 1794.85 | 1794.84 | LSTCHIEGDDLHIQR |
| T13C | 111-114 | 6 | 488.28 | 488.28 | NVQK |
| T14C | 115-116 | — | ND | 260.20 | LK |
| T15C | 117-120 | 4 | 462.26 | 462.26 | DTVK |
| T14-15C^{e} | 115-120 | 18 | 703.44 | 703.44 | LKDTVK |
| T16C | 121-121 | — | ND | 147.11 | K |
| T14-16C^{e} | 115-121 | 17 | 831.53 | 831.53 | LKDTVKK |
| T17C | 122-129 | 22 | 832.44 | 832.44 | LGESGEIK |
| T16-17C^{e} | 121-129 | 23 | 960.54 | 960.54 | KLGESGEIK |
| T18C | 130-142 | 55 | 1477.81 | 1477.81 | AIGELDLLFMSLR |
| T19C | 143-146 | 16 | 634.30 | 634.30 | NACIR^{f} |
| T19C | 143-146 | 15 | Multiple | Multiple | NACIR^{f} + N-linked carbohydrates |

Abbreviations: ND = not detected.
^{a}Peptides are identified by a three-part code in which the first letter designates the enzyme used to digest the sample (T for trypsin), the middle number signifies the fragment number beginning with the amino terminal, and the last letter identifies the origin of the peptide (C for cytokine).
^{b}See Fig. 3A and Fig. 3B for peak assignments.
^{c}Observed and predicted values represent monoisotopic masses.
^{d}Cysteine residues are carboxymethlyated-cysteine.
^{e}Nonspecific cleavage or missed cleavage.
^{f}The arginine (R₁₄₆) on peptide T19C is part of the Fc.

TABLE 4
Tryptic Peptides from the Human Immunoglobulin G4 (IgG4) Fc
of the IL-22 Fc Fusion Protein

| | | | Observed | Predicted | |
|---|---|---|---|---|---|
| | | Peak | Mass | Mass | |
| Peptide^{a} | Residues | No.^{b} | (Da)^{c} | (Da)^{c} | Peptide Sequence^{d} |
| T1F | 1-1 | — | ND | 175.11 | R |
| T2F | 2-5 | 7 | 462.26 | 462.26 | VESK |
| T3F | 6-32 | 54 | 2955.46 | 2955.45 | YGPPCPPCPAPEFLGGPSVFLFPPKPK |
| T4F | 33-39 | 30 | 835.44 | 835.43 | DTLMISR |
| T5F | 40-72 | 50 | 3789.77 | 3789.76 | TPEVTCVVVDVSQEDPEVQFNWYVDGVEVHNAK |
| T5F^{e} | 40-69 | 52 | 3476.59 | 3476.58 | TPEVTCVVVDVSQEDPEVQFNWYVDGVEVH |
| T5F^{e} | 40-60 | 44 | 2392.09 | 2392.09 | TPEVTCVVVDVSQEDPEVQFN |
| T6F | 73-76 | 5 | 501.31 | 501.31 | TKPR |
| T7F | 77-85 | 24 | 1116.50 | 1116.50 | EEQFGSTYR |
| T6-7F^{e} | 73-85 | 25 | 1598.80 | 1598.80 | TKPREEQFGSTYR |
| T8F | 86-101 | 53 | 1808.01 | 1808.01 | VVSVLTVLHQDWLNGK |
| T9F | 102-104 | 8 | 439.22 | 439.22 | EYK |
| T8-9F^{e} | 86-104 | 51 | 2228.21 | 2228.21 | VVSVLTVLHQDWLNGKEYK |
| T10F | 105-106 | 2 | 308.13 | 308.13 | CK |
| T11F | 107-110 | 3 | 447.26 | 447.26 | VSNK |
| T10-11 F^{e} | 105-110 | 14 | 736.37 | 736.37 | CKVSNK |
| T12F | 111-118 | 29 | 830.46 | 830.46 | GLPSSIEK |
| T13F | 119-122 | 12 | 448.28 | 448.28 | TISK |
| T14F | 123-124 | 1 | 218.15 | 218.15 | AK |
| T15F | 125-128 | 7 | 457.25 | 457.25 | GQPR |
| T14-15F^{e} | 123-128 | 10 | 656.38 | 656.38 | AKGQPR |
| T16F | 129-144 | 35 | 1876.90 | 1876.90 | EPQVYTLPPSQEEMTK |
| T17F | 145-154 | 38 | 1162.61 | 1162.61 | NQVSLTCLVK |
| T18F | 155-176 | 42 | 2544.14 | 2544.13 | GFYPSDIAVEWESNGQPENNYK |
| T19F | 177-193 | 45 | 1901.93 | 1901.93 | TTPPVLDSDGSFFLYSR |
| T19F^{e} | 177-190 | 47 | 1658.80 | 1658.80 | TTPPVLDSDGSFFLY |
| T20F | 194-198 | 20 | 575.34 | 575.34 | LTVDK |
| T21F | 199-200 | 1 | 262.15 | 262.15 | SR |
| T22F | 201-223 | 41 | 2803.25 | 2803.24 | WQEGNVFSCSVMHEALHNHYTQK |
| T21-22F^{e} | 199-223 | 40 | 3046.38 | 3046.37 | SRWQEGNVFSCSVMHEALHNHYTQK |
| T23F | 224-231 | 36 | 804.48 | 804.48 | SLSLSLGK |
| T23F^{e} | 224-230 | 37 | 676.39 | 676.39 | SLSLSLG |

Abbreviations: ND = not detected.
^{a}Peptides are identified by a three-part code in which the first letter designates the enzyme used to digest the sample (T for trypsin), the middle number signifies the fragment number beginning with the amino terminal, and the last letter identifies the origin of the peptide (F for Fc).
^{b}See Fig. 3A and Fig. 3B for peak assignments.
^{c}Observed and predicted values represent monoisotopic masses.
^{d}Cysteine residues are carboxymethlyated-cysteine.
^{e}Nonspecific cleavage or missed cleavage.

The peptide maps were compared for the IL-22 Fc fusion protein Reference Standard Batch and all Clinical Batches (FIG. 3C and FIG. 3D). The peptide maps of the Reference Standard Batch and all Clinical Batches were consistent with respect to the peptide pattern, demonstrating the batch-to-batch consistency of the manufacturing process.

SE-HPLC

Size-exclusion high-performance liquid chromatography (SE-HPLC) was performed as part of batch release testing. Quantitative release data for the Clinical Batches and for the Reference Standard Batch are shown side-by-side in Table 5.

TABLE 5
Molecular Size Distribution of the IL-22 Fc Fusion Protein
by SE-HPLC (Peak Area %)

| | Percent Composition | | |
|---|---|---|---|
| | Sum of | | Sum of |
| | HMW | Main | LMW |
| IL-22 Fc Fusion Protein Batch | Forms | Peak | Forms |
| Reference Standard Batch | 0.3 | 99.6 | 0.1 |
| Clinical Batch 1 | 0.3 | 99.6 | 0.1 |
| Clinical Batch 2 | 0.3 | 99.6 | 0.1 |
| Clinical Batch 3 | 0.2 | 99.7 | 0.1 |

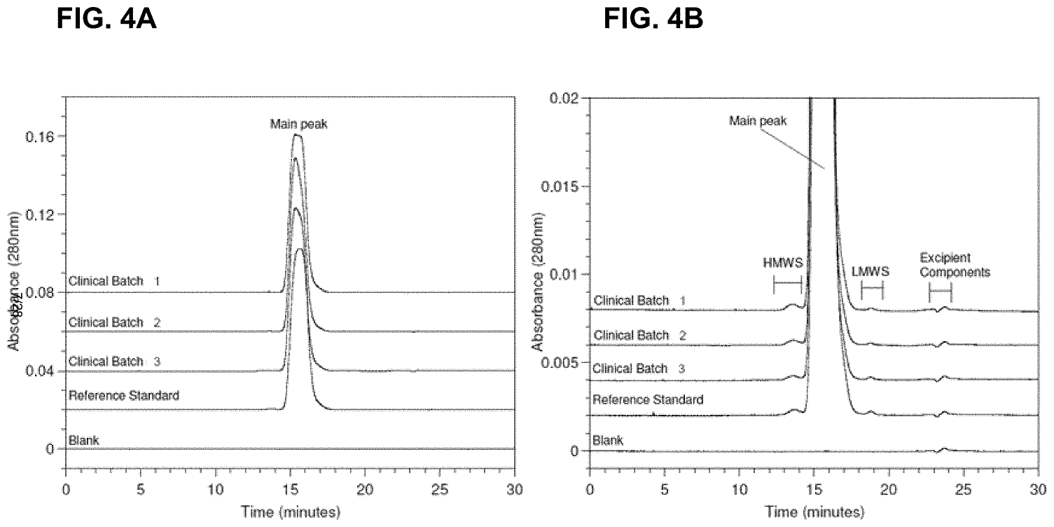

Note:
See FIG. 4A and FIG. 4B for the chromatograms.
Abbreviations: SE-HPLC = size-exclusion high-performance liquid chromatography.

The IL-22 Fc fusion protein elutes as a major peak with a retention time of about 16 minutes. The full-scale and expanded views of the SE-HPLC profiles for the IL-22 Fc fusion protein batches demonstrated that the Clinical Batches were consistent with regard to peak pattern and main peak content (FIG. 4A and FIG. 4B). In addition, analytical ultracentrifugation (AUC) was utilized as an orthogonal size heterogeneity method as part of extended characterization. Data from AUC correlate well with SE-HPLC when analyzing a series of stressed samples of varying levels of aggregate.

CE-SDS-NGS

Capillary electrophoresis sodium dodecyl sulfate-non-gel sieving (CE-SDS-NGS) under non-reduced conditions was performed as part of batch release testing. Quantitative release data are shown side-by-side in Table 6. CE-SDS-NGS under reduced conditions (in the presence of dithiothreitol) was performed as extended characterization. Additional species were assessed as part of extended characterization testing (Table 7).

TABLE 6
Molecular Size Distribution of Non-Reduced IL-22 Fc Fusion
Protein by CE-SDS-NGS (% CPA)

| | Percent Composition by Peak | | | | | | | | |
|---|---|---|---|---|---|---|---|---|---|
| IL-22 Fc Fusion | | | | | | | | Sum of | Sum of |
| Protein Batch | 1 | 2 | 3 | 4 | 5 | 6 | Main | Pre-peaks | Post-peaks |
| Reference Standard | 0.02 | 0.05 | 0.02 | 0.10 | 0.07 | 2.9 | 96.7 | 3.1 | 0.1 |
| Batch | | | | | | | | | |
| Clinical Batch 1 | 0.01 | 0.04 | 0.01 | 0.07 | 0.04 | 2.8 | 96.9 | 3.0 | 0.1 |
| Clinical Batch 2 | 0.01 | 0.04 | 0.01 | 0.06 | 0.03 | 2.5 | 97.2 | 2.7 | 0.1 |
| Clinical Batch 3 | 0.01 | 0.04 | 0.01 | 0.07 | 0.04 | 2.8 | 96.9 | 2.9 | 0.2 |

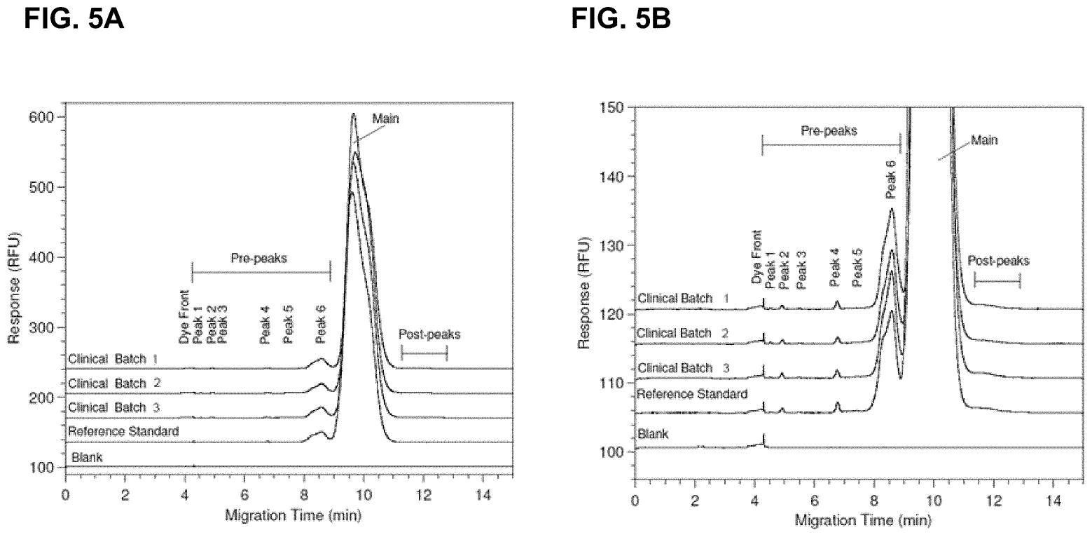

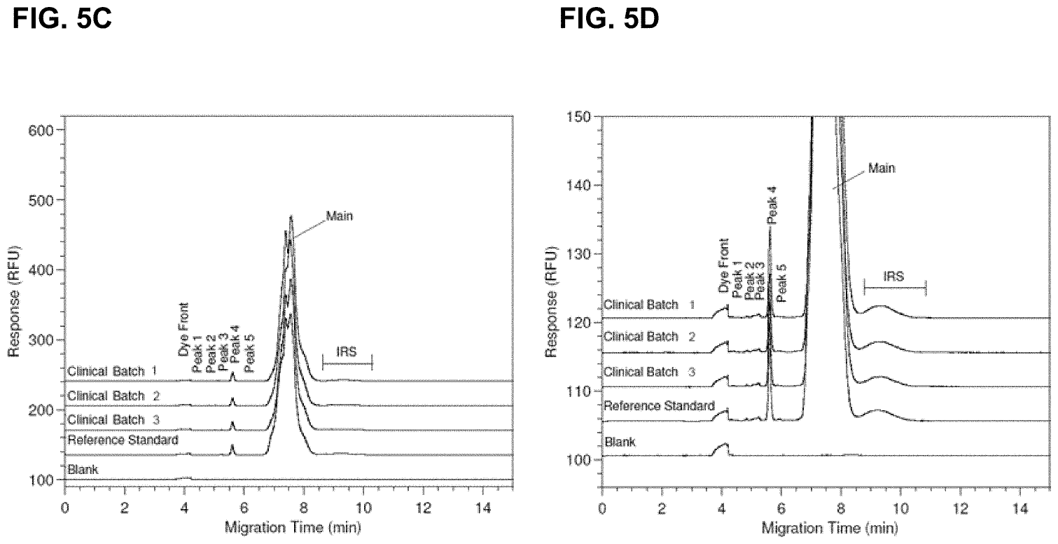

Note:
See FIG. 5A and FIG. 5B for CE-SDS-NGS non-reduced electropherograms and peak identification.
Abbreviations:
CE-SDS-NGS = capillary electrophoresis sodium dodecyl sulfate-non-gel sieving.

TABLE 7
Molecular Size Distribution of Reduced IL-22 Fc
Fusion Protein by CE-SDS-NGS (% CPA)

| IL-22 Fc Fusion | Percent Composition by Peak | | | | | | |
|---|---|---|---|---|---|---|---|
| Protein Batch | 1 | 2 | 3 | 4 | 5 | Main | IRS |
| Reference Standard Batch | 0.01 | 0.04 | 0.12 | 1.3 | 0.05 | 97.4 | 1.1 |
| Clinical Batch 1 | 0.01 | 0.03 | 0.13 | 1.2 | 0.06 | 97.4 | 1.1 |
| Clinical Batch 2 | 0.02 | 0.04 | 0.12 | 1.1 | 0.05 | 97.6 | 1.0 |
| Clinical Batch 3 | 0.02 | 0.04 | 0.13 | 1.2 | 0.05 | 97.6 | 1.0 |

Notes:
See FIG. 5C and FIG. 5D for CE-SDS-NGS reduced electropherograms and peak identification.
Abbreviations:
CE-SDS-NGS = capillary electrophoresis sodium dodecyl sulfate-non-gel sieving;
IRS = incompletely reduced species.

Non-reduced IL-22 Fc fusion protein migrated as a prominent peak, with the remaining minor peaks representing species with an apparent lower or higher molecular weight (FIG. 5A (full-scale view) and FIG. 5B (expanded view)). The relative distribution of the variants separated by CE-SDS-NGS of non-reduced samples is provided in Table 6. The CE-SDS-NGS profiles for the IL-22 Fc fusion protein batches showed consistent peak patterns and percent corrected peak areas (CPA). In addition, this method was capable of detecting protein disulfide reduction, when present.

The electropherograms from the CE-SDS-NGS analysis of reduced IL-22 Fc fusion protein showed the presence of one major peak, corresponding to the single chain molecule (FIG. 5C and FIG. 5D). The relative distributions of the reduced forms are listed in Table 7. The CE-SDS-NGS profiles for the IL-22 Fc fusion protein batches showed consistent peak patterns and corrected percent CPA.

SDS-PAGE Analysis

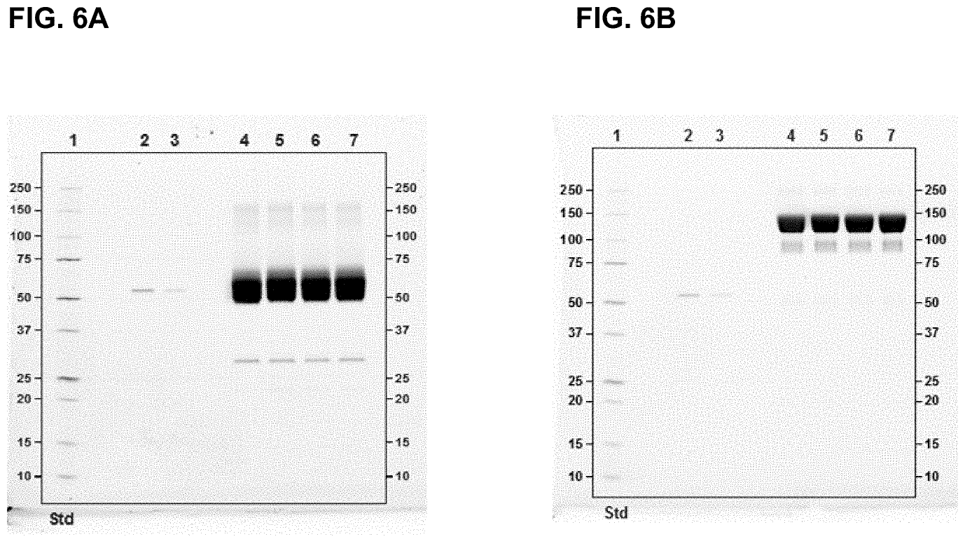

Sodium dodecyl sulfate polyacrylamide gel electrophoresis (SDS-PAGE) analysis with SYPRO® Ruby staining was used to evaluate the purity of IL-22 Fc fusion protein lots. Although this method is not quantitative, it can be used to detect minor protein impurities. Both reduced (FIG. 6A) and non-reduced (FIG. 6B) IL-22 Fc fusion protein samples were analyzed by SDS-PAGE. Samples were denatured by heating in the presence of SDS-PAGE sample buffer. Non-reduced samples were heated to 60° C. for 5 minutes in the presence of iodoacetamide, while reduced samples were heated to 60° C. for 10 minutes with a reducing agent (DTT) added. All samples were loaded at 5 μg. The prepared samples, molecular weight standards, and sensitivity standards (2 and 8 ng per lane of bovine serum albumin) were separated on a 4%-20% polyacrylamide gradient gel. The protein components were then visualized by SYPRO® Ruby staining.

Consistent banding patterns were observed for the IL-22 Fc fusion protein Reference Standard Batch and IL-22 Fc fusion protein Clinical Batches in both reduced (FIG. 6A, lanes 4-7) and non-reduced (FIG. 6B, lanes 4-7) samples.

For the reduced samples (FIG. 6A, lanes 4-7), one major band migrated with the apparent mass of approximately 50 kDa, which is consistent with the single chain of the IL-22 Fc fusion protein. The banding pattern observed in the reduced samples was also consistent with the migration pattern observed in the CE-SDS-NGS analysis of reduced IL-22 Fc fusion protein (FIG. 5C and FIG. 5D). All of the bands in the gel were excised, digested with trypsin, and analyzed by matrix-assisted laser desorption/ionization time-of-flight mass spectrometry (MALDI-TOF MS). The results of the tryptic map mass analysis indicated that all of the bands in the gel were product related.

For the non-reduced samples (FIG. 6B, lanes 4-7), intact IL-22 Fc fusion protein is the main band and migrates at an apparent mass of approximately 125 kDa. The banding pattern observed in the non-reduced samples was also consistent with the migration pattern observed in the CE-SDS-NGS analysis of non-reduced IL-22 Fc fusion protein (FIG. 5A and FIG. 5B). All of the bands in the gel were excised, digested with trypsin, and analyzed by MALDI-TOF MS. The results of the tryptic map mass analysis indicated that all of the bands in the gel were product related.

ICIEF

Imaged capillary isoelectric focusing (ICIEF) provides a means of quantitatively assessing the charge heterogeneity of a protein. The IL-22 Fc fusion protein batches were analyzed with and without CpB treatment. CpB is an enzyme that removes C-terminal lysine residues. Heterogeneity of C-terminal lysine residues is believed to be the result of proteolysis by endogenous CHO basic carboxypeptidase(s) during the cell culture operation. By removing the charge heterogeneity imparted by the C-terminal lysine residues, a more thorough assessment of the remaining charge variants present in the protein is possible.

ICIEF, post-CpB and sialidase treatment, was performed as part of batch release testing. Quantitative release data are shown side-by-side in Table 8. In addition, ICIEF without CpB treatment (native IL-22 Fc fusion protein with C-terminal lysine heterogeneity) was performed as extended characterization.

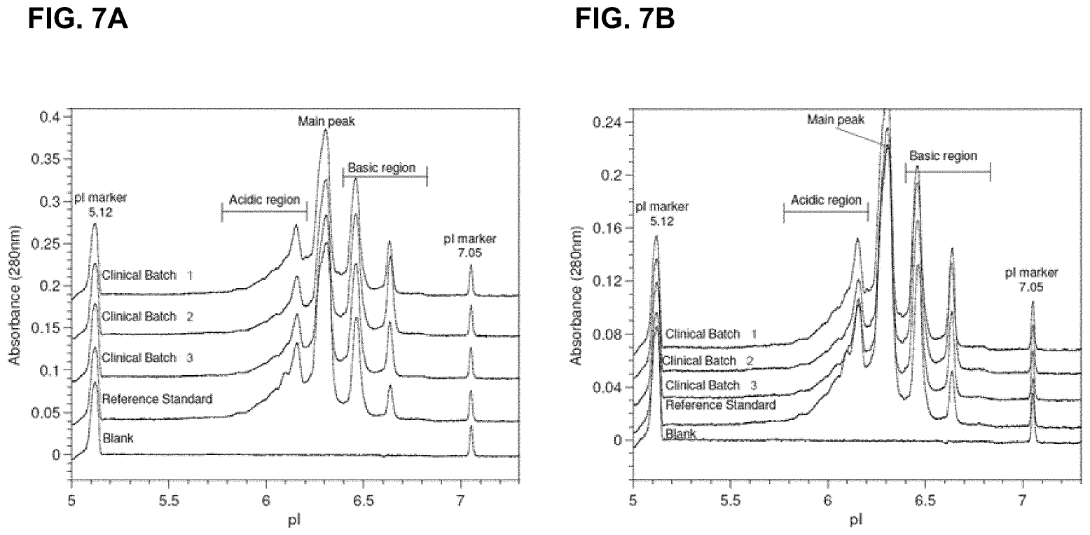

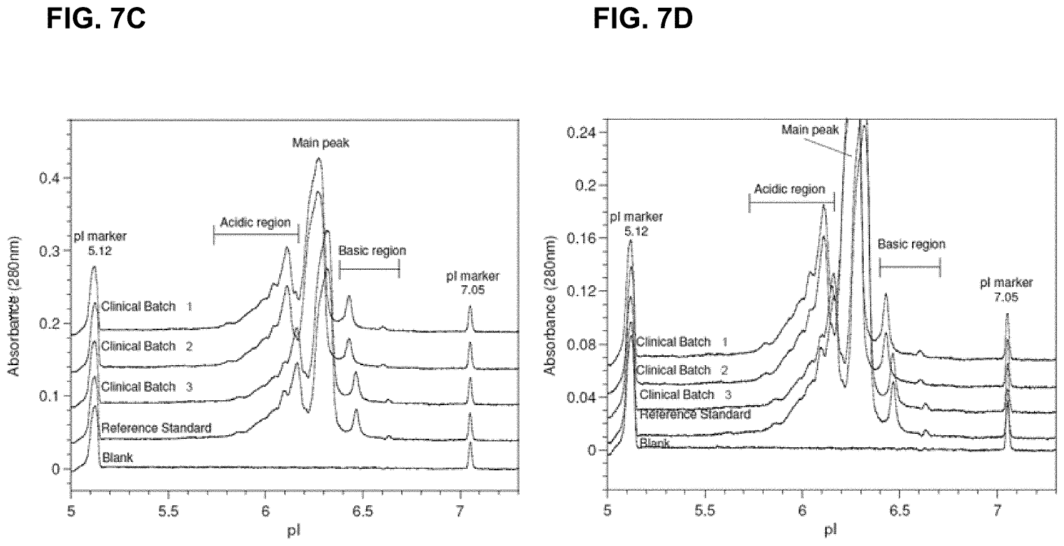

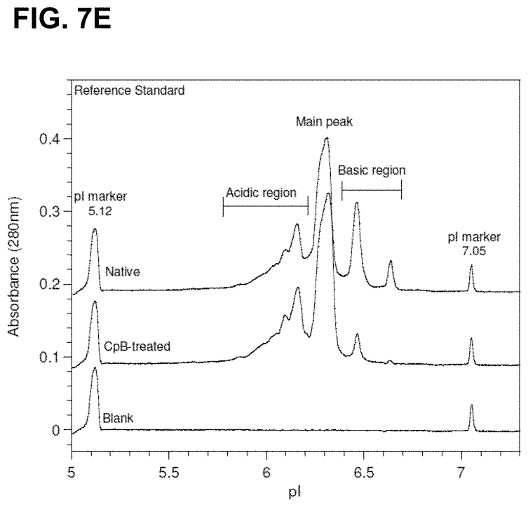

Results from the ICIEF analysis of native IL-22 Fc fusion protein without CpB treatment, summarized in Table 8 and shown in FIG. 7A (full-scale view) and FIG. 7B (expanded view), shows that the batches have variable charge variant distribution due to the C-terminal lysine charge heterogeneity. Results from the analysis of CpB-treated IL-22 Fc fusion protein, summarized in Table 9 and shown in FIG. 7C (full-scale view) and FIG. 7D (expanded view), demonstrated batch-to-batch consistency in the distribution of charge variants. A comparison of the results obtained with and without CpB treatment indicates that basic variants are mostly due to lysine heterogeneity (FIG. 7E).

TABLE 8
Distribution of Charge Variants by ICIEF of Native IL-22 Fc
Fusion Protein (Peak Area %)

| IL-22 Fc Fusion | Percent Charge Variants | | |
|---|---|---|---|
| protein Batch | Acidic Region | Main Peak | Basic Region |
| Reference Standard Batch | 31.0 | 47.7 | 21.3 |
| Clinical Batch 1 | 29.7 | 40.8 | 29.5 |
| Clinical Batch 2 | 24.8 | 38.6 | 36.6 |
| Clinical Batch 3 | 28.2 | 41.2 | 30.6 |

Abbreviations: ICIEF = imaged capillary isoelectric focusing.

TABLE 9
Distribution of Charge Variants by ICIEF of CpB-Treated IL-22 Fc
Fusion Protein (Peak Area %)

| IL-22 Fc Fusion | Percent Charge Variant | | |
|---|---|---|---|
| Protein Batch | Acidic Region | Main Peak | Basic Region |
| Reference Standard Batch | 40.6 | 53.6 | 5.8 |
| Clinical Batch 1 | 37.9 | 56.3 | 5.8 |
| Clinical Batch 2 | 40.6 | 53.6 | 5.8 |
| Clinical Batch 3 | 40.7 | 52.9 | 6.4 |

Abbreviations: ICIEF = imaged capillary isoelectric focusing.

Isoelectric Point

The pl is the pH at which the protein has no net charge. The pl of native IL-22 Fc fusion protein was determined by ICIEF after treatment with sialidase. From this analysis the pl of the major component was determined to be 6.5. The pl observed for the main peak in the ICIEF charge heterogeneity method may differ slightly from this value because the charge heterogeneity method employs narrow range ampholytes that produce a pH gradient calibrated by two bracketing pl markers.

Extinction Coefficient Determination

The protein concentration of the IL-22 Fc fusion protein solution was determined by comparing the spectrum of the proteolytically cleaved and unfolded IL-22 Fc fusion protein to the spectrum calculated from the amino acid sequence. This calculation was based on the known absorbance values of the individual amino acids (Bewley et al. Analytical Biochemistry 123:55-65, 1982). Using this method, the extinction coefficient of IL-22 Fc fusion protein was determined to be 0.98 mL mg⁻¹ cm⁻¹ at 280 nm. This extinction coefficient was used in the ultraviolet-visible spectrophotometric scan analysis to calculate IL-22 Fc fusion protein concentrations for all batches tested.

N-Glycosylation Site Occupancy

The IL-22 Fc fusion protein contains four N-glycosylation sites (Asn21, Asn35, Asn64, and Asn143) in each of the two cytokine domains of the molecule. N-glycosylation site occupancy of the IL-22 Fc fusion protein was determined by enzymatic deglycosylation of IL-22 Fc fusion protein followed by Lys-C peptide mapping and LC-MS analysis. To generate the IL-22 Fc fusion protein peptide maps, the protein was digested with endoproteinase Lys-C after subjecting the protein to denaturing conditions with guanidinium hydrochloride, reduction with dithiothreitol, and carboxymethylation of cysteines with iodoacetic acid. The N-glycans were cleaved from the protein using PNGase F enzyme. The resulting peptides were separated by UHPLC coupled to a mass spectrometer.

Percent site occupancy was calculated based on the integrated peak area of the extracted ion chromatogram of the deglycosylated peptide divided by the total peak area of the deglycosylated peptide and the native peptide. (PNGase F converts asparagine to aspartic acid, resulting in a 1 Da mass shift for the deglycosylated peptide.) The most abundant charge states of a peptide were considered for calculation of extracted ion chromatograms.

The percent N-glycosylation site occupancy of Asn21, Asn35, Asn64, and Asn143 are shown in Table 10. The site occupancy was shown to be consistent between the four N-glycosylation sites for the Reference Standard Batch and Clinical Batches 1, 2, and 3.

TABLE 10
Percent N-Glycosylation Site Occupancy of IL-22 Fc Fusion
Protein by Lys-C Peptide Mapping and LC-MS

| | Reference | | | |
|---|---|---|---|---|
| N-Glycosylation | Standard | Clinical | Clinical | Clinical |
| Site | Batch | Batch 1 | Batch 2 | Batch 3 |
| Asn-21 | 78 | 84 | 82 | 81 |
| Asn-35 | 100 | 100 | 100 | 100 |
| Asn-64 | 100 | 100 | 100 | 100 |
| Asn-143 | 30 | 34 | 33 | 33 |

Additional characterization of the percent N-glycosylation site occupancy of Asn21, Asn35, Asn64, and Asn143 was performed on Clinical Batches 4, 5, and 6. All six Clinical Batches demonstrated consistent site occupancy (Table 11).

TABLE 11
Percent N-Glycosylation Site Occupancy of IL-22 Fc Fusion Protein
Clinical Batches 1-6 by Lys-C Peptide Mapping and LC-MS

| N- | | | | | | | |
|---|---|---|---|---|---|---|---|
| Glyco- | | | | | | | |
| sylation | Clinical | Clinical | Clinical | Clinical | Clinical | Clinical | |
| Site | Batch 1 | Batch 2 | Batch 3 | Batch 4 | Batch 5 | Batch 6 | Mean |
| Asn-21 | 84 | 82 | 81 | 82 | 82 | 81 | 82 |
| Asn-35 | 100 | 100 | 100 | 100 | 100 | 100 | 100 |
| Asn-64 | 100 | 100 | 100 | 100 | 100 | 100 | 100 |
| Asn-143 | 34 | 33 | 33 | 35 | 33 | 32 | 33 |

N-Linked Glycan Analysis by 2-AA HILIC-UHPLC

The IL-22 Fc fusion protein contains four N-glycosylation sites per single chain molecule, all of which are located in the cytokine domain of the molecule at Asn21, Asn35, Asn64, and Asn143. The site occupancy was shown to be consistent between the four N-glycosylation sites for the Reference Standard Batch and Clinical Batches 1, 2, 3, 4, 5, and 6.

The relative distribution of the N-linked glycans of IL-22 Fc fusion protein was quantitatively assessed by HILIC-UHPLC with fluorescence detection. For this method, the N-glycans were cleaved from the protein under denaturing conditions using PNGase F enzyme. Released glycans were derivatized with the fluorescent label 2-AA and separated and detected by HILIC-UHPLC combined with fluorescence detection.

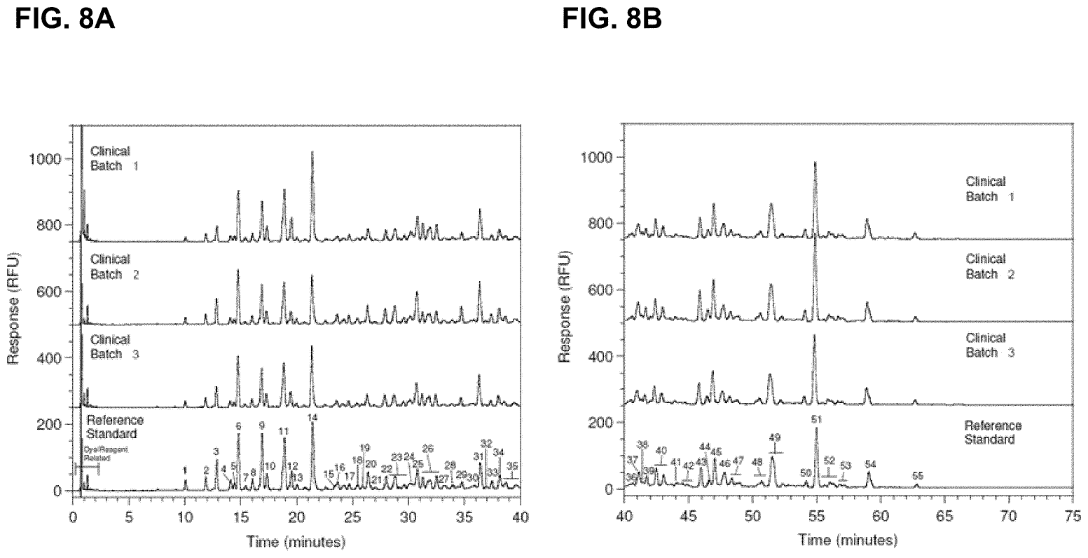

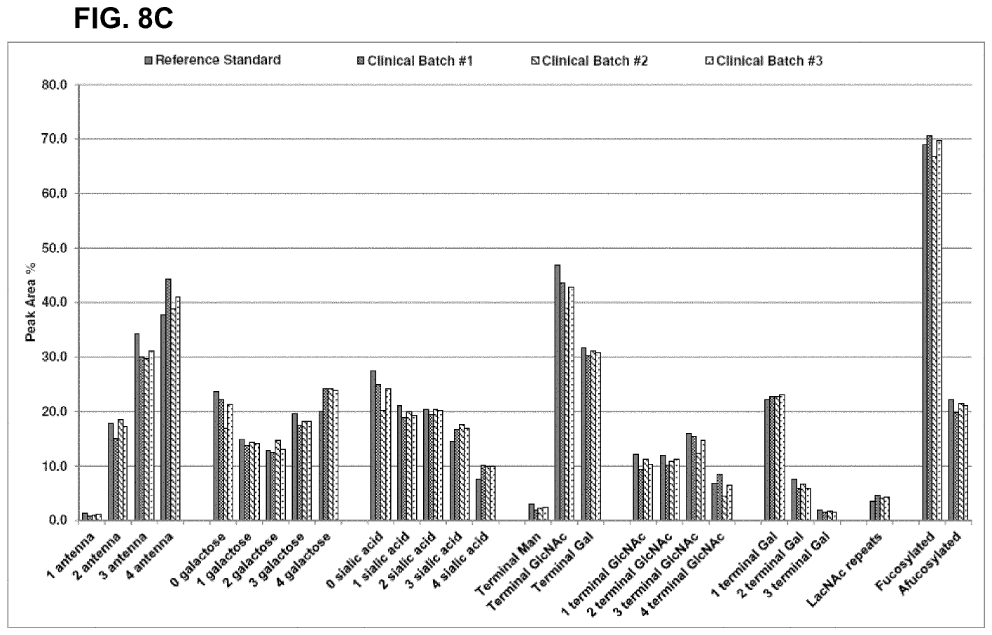

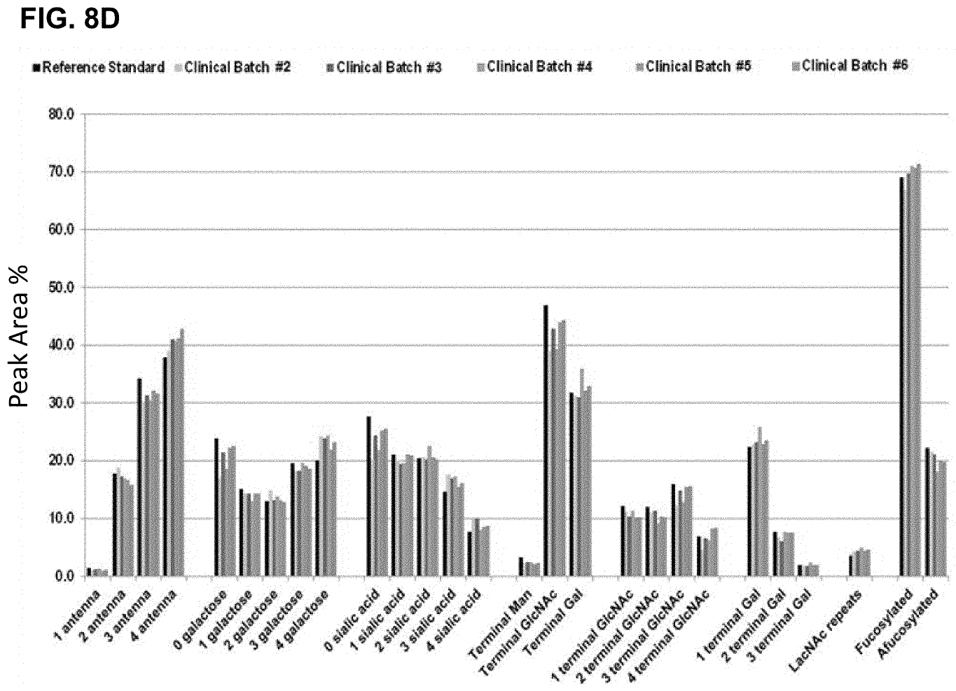

The chromatograms of the glycans observed in IL-22 Fc fusion protein Reference Standard Batch and Clinical Batches 1, 2, and 3 are shown in FIG. 8A and FIG. 8B. The relative N-linked glycan distributions of the IL-22 Fc fusion protein batches are provided in Table 12 and shown in FIG. 8C. FIG. 8D provides the relative N-linked glycan distributions of the IL-22 Fc fusion protein Reference Standard Batch and Clinical Batches 2, 3, 4, 5, and 6. The N-linked glycans were grouped according to attribute (FIG. 8A and FIG. 8B). Consistency of the glycosylation pattern and glycosylation attributes for the IL-22 Fc fusion protein Clinical Batches was demonstrated. All six Clinical Batches showed similar distribution as represented as percent (%) peak area (Table 13). Results from these extended characterization analyses demonstrated that the IL-22 Fc fusion protein batches have consistent glycan profiles.

In addition, analysis for galactose-alpha-1,3-galactose was also performed as part of extended characterization. Galactose-alpha-1,3-galactose was quantitatively assessed by high-performance anion-exchange chromatography-pulsed amperometric detection (HPAEC-PAD). No galactose-alpha-1,3-galactose was detected in the Reference Standard Batch and Clinical Batches using this method.

TABLE 12
Relative N-Glycan Distribution of IL-22 Fc Fusion Protein by 2-AA HILIC-UHPLC (Peak Area %)

| | | Reference | | | |
|---|---|---|---|---|---|
| | | Standard | Clinical | Clinical | Clinical |
| Area | Composition^{a} | Batch | Batch 1 | Batch 2 | Batch 3 |
| 1 | (HexNAc)₁ + (Man)₃(GlcNAc)₂ | 0.6 | 0.2 | 0.4 | 0.4 |
| 2 | (HexNAc)₁ (Deoxyhexose)₁ + | 0.9 | 0.5 | 0.7 | 0.7 |
| | (Man)₃(GlcNAc)₂ | | | | |
| 3 | (HexNAc)₂ + (Man)₃(GlcNAc)₂ | 1.9 | 1.0 | 1.5 | 1.5 |
| 4 | (HexNAc)₂ + (Man)₃(GlcNAc)₂ | 0.8 | 0.4 | 0.5 | 0.5 |
| 5 | (Hex)₁ (HexNAc)₁ + (Man)₃(GlcNAc)₂ | 0.5 | 0.4 | 0.0 | 0.4 |
| 6 | (HexNAc)₂ (Deoxyhexose)₁ + | 3.8 | 3.4 | 3.3 | 3.8 |
| | (Man)₃(GlcNAc)₂ | | | | |
| 7 | (HexNAc)₃ + (Man)₃(GlcNAc)₂ | 0.4 | 0.3 | 0.2 | 0.3 |
| 8 | (HexNAc)₂ (Deoxyhexose)₁ + | 0.9 | 0.6 | 0.6 | 0.7 |
| | (Man)₃(GlcNAc)₂ | | | | |
| 9 | (HexNAc)₃ + (Man)₃(GlcNAc)₂ | 4.4 | 3.1 | 2.9 | 3.4 |
| 10 | (HexNAc)₃ (Deoxyhexose)₁ + | 1.4 | 1.2 | 1.0 | 1.2 |
| | (Man)₃(GlcNAc)₂ | | | | |
| 11 | (HexNAc)₃ (Deoxyhexose)₁ + | 5.0 | 4.9 | 3.6 | 4.6 |
| | (Man)₃(GlcNAc)₂ | | | | |
| 12 | (HexNAc)₄ + (Man)₃(GlcNAc)₂ | 1.7 | 1.9 | 1.1 | 1.5 |
| 13 | (Hex)₁ (HexNAc)₂ (Deoxyhexose)₁ + | 0.8 | 0.5 | 0.7 | 0.6 |
| | (Man)₃(GlcNAc)₂ | | | | |
| 14 | (HexNAc)₄ (Deoxyhexose)₁ + | 5.6 | 7.1 | 3.9 | 5.5 |
| | (Man)₃(GlcNAc)₂ | | | | |
| 15 | (Hex)₁ (HexNAc)₃ (Deoxyhexose)₁ + | 0.3 | 0.2 | 0.2 | 0.2 |
| | (Man)₃(GlcNAc)₂ | | | | |
| 16 | (Hex)₁ (HexNAc)₂ (NeuAc)₁ + | 0.7 | 0.4 | 0.8 | 0.6 |
| | (Man)₃(GlcNAc)₂ | | | | |
| 17 | (Hex)₂ (HexNAc)₂ (Deoxyhexose)₁ + | 0.6 | 0.6 | 0.7 | 0.6 |
| | (Man)₃(GlcNAc)₂ | | | | |
| 18 | (Hex)₁ (HexNAc)₂ (Deoxyhexose)₁ | 0.7 | 0.5 | 0.6 | 0.6 |
| | (NeuAc)₁ + (Man)₃(GlcNAc)₂ | | | | |
| 19 | (Hex)₁ (HexNAc)₄ (Deoxyhexose)₁ + | 0.4 | 0.5 | 0.3 | 0.4 |
| | (Man)₃(GlcNAc)₂ | | | | |
| 20 | (Hex)₁ (HexNAc)₂ (Deoxyhexose)₁ | 1.9 | 1.4 | 1.7 | 1.5 |
| | (NeuAc)₁ + (Man)₃(GlcNAc)₂ | | | | |
| 21 | (Hex)₁ (HexNAc)₄ (Deoxyhexose)₁ + | 0.4 | 0.3 | 0.4 | 0.3 |
| | (Man)₃(GlcNAc)₂ | | | | |
| 22 | (Hex)₁ (HexNAc)₃ (NeuAc)₁ + | 1.5 | 1.2 | 1.5 | 1.4 |
| | (Man)₃(GlcNAc)₂ | | | | |
| 23 | (Hex)₁ (HexNAc)₃ (Deoxyhexose)₁ | 2.6 | 2.6 | 2.8 | 2.7 |
| | (NeuAc)₁ + (Man)₃(GlcNAc)₂ | | | | |
| 24 | (Hex)₁ (HexNAc)₄ (NeuAc)₁ + | 0.9 | 1.5 | 1.2 | 1.4 |
| | (Man)₃(GlcNAc)₂ | | | | |
| 25 | (Hex)₂ (HexNAc)₂ (Deoxyhexose)₁ | 2.0 | 2.5 | 3.1 | 2.7 |
| | (NeuAc)₁ + (Man)₃(GlcNAc)₂ | | | | |
| 26 | (Hex)₁ (HexNAc)₄ (Deoxyhexose)₁ | 3.1 | 5.1 | 3.9 | 4.6 |
| | (NeuAc)₁ + (Man)₃(GlcNAc)₂ | | | | |
| 27 | (Hex)₃ (HexNAc)₃ (Deoxyhexose)₁ + | 0.8 | 0.7 | 0.7 | 0.7 |
| | (Man)₃(GlcNAc)₂ | | | | |
| 28 | (Hex)₂ (HexNAc)₃ (Deoxyhexose)₁ | 0.7 | 0.6 | 0.6 | 0.6 |
| | (NeuAc)₁ + (Man)₃(GlcNAc)₂ | | | | |
| 29 | (Hex)₂ (HexNAc)₂ (NeuAc)₂ + | 0.8 | 0.8 | 1.5 | 1.0 |
| | (Man)₃(GlcNAc)₂ | | | | |
| 30 | (Hex)₂ (HexNAc)₄ (Deoxyhexose)₁ | 0.5 | 0.4 | 0.4 | 0.4 |
| | (NeuAc)₁ + (Man)₃(GlcNAc)₂ | | | | |
| 31 | (Hex)₂ (HexNAc)₂ (Deoxyhexose)₁ | 2.8 | 3.1 | 3.8 | 3.4 |
| | (NeuAc)₂ + (Man)₃(GlcNAc)₂ | | | | |
| 32 | (Hex)₃ (HexNAc)₃ (NeuAc)₁ + | 0.5 | 0.3 | 0.4 | 0.4 |
| | (Man)₃(GlcNAc)₂ | | | | |
| 33 | (Hex)₂ (HexNAc)₃ (NeuAc)₂ + | 0.9 | 0.7 | 1.0 | 0.8 |
| | (Man)₃(GlcNAc)₂ | | | | |
| 34 | (Hex)₃ (HexNAc)₃ (Deoxyhexose)₁ | 1.7 | 1.3 | 1.6 | 1.4 |
| | (NeuAc)₁ + (Man)₃(GlcNAc)₂ | | | | |
| 35 | (Hex)₂ (HexNAc)₃ (Deoxyhexose)₁ | 2.2 | 1.7 | 2.1 | 1.9 |
| | (NeuAc)₂ + (Man)₃(GlcNAc)₂ | | | | |
| 36 | (Hex)₂ (HexNAc)₄ (Deoxyhexose)₁ | 0.5 | 0.5 | 0.6 | 0.5 |
| | (NeuAc)₂ + (Man)₃(GlcNAc)₂ | | | | |
| 37 | (Hex)₃ (HexNAc)₃ (NeuAc)₂ + | 2.0 | 1.9 | 2.2 | 2.0 |
| | (Man)₃(GlcNAc)₂ | | | | |
| 38 | (Hex)₂ (HexNAc)₄ (Deoxyhexose)₁ | 0.5 | 0.6 | 0.5 | 0.6 |
| | (NeuAc)₂ + (Man)₃(GlcNAc)₂ | | | | |
| 39 | (Hex)₃ (HexNAc)₃ (NeuAc)₂ + | 1.1 | 0.9 | 1.2 | 1.0 |
| | (Man)₃(GlcNAc)₂ | | | | |
| 40 | (Hex)₃ (HexNAc)₃ (Deoxyhexose)₁ | 3.4 | 3.3 | 3.8 | 3.7 |
| | (NeuAc)₂ + (Man)₃(GlcNAc)₂ | | | | |
| 41 | (Hex)₄ (HexNAc)₄ (Deoxyhexose)₁ | 0.8 | 0.8 | 0.8 | 0.7 |
| | (NeuAc)₁ + (Man)₃(GlcNAc)₂ | | | | |
| 42 | (Hex)₃ (HexNAc)₄ (Deoxyhexose)₁ | 1.2 | 1.0 | 1.1 | 1.0 |
| | (NeuAc)₂ + (Man)₃(GlcNAc)₂ | | | | |
| 43 | (Hex)₃ (HexNAc)₃ (NeuAc)₃ + | 2.0 | 2.1 | 2.7 | 2.2 |
| | (Man)₃(GlcNAc)₂ | | | | |
| 44 | (Hex)₄ (HexNAc)₄ (NeuAc)₂ + | 0.9 | 1.0 | 1.2 | 1.0 |
| | (Man)₃(GlcNAc)₂ | | | | |
| 45 | (Hex)₃ (HexNAc)₃ (Deoxyhexose)₁ | 2.9 | 3.4 | 3.8 | 3.5 |
| | (NeuAc)₃ + (Man)₃(GlcNAc)₂ | | | | |
| 46 | (Hex)₄ (HexNAc)₄ (Deoxyhexose)₁ | 2.2 | 2.2 | 2.2 | 2.1 |
| | (NeuAc)₂ + (Man)₃(GlcNAc)₂ | | | | |
| 47 | (Hex)₃ (HexNAc)₄ (Deoxyhexose)₁ | 1.7 | 1.6 | 1.7 | 1.5 |
| | (NeuAc)₃ + (Man)₃(GlcNAc)₂ | | | | |
| 48 | (Hex)₄ (HexNAc)₄ (NeuAc)₃ + | 1.4 | 1.4 | 1.5 | 1.4 |
| | (Man)₃(GlcNAc)₂ | | | | |
| 49 | (Hex)₄ (HexNAc)₄ (Deoxyhexose)₁ | 5.0 | 6.0 | 6.0 | 5.1 |
| | (NeuAc)₃ + (Man)₃(GlcNAc)₂ | | | | |
| 50 | (Hex)₄ (HexNAc)₄ (NeuAc)₄ + | 0.7 | 0.9 | 1.1 | 0.9 |
| | (Man)₃(GlcNAc)₂ | | | | |
| 51 | (Hex)₄ (HexNAc)₄ (Deoxyhexose)₁ | 5.1 | 6.8 | 7.0 | 6.8 |
| | (NeuAc)₄ + (Man)₃(GlcNAc)₂ | | | | |
| 52^{b} | (Hex)₅ (HexNAc)₅ (Deoxyhexose)₁ | 1.3 | 1.7 | 1.6 | 1.4 |
| | (NeuAc)₃ + (Man)₃(GlcNAc)₂ | | | | |
| 53^{c} | (Hex)₄ (HexNAc)₄ (Deoxyhexose)₁ | 0.4 | 0.6 | 0.5 | 0.5 |
| | (NeuAc)₃ (NeuGc)₁ + (Man)₃(GlcNAc)₂ | | | | |
| 54^{b} | (Hex)₅ (HexNAc)₅ (Deoxyhexose)₁ | 1.8 | 2.5 | 2.3 | 2.3 |
| | (NeuAc)₄ + (Man)₃(GlcNAc)₂ | | | | |
| 55^{b} | (Hex)₆ (HexNAc)₆ (Deoxyhexose)₁ | 0.4 | 0.7 | 0.6 | 0.6 |
| | (NeuAc)₄ + (Man)₃(GlcNAc)₂ | | | | |

Note:
See to FIG. 8A and FIG. 8B for chromatograms.
Abbreviations: 2-AA HILIC-UHPLC = 2- aminobenzoic acid hydrophilic interaction liquid chromatography - ultra-high-performance liquid chromatography;
Hex = hexose;
HexNAc = N-acetylhexosamine;
NeuAc = N-acetylneuraminic acid;
NeuGc = N-glycolylneuraminic acid;
Man = mannose;
GlcNAc = N-acetylglucosamine.
^{a}Glycan composition assignments are based on the experimental mass of intact glycan using GlycoMod prediction tool (http://web.expasy.org/glycomod), which assumes a presence of standard N-linked pentasaccharide core consisting of (Man)₃(GlcNAc)₂.
^{b}Contains lactosaminic (galactose-N-acetylglucosamine) repeat(s).
^{c}Contains three NeuAc residues and one NeuGc residue.

TABLE 13
Relative N-Glycan Distribution of IL-22 Fc Fusion Protein Clinical Batches 1-6 by 2-AA HILIC UHPLC

| | Clinical | Clinical | Clinical | Clinical | Clinical | Clinical | | | | | | |
|---|---|---|---|---|---|---|---|---|---|---|---|---|
| | Batch | Batch | Batch | Batch | Batch | Batch | | | | | | |
| | 1 | 2 | 3 | 4 | 5 | 6 | | | −3 | +3 | −5 | +5 |
| | Peak Area % | | | | | | Mean | SD | SD | SD | SD | SD |
| N-Glycan Antennarity | | | | | | | | | | | | |
| 1 antenna | 0.9 | 1.1 | 1.2 | 1.1 | 0.9 | 1.0 | 1.0 | 0.1 | 0.6 | 1.5 | 0.3 | 1.7 |
| 2 antenna | 15.2 | 18.7 | 17.3 | 16.9 | 16.6 | 15.8 | 16.7 | 1.2 | 13.1 | 20.4 | 10.6 | 22.8 |
| 3 antenna | 30.2 | 29.8 | 31.3 | 30.5 | 32.0 | 31.6 | 30.9 | 0.9 | 28.2 | 33.5 | 26.5 | 35.3 |
| 4 antenna | 44.3 | 38.9 | 41.0 | 40.6 | 41.2 | 42.7 | 41.5 | 1.8 | 35.9 | 47.0 | 32.2 | 50.7 |
| Level of Galactosylation | | | | | | | | | | | | |
| 0 galactose | 22.3 | 17.0 | 21.3 | 18.6 | 22.2 | 22.4 | 20.6 | 2.3 | 13.7 | 27.5 | 9.1 | 32.1 |
| 1 galactose | 13.9 | 14.3 | 14.3 | 12.9 | 14.3 | 14.2 | 14.0 | 0.6 | 12.3 | 15.6 | 11.2 | 16.7 |
| 2 galactose | 12.5 | 14.7 | 13.1 | 13.8 | 13.1 | 12.8 | 13.3 | 0.8 | 10.9 | 15.7 | 9.3 | 17.4 |
| 3 galactose | 17.6 | 18.2 | 18.2 | 19.5 | 19.1 | 18.5 | 18.5 | 0.7 | 16.4 | 20.6 | 15.0 | 22.0 |
| 4 galactose | 24.3 | 24.2 | 23.9 | 24.3 | 21.9 | 23.1 | 23.6 | 0.9 | 20.8 | 26.4 | 18.9 | 28.3 |
| Level of Sialylation | | | | | | | | | | | | |
| 0 sialic acid | 25.0 | 20.3 | 24.3 | 21.8 | 25.2 | 25.4 | 23.7 | 2.1 | 17.3 | 30.0 | 13.1 | 34.3 |
| 1 sialic acid | 19.0 | 20.1 | 19.3 | 19.6 | 21.0 | 20.7 | 20.0 | 0.8 | 17.6 | 22.3 | 16.0 | 23.9 |
| 2 sialic acid | 19.5 | 20.5 | 20.2 | 22.6 | 20.6 | 20.3 | 20.6 | 1.0 | 17.5 | 23.7 | 15.5 | 25.8 |
| 3 sialic acid | 16.8 | 17.6 | 16.9 | 17.2 | 15.4 | 16.0 | 16.6 | 0.8 | 14.2 | 19.1 | 12.5 | 20.7 |
| 4 sialic acid | 10.1 | 10.0 | 10.0 | 8.0 | 8.5 | 8.6 | 9.2 | 0.9 | 6.4 | 12.0 | 4.5 | 13.9 |
| Terminal Man/GlcNAc/Gal Residues | | | | | | | | | | | | |
| Terminal Man | 1.9 | 2.2 | 2.5 | 2.4 | 2.0 | 2.2 | 2.2 | 0.2 | 1.6 | 2.9 | 1.2 | 3.3 |
| Terminal GlcNAc | 43.6 | 39.0 | 42.8 | 39.3 | 43.9 | 44.2 | 42.1 | 2.3 | 35.1 | 49.2 | 30.4 | 53.8 |
| Terminal Gal | 30.3 | 31.2 | 30.8 | 35.9 | 32.1 | 32.8 | 32.2 | 2.0 | 26.1 | 38.3 | 22.1 | 42.3 |
| Level of Terminal GlcNAc | | | | | | | | | | | | |
| 1 terminal GlcNAc | 9.5 | 11.2 | 10.4 | 11.3 | 10.1 | 10.2 | 10.4 | 0.7 | 8.4 | 12.5 | 7.0 | 13.8 |
| 2 terminal GlcNAc | 10.1 | 11.0 | 11.3 | 9.2 | 10.3 | 10.2 | 10.3 | 0.7 | 8.1 | 12.5 | 6.7 | 14.0 |
| 3 terminal GlcNAc | 15.4 | 12.3 | 14.7 | 12.8 | 15.3 | 15.6 | 14.3 | 1.4 | 10.1 | 18.6 | 7.2 | 21.5 |
| 4 terminal GlcNAc | 8.6 | 4.5 | 6.5 | 6.1 | 8.2 | 8.3 | 7.0 | 1.6 | 2.3 | 11.8 | -0.9 | 15.0 |
| Level of Terminal Galactose | | | | | | | | | | | | |
| 1 terminal Gal | 22.7 | 22.7 | 23.1 | 25.9 | 22.8 | 23.5 | 23.4 | 1.2 | 19.8 | 27.1 | 17.4 | 29.5 |
| 2 terminal Gal | 5.9 | 6.7 | 6.1 | 7.7 | 7.4 | 7.5 | 6.9 | 0.8 | 4.6 | 9.2 | 3.0 | 10.8 |
| 3 terminal Gal | 1.7 | 1.8 | 1.7 | 2.3 | 1.9 | 1.9 | 1.9 | 0.2 | 1.1 | 2.6 | 0.7 | 3.0 |
| 4 terminal Gal | ND | ND | ND | ND | ND | ND | ND | ND | ND | ND | ND | ND |
| LacNAc Repeats | | | | | | | | | | | | |
| LacNAc repeats | 4.6 | 4.2 | 4.3 | 4.9 | 4.3 | 4.5 | 4.5 | 0.3 | 3.7 | 5.2 | 3.2 | 5.7 |
| Core Fucosylation | | | | | | | | | | | | |
| Fucosylated | 70.7 | 66.8 | 69.7 | 71.0 | 70.6 | 71.4 | 70.0 | 1.7 | 65.1 | 75.0 | 61.7 | 78.3 |
| Afucosylated | 19.8 | 21.6 | 21.0 | 18.1 | 20.0 | 19.7 | 20.0 | 1.2 | 16.4 | 23.7 | 14.0 | 26.1 |

Man = mannose;
GlcNAc = N-acetylglucosamine;
Gal = galactose;
LacNAc = lactosaminic (galactose-N-acetylglucosamine) repeat(s);
ND = notdetected.
Note:
Peak area % values do not equal 100% as each value represents a relative percentage of total glycans.

### Site-Specific N-Glycosylation

The Asn21 N-glycosylation site in the cytokine domain of the IL-22 Fc fusion protein is located at or near the interaction interface with the IL-22 receptor (Jones et al. Structure 16:1333-44, 2008; Logsdon et al. J Mol. Biol. 342(2):503-14, 2004).

The relative distribution of N-linked glycans at site Asn21 was determined by Lys-C peptide mapping and LC-MS analysis. To generate the IL-22 Fc fusion protein peptide maps, the protein was digested with endoproteinase Lys-C after subjecting the protein to denaturing conditions with guanidinium hydrochloride, reduction with dithiothreitol, and carboxymethylation of cysteines with iodoacetic acid. The resulting peptides were separated by UHPLC coupled to a mass spectrometer.

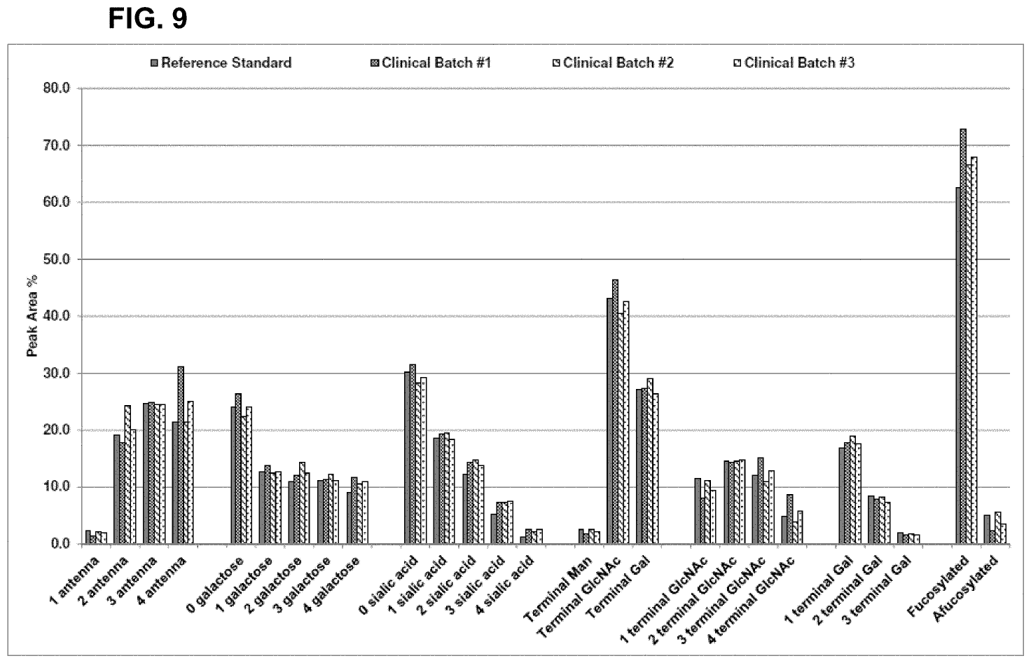

The Lys-C peptide mapping by LC-MS method provides information regarding the identification and relative abundance of N-linked glycans at a given N-glycosylation site. Due to potential differences in ionization efficiency of the many glycopeptides in IL-22 Fc fusion protein, relative quantitation was used to compare glycopeptide abundance between batches. The relative N-linked glycan distributions for Asn21 are shown in FIG. 9. N-linked glycans were grouped according to select major glycosylation attributes. Consistency of the glycosylation pattern and glycosylation attributes at Asn21 for the IL-22 Fc fusion protein Clinical Batches was demonstrated.

Sialic Acid Analysis for NANA Content

The sialic acid RP-HPLC method was used to determine N-acetylneuraminic acid (NANA) content and was performed as part of batch release testing. Quantitative release data for the Clinical Batches and for the Reference Standard Batch are shown side-by-side in Table 14. Results from the sialic acid analysis demonstrated that the batches had consistent NANA content within IL-22 Fc fusion protein release specifications (8-12 moles NANA/mole IL-22 Fc fusion protein). In addition, analysis for N-glycolylneuraminic acid (NGNA) was performed as part of extended characterization. The amount of NGNA remained consistently low between the Reference Standard Batch and the Clinical Batches (Table 14).

TABLE 14
N-acetylneuraminic Acid and N-glycolylneuraminic Acid Content
of IL-22 Fc Fusion Protein by RP HPLC

| IL-22 Fc Fusion | Moles NANA/Mole IL-22 | Moles NGNA/Mole IL-22 |
|---|---|---|
| Protein Batch | Fc Fusion Protein | Fc Fusion Protein |
| Reference | 7.8 | 0.06 |
| Standard Batch | | |
| Clinical Batch 1 | 8.7 | 0.08 |
| Clinical Batch 2 | 9.0 | 0.08 |
| Clinical Batch 3 | 9.3 | 0.07 |

Abbreviations:
NANA = N-acetylneuraminic acid;
NGNA = N-glycolylneuraminic acid;
RP-HPLC = reversed-phase high-performance liquid chromatography.

### Structural Characterization

Disulfide linkages contribute to the higher order structure of a protein. From the consensus sequence, four total intra-chain disulfide linkages per single chain with two in the cytokine (Cys7-Cys99 and Cys56-Cys145) and two in the Fc (Cys45-Cys105 and Cys151-Cys209) were deduced. In the intact molecule, two cysteine residues per single chain are expected to be involved in inter-chain disulfide linkages. These linkages are in the Fc and can be deduced from the consensus sequences: two disulfide linkages between the two single chains (Cys10-Cys10 and Cys13-Cys13).

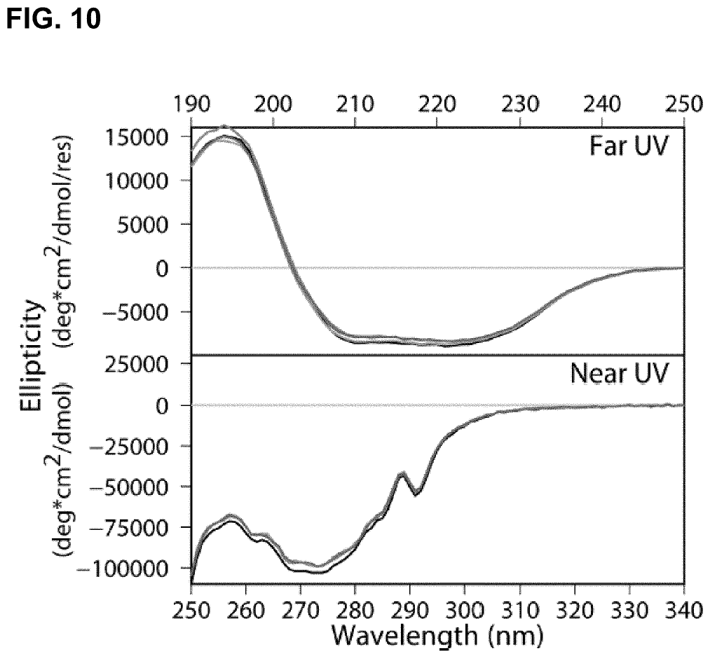

The higher-order structures of proteins are dictated by the amino acid sequence and post-translation modifications. Therefore, confirmation of primary structure of a protein is fundamental to the characterization of its structural properties. Methods that provide a direct assessment of the covalent structure and functional properties of the molecule were employed along with methods that are sensitive and quantitative to subtle variations in the properties of the surface of the molecule. Circular dichroism (CD) spectroscopy was used as part of extended characterization to look for the presence of higher order structural elements in the IL-22 Fc fusion protein. The secondary structural features, including α helices and β sheets, appear in the far-ultraviolet (UV) region of the CD spectrum (190-250 nm). These bands are caused by the relative orientation of the peptide bond along the protein backbone compared to the rest of the protein. In addition, the near-UV region of the CD spectrum (250-340 nm) provides information on the change in chiral orientation of aromatic residues (e.g. tryptophan, tyrosine and phenylalanine) that could be involved in hydrophobic, tertiary-structure contacts. The spectra of the Reference Standard Batch and Clinical Batches were similar to each other, which indicated no discernable differences in higher order structure of the IL-22 Fc fusion protein by CD analysis (FIG. 10).

### Example 2: IL-22 Fc Fusion Protein Potency and the Effect of Sialylation

### In Vitro Studies

The IL-22 Fc fusion protein potency assay measures the ability of IL-22 Fc fusion protein to bind to the IL-22 RA1 ECD. In the assay, varying concentrations of IL-22 Fc fusion protein Reference Standard Batch, control, and samples are added to a 96-well plate coated with IL-22 RA1 ECD. Bound IL-22 Fc fusion protein is detected with goat anti-human IgG-horseradish peroxidase (HRP) antibody and a tetramethylbenzidine substrate solution. The results, expressed in optical density (OD) units, are plotted against IL-22 Fc fusion protein concentrations, and a parallel curve program is used to calculate the measured potency of IL-22 Fc fusion protein sample(s) relative to the Reference Standard Batch.

Results from potency measurements demonstrated that the batches had consistent potency, met the acceptance criterion (40%-130% relative potency), and were suitable for the intended use (Table 15).

TABLE 15
IL-22 Fc Fusion Protein Potency

| | Potency by Binding Assay |
|---|---|
| IL-22 Fc Fusion Protein Batch | % Relative Potency^{a} |
| Reference Standard Batch | 100 |
| Clinical Batch 1 | 64 |
| Clinical Batch 2 | 70 |
| Clinical Batch 3 | 71 |

^{a}% Relative potency is reported as activity relative to the Reference Standard Batch, which is assigned a relative potency of 100%.

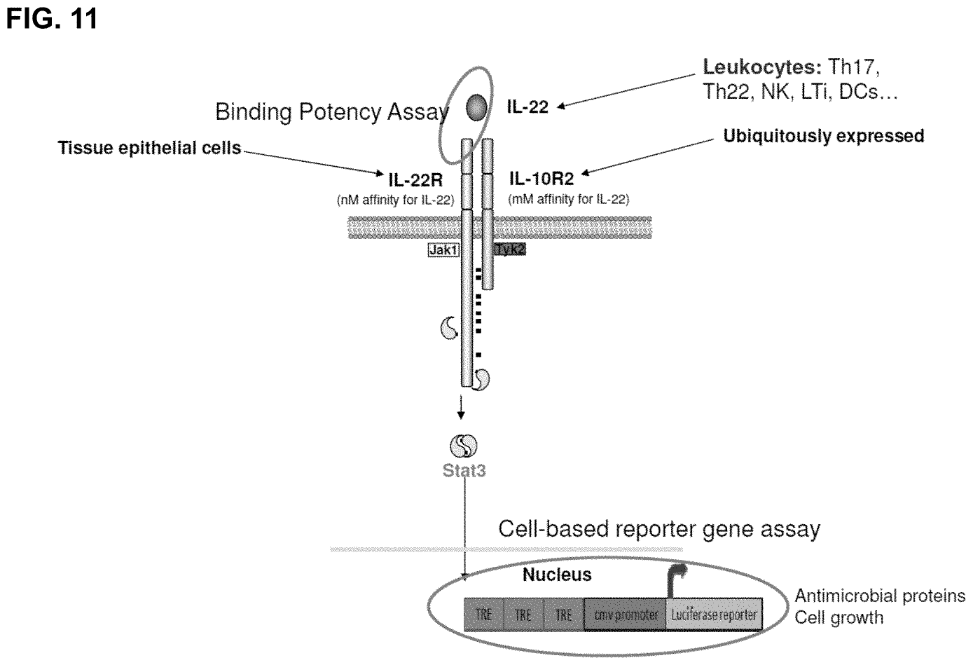

The presence and level of sialylation is known to have an impact on the interactions of glycoproteins, such as IL-22 (Marchal et al. Biol Chem 382:151-9, 2001). To study the impact of sialic acid on the interaction between IL-22 Fc fusion protein and the IL-22 receptor, IL-22 Fc fusion protein samples from different development batches with varying levels of sialic acid (quantitation limit of assay 3 mol/mol), 0.7, 4.6, 8.1, 12.0, or 15.4 mol sialic acid/mol IL-22 Fc fusion protein, were generated and tested in the binding assay and a cell-based reporter gene assay. The cell-based assay is a reporter gene assay that measures the ability of IL-22 Fc fusion protein to activate luciferase expression in engineered stable colo205 cells, which endogenously express IL-22 receptor. In the engineered stable colo205 cell reporter cell line, binding of a signal transducer and activator of transcription 3 (STAT3), to its DNA response elements in the promoter of the reporter gene induces firefly Luciferase expression. In the assay, colo205 reporter gene-expressing cells were incubated with prepared dilutions of IL-22 Fc fusion protein Reference Standard, Assay Control, and test samples in a 96-well assay plate. After a timed incubation, Luciferase Reagent was added to the wells of the assay plate, and reporter gene activity was measured using a luminescent plate reader. The amount of light emitted in each well of the assay plate is directly proportional to the amount of Luciferase induced by IL-22 Fc fusion protein Reference Standard, Assay Control, and test sample. The results, expressed as Luminescent units (LUM), were plotted against the IL-22 Fc fusion protein concentrations and parallel line analysis was used to estimate the activity of the IL-22 Fc fusion protein sample(s) relative to the Reference Standard. A schematic of this assay is shown in FIG. 11. To generate the material with varying levels of sialic acid, cell culture and purification processes were modified.

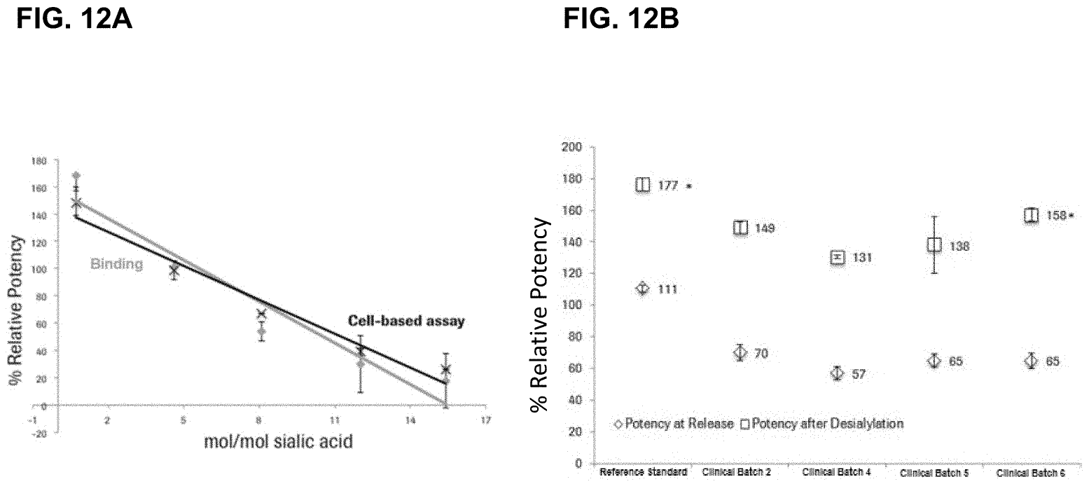

The correlation between sialic acid content and binding is maintained when activity was measured in the cell-based assay (FIG. 12A). The lines of correlation are not parallel, but show the same trend.

Treatment of a low level sialic acid variant and a high sialic acid variant with sialidase prior to potency examination using the binding assay and cell-based assay demonstrated that potency is not determined by sialic acid alone, but is also impacted by the underlying glycans (Table 16). SA Varriants were incubated with sialidase A at 0.01 U/mg, processed to remove the sialidase A, and formulated. The sialic acid (SA) Variant 15 (mol/mol) material was desialylated to produce the SA Variant 0 High material. The SA Variant 4 material was desialylated to produce the SA Variant 0 low material. The SA Variant 0 High material contains more tetraantennary glycans (i.e., more branching, hence the designation “High”), more galactosylated glycans, and less terminal GlcNAc-containing glycans than the SA level 0 low material. In other words, the SA Variant 0 High material contains more complete glycan structures than the SA Variant 0 Low material. The SA Variant 0 High material, having more branching and galactosylation, allows for the addition of more sialic acid, which can be added only to galactose residues. The increased branching and extent of galactosylation (available galactose residues) are considered to be involved in achieving to sialic acid levels of 15 and greater.

TABLE 16
Potency for IL-22 Fc Fusion Protein Sialic Acid Variants

| | Binding | | | Cell-based | |
|---|---|---|---|---|---|
| | Result | | | Result | |
| | (% Relative Potency) | | | (% Relative Activity) | |
| Sample | R&D | | % | | % |
| Description | SA | Mean | difference | Mean | difference |
| SA Variant 0 Low | 0.7 | 168* | 11 | 148 | 9 |
| mol/mol | | | | | |
| SA Variant 0 High | 0.9 | 120 | 0 | 121 | 12 |
| mol/mol | | | | | |
| SA Variant | 4.6 | 101 | 0 | 99 | 7 |
| 4 mol/mol | | | | | |
| SA Variant | 8.1 | 54* | 7 | 67 | 0 |
| 8 mol/mol | | | | | |
| SA Variant | 12.0 | 30* | 21 | 39 | 3 |
| 12 mol/mol | | | | | |
| SA Variant | 15.4 | 18* | 20 | 26 | 0 |
| 15 mol/mol | | | | | |

% difference: the difference between assays (n = 2)
*Estimates of potency

To further explore the relationship between sialic acid content and binding, several Clinical Batches were treated with sialidase to remove sialic acid. The desialylated samples were analyzed in the binding assay. Desialylated material from the Clinical Batches (2, 4, 5, and 6) and the Reference Standard Batch do not converge to a uniform potency value, suggesting that other product quality attributes contribute to potency differences (FIG. 12B). Glycan attributes, other than total sialic acid content, that may impact binding of IL-22 Fc fusion protein to IL-22 RA1 include branching (antennarity) and levels of galactosylation and sialylation. Increased potency for the Reference Standard Batch compared to the Clinical Batches was attributed to more terminal mannose and terminal GlcNAc, less branching, less galactosylation, and less sialylation, thereby indicating less complete glycan structures than those observed for Clinical Batches. Consistency of the glycosylation pattern and glycosylation attributes for all Clinical Batches was demonstrated.

To investigate the role of the underlying glycan structure on binding activity, the same Clinical Batches as above were treated with PNGase F enzyme to remove all N-glycans and analyzed in the potency assay. The potency of the process control sample, prepared from the Reference Standard Batch through the same treatment as the samples excepting PNGase F addition, was different than the potency of the Reference Standard Batch. In addition, differences in molecular size heterogeneity for the process control compared to the Reference Standard Batch were observed. The process control contained more high molecular weight (HMW) forms and less low molecular weight (LMW) forms than the Reference Standard Batch, as measured by size-exclusion ultra-high-performance liquid chromatography (SE-UHPLC). The SE-UHPLC chromatogram for the process control demonstrated a change in peak shape and retention time indicative of a change in glycan composition following the incubation and purification process.

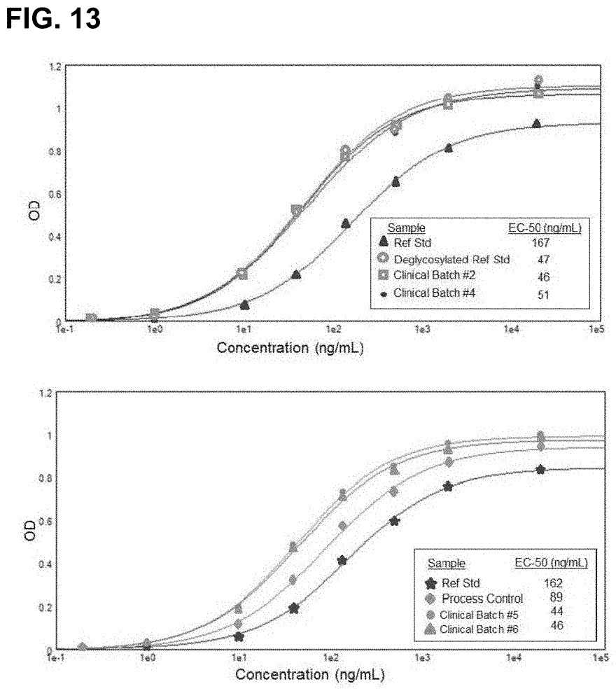

All deglycosylated samples, including the Reference Standard Batch, had levels of binding that converged to a level that was beyond the validated range of the assay. The EC50s (see FIG. 13) for all the deglycosylated samples were similar, suggesting that underlying glycans, beyond sialic acid, also contributed to binding activity. The potency shift of the process control may have been due to differences in glycan composition as indicated by the change in SE-UHPLC peak. The above results showed that the potency assay was sensitive to product quality attributes that impact the ability of the IL-22 Fc fusion protein to bind to the IL-22 RA1.

### In Vivo Studies

The impact of sialic acid content of IL-22 Fc fusion protein on the pharmacokinetic (PK) and serum REG3β PD response were evaluated in mice. Ninety-six female mice of the strain CD1 were assigned to one of six groups (n=16 mice/group). Animals in group 1 were given a single bolus dose of vehicle control and animals in groups 2-6 were given a single 1,000 μg/kg (1 mg/kg) IV bolus dose of IL-22 Fc fusion protein variant with sialic acid levels of 0.7, 4.6, 8.1, 12.0, or 15.4 mol sialic acid/mol IL-22 Fc fusion protein, via the tail vein. At various time points up to 21 days post-dose, serum samples (n=4/time point) were collected and analyzed for IL-22 Fc fusion protein concentrations and serum REG3β concentrations. Serum concentration-time data from individual animals were used to estimate PK parameters using a non-compartmental sparse analysis.

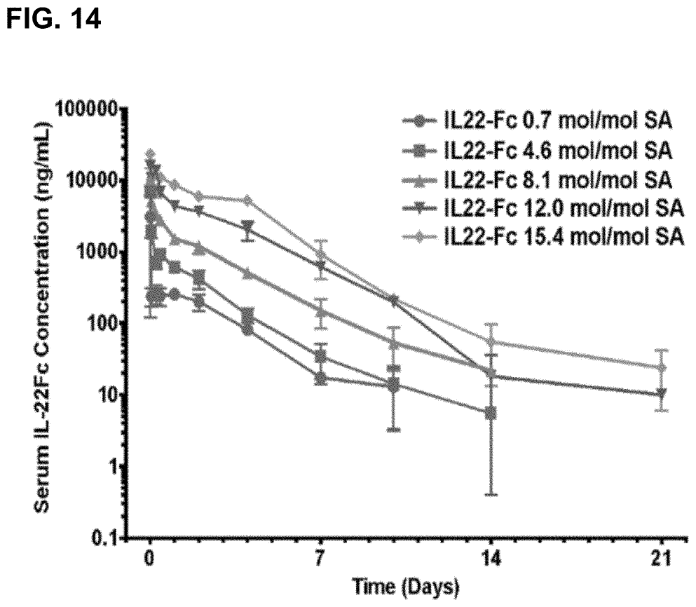

The mean±SD (standard deviation) serum IL-22 Fc fusion protein concentration-time profiles are presented in FIG. 14, and group mean PK parameter estimates are provided in Table 17.

TABLE 17
Non-Compartmental Pharmacokinetic Parameter Estimates following 1,000 μg/kg
Intravenous Administration of IL-22 Fc Fusion Protein Variant in CD1 Mice

| | Group 2 | Group 3 | Group 4 | Group 5 | Group 6 |
|---|---|---|---|---|---|
| | IL-22 Fc, | IL-22 Fc, | IL-22 Fc, | IL-22 Fc, | IL-22 Fc, |
| | 0.7 mol/mol | 4.6 mol/mol | 8.1 mol/mol | 12.0 mol/mol | 15.4 mol/mol |
| PK Parameter | SA | SA | SA | SA | SA |
| Cₘₐₓ (ng/mL) | 3,100 ± 1,490 | 6,850 ± 339 | 10,300 ± 1,040 | 15,800 ± 1,330 | 23,200 ± 743 |
| AUCₗₐₛₜ (day · ng/mL) | 1,020 ± 59.5 | 2,480 ± 129 | 7,500 ± 306 | 23,500 ± 925 | 39,800 ± 1230 |
| CL (mL/kg/day) | 945 | 399 | 132 | 42.6 | 25.1 |
| Vₛₛ (mL/kg) | 2,430 | 797 | 301 | 107 | 71.2 |
| T_{1/2,λz} (day) | 2.01 | 2.66 | 2.04 | 1.93 | 2.15 |

Abbreviations:
AUCₗₐₛₜ = area under the serum concentration-time curve from time 0 to the last measurable time point;
CL = clearance;
Cₘₐₓ = maximum observed concentration;
IL = interleukin;
mol = mole;
PK = pharmacokinetic;
SA = sialic acid;
T_{1/2,λz} = terminal elimination half-life;
Vₛₛ = volume of distribution at steady state.

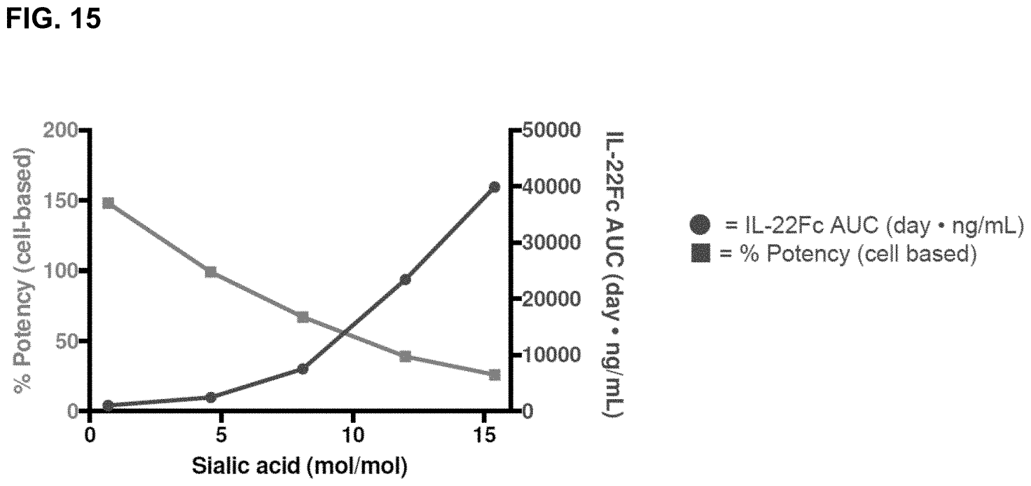

Following a single IV bolus dose of IL-22 Fc fusion protein with sialic acid level of 0.7, 4.6, 8.1, 12.0, or 15.4 mol/mol at 1,000 μg/kg administered to CD1 mice, mean clearance (CL) estimates were 945, 399, 132, 42.6, and 25.1 mL/kg/day; maximum observed serum concentrations (Cₘₐₓ) were 3,100, 6,850, 10,300, 15,800, and 23,200 ng/mL; and volume of distribution at steady state (Vₛₛ) estimates were 2,430, 797, 301, 107, and 71.2 mL/kg, respectively. Terminal half-life estimates were similar across the materials with different sialic acid levels and ranged between 1.93 to 2.66 days. Overall, IL-22 Fc fusion protein exposure increased, Vₛₛ increased, and CL decreased with increase in sialic acid levels (FIG. 15), likely mediated by liver uptake through recognition of exposed galactose residues by asialoglycoprotein (ASGP) receptors (Stefanich et al. J Pharmacol Exp Ther 327:308-15, 2008).

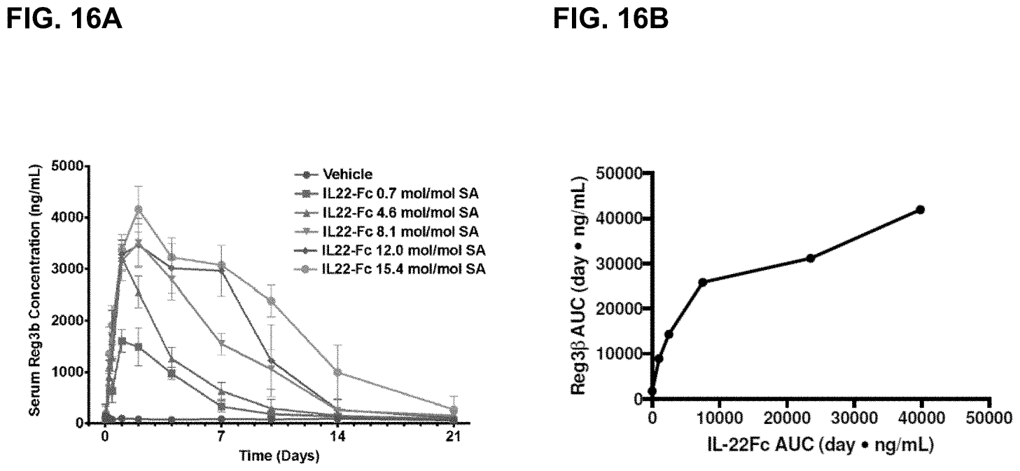

REG3a is an antimicrobial peptide produced by intestinal epithelial cells and pancreatic acinar cells and is a relevant PD biomarker indicative of IL-22R target engagement. REG3β is the mouse ortholog of human and cynomolgus monkey REG3a. The mean±SD serum REG3β concentration-time profiles are presented in FIG. 16A. A monotonic increase in serum levels of REG3β with increasing sialic acid levels of IL-22 Fc fusion protein were observed following a single IV bolus dose of 1,000 μg/kg in CD1 mice. The relationship between changes in IL-22 Fc fusion protein area under the curve (AUC) and corresponding changes in serum Reg3β AUC with different sialic acid levels is shown in FIG. 16B. The combined PK/PD data showed that the IL-22 Fc fusion protein exposure and serum REG3β response increased with increasing sialic acid levels of IL-22 Fc fusion protein. This suggests that increase in IL-22 Fc fusion protein exposure with increasing sialic acid content resulted in an increase in serum REG3β PD response in vivo, at a dose of 1,000 μg/kg IV in CD1 mice, despite reduction in in vitro potency with increase in sialic acid content.

These studies demonstrated that the binding potency assay is sensitive to sialic acid content in a manner consistent with a preliminary cell-based assay.

The negative correlation observed between sialic acid content and potency does not result in the reduction in the in vivo pharmacological effect, given the positive correlation observed between sialic acid content and PD response in the mouse PK/PD study. While the potency was reduced with increasing sialic acid, the in vivo clearance and volume of distribution were also reduced leading to higher exposure (in both Cₘₐₓ and AUC). The combined PK/PD data showed that changes in IL-22 Fc fusion protein exposure due to differences in sialic acid content was the predominant driver of the changes observed in the in vivo serum REG3β PD response, despite the opposing effects of sialic acid content on in vitro potency.

### Example 3: Chemistry, Manufacturing Process, and Process Controls of IL-22 Fc Fusion Protein

### Batch and Scale Definition

IL-22 Fc fusion protein was manufactured in a bioreactor using a suspension-adapted CHO cell line. The source of cells was the Master Cell Bank (MCB), and a thaw of the MCB may be used to source several production runs. A single batch of harvested cell culture fluid (HCCF) was produced from each cell culture production run. One or more batches of HCCF were processed through purification and final conditioning to produce a single batch of IL-22 Fc fusion protein. All manufacturing was in accordance with cGMPs. Production using the processes described herein occurred at the scales listed in Table 18.

TABLE 18
Manufacturing Scales for the Cell Culture Process

| Seed Train | Inoculum Train | Production |
|---|---|---|
| Scale (L) | Scale(s) (L) | Scale (L) |
| 20 | 80/400 | 2000 |

### Cell Culture and Harvest

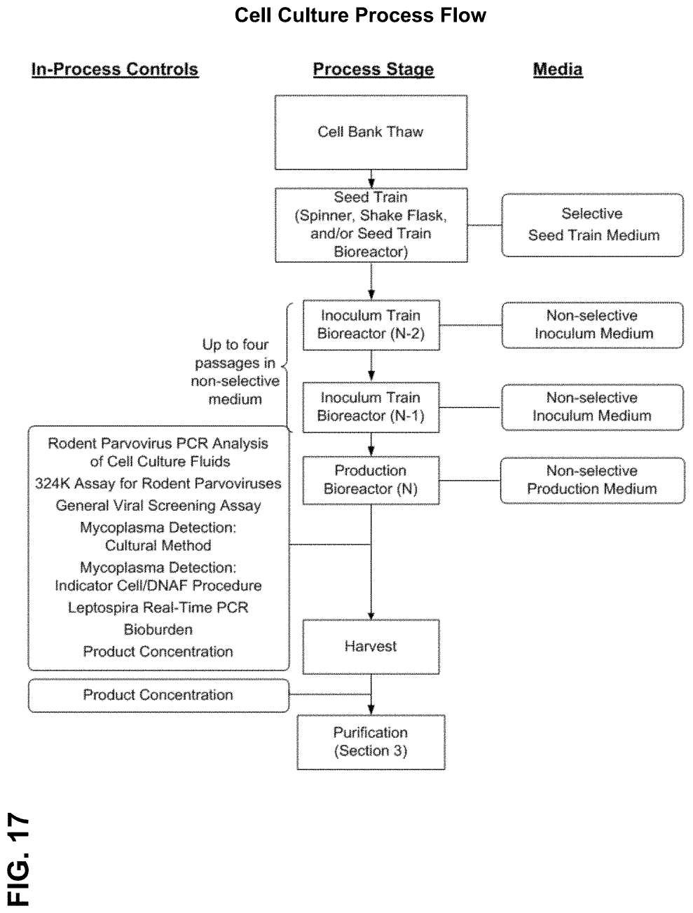

The cell culture process used to produce IL-22 Fc fusion protein consists of four stages: seed train, inoculum train, production, and harvest. The flow diagram in FIG. 17 illustrates the stages, in-process controls (IPCs), and relevant information for the cell culture and harvest processes. Production using the processes described in this section occurred at the scales listed in Table 18. Process parameters are listed in Table 19.

Description of the Cell Culture Process

Cell Culture Media

The cell culture stages used different types of media, all of which are chemically defined media. Selective medium containing methionine sulfoximine (MSX) was used in the seed train stage, while non-selective medium was used in the inoculum and production stages. A non-selective nutrient feed medium was also used at the production stage. The basal medium used during the production cell culture is chemically defined medium, which was selected to minimize the potential risk associated with the use of animal-derived raw materials with regards to adventitious agents. The medium contains amino acids, vitamins, trace elements, and buffer components.

All cell culture media were serum-free, chemically defined, and include cell protective agents, polysaccharides, and osmolality adjustment agents. One raw material containing an animal-derived component was used in the process: 30% simethicone emulsion is added as needed to control foaming.

Seed Train

To initiate a seed train, an ampoule or ampoules of cells from the serum-free IL-22 Fc fusion protein MCB were removed from liquid nitrogen storage, thawed, and used to inoculate either a spinner, a shake flask, or a seed train bioreactor.

The cells were subcultivated following thaw and are subsequently passaged in the selective seed train medium. The culture conditions for the seed train are provided in Table 19. Cells from the seed train were used to inoculate the first inoculum train bioreactor.

In other examples, a rolling seed train can be used for production of IL-22 Fc fusion protein. In this example, the seed train is grown continuously (up to a certain cell age) to inoculate the inoculum train.

Inoculum Train

To provide inoculum for IL-22 Fc fusion protein production cultures, the seed train cell mass was expanded by subcultivation in non-selective medium into a larger-sized bioreactor or bioreactors. The subcultivation between the seed train and the production stage is designated as the inoculum train (N-2, and N-1 cultures). The maximum number of passages in the inoculum train, currently limited to four or fewer, will be defined by future studies on the stability of IL-22 Fc fusion protein expression in non-selective medium. The culture conditions for the inoculum train are provided in Table 19.

Production Stage

The production stage of IL-22 Fc fusion proteins was conducted in a bioreactor using non-selective medium. To inoculate a production culture, cells from the final stage of the inoculum train (referred to as the N-1 culture) were transferred into a production bioreactor containing production medium. To maintain cell viability and productivity, nutrient feeds were added to the production bioreactor over the course of the culture. The production process also employed a temperature shift to extend culture viability and enhance productivity. The production culture conditions are summarized in Table 19.

Prior to the harvest of the production culture, samples were taken and analyzed to confirm product safety with respect to microorganisms and viruses.

Process Controls

Cell culture performance indicators (e.g., cell density, viability, and titer), and process parameters (culture pH, temperature, and dissolved oxygen) were monitored. Process parameters that were monitored and controlled are shown in Table 19. Action and rejection limits for in-process control tests are provided in Table 20. The pH of bioreactor cultures was adjusted with the addition of CO₂ gas (acid) and/or Na₂CO₃, NaOH, or other suitable base as needed. Bioreactor cultures were supplemented with antifoam (simethicone emulsion) to minimize foam formation. All media solutions were filtered through sterilizing-grade membrane filters (pore size rating 0.1 μm) prior to use. All gases used for pH and dissolved oxygen control were filtered through sterilizing-grade membrane filters (pore size rating 0.22 μm) prior to use. Action and rejection limits for in-process control tests are provided in Table 20. The manufacturing process was designed to operate using fed batch culture processes. There were no intermediates in the processing of IL-22 Fc fusion protein.

TABLE 19
Process Parameter Targets for Each Cell Culture Process Stage

| Process Stage and Vessel | Process Parameter | Target^{a} |
|---|---|---|
| Seed Train Spinners or | Temperature | 37° C. |
| Shake Flasks | Culture Duration | 2-7 days |
| | | for each passage |
| Seed Train Bioreactor | Temperature | 37° C. |
| | pH | 7.15 |
| | Dissolved Oxygen^{b} | 30% |
| | Culture Duration | 2-5 days |
| Inoculum Train | Temperature | 37° C. |
| Bioreactor(s) | pH | 7.10 |
| | Dissolved Oxygen^{b} | 30% |
| | Culture Duration | 2-3 days for |
| | | each bioreactor |
| Production Bioreactor | Initial Temperature | 37° C. |
| | Temperature Post shift | 33° C. |
| | Temperature Shift | 72 hours |
| | Timing | |
| | pH | 7.00 |
| | Dissolved Oxygen^{b} | 30% |
| | Culture Duration | 12 days |

^{a}Acceptable ranges will be established through development to ensure that IL-22 Fc Fusion Protein consistently meets product release specifications.
^{b}Dissolved oxygen is maintained at set point by direct sparging with air and/or oxygen gas and/or oxygen-enriched air.

TABLE 20
In Process Controls with Limit

| Process Step | Action | Acceptance |
|---|---|---|
| Test Name | Limit | Criteria^{a} |
| Preharvest Cell Culture Fluid | | |
| (sampling performed at the time of harvest) | | |
| Rodent Parvovirus PCR Analysis of | NA | Negative |
| Cell Culture Fluids | | |
| 324K Assay for Rodent Parvoviruses | NA | None Detected |
| General Viral Screening Assay | NA | None Detected |
| Mycoplasma Detection: Culture Method | NA | None Detected |
| Mycoplasma Detection: Indicator Cell/DNAF | NA | None Detected |
| Procedure | | |
| Leptospira Real-Time PCR | NA | None Detected |
| Bioburden (CFU/10 mL) | NA | ≤10 |
| Harvested Cell Culture Fluid | | |
| Bioburden (post-filtration) (CFU/10 mL) | >10 | NA |
| In-Process Steps prior to Final UFDF Pool | | |
| Bioburden (pre-filtration) (CFU/10 mL) | >100 | NA |
| Bioburden (post-filtration) (CFU/10 mL) | >10 | NA |
| UFDF Pool (unconditioned bulk) | | |
| Host Cell Protein Content (ng/mg) | >60.0 | ≤200.0 |
| Protein A Content (ng/mg) | >10 | NA |
| DNA Content (pg/mg) | NA | ≤238.1 pg/mg |
| Bioburden (pre-filtration) (CFU/10 mL) | >50 | NA |
| UFDF Pool (conditioned) | | |
| Bioburden (post-filtration) (CFU/10 mL) | >10 | NA |

CFU = colony forming unit;
DNAF = DNA-binding fluorochrome;
NA = not applicable;
PCR = polymerase chain reaction;
UFDF = ultrafiltration and diafiltration.
^{a}The current Clinical Batches were manufactured with specified rejection limits. For future batches, acceptance criteria are specified. If an acceptance criterion was exceeded, the batch was rejected.

Process-Related Impurities

Process-related impurities including host-cell DNA, residual protein A, and host-cell protein (HCP) were monitored routinely in the IL-22 Fc fusion protein as part of in-process-control testing. An assessment of the capabilities of removal of impurities such as methionine sulfoximine also known as MSX, antifoam (simethicone emulsion), and Kolliphor P 188 otherwise known as poloxamer 188 in the IL-22 Fc fusion protein purification process is presented in this section, either by documentation that the impurities are significantly diluted in the process to acceptably low levels (MSX) or that the concentration of impurities are significantly reduced in the purification process to acceptably low levels (simethicone and poloxamer 188).

MSX was added for selective pressure to seed train cultures at a level of 50 μM. MSX was not added to the inoculum train or to the production bioreactor; therefore, the maximum concentration of MSX in the production culture medium is 81 μg/L or 54 ng MSX per mg of IL-22 Fc fusion protein based on the largest volume and lowest expected titer of 1.5 g/L. When assuming no clearance of MSX in the purification process, the maximum amount of MSX potentially remaining would be 2.3 μg MSX per the proposed maximum dose (42 mg) for the phase I clinical study. However, it was expected that the chromatography and ultrafiltration and diafiltration (UFDF) steps would further reduce the level of small molecules such as MSX.

Process-related impurities such as simethicone and poloxamer 188 in the IL-22 Fc fusion protein purification process were measured by nuclear magnetic resonance (NMR) after the first chromatography step, in the affinity pool. The simethicone and poloxamer 188 were below the limit of quantitation (LOQ) of the assay (10 μg/mL) in the affinity pools (refer to Table 21).

TABLE 21
Process-Related Impurities Levels in Affinity Pools

| | Poloxamer 188 | Simethicone |
|---|---|---|
| IL-22 Fc Fusion Protein Batch | (μg/mL) | (μg/mL) |
| Clinical Batch 1 | <10 | <10 |
| Clinical Batch 2 | <10 | <10 |
| Clinical Batch 3 | <10 | <10 |

Residual Solvents

No class 1 or class 2 solvents were used in the production of the IL-22 Fc fusion protein. A low concentration of glacial acetic acid was used in the IL-22 Fc fusion protein purification. According to The International Council for Harmonisation of Technical Requirements for Pharmaceuticals for Human Use (ICH) Guideline for residual solvents (Q3C), glacial acetic acid is low in toxicity and a low risk to human health.

Harvest

At the end of the production culture, the cell culture fluid was separated from the cells. For harvesting, the culture was cooled down in the production bioreactor. The cells were then removed by centrifugation using a disk stack separator and subsequently filtered using single-use depth and microbial retention filters.

There is a potential for disulfide bond reduction to occur with mAbs or mAb related formats. To date, this phenomenon has not been observed for IL-22 Fc fusion protein. However, several mitigation strategies have been implemented during the development of IL-22 Fc fusion protein as precautionary measures. IL-22 Fc fusion protein was purified from the harvested cell culture fluid as described below.

Purification and Modification Reactions

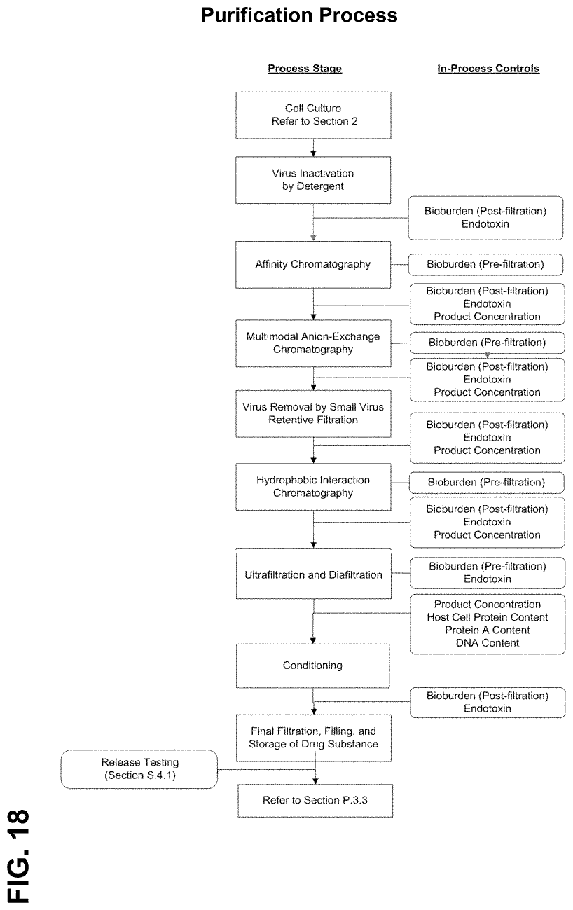

The process steps and IPCs used for purification and final conditioning IL-22 Fc fusion protein are illustrated in FIG. 18.

The composition of buffers used in the purification process steps is provided in Table 22, Table 23, Table 24, Table 25, and Table 26.

TABLE 22
Composition of Detergent Virus Inactivation Solution Used
in the IL-22 Fc Fusion Protein Purification Process

| | |
|---|---|
| Process Step | Composition |

Detergent Virus Inactivation

| | |
|---|---|
| Detergent Stock Solution | 10% Triton X-100 |

TABLE 23
Composition of Affinity Chromatography Buffers Used
in the IL-22 Fc Fusion Protein Purification Process

| Process Step | Composition |
|---|---|
| Equilibration Buffer | 0.025M Tris, 0.025M sodium |
| | chloride, pH 7.7 |
| Wash Buffer | 0.4M potassium phosphate, pH 7.0 |
| Elution Buffer | 0.30M L-arginine hydrochloride, |
| | 0.013M sodium phosphate, pH 3.8 |
| Conductivity Adjustment | Water |
| Solution for pool | |
| pH Adjustment Solution | 1.5M Tris base, pH 11.0 |
| for pool | 2.0M acetic acid, pH 2.2 |

TABLE 24
Composition of Multimodal Anion-Exchange Chromatography
Buffers Used in the IL-22 Fc Fusion Protein Purification Process

| Process Step | Composition |
|---|---|
| Equilibration Buffer | 0.040M sodium acetate, pH 5.8 |
| Gradient Elution Buffer | 0.040M sodium acetate, |
| | pH 5.8 and 0.04M sodium acetate, |
| | 0.30M sodium sulfate pH 5.8 |

TABLE 25
Composition of Hydrophobic-Interaction Chromatography Buffers
Used in the IL-22 Fc Fusion Protein Purification Process

| Process Step | Composition |
|---|---|
| Conductivity Adjustment | 1.2M sodium sulfate |
| Solution for load | |
| pH Adjustment Buffer for load | 1.5M Tris base, pH 11.0 |
| | 2.0M acetic acid, pH 2.2 |
| Equilibration Buffer | 0.02M MOPS, 0.30M |
| | sodium sulfate, pH 7.0 |

TABLE 26
Composition of Ultrafiltration and Diafiltration Buffers Used
in the IL-22 Fc Fusion Protein Purification Process

| Process Step | Composition |
|---|---|
| Diafiltration Buffer | 0.010M sodium phosphate, pH 7.2 |
| Conditioning Buffer | 0.010M sodium phosphate, 1.2M sucrose, |
| | 0.025M Methionine, |
| | 0.1% polysorbate 20, pH 6.8 |

Virus Inactivation by Detergent Addition

A 10% stock solution of detergent Triton X-100 was added to generate the harvested cell culture fluid (HCCF) to achieve a final concentration of 0.5% Triton X-100. The HCCF was held for 1 hour at 20° C.-24° C. to inactivate potential virus particles.

Affinity Chromatography

The affinity chromatography step was a bind-and-step-elute process using MABSELECT SURE® resin. After cell separation and Triton addition, the HCCF was applied onto the equilibrated column. Proteinaceous and non-proteinaceous impurities were removed by washing the column. The product was recovered from the column with a low pH elution buffer. Affinity pooling was initiated by volume and terminated based on absorbance at 280 nm. This chromatography step removed residual impurities such as DNA, host cell protein, endotoxin, virus, and small molecules.

Multimodal Anion-Exchange Chromatography

The multimodal anion-exchange step was a bind-and-gradient elution process using CAPTO™ adhere resin. After equilibration of the multimodal anion-exchange column with equilibration buffer, the conductivity- and pH-adjusted affinity pool was loaded onto the column. After IL-22 Fc fusion protein binds to the resin, the column was washed with equilibration buffer. IL-22 Fc fusion protein was eluted off the column with elution buffer using an increasing salt gradient. Multimodal anion-exchange pooling was initiated and terminated based on absorbance at 280 nm. This chromatography step removed residual impurities such as DNA, host cell protein, virus, and high-molecular-weight forms (HMWF).

Virus Removal by Small Virus Retentive Filtration The product pool from the preceding step was filtered through a single-use normal-flow small virus retentive filter (VIRESOLVE® Pro Magnus). An integrity test was performed on the filters before and after use.

Hydrophobic Interaction Chromatography

The hydrophobic-interaction step is operated was a flow-through mode using Phenyl SEPHAROSE™ FF resin. After equilibration of the hydrophobic-interaction column with equilibration buffer, the conductivity and pH-adjusted product pool from the preceding step was loaded onto the column. IL-22 Fc fusion protein flowed through the column, which was then washed with equilibration buffer. Hydrophobic-interaction pooling was initiated and terminated based on absorbance at 280 nm. This chromatography step removed residual impurities such as host cell protein, virus, and HMWF.

Ultrafiltration and Diafiltration

The product pool was concentrated to approximately 20 g/L by ultrafiltration using a 10 kDa composite regenerated cellulose ultrafiltration membrane. The concentrated pool was then diafiltered (buffer exchanged) into diafiltration buffer.

Conditioning

The ultrafiltration and diafiltration (UFDF) pool was diluted with diafiltration buffer, and conditioned to a final concentration of 10.0±1.0 g/L IL-22 Fc fusion protein in 0.010 M sodium phosphate, 0.24 M sucrose, 0.005 M Methionine, 0.02% polysorbate 20, pH 7.1.

Final Filtration, Filling, and Storage of IL-22 Fc Fusion Protein

The conditioned UFDF pool was filtered through a 0.22 μm membrane to yield IL-22 Fc fusion protein that is stored at −20° C.

Combining Same-Step in-Process Pools

The product-containing in-process pools may be stored at room temperature or at 2° C.-8° C. between process steps and may be combined for further processing. For the resulting IL-22 Fc fusion protein to be acceptable for release, the individual pools that were combined must each individually meet in-process limits. Combining pools to address quality issues is not acceptable

Refiltration

Refiltration is a proactive measure that was permitted only to prevent compromise of the in-process pools. On rare occasion, refiltration may be required in the process when an in-process pool is at risk due to an operational event such as the following:
a.) An unacceptable post-use filter integrity test for a previous filtration step with the exemption of the filtration steps.
b.) Equipment problems that could potentially compromise the integrity of the storage container (e.g., valve failure or improper vent filter installation).
c.) Exceeding a validated hold time for cleaned or steamed equipment

Refiltration was not allowed for removal of microbial contaminations or resolution of any other product quality problem.

### Reprocessing

Reprocessing of a IL-22 Fc fusion protein batch may be performed under limited circumstances such as an equipment malfunction that can be clearly identified. Examples include:
a.) non-integral column bed
b.) defective gradient pump
c.) An unacceptable post-use filter integrity test for a previous filtration step resulting in the repetition of filtration steps that are described in the process description (e.g., depth filtration, nanofiltration [small virus retentive filter], or final IL-22 Fc fusion protein filtration).

Reprocessing was conducted by repeating one or more of the manufacturing steps described in this section. All relevant IPC limits for the reprocessed step(s) must be met.

The quality of the batch must be investigated and demonstrated to be unaffected by the reprocessing. Therefore, all IPC limits and release specifications must be met. If applicable, extended characterization and stability of the reprocessed material were assessed to exclude a quality impact.

### Filling and Storage

The conditioned UFDF pool was filtered into disposable bioprocess bags to produce IL-22 Fc fusion protein, which was stored at 2° C.-8° C. for further processing or frozen for long-term storage at −20° C. IL-22 Fc fusion protein may be stored at the manufacturing site or transported under controlled temperature conditions to other sponsor sites/contract manufacturing organization sites for long-term storage or for the IL-22 Fc fusion protein pharmaceutical composition manufacture in accordance with shipping procedures.

### Specification

The release specification and acceptance criteria used for IL-22 Fc fusion protein are listed in Table 27.

TABLE 27
IL-22 Fc Fusion Protein Release Specification

| Analytical Procedure | Acceptance Criteria |
|---|---|
| Color (Ph. Eur. Color Scale) | Not more colored than BY5 |
| Clarity/Opalescence | ≤Ref III |
| pH | 7.1 ± 0.3 |
| Osmolality (mOsmol/kg) | 290 ± 50 |
| Content of Polysorbate 20 by ELSD (mg/mL) | 0.2 ± 0.1 |
| Content of Methionine (mM) | 5.0 ± 2.0 |
| Identity of IL-22 Fc Fusion Protein by | Positive identity^{a} |
| MALDI-TOF Peptide Mass Fingerprinting | |
| Purity by SE-HPLC | |
| Main Peak (area %) | ≥95.0 |
| Sum of HMW Forms (area %) | ≤5.0^{b} |
| Sum of LMW Forms (area %) | Report^{d} |
| Purity by CE-SDS-NGS | |
| Main Peak (non-reduced) (% CPA) | ≥85.0 |
| Sum of LMW Forms (Pre-Peak) | ≤15.0^{b} |
| (non-reduced) (% CPA) | |
| Sum of HMW Forms (Post-Peak) | Report^{c} |
| (non-reduced) (% CPA) | |
| Purity by ICIEF | |
| Main Peak (area %) | ≥42.9 |
| Acidic Region (area %) | ≤55.6^{b} |
| Basic Region (area %) | ≤20.8^{b} |
| Sialic Acid (mol/mol NANA) | 8-12 |
| Bioburden (CFU/10 mL) | ≤10 |
| Bacterial Endotoxins (EU/mL) | ≤15 |

### Example 4: IL-22 Fc Fusion Protein Reference Standard

This example provides data concerning the use of Reference Standard Batch No. 1 as the IL-22 Fc fusion protein Reference Standard. This batch was used in all release and stability assays that require the IL-22 Fc fusion protein Reference Standard.

The Reference Standard was used in qualitative, quantitative, and semi-quantitative in-process sample testing, and in IL-22 Fc fusion protein and the IL-22 Fc fusion protein pharmaceutical composition release and stability testing to verify consistent product quality. The Reference Standard was also used for system suitability as applicable.

Each Reference Standard Batch was analyzed using appropriate release tests to demonstrate acceptable composition, purity, and strength appropriate for use as the Reference Standard for IL-22 Fc fusion protein.

The results for Reference Standard Batch testing are provided in Table 28 and were based on the release test procedures in place at the time of release of the reference batch. The potency of Reference Standard Batch 1 was assigned a value of 100%. Subsequent batches of Reference Standard Batch were quantitated relative to the previous reference and assigned a new activity (i.e., new relative potency value).

TABLE 28
IL-22 Fc Fusion Protein Reference Standard Batch Release Testing Results

| Analytical Procedure | Acceptance Criteria | Result |
|---|---|---|
| Appearance and Description | | |
| Color | Not more colored | BY6 |
| Ph. Eur. Color Scale | than BY5 | |
| Clarity/Opalescence | ≤Ref III | ≤Ref I |
| General Tests | | |
| pH | 7.1 ± 0.3 | 7.1 |
| Osmolality (mOsmol/kg) | 290 ± 50 | 292 |
| Content of PS20 by ELSD (mg/mL) | 0.20 ± 0.10 | 0.20 |
| Content of Methionine (mM) | 5.0 ± 2.0 | 5.0 |
| Identity | | |
| Identity of IL-22 Fc Fusion Protein | Positive Identity | Positive Identity |
| by MALDI-TOF-PMF | | |
| Purity | | |
| Purity by SE-HPLC | | |
| Main Peak (area %) | ≥95.0 | 99.6 |
| Sum of HMW Forms (area %) | Report | 0.3 |
| Sum of LMW Forms (area %) | Report | 0.1 |
| Purity by CE-SDS-NGS | | |
| Main Peak (non-reduced) (% CPA) | ≥85.0 | 96.7 |
| Purity by Imaged cIEF | | |
| Main Peak (area %) | ≥42.9 | 53.6 |
| Acidic Region (area %) | Report | 40.6 |
| Basic Region (area %) | Report | 5.8 |
| Sialic Acid (mol/mol) | 8-12 | 8 |
| Quantity | | |
| Content of Protein by UV Spec Scan (mg/mL) | 10.0 ± 1.0 | 10.7 |

CE-SDS-NGS = capillary electrophoresis sodium dodecyl sulfate non-gel sieving;
cIEF = capillary isoelectric focusing;
CPA = corrected peak area;
ELSD = evaporative light scattering detector;
HMW = high molecular weight;
MALDI-TOF-PMF = matrix-assisted laser desorption/ionization-time of flight peptide mass fingerprinting;
SE-HPLC = size-exclusion high-performance liquid chromatography;
UV Spec Scan = ultraviolet-visible spectrophotometric scan.

### Example 5: Changes in Sialic Acid Levels with Cell Culture Duration

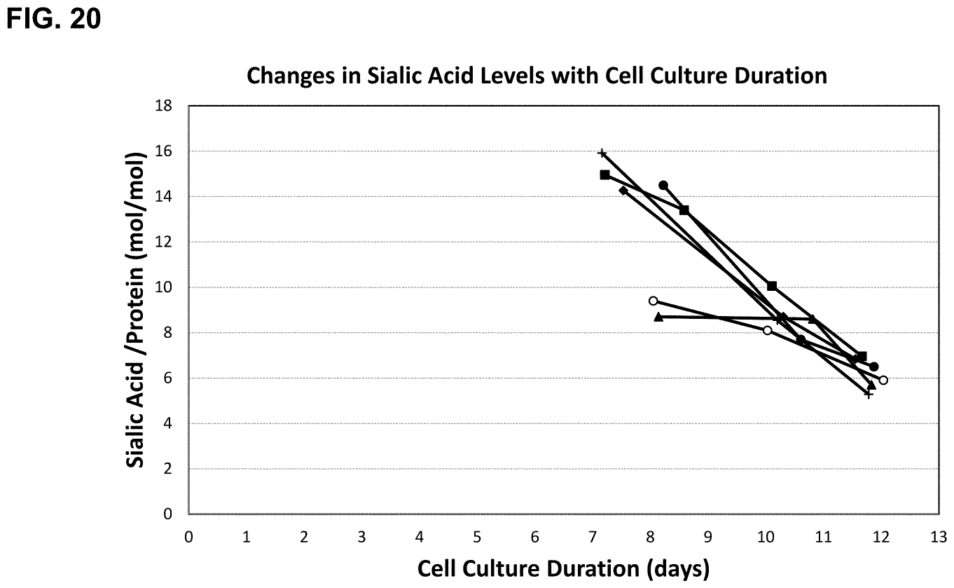

The effect of cell culture duration on IL-22 Fc fusion protein sialic acid levels was evaluated. IL-22 Fc fusion protein was produced as described herein (see, e.g., Example 3). Sialic levels were assessed at a number of time points during the culture in the production bioreactor using RP-HPLC. Sialic levels per mole of dimeric IL-22 Fc fusion protein decreased with increasing cell culture duration (FIG. 20). These results show that for a cell culture duration of 10 days, the sialic acid content is about 8 mol/mol, whereas after 12 days of cell culture, the sialic acid content was about 6 mol/mol. The sialic acid content is further enriched by the purification process described herein (see, e.g., Example 3), for example, using an affinity chromatography resin such as MABSELECT SURE® resin and multimodal anion-exchange chromatography, e.g., using CAPTO™ adhere resin. Thus, the approximately 8 mol/mol sialic acid content of IL-22 Fc fusion proteins produced using a cell culture duration of 10 days for the production phase in the production bioreactor can be enriched to 8 to 12 mol/mol sialic acid (e.g., 8 to 9 mol/mol sialic acid) by purification as described herein. Similarly, the approximately 6 mol/mol sialic acid content of IL-22 Fc fusion proteins produced using a cell culture duration of 12 days for the production phase in the production bioreactor can also be enriched to 8 to 12 mol/mol sialic acid (e.g., 8 to 9 mol/mol sialic acid) by purification as described herein.

These data demonstrate that cell culture duration, for example, during culture in the production bioreactor, may be used a process lever to modulate the sialic acid content of IL-22 Fc fusion proteins produced as described herein. The cell culture duration can be used in combination with the purification process described herein to enrich for IL-22 Fc fusion protein compositions having an average sialic acid content of 8 to 12 moles of sialic acid per mole of IL-22 Fc fusion protein (e.g., 8 to 9 moles of sialic acid per mole of IL-22 Fc fusion protein).

### Other Embodiments

Some embodiments of the technology described herein can be defined according to any of the following numbered embodiments:

1. An interleukin (IL)-22 Fc fusion protein comprising an IL-22 polypeptide linked to an Fc region by a linker, wherein the IL-22 polypeptide is glycosylated, and wherein the IL-22 Fc fusion protein has a sialic acid content in the range of from about 8 to about 12 moles of sialic acid per mole of the IL-22 Fc fusion protein.

2. An IL-22 Fc fusion protein comprising an IL-22 polypeptide linked to an Fc region by a linker, wherein the IL-22 polypeptide is glycosylated, and wherein the IL-22 Fc fusion protein has a potency of about 40% to about 130% relative to a reference IL-22 Fc fusion protein having a sialic acid content of about 8 moles of sialic acid per mole of the IL-22 Fc fusion protein, optionally wherein the reference IL-22 Fc fusion protein has the N-glycan distribution shown in Table 12 and/or Table 13.

3. The IL-22 Fc fusion protein of embodiment 2, wherein the IL-22 Fc fusion protein has a potency of about 80% to about 120% relative to a reference IL-22 Fc fusion protein having a sialic acid content of about 8 moles of sialic acid per mole of the IL-22 Fc fusion protein.

4. The IL-22 Fc fusion protein of embodiment 2 or 3, wherein the IL-22 Fc fusion protein has a potency of about 60% to about 110% relative to a reference IL-22 Fc fusion protein having a sialic acid content of about 8 moles of sialic acid per mole of the IL-22 Fc fusion protein.

5. The IL-22 Fc fusion protein of any one of embodiments 2-4, wherein the IL-22 Fc fusion protein has a potency of about 80% to about 100% relative to a reference IL-22 Fc fusion protein having a sialic acid content of about 8 moles of sialic acid per mole of the IL-22 Fc fusion protein.

6. The IL-22 Fc fusion protein of any one of embodiments 2-5, wherein potency is assessed in a receptor binding assay or a cell-based binding assay.

7. The IL-22 Fc fusion protein of any one of embodiments 2-6, wherein the IL-22 Fc fusion protein has a sialic acid content in the range of from about 8 to about 12 moles of sialic acid per mole of the IL-22 Fc fusion protein.

8. The IL-22 Fc fusion protein of embodiment 1 or 7, wherein the IL-22 Fc fusion protein has a sialic acid content in the range of from about 8 to about 11 moles of sialic acid per mole of the IL-22 Fc fusion protein.

9. The IL-22 Fc fusion protein of embodiment 1 or 8, wherein the IL-22 Fc fusion protein has a sialic acid content in the range of from about 8 to about 10 moles of sialic acid per mole of the IL-22 Fc fusion protein.

10. The IL-22 Fc fusion protein of embodiment 1, 8, or 9, wherein the IL-22 Fc fusion protein has a sialic acid content in the range of from about 8 to about 9 moles of sialic acid per mole of the IL-22 Fc fusion protein.

11. The IL-22 Fc fusion protein of any one of embodiments 1 or 8-10, wherein the IL-22 Fc fusion protein has a sialic acid content of about 8 moles of sialic acid per mole of the IL-22 Fc fusion protein.

12. The IL-22 Fc fusion protein of any one of embodiments 1 or 8-10, wherein the IL-22 Fc fusion protein has a sialic acid content of about 9 moles of sialic acid per mole of the IL-22 Fc fusion protein.

13. The IL-22 Fc fusion protein of any one of embodiments 1 or 8-11, wherein the sialic acid is N-acetylneuraminic acid (NANA).

14. The IL-22 Fc fusion protein of any one of embodiments 1-13, wherein the IL-22 Fc fusion protein has a maximum observed concentration (Cₘₐₓ) of about 8,000 ng/mL to about 19,000 ng/mL.

15. The method of embodiment 14, wherein the Cₘₐₓ is assessed following intravenous administration of about 1,000 μg/kg of the IL-22 Fc fusion protein to a CD1 mouse.

16. The IL-22 Fc fusion protein of any one of embodiments 1-15, wherein the IL-22 Fc fusion protein has an area under the serum concentration-time curve from time 0 to the last measureable time point (AUCₗₐₛₜ) of about 7,000 day·ng/mL to about 25,000 day·ng/mL.

17. The method of embodiment 16, wherein the AUCₗₐₛₜ is assessed following intravenous administration of about 1,000 μg/kg of the IL-22 Fc fusion protein to a CD1 mouse.

18. The IL-22 Fc fusion protein of any one of embodiments 1-17, wherein the IL-22 Fc fusion protein has a clearance (CL) of about 40 mL/kg/day to about 140 mL/kg/day.

19. The IL-22 Fc fusion protein of embodiment 18, wherein the CL is assessed following intravenous administration of about 1,000 μg/kg of the IL-22 Fc fusion protein to a CD1 mouse.

20. The IL-22 Fc fusion protein of any one of embodiments 1-19, wherein the IL-22 polypeptide is N glycosylated.

21. The IL-22 Fc fusion protein of embodiment 20, wherein the IL-22 polypeptide comprises N-glycans having monoantennary, biantennary, triantennary, and/or tetraantennary structure.

22. The IL-22 Fc fusion protein of embodiment 21, wherein about 0.1% to about 2% of the N-glycans have monoantennary structure.

23. The IL-22 Fc fusion protein of embodiment 22, wherein about 0.5% to about 1.5% of the

N-glycans have monoantennary structure.

24. The IL-22 Fc fusion protein of embodiment 23, wherein about 1% of the N-glycans have monoantennary structure.

25. The IL-22 Fc fusion protein of any one of embodiments 21-24, wherein about 10% to about 25% of the N-glycans have biantennary structure.

26. The IL-22 Fc fusion protein of embodiment 25, wherein about 12% to about 21% of the N-glycans have biantennary structure.

27. The IL-22 Fc fusion protein of embodiment 26, wherein about 17% of the N-glycans have biantennary structure.

28. The IL-22 Fc fusion protein of any one of embodiments 21-27, wherein about 25% to about 40% of the N-glycans have triantennary structure.

29. The IL-22 Fc fusion protein of embodiment 28, wherein about 28% to about 35% of the N-glycans have triantennary structure.

30. The IL-22 Fc fusion protein of embodiment 29, wherein about 31% of the N-glycans have triantennary structure.

31. The IL-22 Fc fusion protein of any one of embodiments 21-30, wherein about 30% to about 51% of the N-glycans have tetraantennary structure.

32. The IL-22 Fc fusion protein of embodiment 31, wherein about 35% to about 48% of the N-glycans have tetraantennary structure.

33. The IL-22 Fc fusion protein of embodiment 32, wherein about 42% of the N-glycans have tetraantennary structure.

34. The IL-22 Fc fusion protein of any one of embodiments 20-33, wherein the IL-22 Fc fusion protein comprises N-glycans comprising zero, one, two, three, or four galactose moieties.

35. The IL-22 Fc fusion protein of embodiment 34, wherein about 9% to about 32% of the N-glycans comprise zero galactose moieties.

36. The IL-22 Fc fusion protein of embodiment 35, wherein about 15% to about 25% of the N-glycans comprise zero galactose moieties.

37. The IL-22 Fc fusion protein of embodiment 36, wherein about 21% of the N-glycans comprise zero galactose moieties.

38. The IL-22 Fc fusion protein of any one of embodiments 34-37, wherein about 10% to about 20% of the N-glycans comprise one galactose moiety.

39. The IL-22 Fc fusion protein of embodiment 38, wherein about 12% to about 16% of the N-glycans comprise one galactose moiety.

40. The IL-22 Fc fusion protein of embodiment 39, wherein about 14% of the N-glycans comprise one galactose moiety.

41. The IL-22 Fc fusion protein of any one of embodiments 34-40, wherein about 8% to about 25% of the N-glycans comprise two galactose moieties.

42. The IL-22 Fc fusion protein of embodiment 41, wherein about 10% to about 16% of the N-glycans comprise two galactose moieties.

43. The IL-22 Fc fusion protein of embodiment 42, wherein about 13% of the N-glycans comprise two galactose moieties.

44. The IL-22 Fc fusion protein of any one of embodiments 34-43, wherein about 12% to about 25% of the N-glycans comprise three galactose moieties.

45. The IL-22 Fc fusion protein of embodiment 44, wherein about 15% to about 22% of the N-glycans comprise three galactose moieties.

46. The IL-22 Fc fusion protein of embodiment 45, wherein about 19% of the N-glycans comprise three galactose moieties.

47. The IL-22 Fc fusion protein of any one of embodiments 34-46, wherein about 12% to about 30% of the N-glycans comprise four galactose moieties.

48. The IL-22 Fc fusion protein of embodiment 47, wherein about 15% to about 25% of the N-glycans comprise four galactose moieties.

49. The IL-22 Fc fusion protein of embodiment 48, wherein about 24% of the N-glycans comprise four galactose moieties.

50. The IL-22 Fc fusion protein of any one of embodiments 20-49, wherein the IL-22 Fc fusion protein comprises N-glycans comprising zero, one, two, three, or four sialic acid moieties.

51. The IL-22 Fc fusion protein of embodiment 50, wherein about 12% to about 35% of the N-glycans comprise zero sialic acid moieties.

52. The IL-22 Fc fusion protein of embodiment 51, wherein about 20% to about 30% of the N-glycans comprise zero sialic acid moieties.

53. The IL-22 Fc fusion protein of embodiment 52, wherein about 24% of the N-glycans comprise zero sialic acid moieties.

54. The IL-22 Fc fusion protein of any one of embodiments 50-53, wherein about 10% to about 30% of the N-glycans comprise one sialic acid moiety.

55. The IL-22 Fc fusion protein of embodiment 54, wherein about 15% to about 25% of the N-glycans comprise one sialic acid moiety.

56. The IL-22 Fc fusion protein of embodiment 55, wherein about 20% of the N-glycans comprise one sialic acid moiety.

57. The IL-22 Fc fusion protein of any one of embodiments 50-56, wherein about 10% to about 30% of the N-glycans comprise two sialic acid moieties.

58. The IL-22 Fc fusion protein of embodiment 57, wherein about 15% to about 25% of the N-glycans comprise two sialic acid moieties.

59. The IL-22 Fc fusion protein of embodiment 58, wherein about 21% of the N-glycans comprise two sialic acid moieties.

60. The IL-22 Fc fusion protein of any one of embodiments 50-59, wherein about 10% to about 30% of the N-glycans comprise three sialic acid moieties.

61. The IL-22 Fc fusion protein of embodiment 60, wherein about 12% to about 24% of the N-glycans comprise three sialic acid moieties.

62. The IL-22 Fc fusion protein of embodiment 61, wherein about 17% of the N-glycans comprise three sialic acid moieties.

63. The IL-22 Fc fusion protein of any one of embodiments 50-62, wherein about 1% to about 20% of the N-glycans comprise four sialic acid moieties.

64. The IL-22 Fc fusion protein of embodiment 63, wherein about 5% to about 15% of the N-glycans comprise four sialic acid moieties.

65. The IL-22 Fc fusion protein of embodiment 64, wherein about 9% of the N-glycans comprise four sialic acid moieties.

66. The IL-22 Fc fusion protein of any one of embodiments 20-65, wherein the IL-22 polypeptide comprises about 0% to about 10% N-glycans comprising a terminal mannose moiety.

67. The IL-22 Fc fusion protein of embodiment 66, wherein about 1% to about 4% of the N-glycans comprise a terminal mannose moiety.

68. The IL-22 Fc fusion protein of embodiment 67, wherein about 2% of the N-glycans comprise a terminal mannose moiety.

69. The IL-22 Fc fusion protein of any one of embodiments 20-68, wherein the IL-22 polypeptide comprises about 30% to about 55% N-glycans comprising a terminal N-acetylglucosamine (GlcNAc) moiety.

70. The IL-22 Fc fusion protein of embodiment 69, wherein about 35% to about 50% of the N-glycans comprise a terminal GlcNAc moiety.

71. The IL-22 Fc fusion protein of embodiment 70, wherein about 42% of the N-glycans comprise a terminal GlcNAc moiety.

72. The IL-22 Fc fusion protein of any one of embodiments 69-71, wherein the N-glycans comprise one, two, three, or four terminal GlcNAc moieties.

73. The IL-22 Fc fusion protein of embodiment 72, wherein about 1% to about 20% of the N-glycans comprise one terminal GlcNAc moiety.

74. The IL-22 Fc fusion protein of embodiment 73, wherein about 5% to about 15% of the N-glycans comprise one terminal GlcNAc moiety.

75. The IL-22 Fc fusion protein of embodiment 74, wherein about 10% of the N-glycans comprise one terminal GlcNAc moiety.

76. The IL-22 Fc fusion protein of any one of embodiments 72-75, wherein about 1% to about 20% of the N-glycans comprise two terminal GlcNAc moieties.

77. The IL-22 Fc fusion protein of embodiment 76, wherein about 5% to about 15% of the N-glycans comprise two terminal GlcNAc moieties.

78. The IL-22 Fc fusion protein of embodiment 77, wherein about 10% of the N-glycans comprise two terminal GlcNAc moieties.

79. The IL-22 Fc fusion protein of any one of embodiments 72-78, wherein about 5% to about 25% of the N-glycans comprise three terminal GlcNAc moieties.

80. The IL-22 Fc fusion protein of embodiment 79, wherein about 10% to about 20% of the N-glycans comprise three terminal GlcNAc moieties.

81. The IL-22 Fc fusion protein of embodiment 80, wherein about 14% of the N-glycans comprise three terminal GlcNAc moieties.

82. The IL-22 Fc fusion protein of any one of embodiments 72-81, wherein about 0% to about 15% of the N-glycans comprise four terminal GlcNAc moieties.

83. The IL-22 Fc fusion protein of embodiment 82, wherein about 4% to about 12% of the N-glycans comprise four terminal GlcNAc moieties.

84. The IL-22 Fc fusion protein of embodiment 83, wherein about 7% of the N-glycans comprise four terminal GlcNAc moieties.

85. The IL-22 Fc fusion protein of any one of embodiments 20-84, wherein the IL-22 polypeptide comprises about 20% to about 45% N-glycans comprising a terminal galactose (Gal) moiety.

86. The IL-22 Fc fusion protein of embodiment 85, wherein about 25% to about 35% of the N-glycans comprise a terminal Gal moiety.

87. The IL-22 Fc fusion protein of embodiment 86, wherein about 32% of the N-glycans comprise a terminal Gal moiety.

88. The IL-22 Fc fusion protein of any one of embodiments 85-87, wherein the N-glycans comprise one, two, or three terminal Gal moieties.

89. The IL-22 Fc fusion protein of embodiment 88, wherein about 15% to about 30% of the N-glycans comprise one terminal Gal moiety.

90. The IL-22 Fc fusion protein of embodiment 89, wherein about 20% to about 25% of the N-glycans comprise one terminal Gal moiety.

91. The IL-22 Fc fusion protein of embodiment 90, wherein about 23% of the N-glycans comprise one terminal Gal moiety.

92. The IL-22 Fc fusion protein of any one of embodiments 88-91, wherein about 1% to about 15% of the N-glycans comprise two terminal Gal moieties.

93. The IL-22 Fc fusion protein of embodiment 92, wherein about 2% to about 12% of the N-glycans comprise two terminal Gal moieties.

94. The IL-22 Fc fusion protein of embodiment 93, wherein about 7% of the N-glycans comprise two terminal Gal moieties.

95. The IL-22 Fc fusion protein of any one of embodiments 88-94, wherein about 0.1% to about 6% of the N-glycans comprise three terminal Gal moieties.

96. The IL-22 Fc fusion protein of embodiment 95, wherein about 1% to about 3% of the N-glycans comprise three terminal Gal moieties.

97. The IL-22 Fc fusion protein of embodiment 96, wherein about 2% of the N-glycans comprise three terminal Gal moieties.

98. The IL-22 Fc fusion protein of any one of embodiments 20-97, wherein the IL-22 polypeptide comprises N-glycans comprising galactose N-acetylglucosamine (LacNAc) repeats.

99. The IL-22 Fc fusion protein of embodiment 98, wherein about 1% to about 10% of the N-glycans comprise LacNAc repeats.

100. The IL-22 Fc fusion protein of embodiment 99, wherein about 3% to about 6% of the N-glycans comprise LacNAc repeats.

101. The IL-22 Fc fusion protein of embodiment 100, wherein about 5% of the N-glycans comprise LacNAc repeats.

102. The IL-22 Fc fusion protein of any one of embodiments 20-101, wherein the IL-22 polypeptide comprises N-glycans comprising fucosylated N-glycans.

103. The IL-22 Fc fusion protein of embodiment 102, wherein about 60% to about 80% of the N-glycans are fucosylated.

104. The IL-22 Fc fusion protein of embodiment 103, wherein about 65% to about 75% of the N-glycans are fucosylated.

105. The IL-22 Fc fusion protein of embodiment 104, wherein about 70% of the N-glycans are fucosylated.

106. The IL-22 Fc fusion protein of any one of embodiments 20-105, wherein the IL-22 polypeptide comprises N-glycans comprising afucosylated N-glycans.

107. The IL-22 Fc fusion protein of embodiment 106, wherein about 10% to about 30% of the N-glycans are afucosylated.

108. The IL-22 Fc fusion protein of embodiment 107, wherein about 15% to about 25% of the N-glycans are afucosylated.

109. The IL-22 Fc fusion protein of embodiment 108, wherein about 20% of the N-glycans are afucosylated.

110. The IL-22 Fc fusion protein of any one of embodiments 1-109, wherein the IL-22 polypeptide is glycosylated on amino acid residues Asn21, Asn35, Asn64, and/or Asn143 of SEQ ID NO:4.

111. The IL-22 Fc fusion protein of embodiment 110, wherein the IL-22 polypeptide is glycosylated on amino acid residues Asn21, Asn35, Asn64, and Asn143 of SEQ ID NO:4.

112. The IL-22 Fc fusion protein of embodiment 110 or 111, wherein the glycosylation occupancy on amino acid residue Asn21 of SEQ ID NO:4 is about 70% to about 90%.

113. The IL-22 Fc fusion protein of embodiment 112, wherein the glycosylation occupancy on amino acid residue Asn21 of SEQ ID NO:4 is about 75% to about 85%.

114. The IL-22 Fc fusion protein of embodiment 113, wherein the glycosylation occupancy on amino acid residue Asn21 of SEQ ID NO:4 is about 82%.

115. The IL-22 Fc fusion protein of any one of embodiments 111-114, wherein the glycosylation occupancy on amino acid residue Asn35 of SEQ ID NO:4 is about 90% to about 100%.

116. The IL-22 Fc fusion protein of embodiment 115, wherein the glycosylation occupancy on amino acid residue Asn35 of SEQ ID NO:4 is about 95% to about 100%.

117. The IL-22 Fc fusion protein of embodiment 116, wherein the glycosylation occupancy on amino acid residue Asn35 of SEQ ID NO:4 is about 100%.

118. The IL-22 Fc fusion protein of any one of embodiments 111-117, wherein the glycosylation occupancy on amino acid residue Asn64 of SEQ ID NO:4 is about 90% to about 100%.

119. The IL-22 Fc fusion protein of embodiment 118, wherein the glycosylation occupancy on amino acid residue Asn64 of SEQ ID NO:4 is about 95% to about 100%.

120. The IL-22 Fc fusion protein of embodiment 119, wherein the glycosylation occupancy on amino acid residue Asn64 of SEQ ID NO:4 is about 100%.

121. The IL-22 Fc fusion protein of any one of embodiments 111-120, wherein the glycosylation occupancy on amino acid residue Asn143 of SEQ ID NO:4 is about 15% to about 45%.

122. The IL-22 Fc fusion protein of embodiment 121, wherein the glycosylation occupancy on amino acid residue Asn143 of SEQ ID NO:4 is about 25% to about 35%.

123. The IL-22 Fc fusion protein of embodiment 122, wherein the glycosylation occupancy on amino acid residue Asn143 of SEQ ID NO:4 is about 33%.

124. The IL-22 Fc fusion protein of any one of embodiments 1-123, wherein the Fc region is not glycosylated.

125. The IL-22 Fc fusion protein of embodiment 124, wherein the amino acid residue at position 297 as in the EU index of the Fc region is Gly.

126. The IL-22 Fc fusion protein of embodiment 124, wherein the amino acid residue at position 297 as in the EU index of the Fc region is Ala.

127. The IL-22 Fc fusion protein of any one of embodiments 124-126, wherein the amino acid residue at position 299 as in the EU index of the Fc region is Ala, Gly, or Val

128. The IL-22 Fc fusion protein of any one of embodiments 1-127, wherein the Fc region comprises the CH2 and CH3 domain of IgG1 or IgG4.

129. The IL-22 Fc fusion protein of embodiment 128, wherein the Fc region comprises the CH2 and CH3 domain of IgG4.

130. The IL-22 Fc fusion protein of any one of embodiments 1-129, wherein the IL-22 Fc fusion protein comprises an amino acid sequence having at least 95% sequence identity to the amino acid sequence of SEQ ID NO:8.

131. The IL-22 Fc fusion protein of embodiment 130, wherein the IL-22 Fc fusion protein comprises an amino acid sequence having at least 96% sequence identity to the amino acid sequence of SEQ ID NO:8.

132. The IL-22 Fc fusion protein of embodiment 131, wherein the IL-22 Fc fusion protein comprises an amino acid sequence having at least 97% sequence identity to the amino acid sequence of SEQ ID NO:8.

133. The IL-22 Fc fusion protein of embodiment 132, wherein the IL-22 Fc fusion protein comprises an amino acid sequence having at least 98% sequence identity to the amino acid sequence of SEQ ID NO:8.

134. The IL-22 Fc fusion protein of embodiment 133, wherein the IL-22 Fc fusion protein comprises an amino acid sequence having at least 99% sequence identity to the amino acid of SEQ ID NO:8.

135. The IL-22 Fc fusion protein of any one of embodiments 1-134, wherein the IL-22 Fc fusion protein comprises the amino acid sequence of SEQ ID NO:8, SEQ ID NO:10, or SEQ ID NO:16.

136. The IL-22 Fc fusion protein of embodiment 135, wherein the IL-22 Fc fusion protein comprises the amino acid sequence of SEQ ID NO:8.

137. The IL-22 Fc fusion protein of embodiment 136, wherein the IL-22 Fc fusion protein consists of the amino acid sequence of SEQ ID NO:8.

138. The IL-22 Fc fusion protein of embodiment 135, wherein the IL-22 Fc fusion protein comprises the amino acid sequence of SEQ ID NO:10.

139. The IL-22 Fc fusion protein of embodiment 138, wherein the IL-22 Fc fusion protein consists of the amino acid sequence of SEQ ID NO:10.

140. The IL-22 Fc fusion protein of embodiment 135, wherein the IL-22 Fc fusion protein comprises the amino acid sequence of SEQ ID NO:16.

141. The IL-22 Fc fusion protein of embodiment 140, wherein the IL-22 Fc fusion protein consists of the amino acid sequence of SEQ ID NO:16.

142. The IL-22 Fc fusion protein of any one of embodiments 124-141, wherein the Fc region is not N-glycosylated.

143. The IL-22 Fc fusion protein of any one of embodiments 1-142, wherein the IL-22 Fc fusion protein is a dimeric IL-22 Fc fusion protein.

144. The IL-22 Fc fusion protein of any one of embodiments 1-142, wherein the IL-22 Fc fusion protein is a monomeric IL-22 Fc fusion protein.

145. The IL-22 Fc fusion protein of any one of embodiments 1-144, wherein the IL-22 polypeptide is a human IL-22 polypeptide.

146. The IL-22 Fc fusion protein of embodiment 145, wherein the IL-22 polypeptide comprises the amino acid sequence of SEQ ID NO:4.

147. The IL-22 Fc fusion protein of any one of embodiments 1-146, wherein the linker comprises the amino acid sequence RVESKYGPP (SEQ ID NO: 44).

148. The IL-22 Fc fusion protein of embodiment 147, wherein the linker consists of the amino acid sequence RVESKYGPP (SEQ ID NO: 44).

149. The IL-22 Fc fusion protein of any one of embodiments 1-148, wherein the IL-22 Fc fusion protein binds to IL-22 receptor.

150. The IL-22 Fc fusion protein of embodiment 149, wherein the IL-22 receptor is human IL-22 receptor.

151. The IL-22 Fc fusion protein of embodiment 149 or 150, wherein the IL-22 Fc fusion protein binds to IL-22RA1 and/or IL-10R2.

152. The IL-22 Fc fusion protein of embodiment 151, wherein the IL-22 Fc fusion protein binds to IL-22RA1.

153. The IL-22 Fc fusion protein of any one of embodiments 1-152 produced by the method comprising the step of culturing a host cell capable of expressing the IL-22 Fc fusion protein under conditions suitable for expression of the IL-22 Fc fusion protein.

154. The IL-22 Fc fusion protein of embodiment 153, wherein the method further comprises the step of obtaining the IL-22 Fc fusion protein from the cell culture or culture medium.

155. The IL-22 Fc fusion protein of embodiment 153 or 154, wherein the host cell is a CHO cell.

156. The IL-22 Fc fusion protein of any one of embodiments 1-155, wherein the IL-22 Fc fusion protein has an NGNA content of less than about 5 moles of NGNA per mole of the IL-22 Fc fusion protein.

157. The IL-22 Fc fusion protein of embodiment 156, wherein the IL-22 Fc fusion protein has an NGNA content of less than 1 mole of NGNA per mole of the IL-22 Fc fusion protein.

158. A pharmaceutical composition comprising the IL-22 Fc fusion protein of any one of embodiments 1-157 and at least one pharmaceutically acceptable carrier.

159. The pharmaceutical composition of embodiment 158, wherein the IL-22 Fc fusion protein has a sialic acid content in the range of from about 8 to about 12 moles of sialic acid per mole of the IL-22 Fc fusion protein.

160. The pharmaceutical composition of embodiment 158 or 159, wherein the IL-22 Fc fusion protein has a sialic acid content in the range of from about 8 to about 10 moles of sialic acid per mole of the IL-22 Fc fusion protein.

161. The pharmaceutical composition of any one of embodiments 158-160, wherein the IL-22 Fc fusion protein has a sialic acid content in the range of from about 8 to about 9 moles of sialic acid per mole of the IL-22 Fc fusion protein.

162. The pharmaceutical composition of any one of embodiments 158-161, wherein the IL-22 Fc fusion protein has a sialic acid content of about 8 moles of sialic acid per mole of the IL-22 Fc fusion protein.

163. The pharmaceutical composition of any one of embodiments 158-162, wherein the IL-22 Fc fusion protein has a sialic acid content of about 9 moles of sialic acid per mole of the IL-22 Fc fusion protein.

164. The pharmaceutical composition of any one of embodiments 158-163, wherein the sialic acid is N-acetylneuraminic acid (NANA).

165. The pharmaceutical composition of any one of embodiments 158-164, wherein the IL-22 Fc fusion protein comprises the amino acid sequence of SEQ ID NO:8, SEQ ID NO:10, or SEQ ID NO:16.

166. The pharmaceutical composition of embodiment 165, wherein the IL-22 Fc fusion protein comprises the amino acid sequence of SEQ ID NO:8.

167. The pharmaceutical composition of embodiment 165, wherein the IL-22 Fc fusion protein comprises the amino acid sequence of SEQ ID NO:16.

168. The pharmaceutical composition of any one of embodiments 158-167, further comprising an additional therapeutic agent.

169. The pharmaceutical composition of any one of embodiments 158-168, further comprising a gelling agent.

170. The pharmaceutical composition of embodiment 169, wherein the gelling agent is a polysaccharide.

171. The pharmaceutical composition of embodiment 169 or 170, wherein the gelling agent is a cellulosic agent.

172. The pharmaceutical composition of any one of embodiments 169-171, wherein the gelling agent is methylcellulose, hydroxyethyl cellulose, carboxymethyl cellulose, hydroxypropyl cellulose, POE-POP block polymers, alginate, hyaluronic acid, polyacrylic acid, hydroxyethyl methylcellulose or hydroxypropyl methylcellulose.

173. The pharmaceutical composition of embodiment 172, wherein the gelling agent is a hydroxypropyl methylcellulose.

174. The pharmaceutical composition of embodiment 173, wherein the pharmaceutical composition is for topical administration.

175. A method of treating inflammatory bowel disease (IBD) in a subject in need thereof, the method comprising administering to the subject the IL-22 Fc fusion protein of any one of embodiments 1-157 or the pharmaceutical composition of any one of embodiments 158-168.

176. The method of embodiment 175, wherein the IBD is ulcerative colitis or Crohn's disease.

177. The method of embodiment 176, wherein the IBD is ulcerative colitis.

178. The method of embodiment 177, wherein the ulcerative colitis is moderate to severe ulcerative colitis.

179. The method of embodiment 176, wherein the IBD is Crohn's disease.

180. A method of inhibiting microbial infection in the intestine, preserving goblet cells in the intestine during a microbial infection, enhancing epithelial cell integrity, epithelial cell proliferation, epithelial cell differentiation, epithelial cell migration or epithelial wound healing in the intestine, of a subject in need thereof, the method comprising administering to the subject the IL-22 Fc fusion protein of any one of embodiments 1-157 or the pharmaceutical composition of any one of embodiments 158-168.

181. The method of embodiment 180, wherein the epithelial cell is an intestinal epithelial cell.

182. A method of treating acute kidney injury or acute pancreatitis in a subject in need thereof, the method comprising administering to the subject the IL-22 Fc fusion protein of any one of embodiments 1-157 or the pharmaceutical composition of any one of embodiments 158-168.

183. A method of accelerating or improving wound healing in a subject in need thereof, the method comprising administering to the subject the IL-22 Fc fusion protein of any one of embodiments 1-157 or the pharmaceutical composition of any one of embodiments 158-174.

184. The method of embodiment 183, wherein the wound is a chronic wound or an infected wound.

185. The method of embodiment 183 or 184, wherein the subject is diabetic.

186. The method of embodiment 185, wherein the diabetic subject has type II diabetes.

187. The method of any one of embodiments 183-186, wherein the wound is a diabetic foot ulcer.

188. The method of any one of embodiments 183-187, wherein the IL-22 Fc fusion protein or the pharmaceutical composition is administered until there is complete wound closure.

189. A method for preventing or treating a cardiovascular condition in a subject in need thereof, which condition includes a pathology of atherosclerotic plaque formation, the method comprising administering to the subject the IL-22 Fc fusion protein of any one of embodiments 1-157 or the pharmaceutical composition of any one of embodiments 158-168.

190. The method of embodiment 189, wherein the cardiovascular disease is coronary artery disease, coronary microvascular disease, stroke, carotid artery disease, peripheral artery disease, or chronic kidney disease.

191. The method of embodiment 189 or 190, further comprising slowing down the progression of atherosclerotic plaque formation or preventing indicia of atherosclerosis.

192. The method of embodiment 191, wherein the indicia of atherosclerosis includes plaque accumulation or vascular inflammation.

193. A method for treating metabolic syndrome in a subject in need thereof, the method comprising administering to the subject the IL-22 Fc fusion protein of any one of embodiments 1-157 or the pharmaceutical composition of any one of embodiments 158-168.

194. The method of embodiment 193, further comprising reducing one or more risk factors associated with metabolic syndrome, including one or more of abdominal obesity, hyperglycemia, dyslipidemia, and hypertension.

195. The method of embodiment 193 or 194, further comprising reducing the level of bacterial lipopolysaccharide in the subject.

196. A method of treating acute endotoxemia, sepsis, or both, in a subject in need thereof, the method comprising administering the subject the IL-22 Fc fusion protein of any one of embodiments 1-157 or the pharmaceutical composition of any one of embodiments 158-168.

197. The method of any one of embodiments 193-196, wherein the subject is in need of a change in HDL/LDL lipid profile.

198. The method of any one of embodiments 175-197, wherein the IL-22 Fc fusion protein has a sialic acid content in the range of from about 8 to about 12 moles of sialic acid per mole of the IL-22 Fc fusion protein.

199. The method of embodiment 198, wherein the IL-22 Fc fusion protein has a sialic acid content in the range of from about 8 to about 10 moles of sialic acid per mole of the IL-22 Fc fusion protein.

200. The method of any one of embodiments 198 or 199, wherein the IL-22 Fc fusion protein has a sialic acid content in the range of from about 8 to about 9 moles of sialic acid per mole of the IL-22 Fc fusion protein.

201. The method of any one of embodiments 198-200, wherein the IL-22 Fc fusion protein has a sialic acid content of about 8 moles of sialic acid per mole of the IL-22 Fc fusion protein.

202. The method of any one of embodiments 198-200, wherein the IL-22 Fc fusion protein has a sialic acid content of about 9 moles of sialic acid per mole of the IL-22 Fc fusion protein.

203. The method of any one of embodiments 198-202, wherein the sialic acid is N-acetylneuraminic acid (NANA).

204. The method of any one of embodiments 175-203, wherein the IL-22 Fc fusion protein comprises the amino acid sequence of SEQ ID NO:8, SEQ ID NO:10, or SEQ ID NO:16.

205. The method of embodiment 204, wherein the IL-22 Fc fusion protein comprises the amino acid sequence of SEQ ID NO:8.

206. The method of embodiment 204, wherein the IL-22 Fc fusion protein comprises the amino acid sequence of SEQ ID NO:16.

207. The method of any one of embodiments 175-206, wherein the IL-22 Fc fusion protein or the pharmaceutical composition is administered intravenously, subcutaneously, intraperitoneally, or topically.

208. The method of embodiment 207, wherein the IL-22 Fc fusion protein or the pharmaceutical composition is administered intravenously.

209. The method of embodiment 207, wherein the IL-22 Fc fusion protein or the pharmaceutical composition is administered subcutaneously.

210. The method of any one of embodiments 175-209, wherein the subject is co-administered with at least one additional therapeutic agent.

211. The method of any one of embodiments 175-210, wherein the subject is a human.

212. A method of making the IL-22 Fc fusion protein of any one of embodiments 1-157, the method comprising the following steps:

(a) providing a host cell comprising a nucleic acid encoding the IL-22 Fc fusion protein of any one of embodiments 1-157;

(b) culturing the host cell in a seed train medium under conditions suitable to form a seed train culture;

(c) inoculating the seed train into an inoculum medium and culturing under conditions suitable to form an inoculum train culture; and

(d) culturing the inoculum train in a production medium under conditions suitable to form a production culture, wherein the host cells of the production culture express the IL-22 Fc fusion protein, thereby making the IL-22 Fc fusion protein.

213. A method of making an IL-22 Fc fusion protein, the method comprising the following steps:

(a) providing a host cell comprising a nucleic acid encoding a IL-22 Fc fusion protein, the IL-22 Fc fusion protein comprising an IL-22 polypeptide linked to an Fc region by a linker;

(b) culturing the host cell in a seed train medium under conditions suitable to form a seed train culture;

(c) inoculating the seed train in an inoculum medium under conditions suitable to form an inoculum train culture; and

(d) culturing the inoculum train in a production medium under conditions time suitable to form a production culture, wherein the host cells of the production culture express the IL-22 Fc fusion protein, thereby making the IL-22 Fc fusion protein,

wherein the IL-22 polypeptide is glycosylated, and wherein the IL-22 Fc fusion protein has a sialic acid content of from about 8 to about 12 moles of sialic acid per mole of the IL-22 Fc fusion protein.

214. The method of embodiment 212 or 213, wherein the host cell is a frozen host cell, and step (a) further comprises thawing the frozen host cell in a seed train medium.

215. The method of any one of embodiments 212-214, wherein the method further comprises passaging the inoculum train about 1 to about 10 times prior to step (d).

216. The method of embodiment 215, wherein the inoculum train is passaged about 2 to about 6 times prior to step (d).

217. The method of embodiment 216, wherein the inoculum train is passaged about 2 times prior to step (d).

218. The method of any one of embodiments 212-217, wherein the seed train medium comprises a selection agent capable of selecting for the host cell.

219. The method of embodiment 218, wherein the selection agent is methionine sulfoximine, methotrexate, or an antibiotic.

220. The method of embodiment 219, wherein the selection agent is methionine sulfoximine.

221. The method of embodiment 219, wherein the selection agent is an antibiotic.

222. The method of embodiment 221, wherein the antibiotic is selected from blasticidin, geneticin, hygromycin B, puromycin, mycophenolic acid, or zeocin.

223. The method of any one of embodiments 212-222, wherein the seed train medium, the inoculum medium, and/or the production medium comprises an antifoaming agent.

224. The method of embodiment 223, wherein the antifoaming agent is simethicone emulsion, antifoam 204, antifoam A, antifoam B, antifoam C, antifoam Y-30, or antifoam SE-15.

225. The method of embodiment 224, wherein the antifoaming agent is simethicone emulsion.

226. The method of any one of embodiments 212-225, wherein the seed train medium, the inoculum medium, and/or the production medium includes a buffering agent, a cell protective agent, a polysaccharide, and/or an osmolality adjustment agent.

227. The method of any one of embodiments 212-225, wherein step (b) is performed at a temperature of about 25° C. to about 40° C.

228. The method of embodiment 227, wherein step (b) is performed at a temperature of about 35° C. to about 39° C.

229. The method of embodiment 228, wherein step (b) is performed at a temperature of about 37° C.

230. The method of any one of embodiments 212-229, wherein step (b) is performed in a spinner, a spin tube, a shake flask, or a seed train bioreactor.

231. The method of embodiment 230, wherein step (b) is performed in a seed train spinner, a single-use bioreactor (e.g., a WAVE BIOREACTOR™ or an AMBR® bioreactor (e.g., an AMBR® 15 bioreactor)), or a shake flask.

232. The method of embodiment 231, wherein step (b) has a duration of about 1 day to about 12 days per passage.

233. The method of embodiment 232, wherein step (b) has a duration of about 2 days to about 7 days per passage.

234. The method of embodiment 230, wherein step (b) is performed in a seed train bioreactor.

235. The method of embodiment 234, wherein the pH of the seed train culture is about 6 to about 8.

236. The method of embodiment 235, wherein the pH of the seed train culture is about 6.5 to about 7.5.

237. The method of embodiment 236, wherein the pH of the seed train culture is about 7.15.

238. The method of any one of embodiments 234-237, wherein the dissolved oxygen of the seed train culture is about 15% to about 50%.

239. The method of embodiment 238, wherein the dissolved oxygen of the seed train culture is about 20% to about 40%.

240. The method of embodiment 239, wherein the dissolved oxygen of the seed train culture is about 30%.

241. The method of any one of embodiments 234-240, wherein step (b) has a duration of about 1 day to about 10 days.

242. The method of embodiment 241, wherein step (b) has a duration of about 2 days to about 5 days.

243. The method of any one of embodiments 212-242, wherein step (c) is performed at a temperature of about 25° C. to about 40° C.

244. The method of embodiment 243, wherein step (c) is performed at a temperature of about 35° C. to about 39° C.

245. The method of embodiment 244, wherein step (c) is performed at a temperature of about 37° C.

246. The method of any one of embodiments 212-245, wherein step (c) is performed in one or more bioreactors.

247. The method of embodiment 246, wherein step (c) is performed in three or four bioreactors.

248. The method of embodiment 246 or 247, wherein the pH of the inoculum culture is about 6 to about 8.

249. The method of embodiment 248, wherein the pH of the inoculum culture is about 6.5 to about 7.5.

250. The method of embodiment 249, wherein the pH of the inoculum culture is about 7.1.

251. The method of any one of embodiments 246-250, wherein the dissolved oxygen of the inoculum culture is about 15% to about 50%.

252. The method of embodiment 251, wherein the dissolved oxygen of the inoculum culture is about 20% to about 40%.

253. The method of embodiment 252, wherein the dissolved oxygen of the inoculum culture is about 30%.

254. The method of any one of embodiments 246-253, wherein step (c) has a duration of about 1 day to about 5 days.

255. The method of embodiment 254, wherein step (c) has a duration of about 2 days to about 3 days.

256. The method of any one of embodiments 212-255, wherein step (d) includes a temperature shift from an initial temperature to a post-shift temperature.

257. The method of embodiment 256, wherein the initial temperature is about 25° C. to about 40° C.

258. The method of embodiment 257, wherein the initial temperature is about 35° C. to about 39° C.

259. The method of embodiment 258, wherein the initial temperature is about 37° C.

260. The method of any one of embodiments 256-259, wherein the post-shift temperature is about 25° C. to about 40° C.

261. The method of embodiment 260, wherein the post-shift temperature is about 30° C. to about 35° C.

262. The method of embodiment 261, wherein the post-shift temperature is about 33° C.

263. The method of any one of embodiments 256-262, wherein the temperature shift occurs over a period of about 12 h to about 120 h.

264. The method of embodiment 263, wherein the temperature shift occurs over a period of about 48 h to about 96 h.

265. The method of embodiment 264, wherein the temperature shift occurs over a period of about 72 h.

266. The method of any one of embodiments 212-265, wherein the pH of the production culture is about 6 to about 8.

267. The method of embodiment 266, wherein the pH of the production culture is about 6.5 to about 7.5.

268. The method of embodiment 267, wherein the pH of the production culture is about 7.0.

269. The method of any one of embodiments 212-268, wherein step (d) is performed in a production bioreactor.

270. The method of embodiment 269, wherein the dissolved oxygen of the production culture is about 15% to about 50%.

271. The method of embodiment 270, wherein the dissolved oxygen of the production culture is about 20% to about 40%.

272. The method of embodiment 271, wherein the dissolved oxygen of the production culture is about 30%.

273. The method of any one of embodiments 269-272, wherein step (d) has a duration of about 5 days to about 25 days.

274. The method of embodiment 273, wherein step (d) has a duration of about 7 days to about 16 days.

275. The method of embodiment 274, wherein step (d) has a duration of about 12 days.

276. The method of any one of embodiments 212-275, wherein step (d) further comprises adding nutrients to the production culture by a nutrient feed.

277. The method of any one of embodiments 212-276, wherein the host cell is a prokaryotic cell or a eukaryotic cell.

278. The method of embodiment 277, wherein the host cell is a eukaryotic cell.

279. The method of embodiment 278, wherein the eukaryotic cell is a mammalian cell.

280. The method of embodiment 279, wherein the mammalian cell is a Chinese hamster ovary (CHO) cell.

281. The method of embodiment 280, wherein the CHO cell is a suspension-adapted CHO cell.

282. The method of any one of embodiments 212-281, further comprising the following step: (e) harvesting a cell culture fluid comprising the IL-22 Fc fusion protein from the production culture.

283. The method of embodiment 282, wherein step (e) comprises cooling the production culture.

284. The method of embodiment 283, wherein step (e) comprises cooling the production culture to about 2° C. to about 8° C.

285. The method of embodiment 282-284, wherein step (e) comprises removing the host cells from the production medium by centrifugation to form the cell culture fluid.

286. The method of embodiment 285, wherein step (e) further comprises filtering the cell culture fluid.

287. The method of any one of embodiments 282-286, further comprising the following step: (f) purifying the IL-22 Fc fusion protein in the cell culture fluid.

288. The method of embodiment 287, wherein step (f) comprises the following substeps:

(i) contacting the cell culture fluid to an affinity chromatographic support, optionally washing the affinity chromatographic support with a wash buffer, eluting the IL-22 Fc fusion protein from the affinity chromatographic support with a first elution buffer to form an affinity pool, and optionally inactivating viruses in the affinity pool;

(ii) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and

(iii) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool.

289. The method of embodiment 288, wherein step (f) further comprises the following substep:

(iv) concentrating the purified product pool to form a concentrated product pool.

290. The method of embodiment 289, wherein step (f) further comprises the following substep:

(v) ultrafiltering the purified product pool.

291. The method of embodiment 290, wherein ultrafiltering comprises filtering the purified product pool with a 10 kDa composite regenerated cellulose ultrafiltration membrane.

292. The method of any one of embodiments 289-291, wherein step (f) further comprises the following substep:

(vi) exchanging the buffer of the concentrated product pool to form a ultrafiltration and diafiltration (UFDF) pool comprising the IL-22 Fc fusion protein.

293. The method of embodiment 292, wherein the buffer of the concentrated product pool is exchanged with a diafiltration buffer comprising 0.01 M sodium phosphate, pH 7.2, final concentration.

294. The method of embodiment 292 or 293, wherein step (f) further comprises the following substep:

(vii) conditioning the UFDF pool with a formulation buffer to form a conditioned UFDF pool comprising the IL-22 Fc fusion protein.

295. The method of any one of embodiments 288-294, wherein substep (i) further comprises inactivating viruses by adding a detergent to the cell culture fluid prior to contacting the cell culture fluid to the affinity column.

296. The method of any one of embodiments 288-294, wherein substep (i) comprises inactivating viruses by adding a detergent to the affinity pool.

297. The method of embodiment 295 or 296, wherein the detergent is TRITON® X-100 or TRITON® CG110.

298. The method of embodiment 295-297, wherein the final concentration of the detergent is about 0.01% to about 2% (v/v).

299. The method of embodiment 298, wherein the final concentration of the detergent is about 0.1% to about 1% (v/v).

300. The method of embodiment 299, wherein the final concentration of the detergent is about 0.3% to about 0.5% (v/v).

301. The method of embodiment 300, wherein the final concentration of the detergent is about 0.5%

302. The method of any one of embodiments 295-301, wherein the virus inactivation is performed at about 12° to about 25° C.

303. The method of any one of embodiments 288-302, wherein inactivating viruses has a duration of greater than about 0.5 h.

304. The method of any one of embodiments 288-303, wherein the affinity chromatographic support comprises a protein A resin, a protein G resin, or an IL-22 receptor resin.

305. The method of embodiment 304, wherein the protein A resin is a MABSELECT SURE® resin.

306. The method of any one of embodiments 288-305, wherein the wash buffer comprises 0.4 M potassium phosphate, pH 7.0, final concentration.

307. The method of any one of embodiments 288-306, wherein the first elution buffer comprises 0.3 M L-arginine hydrochloride, 0.013 M sodium phosphate, pH 3.8, final concentration.

308. The method of any one of embodiments 288-307, wherein the anion-exchange chromatographic support comprises a strong anion exchanger with multimodal functionality resin.

309. The method of embodiment 308, wherein the anion-exchange chromatographic support comprises a CAPTO™ adhere resin.

310. The method of any one of embodiments 288-309, wherein the first equilibration buffer comprises 0.04 M sodium acetate, pH 5.8, final concentration.

311. The method of any one of embodiments 288-310, wherein the second elution buffer is a gradient elution buffer.

312. The method of embodiment 311, wherein the gradient elution buffer comprises 0.04 M sodium acetate, pH 5.8 as Buffer A of the gradient elution buffer and 0.04 M sodium acetate, 0.3M sodium sulfate pH 5.8 as Buffer B of the gradient, wherein the gradient starts at 10% of Buffer B.

313. The method of any one of embodiments 288-312, wherein the second equilibration buffer comprises 0.025 M MOPS, 0.3 M sodium sulfate, pH 7.0, final concentration.

314. A method of purifying an IL-22 Fc fusion protein, the method comprising:

(a) providing a cell culture fluid comprising an IL-22 Fc fusion protein and optionally inactivating viruses in the cell culture fluid;

(b) contacting the cell culture fluid to an affinity chromatographic support, optionally washing the affinity chromatographic support with a wash buffer, and eluting the IL-22 Fc fusion protein from the affinity chromatographic support with a first elution buffer to form an affinity pool, and optionally inactivating viruses in the affinity pool;

(c) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and

(d) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool.

315. The method of embodiment 314, wherein the IL-22 polypeptide is glycosylated, and wherein the IL-22 Fc fusion protein has a sialic acid content of from about 8 to about 12 moles of sialic acid per mole of the IL-22 Fc fusion protein.

The specification is considered to be sufficient to enable one skilled in the art to practice the invention. Although the foregoing invention has been described in some detail by way of illustration and example for purposes of clarity of understanding, the descriptions and examples should not be construed as limiting the scope of the invention. Indeed, various modifications of the invention in addition to those shown and described herein will become apparent to those skilled in the art from the foregoing description and fall within the scope of the appended claims.

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

What is claimed is:

1. A composition comprising an interleukin-22 (IL-22) Fc fusion protein, wherein the IL-22 Fc fusion protein comprises a glycosylated IL-22 polypeptide linked to an antibody Fc region by a linker, and wherein the composition has an average sialic acid content in the range of 8 to 12 moles of sialic acid per mole of the IL-22 Fc fusion protein.

2. The composition of claim 1, wherein the IL-22 polypeptide is N-glycosylated.

3. The composition of claim 1 or 2, wherein the IL-22 polypeptide is glycosylated at one or more locations corresponding to amino acid residues Asn21, Asn35, Asn64, and/or Asn143 of SEQ ID NO: 4.

4. A composition comprising an IL-22 Fc fusion protein, wherein the IL-22 Fc fusion protein comprises a glycosylated IL-22 polypeptide linked to an antibody Fc region by a linker, wherein the IL-22 polypeptide is glycosylated at one or more locations corresponding to amino acid residues Asn21, Asn35, Asn64, and/or Asn143 of SEQ ID NO: 4, and wherein:
(a) the percent N-glycosylation site occupancy at residue Asn21 is in the range of 70 to 90;
(b) the percent N-glycosylation site occupancy at residue Asn35 is in the range of 90 to 100;
(c) the percent N-glycosylation site occupancy at residue Asn64 is in the range of 90 to 100; and/or
(d) the percent N-glycosylation site occupancy at residue Asn143 is in the range of 25 to 35.

5. The composition of any one of claims 1-3, wherein the composition has an average sialic acid content in the range of 8 to 9 moles of sialic acid per mole of the IL-22 Fc fusion protein.

6. The composition of claim 4, wherein the composition has an average sialic acid content of 8 or 9 moles of sialic acid per mole of the IL-22 Fc fusion protein.

7. The composition of any one of claims 1-5, wherein the sialic acid glycosylation comprises N-acetylneuraminic acid (NANA).

8. The composition of any one of claims 1-5, wherein the composition has an average N glycolylneuraminic acid (NGNA) content of less than 1 mole of NGNA per mole of the IL-22 Fc fusion protein.

9. The composition of any one of claims 1-8, wherein the composition is a liquid composition.

10. The composition of any one of claims 1-9, wherein:
(i) the IL-22 Fc fusion protein has a maximum observed concentration (Cₘₐₓ) of about 8,000 ng/mL to about 19,000 ng; and/or
(ii) the IL-22 Fc fusion protein has an area under the serum concentration-time curve from time 0 to the last measureable time point (AUCₗₐₛₜ) of about 7,000 day·ng/mL to about 25,000 day·ng/mL; and/or
(iii) the IL-22 Fc fusion protein has a clearance (CL) of about 40 mL/kg/day to about 140 m L/kg/day.

11. The composition of claim 10, wherein the Cₘₐₓ, AUCₗₐₛₜ, and/or CL is assessed following intravenous administration of about 1,000 μg/kg of the IL-22 Fc fusion protein to a CD1 mouse.

12. The composition of any one of claims 2-11, wherein the IL-22 polypeptide comprises N-glycans having monoantennary, biantennary, triantennary, and/or tetraantennary structure.

13. The composition of claim 12, wherein:
(i) about 0.1% to about 2% of the N-glycans have monoantennary structure;
(ii) about 10% to about 25% of the N-glycans have biantennary structure;
(iii) about 25% to about 40% of the N-glycans have triantennary structure; and/or
(iv) about 30% to about 51% of the N-glycans have tetraantennary structure.

14. The composition of any one of claims 2-13, wherein the IL-22 Fc fusion protein comprises N-glycans comprising zero, one, two, three, or four galactose moieties.

15. The composition of claim 14, wherein:
(i) about 9% to about 32% of the N-glycans comprise zero galactose moieties;
(ii) about 10% to about 20% of the N-glycans comprise one galactose moiety;
(iii) about 8% to about 25% of the N-glycans comprise two galactose moieties;
(iv) about 12% to about 25% of the N-glycans comprise three galactose moieties; and/or
(v) about 12% to about 30% of the N-glycans comprise four galactose moieties.

16. The composition of any one of claims 2-15, wherein the IL-22 Fc fusion protein comprises N-glycans comprising zero, one, two, three, or four sialic acid moieties.

17. The composition of claim 16, wherein:
(i) about 12% to about 35% of the N-glycans comprise zero sialic acid moieties;
(ii) about 10% to about 30% of the N-glycans comprise one sialic acid moiety;
(iii) about 10% to about 30% of the N-glycans comprise two sialic acid moieties;
(iv) about 10% to about 30% of the N-glycans comprise three sialic acid moieties; and/or
(v) about 1% to about 20% of the N-glycans comprise four sialic acid moieties.

18. The composition of any one of claims 2-17, wherein (i) the IL-22 polypeptide comprises about 0% to about 10% N-glycans comprising a terminal mannose moiety; and/or (ii) the IL-22 polypeptide comprises about 30% to about 55% N-glycans comprising a terminal N-acetylglucosamine (GlcNAc) moiety.

19. The composition of claim 18, wherein the N-glycans comprise one, two, three, or four terminal GlcNAc moieties.

20. The composition of claim 19, wherein:
(i) about 1% to about 20% of the N-glycans comprise one terminal GlcNAc moiety;
(ii) about 1% to about 20% of the N-glycans comprise two terminal GlcNAc moieties;
(iii) about 5% to about 25% of the N-glycans comprise three terminal GlcNAc moieties; and/or
(iv) about 0% to about 15% of the N-glycans comprise four terminal GlcNAc moieties.

21. The composition of any one of claims 2-20, wherein (i) the IL-22 polypeptide comprises about 20% to about 45% N-glycans comprising a terminal galactose (Gal) moiety; and/or (ii) the N-glycans comprise one, two, or three terminal Gal moieties.

22. The composition of claim 21, wherein:
(i) about 15% to about 30% of the N-glycans comprise one terminal Gal moiety;
(ii) about 1% to about 15% of the N-glycans comprise two terminal Gal moieties; and/or
(iii) about 0.1% to about 6% of the N-glycans comprise three terminal Gal moieties.

23. The composition of any one of claims 2-22, wherein: (i) the IL-22 polypeptide comprises N-glycans comprising galactose N-acetylglucosamine (LacNAc) repeats; (ii) the IL-22 polypeptide comprises N-glycans comprising fucosylated N-glycans; and/or (iii) the IL-22 polypeptide comprises N-glycans comprising afucosylated N-glycans.

24. The composition of any one of claims 1-23, wherein the Fc region is not glycosylated.

25. The composition of claim 24, wherein: (i) the amino acid residue at position 297 as in the EU index of the Fc region is Gly or Ala; and/or (ii) the amino acid residue at position 299 as in the EU index of the Fc region is Ala, Gly, or Val.

26. The composition of any one of claims 1-25, wherein the Fc region comprises the CH2 and CH3 domain of IgG1 or IgG4.

27. The composition of claim 26, wherein the Fc region comprises the CH2 and CH3 domain of IgG4.

28. The composition of any one of claims 1-27, wherein the IL-22 Fc fusion protein comprises an amino acid sequence having at least 95% sequence identity to the amino acid sequence of SEQ ID NO:8.

29. The composition of any one of claims 1-28, wherein the IL-22 Fc fusion protein comprises or consists of the amino acid sequence of SEQ ID NO:8, SEQ ID NO:10, or SEQ ID NO:16.

30. The composition of any one of claims 1-29, wherein the IL-22 polypeptide is a human IL-22 polypeptide.

31. The composition of claim 30, wherein the IL-22 polypeptide comprises the amino acid sequence of SEQ ID NO:4.

32. The composition of any one of claims 1-31, wherein the linker comprises or consists of the amino acid sequence RVESKYGPP (SEQ ID NO: 44).

33. The composition of any one of claims 1-32, wherein the IL-22 Fc fusion protein binds to IL-22 receptor.

34. The composition of claim 33, wherein the IL-22 receptor is human IL-22 receptor.

35. The composition of claim 34, wherein the human IL-22 receptor comprises a heterodimer consisting of an IL-22R1 polypeptide and an IL-10R2 polypeptide.

36. The composition of claim 35, wherein the IL-22R1 polypeptide comprises the amino acid sequence of SEQ ID NO:82 and the IL-10R2 polypeptide comprises the amino acid sequence of SEQ ID NO:84.

37. The composition of any one of claims 1-36, wherein the IL-22 Fc fusion protein consists of two single-chain units linked by two inter-chain disulfide bridges, wherein each single chain unit consists of a human IL-22 fusion protein comprising IL-22 fused with the Fc region of a human immunoglobulin IgG4.

38. The composition of any one of claims 1-37, wherein the composition is a pharmaceutical composition.

39. The composition of claim 38, wherein the composition is aqueous and/or sterile.

40. The composition of claim 38 or 39, further comprising an additional therapeutic agent.

41. The composition of any one of claims 38-40, further comprising a gelling agent.

42. A method of treating inflammatory bowel disease (IBD) in a subject in need thereof, the method comprising administering to the subject the composition of any one of claims 1-41.

43. The method of claim 42, wherein the IBD is ulcerative colitis or Crohn's disease.

44. The method of claim 43, wherein the IBD is ulcerative colitis.

45. The method of claim 44, wherein the ulcerative colitis is moderate to severe ulcerative colitis.

46. The method of claim 43, wherein the IBD is Crohn's disease.

47. A composition comprising an interleukin (IL)-22 Fc fusion protein of any one of claims 1 to 41 for use as a medicament.

48. A composition comprising an interleukin (IL)-22 Fc fusion protein of any one of claims 1 to 41 for use in
(i) treating inflammatory bowel disease (IBD),
(ii) inhibiting microbial infection in the intestine, preserving goblet cells in the intestine during a microbial infection, enhancing epithelial cell integrity, epithelial cell proliferation, epithelial cell differentiation, epithelial cell migration or epithelial wound healing in the intestine,
(iii) treating acute kidney injury or acute pancreatitis,
(iv) accelerating or improving wound healing in a subject in need thereof,
(v) preventing or treating a cardiovascular disease such as coronary artery disease, coronary microvascular disease, stroke, carotid artery disease, peripheral artery disease, or chronic kidney disease,
(vi) treating metabolic syndrome, or
(vii) treating acute endotoxemia or sepsis.

49. Use of a composition comprising an interleukin (IL)-22 Fc fusion protein of any one of claims 1 to 41 for the preparation of a medicament for use in
(i) treating inflammatory bowel disease (IBD),
(ii) inhibiting microbial infection in the intestine, preserving goblet cells in the intestine during a microbial infection, enhancing epithelial cell integrity, epithelial cell proliferation, epithelial cell differentiation, epithelial cell migration or epithelial wound healing in the intestine,
(iii) treating acute kidney injury or acute pancreatitis,
(iv) accelerating or improving wound healing in a subject in need thereof,
(v) preventing or treating a cardiovascular disease such as coronary artery disease, coronary microvascular disease, stroke, carotid artery disease, peripheral artery disease, or chronic kidney disease,
(vi) treating metabolic syndrome, or
(vii) treating acute endotoxemia or sepsis.

50. A method of inhibiting microbial infection in the intestine, preserving goblet cells in the intestine during a microbial infection, enhancing epithelial cell integrity, epithelial cell proliferation, epithelial cell differentiation, epithelial cell migration or epithelial wound healing in the intestine, of a subject in need thereof, the method comprising administering to the subject the composition of any one of claims 1-41.

51. A method of treating acute kidney injury or acute pancreatitis in a subject in need thereof, the method comprising administering to the subject the composition of any one of claims 1-41.

52. A method of accelerating or improving wound healing in a subject in need thereof, the method comprising administering to the subject the composition of any one of claims 1-41.

53. A method for preventing or treating a cardiovascular condition in a subject in need thereof, which condition includes a pathology of atherosclerotic plaque formation, the method comprising administering to the subject the composition of any one of claims 1-41.

54. A method for treating metabolic syndrome in a subject in need thereof, the method comprising administering to the subject the composition of any one of claims 1-41.

55. A method of treating acute endotoxemia, sepsis, or both, in a subject in need thereof, the method comprising administering to the subject the composition of any one of claims 1-41.

56. The method, composition, or use of any one of claims 42-55, wherein the composition is administered intravenously, subcutaneously, intraperitoneally, or topically.

57. The method, composition, or use of any one of claims 42-56, wherein the subject is co-administered with at least one additional therapeutic agent.

58. A method of making a composition comprising an IL-22 Fc fusion protein, the method comprising:
culturing an inoculum train culture comprising a plurality of host cells in a production medium under conditions suitable to form a production culture for at least about 10 days, wherein the host cells comprise a nucleic acid encoding an IL-22 Fc fusion protein, the IL-22 Fc fusion protein comprising an IL-22 polypeptide linked to an Fc region by a linker, wherein the host cells express the IL-22 Fc fusion protein, thereby making the composition comprising an IL-22 Fc fusion protein,
wherein the IL-22 polypeptide is glycosylated, and wherein the composition has an average sialic acid content in the range of 6 to 12 moles of sialic acid per mole of the IL-22 Fc fusion protein.

59. The method of claim 58, wherein the duration of the culturing is at least 11 days, at least 12 days, or at least 13 days.

60. The method of claim 58 or 59, wherein the duration of the culturing is 12 days.

61. The method of any one of claims 58-60, further comprising generating a seed train culture by culturing a host cell comprising a nucleic acid encoding the IL-22 Fc fusion protein in a seed train medium under conditions suitable to form the seed train culture prior to culturing the inoculum train culture in the production medium.

62. The method of claim 61, further comprising inoculating the seed train culture in an inoculum medium under conditions suitable to form an inoculum train culture prior to culturing the inoculum train culture in the production medium.

63. The method of any one of claims 58-62, wherein the host cells are eukaryotic host cells.

64. The method of claim 63, wherein the eukaryotic host cells are mammalian host cells.

65. The method of claim 64, wherein the mammalian host cells are Chinese hamster ovary (CHO) cells.

66. The method of any one of claims 58-65, further comprising:
harvesting a cell culture fluid comprising the IL-22 Fc fusion protein from the production culture.

67. The method of claim 66, wherein harvesting the cell culture fluid comprises: (i) cooling the production culture; (ii) removing the host cells from the production medium by centrifugation to form the cell culture fluid; and/or (iii) filtering the cell culture fluid.

68. The method of any one of claims 58-67, further comprising:
purifying the IL-22 Fc fusion protein in the cell culture fluid.

69. The method of claim 68, wherein purifying the IL-22 Fc fusion protein comprises the following substeps:
(i) contacting the cell culture fluid to an affinity chromatographic support, optionally washing the affinity chromatographic support with a wash buffer, eluting the IL-22 Fc fusion protein from the affinity chromatographic support with a first elution buffer to form an affinity pool, and optionally inactivating viruses in the affinity pool;
(ii) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and
(iii) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool.

70. The method of claim 69, wherein purifying the IL-22 Fc fusion protein further comprises one or more of the following substeps:
(iv) concentrating the purified product pool to form a concentrated product pool;
(v) ultrafiltering the purified product pool;
(vi) exchanging the buffer of the concentrated product pool to form a ultrafiltration and diafiltration (UFDF) pool comprising the IL-22 Fc fusion protein; and/or
(vii) conditioning the UFDF pool with a formulation buffer to form a conditioned UFDF pool comprising the IL-22 Fc fusion protein.

71. The method of claim 69 or 70, wherein substep (i) further comprises inactivating viruses by adding a detergent to the cell culture fluid prior to contacting the cell culture fluid to the affinity column.

72. The method of any one of claims 58-71, wherein the method further comprises enriching the sialic acid content of the composition.

73. The method of claim 72, wherein the composition has an initial average sialic acid content in the range of 6 to 8 moles of sialic acid per mole of the IL-22 Fc fusion protein.

74. The method of claim 72, wherein the composition has an initial average sialic acid content of 6, 7, or 8 moles of sialic acid per mole of the IL-22 Fc fusion protein.

75. The method of any one of claims 72-74, wherein the method comprises enriching the average sialic acid content to the range of 8 to 12 moles of sialic acid per mole of the IL-22 Fc fusion protein.

76. The method of any one of claims 62-75, wherein the method further comprises enriching the average sialic acid content to the range of 8 to 9 moles of sialic acid per mole of the IL-22 Fc fusion protein.

77. The method of any one of claims 70-76, wherein the affinity chromatographic support comprises a protein A resin, a protein G resin, or an IL-22 receptor resin.

78. The method of claim 77, wherein the protein A resin is a MABSELECT SURE® resin.

79. The method of any one of claims 70-78, wherein the anion-exchange chromatographic support comprises a strong anion exchanger with multimodal functionality resin.

80. The method of claim 79, wherein the anion-exchange chromatographic support comprises a CAPTO™ adhere resin.

81. The method of any one of claims 58-80, wherein the composition has an average sialic acid content of 8 to 12 moles of sialic acid per mole of the IL-22 Fc fusion protein.

82. The method of any one of claims 58-81, wherein the composition has an average sialic acid content of 8 or 9 moles of sialic acid per mole of the IL-22 Fc fusion protein.

83. The method of any one of claims 58-82, wherein the IL-22 Fc fusion protein consists of two single-chain units linked by two inter-chain disulfide bridges, wherein each single chain unit consists of a human IL-22 fusion protein comprising IL-22 fused with the Fc region of a human immunoglobulin IgG4.

84. A composition produced by the method of any one of claims 58-83.

85. The composition of claim 84, wherein the composition is a pharmaceutical composition.

86. A method of selecting a batch comprising an IL-22 Fc fusion protein for release, the method comprising the following steps:
(a) providing a batch comprising IL-22 Fc fusion proteins;
(b) assessing the levels of sialic acid in the batch; and
(c) selecting the batch for release if the batch has an average sialic acid content in the range of 8 to 12 moles of sialic acid per mole of the IL-22 Fc fusion protein.

87. The method of claim 86, wherein step (c) comprises selecting the batch for release if the batch has an average sialic acid content of 8 to 9 moles of sialic acid per mole of the IL-22 Fc fusion protein.

88. The method of claim 86 or 87, wherein step (c) comprises selecting the batch for release if the batch has an average sialic acid content of 8 moles of sialic acid per mole of the IL-22 Fc fusion protein.

89. The method of claim 86 or 87, wherein step (c) comprises selecting the batch for release if the batch has an average sialic acid content of 9 moles of sialic acid per mole of the IL-22 Fc fusion protein.

90. The method of any one of claims 86-89, wherein step (b) comprises using high-performance liquid chromatography (HPLC), ultra-high performance liquid chromatography (UHPLC), capillary electrophoresis, or a colorimetric assay to assess the levels of sialic acid in the batch.

91. The method of claim 90, wherein step (b) comprises assessing the levels of sialic acid using HPLC.

92. A method for controlling sialic acid content of a composition comprising an IL-22 Fc fusion protein, the IL-22 Fc fusion protein comprising a glycosylated IL-22 polypeptide linked by a linker to an antibody Fc region, the method comprising:
culturing an inoculum train culture comprising a plurality of host cells in a production medium under conditions suitable to form a production culture for at least 10 days, wherein the host cells comprise a nucleic acid encoding the IL-22 Fc fusion protein and express the IL-22 Fc fusion protein, wherein the composition has an average sialic acid content in the range of 6 to 12 moles of sialic acid per mole of the IL-22 Fc fusion protein; and
enriching the average sialic acid content of the composition to the range of 8 to 12 moles of sialic acid per mole of the IL-22 Fc fusion protein, thereby controlling the sialic acid content of the composition.

93. The method of claim 92, wherein the method comprises enriching the average sialic acid content of the composition to the range of 8 to 9 moles of sialic acid per mole of the IL-22 Fc fusion protein.

94. The method of claim 92 or 93, wherein enriching the average sialic acid content comprises harvesting a cell culture fluid comprising the IL-22 Fc fusion protein from the production culture.

95. The method of claim 94, wherein harvesting the cell culture fluid comprises: (i) cooling the production culture; (ii) removing the host cells from the production medium by centrifugation to form the cell culture fluid; and/or (iii) filtering the cell culture fluid.

96. The method of claim 94 or 95, wherein enriching the average sialic acid content of the composition further comprises purifying the IL-22 Fc fusion protein in the cell culture fluid.

97. The method of claim 96, wherein purifying the IL-22 Fc fusion protein comprises the following substeps:
(i) contacting the cell culture fluid to an affinity chromatographic support, optionally washing the affinity chromatographic support with a wash buffer, eluting the IL-22 Fc fusion protein from the affinity chromatographic support with a first elution buffer to form an affinity pool, and optionally inactivating viruses in the affinity pool;
(ii) contacting the affinity pool to an anion-exchange chromatographic support, optionally washing the anion-exchange chromatographic support with a first equilibration buffer, eluting the IL-22 Fc fusion protein from the anion-exchange chromatographic support with a second elution buffer to form an anion-exchange pool, and optionally filtering the anion-exchange pool to remove viruses; and
(iii) contacting the anion-exchange pool to a hydrophobic-interaction chromatographic support and collecting the flow-through to form a purified product pool comprising the IL-22 Fc fusion protein, and optionally washing the hydrophobic-interaction chromatographic support with a second equilibration buffer, collecting the flow-through, and adding it to the purified product pool.

98. The method of claim 97, wherein purifying the IL-22 Fc fusion protein further comprises one or more of the following substeps:
(iv) concentrating the purified product pool to form a concentrated product pool;
(v) ultrafiltering the purified product pool;
(vi) exchanging the buffer of the concentrated product pool to form a ultrafiltration and diafiltration (UFDF) pool comprising the IL-22 Fc fusion protein; and/or
(vii) conditioning the UFDF pool with a formulation buffer to form a conditioned UFDF pool comprising the IL-22 Fc fusion protein.

99. The method of claim 97 or 98, wherein substep (i) further comprises inactivating viruses by adding a detergent to the cell culture fluid prior to contacting the cell culture fluid to the affinity column.

100. The method of any one of claims 97-99, wherein the affinity chromatographic support comprises a protein A resin, a protein G resin, or an IL-22 receptor resin.

101. The method of claim 100, wherein the protein A resin is a MABSELECT SURE® resin.

102. The method of any one of claims 97-101, wherein the anion-exchange chromatographic support comprises a strong anion exchanger with multimodal functionality resin.

103. The method of claim 102, wherein the anion-exchange chromatographic support comprises a CAPTO™ adhere resin.
